(19) United States
(12) Patent Application Publication
(10) Pub. No.: US 2008/0226615 A1
(43) Pub. Date: Sep. 18, 2008

(54) Vectors having both isoforms of beta-hexosaminidase and uses of the same

(76) Inventor: Stephanos Kyrkanides, Penfield, NY (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/506,184

(22) Filed: Aug. 16, 2006

Related U.S. Application Data
(63) Continuation of application No. PCT/US05/04885, filed on Feb. 16, 2005.
Application No. PCT/US05/04885 is a continuation of application No. 10/781,142, filed on Feb. 18, 2004.

(51) Int. Cl. A61K 35/12; A61K 31/70; A61P 25/00

(52) U.S. Cl. 424/93.73; 514/44

(57) ABSTRACT

Disclosed are compositions and methods related to delivering a nucleic acid to the central nervous system.

### CROSS-REFERENCE TO RELATED APPLICATIONS

This application is a continuation of copending International Patent Application No. PCT/US05/04885, filed Feb. 16, 2005, which is a continuation of U.S. patent application Ser. No. 10/781,142, filed Feb. 18, 2004. International Patent Application No. PCT/US05/04885, U.S. patent application Ser. No. 10/781,142, International Patent Application No. PCT/US03/13672, filed on May 2, 2003, U.S. Provisional Application No. 60/377,503, filed on May 2, 2002 are herein incorporated by reference in their entireties.

### STATEMENT REGARDING FEDERALLY SPONSORED RESEARCH

This application was funded in part by NIH grants R03 DE13680, K08 DE00471 and R21 DE14700. The United States Government may have certain rights in the inventions described herein

### BACKGROUND OF THE INVENTION

Lysosomal storage disorders are disorders that typically arise from the aberrant or non-existent proteins involved in degradation function within the lysosomes. This causes a decrease in the lysosomal activity, which in turn causes an accumulation of unwanted materials in the cell. These unwanted materials can cause severe cellular toxicity and can impair, for example, neuronal function. These diseases severely impair the quality of life of those who have them, and can even result in death. Two diseases, Tay-Sachs and Sandoffs, are related to the functional impairment of the lysosomal protein β-hexosaminidase. β-hexosaminidase is a hetero or homo dimer made up of two subunits arising from two separate genes, HexA and HexB. Mutation of the HexA gene, causing functional problems with the HEX-α (HexA/HexB) polypeptide, results in Tay-Sachs disease, whereas mutation of the HexB gene, causing functional problems in the HEX-α (HexA/HexB) and HEX-β (HexB/HexB) polypeptides, results in Sandhoff's disease. Clinically, it is not uncommon for patients to display only mild features at infancy, but due to increasing lysosomal storage over time, progress to severe forms of the disease by adolescence.

Current treatments include bone marrow transplantation, which has been employed in some cases of individuals during childhood but with modest outcomes. A significant problem with the bone marrow transplantation approach is that it may address the lack of specific metabolic activity in peripheral tissues, but due to the presence of the blood-brain-barrier it fails to avert disease progression in the central nervous system. Hence patients often continue to clinically deteriorate due to central nervous system involvement with subsequent development of neurodegeneration, blindness, mental retardation, paralysis and dementia.

Enzyme replacement strategies targeting peripheral and central nervous system tissues utilizing gene therapy is a logical approach for treating inherited metabolic disorders. In a study by Akli et al. (Akli S, et al., 1996. Gene Therapy 3: 769-774), the authors report successful restoration of β-hexosaminidase in fibroblasts derived from patients with HexA deficiency via adenoviral-mediated gene transfer in vitro. Likewise a HexA transgene and a HexB transgene was successfully introduced into neural progenitor cells utilizing retroviral vectors (Lacorazza et al., 1996. Nat Med 2(4):424-9).

Disclosed herein are vectors and methods which solve the problems associated with enzyme replacement therapies directed to β-hexosaminidase deficiencies. Disclosed are compositions and methods of delivering a nucleic acid to the central nervous system.

### SUMMARY OF THE INVENTION

In accordance with the purposes of this invention, as embodied and broadly described herein, this invention, in one aspect, relates to vector constructs that comprise sequence encoding the HEX-β polypeptide. Also disclosed are vector constructs comprising sequence encoding the HEX-β and the HEX-α polypeptides. Also disclosed are vectors for perinatal gene delivery, including delivery of HEX-α and HEX-β, which can be used for inherited lysosomal disorders such as Tay-Sachs and Sandoffs disease.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows that HEXlacZ encodes for both isoforms of human β-hexosaminidase, HexA & HexB. FIG. 1(A) shows pHEXlacZ vector. BHK^{HexlacZ} are developed by stable HexlacZ transduction. FIG. 1(B) shows cells stain positively by X-gal histochemistry. FIG. 1(C) shows HexA & HexB mRNA is detected by RT-PCR in total RNA extracts. FIG. 1(D₁) shows human HEXA & FIG. 1(E₁) shows human HEXB proteins are detected in BHK^{HexlacZ} by immunocytochemistry. FIG. 1(F₁) shows HEXA & HEXA+HEXB activity is measured by 4MUGS & 4MUG fluorometry, respectively. Figure (G) β-hexosaminidase detection by Fast Garnet histochemistry. (D₂, E₂, G₂ are controls for D₁, E₁, G₁, respectively).

FIG. 2 shows that the β-Hex therapeutic gene cross-corrects. An important property of the β-Hex transgene is the products hHEXA & hHEXB have the ability to cross-correct, specifically, to be released extracellularly and then to be absorbed via paracrine pathways by other cells whereby they contribute to β-hexosaminidase activity. For this purpose, BHK^{HexlacZ} cells were cultured and the supernatant was collected (conditioned medium), filtered (0.45 μm) and applied on normal mouse kidney fibroblasts in culture. Forty-eight hours later, the cells were washed thoroughly with phosphate buffered saline, and briefly treated with a trypsin solution to remove extracellular proteins from the cell surfaces. Following trypsin inactivation with Tris/EDTA buffer, the cells were fixed with 4% paraformaldehyde solution and processed by Fast Garnet histochemistry for β-hexosaminidase activity. Fast Garnet histochemistry of murine fibroblasts exposed to (A) conditioned medium collected from BHK^{HexlacZ} cells compared to cells exposed to medium from normal parent BHK-21 cells (B). These results demonstrate that hHEXA & hHEXB, products of the β-Hex transgene, are released into the extracellular medium and can be absorbed by other cells via paracrine pathways resulting in induction of the cellular β-hexosaminidase. In another example, Fast Garnet histochemistry of cells treated with conditioned medium resulted increased levels of Fast Garnet staining compared to cells treated with supernatant collected from naïve cells. This property is important as it suggests that β-hexosaminidase therapeutic levels can be achieved via cross-correction without the necessity of all cells being directly transduced by the FIV(Hex) virus.

FIG. 3 shows a representation of a lentiviral system containing the HexA and HexB genes. The 3-vector FIV(Hex) system. The FIV(Hex) lentiviral system is comprised of 3 vectors: Packaging vector providing the packaging instructions in trans, -VSV-G envelop vector providing the envelop instructions in trans, -FIV(Hex) vector containing the therapeutic bicistronic gene.

FIG. 4 shows a representation of a Fiv(Hex) vector. Backbone FIV vector constructed by Proeschla et al. (1998)

FIG. 5 shows restriction fragment pattern of Feline immunodeficiency viral vector comprising a β-Hex construct. A maxi prep of FIV(Hex) clone 6.2 in 500 TB with 3× solution run through 2 columns. Yield of DNA was 1.095 mg. Final concentration is 1 microg/microl. Restriction enzyme digest with ScaI, not1, Sal1, and Xho1. The bands are as expected.

FIG. 6 shows fibroblast infection by FIV(Hex) in vitro.

FIG. 7 shows an FIV(Hex) titration experiment.

FIG. 8 shows FIV(lacZ) administration to adult mice. FIV(lacZ) infection of murine fibroblasts (CrfK's) in vitro, as well as of liver cells following direct transdermal intra-hepatic injection. Liver, brain and spleen sections stained for β-galactosidase following intraperitoneal injection to 3 month old mice. lacZ expression was detected by X-gal staining (blue stain) and immunocytochemistry (ICC; black stain) on fixed tissue sections harvested 1 month post-treatment.

FIG. 9 shows FIV(lacZ) administration to P4 mice. Liver, brain, spleen and kidney sections stained for β-galactosidase following intraperitoneal injection to mice of perinatal age (4 days old). lacZ expression was detected by X-gal staining (blue stain) on fixed tissue sections harvested 3 months post-treatment. Likewise, when FIV(lacZ) neonatal intraperitoneal was administered to 2 days old mice (C57BL/6) it resulted in β-galactosidase expression in the liver, spleen and brain that increased with time, suggesting permanent transgene incorporation into the genome (Data not shown) when tested at 6 weeks of age.

FIG. 10 shows dose response of IP injections. Young adult mice (6 weeks old) were injected intra-peritoneally with different doses of FIV(lacZ) {0.1 mL, 0.5 mL, 1.0 mL and 2.0 mL of 10³ infectious particles per mL} viral solution. One month following treatment the animals were sacrificed and lacZ reporter gene expression was measured. It was found that increasing doses of FIV result in increasing levels of gene therapy efficacy. In the clinical, human disease arena, this would optimally translate into intravenous administration of 10⁵-10⁶ infectious FIV particles to ensure similar efficacy levels of gene therapy.

FIG. 11 shows diagrams of the vectors used to make the constructs discussed in Examples 1 and 2. FIV(Hex) is constructed by ligating the backbone part of FIV(LacZ), and the fragment of HexB-IRES-HexA from pHexLacZ. FIV(LacZ) is 12750 bp, after cut with SstII and NotI (generate 4500 bp and 8250 bp bands). Purify the 8250 bp band which contains the FIV backbone with CMV promoter. pHexlacZ is a construct of 10150 bp. Cut with NheI and NotI, there are 4700 bp and 5450 bp fragments. The 4700 bp band contains the structure of HexB-IRES-HexA, which doesn't have CMV.

FIG. 12 shows how the structure of FIV(Hex) was confirmed. The constructs were digested with different restriction enzymes: (Result see FIG. 5). ScaI: cut once in the FIV backbone (generated one band 13 Kb). NotI: the site of ligation, and it is the only site (generated one band 13 Kb). Sal I: one site in HexB-IRFS-HexA and 3 sites in FIV backbone (generated one band t 8.5 Kb, one wide band with 2184 bp and 2400 bp, one band 34 bp which is invisible). Xho I: there is one site in HexB-IRFS-HexA and six sites in the FIV backbone (FIV(LacZ): at 502, 1410, 1453, 7559, 7883 and 9949 bp). These generated 6 bands (908 bp, 43 bp(invisible), 1.7 Kb, 324 bp, 2066 bp, 3.3 Kb, and 2.8 Kb).

FIG. 13 shows a transcription termination cassette (STOP) flanked by 2 loxP sites was inserted between the promoter CMV and the therapeutic gene HexB-IRES-HexA. This results in inhibition of gene expression, until the STOP cassette is exsionally removed via the action of cre recombinase. The termination stop can consist of for example, a neomycin gene, whose termination signal acts as a termination signal for the rest of the transcript. Any reporter gene could be inserted and used in this way.

FIG. 14 shows a dually regulated inducible cre-recombinase system which was constructed. The activity of this construct is regulated exogenously by RU486. Furthermore, a stable cell line for this system was developed, whereby addition of RU486 in the culture media results in activation of cre-recombinase and subsequently excisional recombination of DNA, such as a transcription termination cassette flanked by 2 loxP sites.

FIG. 15 shows an example of the function of stable cell line, named GLVP/CrePr cell line, described in FIG. 14. In this case, the dual reporter vector CMV-lox-Luc-lox-AP was transiently transfected into the cell line. Alkaline phospatase (AP) activity was evaluated in vitro after the addition of RU486 to the culture media by an AP histochemical staining method.

FIG. 16A shows the excisionally activated β-hexosaminidase gene Hex^{XAT} was constructed by placing a floxed transcription termination cassette (STOP) upstream to the first open reading frame: CMV-loxP-STOP-loxP-HexB-IRES-HexA. FIG. 16B shows Hex^{XAT} was transiently transfected into our inducible cre cell line. Activation of cre-recombinase resulted in loxP directed DNA recombination and excision of the STOP cassette. FIG. 16C Cre-mediated activation of Hex^{XAT} resulted in HexA and HexB upregulation (column 1). RU486 stimulation of GLVP/CrePr results in site-directed recombination and subsequent activation of a dormant transcriptional unit. A. shows the p Hex^{XAT}, a bicistronic transgene comprised of a “floxed” transcription-termination cassette (STOP), and both isoforms of the human β-hexosaminidase, was transiently transfected into the GLVP/CrePr cell line. B. RU-486 administration resulted in loxP-directed excisional recombination, C. resulting in transcriptional activation and synthesis of HexA and HexB mRNA.

FIG. 17 shows the semi-quantitative analysis for HexA and HexB showed induction of gene transcription following Hex^{XAT} activation at (A) the mRNA level, (B) enzyme activity level in vitro, as well as (C) histochemical level in situ. RU486 significantly induces β-hexosaminidase expression in the GLVP/CrePr cell line. β-hexosaminidase activity was found significantly upregulated in p Hex^{XAT}-transfected GLVP/CrePr cells 4 days after RU486 administration at the (A) HexA & HexB mRNA, (B) enzyme activity in vitro, as well as (C) in fixed monolayers in situ, as assessed by RTPCR, 4-MUG fluorescence and X-Hex histochemistry, respectively.

FIG. 18 shows Hex^{XAT} was stably expressed in fibroblasts derived from a patient with Tay-Sachs disease (TSD). Gene activation was mediated by infection of the cells with a HSV aplicon viral vector capable of transducing cells with the cre recombinase. This figure demonstrates that activation of the Hex gene results in protection of the TSD cells from death following GM₂ substrate challenge.

FIG. 19 shows that the virus produced in FIG. 3 above can resolve GM2 storage in TSD cells cultured in vitro.

FIG. 20 shows the Hex gene was cloned in the FIV backbone as shown in FIG. 3 producing the virus FIV(Hex), which was then used to infect TSD cells challenged with GM₂ substrate. This figures shows that delivery of our Hex gene with FIV(Hex) in TSD cells in vitro confers protection to cell death following GM₂ administration.

FIG. 21 shows HexB^{-/-} knock out pups (2 days) were injected 100 uL of FIV(Hex) virus intraperitoneally. The animals were monitored weekly while they assumed growth until sacrificed (16-18 weeks of age).

FIG. 22 shows expression of HEXB protein in adult mice that were injected with the FIV(Hex) virus as infants 2 days after birth. HEXB protein expression was detected by immunocytochemistry in the liver and brain of these mice.

FIG. 23 shows locomotive performance in relation to age (in weeks) of 6 mice that were treated 2 days after birth: 3 mice were injected with FIV(Hex) and 3 with FIV(lacZ) and served as controls. At 16 weeks of age, the “classic” stage that the hexB knockout mice display the disease, there was significant disease difference between the two groups.

FIG. 24 shows neonatal FIV administration resulted in widespread distribution of the viral vector. The defective, VSV-G pseudotyped FIV(lacZ) vector was injected intraperitoneally (total of 10⁵ infectious particles) to mouse pups at post-natal day P2. The expression of the reporter gene β-galactosidase was evaluated histologically by X-gal histochemistry, and enzymatically by a chemiluminescent substrate assay. (A) X-gal positive cells were observed in the brain (thalamus), as well as in the (B) liver (portal triads) and (C) spleen (white pulp) of mice 6 weeks following treatment. (D) β-galactosidase enzyme activity was present at all time points examined (3, 6 and 13 weeks) and appeared to increase with time after FIV(lacZ) treatment.

FIG. 25 shows the recombinant β-hexosaminidase feline immunodeficiency virus vector FIV(Hex) successfully transduces normal murine and human Tay-Sachs fibroblasts in vitro. (A) Murine wild type primary fibroblasts were infected with FIV(Hex) at 5×10⁷ infectious particles/mL in vitro, and β-hexosaminidase expression was found increased compared to (B) FIV(lacZ)-infected cells by means of X-Hex histochemistry. (C) The presence and (D) expression of the β-hexosaminidase transgene was determined in FIV(Hex)-infected murine normal fibroblasts by PCR and RT-PCR, respectively. In addition, (E) FIV(Hex) treatment conferred survival in human Tay-Sachs fibroblasts that were previously challenged by exogenous administration of GM₂ ganglioside, (F) which otherwise induces cell death under serum-free conditions in vitro. (G) In contrast, normal human fibroblasts were not affected by GM₂ administration.

FIG. 26 shows neonatal FIV(Hex) intraperitoneal administration to hexB^{-/-} pups results in transduction of brain and peripheral cells. (A) HEXB protein was detected by immunocytochemistry in the liver of 5 weeks old hexB^{-/-} mice treated systemically with FIV(Hex) at post-natal day P2. HEXB expression was observed primarily at the portal triads. (B) Larger magnification of panel A. (C) HEXB-positive cells were also immunolocalized in the cerebral parenchyma adjacent to the third ventricle, as well as (D) in Purkinje-like cortical cerebellar cells.

FIG. 27 shows a HexB expression was restored in the brain of Sandhoff mice following neonatal FIV(Hex) administration. Two day old (P2) hexB^{-/-} pups received a single dose (5×10⁶ infectious particles) of FIV(Hex) intraperitoneally. At 3 months of age, the animals were sacrificed and the mRNA levels of HexB as well as a number of inflammation-related genes were assessed by RT-PCR. HexB expression was detected at the mRNA level in the FIV(Hex)-treated mice and calculated as approximately 21% of the hexB^{+/-} heterozygous littermate. In addition, IL-1β and ICAM-1 mRNA levels normalized in the brain of hexB^{-/-} mice after FIV(Hex) intraperitoneal injection, IL-6 collectively showed no overall change, whereas TNFα was found increased in the FIV-injected mice. *p<0.05

FIG. 28 shows FIV(Hex) neonatal administration attenuated neuro-inflammation, GM2 storage and prevented cell loss in hexB−/− mice. Two day old (P2) hexB^{-/-} mice that received a single dose (5×10⁶ infectious particles) of FIV(Hex) intraperitoneally were sacrificed at 3 months of age and analyzed by immunocytochemistry employing antibodies against glial fibrillary acidic protein (GFAP), major histocompatibility complex-II (MHC-II), GM₂ ganglioside. Cell death was evaluated by the TUNEL method. GFAP immunostaining was found increased in the thalamus of (A) hexB^{-/-} mice compared to (B) FIV(Hex)-treated animals and (C) hexB^{+/-} heterozygotes. MHC-II immunostaining (thalamus) showed no difference between the groups (D-F). GM₂ immunostaining was also decreased in hexB^{-/-} animals after FIV(Hex) treatment compared to saline-injected mice in the brain stem (G versus J), hippocampus (H versus K), as well as thalamus (I versus L). TUNEL-positive cells were identified in the cerebellum of hexB^{-/-} mice but not of FIV(Hex)-treated or wild type controls. Neuronal degeneration was also confirmed by the Fluoro-Jade staining in the cerebellum of hexB^{-/-} mice versus FIV(Hex)-treated animals.

FIG. 29 shows FIV(Hex) neonatal administration attenuated neuro-inflammation, GM2 storage and prevented cell loss in hexB−/− mice. Two day old (P2) hexB^{-/-} mice received a single dose (5×10⁶ infectious particles) of FIV(Hex) or FIV(lacZ) intraperitoneally. At 4 months of age, all mice were sacrificed according to Animal Welfare regulations that were enforced due to the locomotive deterioration of the FIV(lacZ) treated mice. Astroglial and microglial activation was evaluated by immunocytochemistry employing antibodies raised against GFAP and MHC-II antigens, respectively. FIV(lacZ)-injected animals displayed numerous GFAP- and MHC-II—positive cells in the cerebellum (A & C, respectively), thalamus (E & G, respectively), cortex (I & K, respectively), brain stem (M & O, respectively) and the basal ganglia (Q & S, respectively). In contrast, FIV(Hex)-treated animals showed reduced levels of GFAP and MHC-II immunostaining in the cerebellum (B & D, respectively), thalamus (F & H, respectively), cortex (J & L, respectively), brain stem (N & P, respectively) and the basal ganglia (R & T, respectively).

FIG. 30 shows FIV(Hex) neonatal administration ameliorated motor strength in hexB−/− knockout mice. Two day old (P2) hexB^{-/-} mice received a single dose (5×10⁶ infectious particles) of FIV(Hex) or FIV(lacZ) intraperitoneally. From 12-to-16 weeks of age the mice were evaluated for loss of motor strength by the inverted mesh method, at which time point all mice were sacrificed due to Animal Welfare regulations because two of the FIV(lacZ)-injected animals displayed complete locomotive deterioration. (A) The FIV(Hex)-treated animals showed significantly improved locomotive performance compared to FIV(lacZ)-injected mice (P=0.00248).

FIG. 31 shows bar graph.

FIG. 32 shows a bar graph.

### DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular β-Hex vector is disclosed and discussed and a number of modifications that can be made to a number of molecules including the β-Hex vector are discussed, specifically contemplated is each and every combination and permutation of the β-Hex vector and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

### B. COMPOSITIONS AND METHODS

1. Lysosomal Disorders

Lysosomal storage disorders are a group of closely related metabolic diseases resulting from deficiency in enzymes essential for the degradation of gangliosides, mucopolysaccharides, as well as other complex macromolecules. With the dysfunction of a lysosomal enzyme, catabolism of correlate substrates remains incomplete, leading to accumulation of insoluble complex macromolecules within the lysosomes. For example, α-hexosaminidase defects result in lysosomal storage of GM₂ gangliosides leading to the development of Tay-Sachs or Sandhoffs disease. Similarly, mucopolysaccharidoses (MPS) are a group of closely related metabolic disorders that result from deficiencies in lysosomal enzymes involved in glycosaminoglycan metabolism, leading to lysosomal mucopolysaccharide storage. Affected patients, depending on the specific disorder and clinical severity, may present with neurodegeneration, mental retardation, paralysis, dementia and blindness, dysostosis multiplex, craniofacial malformations and facial dysfiguration. Below, some of the most common conditions of this family of diseases are summarized.

Representative examples of common lysosomal storage disorders

| Disease | Enzyme Deficiency | Storage Metabolite |
|---|---|---|
| Glycogenosis-Type 2 | α-1,4-Glucosidase | Glycogen |
| Gangliosidoses | | |
| GM₁ Gangliosidosis | GM₁ ganglioside | GM₁ ganglioside |
| | β-galactosidase | |
| Tay-Sachs disease | Hexosaminidase - α | GM₂ ganglioside |
| | subunit | |
| Sandhoff disease | Hexosaminidase - β | GM₂ ganglioside |
| | subunit | |
| Sulfatidoses | | |
| Krabbe disease | Galactosylceramidase | galactocerebroside |
| Fabry disease | α-Galactosidase A | ceramide trihexoside |
| Gaucher disease | Glucocerebrosidase | glucocerebroside |
| Niemann-Pick - types | Sphingomyelinase | sphingomyelin |
| A & B | | |
| Mucopolysaccharidoses | | |
| Hurler's syndrome | α-L-Iduronidase | dermatan/heparan |
| Hunter's syndrome | L-Iduronosulfate | sulfate |
| | sulfatase | |
| Mucolipidoses | | |
| Mucolipidosis - II | Mannose-6-phosphate | mucopolysaccharide/ |
| Pseudo-Hurler's | kinases | glycolipid |
| Fucosidosis | α-Fucosidase | Glycoproteins |
| Mannosidosis | a-Mannosidase | oligosaccharides |
| Wolman Disease | Acid Lipase | triglycerides |

2. Histopathology & Pathophysiology A Progressive Disorder

In storage diseases, the affected cells become distended and display vacuolated cytoplasms, which appear as swollen lysosomes under the electronic microscope. For example, in the central nervous system, the neurons of the brain, trigeminal and spinal root ganglia in patients suffering from GM₂ gangliodisoses display swollen vacuolated perikarya stored with excessive amounts of lysosomal storage. As a result, these organelles become large in size and numbers, interfering with normal cell functions. The formation of meganeurites, axon hillock enlargements accompanied by secondary neuritic sprouting, present as cardinal histopathological feature of gangliosidoses and mucopolysaccharidoses (Purpura D P, Suzuki K., Brain Res. 1976 Oct. 29; 116(1): 1-21; Walkley S U., Int Rev Neurobiol. 1988; 29:191-244). Purpura and Suzuki proposed that meganeurites, and the synapses they develop, contribute to the onset and progression of neuronal dysfunction in storage diseases, by altering electrical properties of neurons and modifying integrative operations of somatodendritic synaptic inputs. In addition, Walkley et al. (Walkley S U, et al., J Neurol Sci. 1991 July; 104(1):1-8) suggested that this neuroaxonal dystrophy commonly involved GABAergic neurons, and proposed that the resulting defect in neurotransmission in inhibitory circuits may be an important factor underlying brain dysfunction in lysosomal storage diseases. Consequently, the clinical phenotype often includes neurodegeneration, mental retardation, paralysis, dementia and blindness. In addition, some storage disorders also affect peripheral tissues, such as cartilage and bone, resulting in abnormal growth & development of long bones, vertebrae, ribs and jaws, ultimately leading to anomalies of the skeleton, the cranium and dysfiguration of the face (Mucopolysaccharidoses, and Sandhoff's disease to some degree).

One cardinal characteristic of storage disorders is their progressively worsening (progressive) nature. The deficiency of metabolic enzymes results in accumulation of insoluble metabolites in the lysosomes, which becomes excessive and deleterious over time due to the additive effects of accumulating insoluble metabolite storage. For example, patients suffering from mucopolysaccharidoses (Hurler's or Hunter's) display only a mild degree of the disease's phenotype at infancy, but, due to increasing storage over time, progress to severe forms by adolescence, often leading to death (Gorlin R J, Cohen M M, Levin L S (1990). SYNDROMES OF THE HEAD AND NECK, 3rd Edition. Oxford university Press, New York. (1990)). This provides a window of opportunity in mammalian development during which the pathophysiological process of the disease can be attenuated by restoring lysosomal enzymatic activity early enough in life to prevent the development of a “full-blown” disease and, perhaps, to reverse its progression.

3. Tay-Sachs & Sandhoffs Disorders

QM₂ gangliosidosis, including TSD and SD disease, belong to a class of inherited metabolic disorders termed lysosomal storage diseases (LSD). TSD carrier frequency is estimated at 0.0324 (1 in 30) in the North American Jewish population of eastern European descent, higher in those of Austrian descent 0.1092 (1:9), and 0.004 (1:250) in the general population. Affected patients may present with neurodegeneration, mental and motor deterioration, muscular flaccidity, blindness, dysarthria, impaired thermal sensitivity, increasing dementia, and cherry-red spots in the macula of the eye. TSD and SD are progressive disorders, whereby affected patients often display only mild features of the disease at infancy, but progress to severe forms in childhood. Depending on the clinical severity, patients may reach a vegetative state followed by death as early as 3-4 years of age. TSD and SD are employed in this study both as representative examples for the LSD pathobiology and treatment, as well as for their relatively higher prevalence in the general population.

β-hexosaminidase deficiency in humans is pathognomonic in the development of Tay-Sachs (TSD) and Sandhoff (SD) disease, which present with pathologic storage of GM2 ganglioside in the neurons of the brain and spinal cord, leading to brain inflammation and neurodegeneration. The catabolism of the GM₂ ganglioside in mammalian cells is mediated by β-hexosaminidase, a lysosomal acidic hydrolase. The lysosomal enzyme β-hexosaminidase (HEX) is comprised of 2 subunits (peptides), HEX-α and HEX-β, encoded by two distinct genes, HexA and HexB, respectively. β-hexosaminidase exists in 3 isoforms (proteins), HEXA (α/β heterodimer), HEXB (β/β homodimer) and HEXS (α/α homodimer). HEXA is rate limiting in GM₂ catabolism in humans. In humans, HEXA (α/β) catabolizes GM2 when it is presented by a third protein named GM2 activator. Mutation of the HexA gene, causing functional problems with the HEX-α polypeptide in humans results in Tay Sachs disease, whereas mutation of the HexB gene, causing functional problems in the β-Hex polypeptide, in Sandhoff's disease. In Tay Sachs disease, HexA mutation results in loss of HEXA isoform (α/β heterodimer), whereas in Sandhoff's disease, HexB mutation results in loss of both HEXA (α/β heterodimer) and HexB (β/β homodimer) isoforms, leading to a more severe clinical phenotype. Human patients with HexA (Tay-Sachs) or HexB deficiency (Sandhoff disease) develop storage of GM2 gangliosides in the lysosomes primarily of neurons due to the lack of HEXA (α/β) activity (Gravel et al., 1995). Affected patients, depending on the clinical severity, may present with neurodegeneration, mental and motor deterioration, dysarthria, impaired thermal sensitivity, blindness, as well as facial dysfiguration (doll-like and coarse facies), muscular flaccidity, increasing dementia, and the characteristic macular cherry-red spots. Histopathologically, the cells of the brain (neurons and glia), spleen and cartilage appear swollen with vaculolated/clear perikarya suggestive of lysosomal storage. Biochemical analysis reveals a complete lack of β-hexosaminidase activity accompanied by lysosomal accumulation of GM₂ gangliosides. As a result, the lysosomes become large in size and numbers, significantly crippling normal cellular function, presumably interfering with normal cellular functions and ultimately leading to neuronal cell death (apoptosis). Clinically, it is not uncommon for patients to display only mild features at infancy, but due to increasing storage over time, progress to severe forms of the disease by adolescence (Gorlin R J, Cohen M M, Levin L S (1990). SYNDROMES OF THE HEAD AND NECK, 3rd Edition. Oxford university Press, New York. (1990)). Similarly, other affected mammals, such as affected mice pups, display only mild anomalies at birth, but quickly develop their distinct abnormal features (1 month of age).

Although HEXA is present in all cell types and tissues, neurons are characterized by a remarkably higher concentration of gangliosides than other cell types and therefore are highly susceptible to GM₂ lysosomal storage secondary to f-hexosaminidase deficiency, ultimately leading to cellular dysfunction neurodegeneration (Walkley S U, et al., J Neurol Sci 104: 1-8. (1991), 1998; Purpura D P, Suzuki K, Brain Research 116: 1-21. (1976); Huang J Q, et al., Hum Mol Genet. 6: 1879-1885. (1997)).

Histopathologically, affected cells are distended and have vacuolated cytoplasms, which appear as swollen lysosomes under the electronic microscope. Neurons of the brain, cerebellum, trigeminal and spinal root ganglia display swollen vacuolated perikarya stored with excessive amounts of lysosomal storage macromolecules. The formation of meganeurites, axon hillock enlargements accompanied by secondary neuritic sprouting has, also been described in the brain of Tay-Sachs patients (Walkley S U, et al., J Neurol Sci. 1991 July; 104(1):1-8; Purpura D P, Suzuki K., Brain Res. 1976 Oct. 29; 116(1): 1-21). Purpura and Suzuki have proposed that meganeurites, and the synapses they develop, contribute to the onset and progression of neuronal dysfunction in storage diseases, by altering electrical properties of neurons and modifying integrative operations of somatodendritic synaptic inputs. In addition, Walkley et al. (Walkley S U, et al., J Neurol Sci. 1991 July; 104(1):1-8) suggested that this neuroaxonal dystrophy commonly involved GABAergic neurons, and proposed that the resulting defect in neurotransmission of inhibitory circuits may be an important factor in brain dysfunction. Autopsy studies on brain and spinal cord samples obtained from Tay-Sachs and Sandhoff patients revealed neuronal cell death in both instances. Huang et al. (Huang D R, et al., J Exp Med 193: 713-26. (2001)) suggested that neuronal death is caused by unscheduled apoptosis, implicating accumulated GM₂ ganglioside in triggering the apoptotic cascade. Therefore, disruptions in the HexA or HexB loci result in β-hexosaminidase deficiency and ultimately lead to the development of Tay-Sachs or Sandhoff disease in humans.

Patients suffering from lysosomal storage disorders, including Tay-Sachs and Sandhoff's diseases, often present with pathologic storage of insoluble metabolic products in the central nervous system (CNS) due to an inherited metabolic anomaly [C. Chavany, M. Jendoubi, Mol Medicine Today 4 (1998) 158-165]. Treatments for lysosomal storage diseases have included enzyme replacement therapy [J. Barranger, E. O'Rourke, J. Inherit. Metabol. Disord. 24 (2001) 89-96; C. M. Eng, et al., Am. J. Hum. Genet. 68 (2001) 711-722] as well as bone marrow transplantation [E. H. Birkenmeier. Blood 78 (1991) 3081-3092, S. U. Wakley, K. Dobrenis, Lancet 345 (1995) 1382-1383]. Although supported by in vitro studies [S. E. Brooks, et al., 50 (1980) 9-17], enzyme replacement therapy for Tay-Sachs disease has not been proven of therapeutic value [B. U. von Specht, et al., Neurol. 29 (1979) 848-854] presumably due to the presence of the blood brain barrier [C. Chavany, M. Jendoubi, Mol Medicine Today 4 (1998) 158-165]. Bone marrow transplantation has been developed as an alternative method of treatment. In Sandhoff's mice, bone marrow transplantation has been successful in extending life expectancy of affected animals [F. Norflus, J. Clin. Investi. 101 (1998) 1881-1888] concomitantly with a small but significant increase of β-hexosaminidase activity in the brain. Similar findings were previously reported following bone marrow transplantation in an α-mannosidosis mouse model [S. U. Walkley, et al., Proc. Natl. Acad. Sci. U.S.A. 91 (1994) 2970-2974].

Recent studies on the pathophysiology of GM₂ gangliosidosis revealed the presence of activated microglia and macrophages in the brain of hexB^{-/-} knockout mice, along with increased levels of several inflammation-related genes (Wada R, et al., Proc Natl Acad Sci USA 97: 10954-10959. (2002); Myerowitz R, et al., Hum Mol Genet. 11: 1343-1350. (2002); Jeyakumar M, et al., Brain 126: 974-987. (2003)). It was also suggested that glial activation and brain inflammation contributes to neurodegeneration as it appeared to precede temporally and spatially neuronal cell death (Wada R, et al., Proc Natl Acad Sci USA 97: 10954-10959. (2002); Myerowitz R, et al., Hum Mol Genet. 11: 1343-1350. (2002)). To this end, transplantation of healthy bone marrow to hexB^{-/-} pups attenuated the microglia/macrophage activation, reduced the extent of neuronal apoptosis and ameliorated the clinical phenotype (Norflus F, et al., J Clin Invest 101: 1881-1888. (1998)). The aforementioned studies suggest a critical role for peripheral immune cells in the pathogenesis of GM₂ gangliosidosis. However, as discussed herein, the role of the immune cells is temporally dependent. With less immune cell deposition occurring when vectors are systemically delivered to neonates than adults.

4. Craniofacial Development

Craniofacial development involves both endomembranous as well as endochondral mechanisms of bone formation. Ossification of the human mandible begins approximately during the 7^{th} week of gestation, anterior to the Meckel's cartilage at the future site of the lower canine. Interestingly, mandibular formation is based on endomembranous (corpus) as well as endochondral (ramus, coronoid process, condylar process, anterior part of body) mechanisms. Postnatally, the lower jaw grows by apposition of new bone on the posterior surfaces of the body and ramus as well as on the surface of the condylar process. Similarly, the human maxilla begins ossification around the 9^{th}-10^{th} week of gestation in locations proximal to nerve openings and canals (infraorbital, palatine and incisive nerves), which gradually extends to the rest of the bone (Kjaer et al. Munksgaard, Copenhagen, pp 20-51 (1999)). The maxilla articulates with the rest of the facial skeleton through synchondroses, which are considered growth sites and remain uncalcified into early childhood. Postnatally, the maxilla continues to grow by apposition of new bone on its posterior surfaces (tuberosities) as it translates down and forward away from the anterior cranial base. The cranial base forms (occipital-sphenoid) mainly via endochondral mechanisms beginning the 9^{th}-10^{th} week of gestation. Interestingly, the ethmoidal bone, which comprises the most anterior portion of the anterior cranial base, does not form until later in embryogenesis (Kjaer et al. Munksgaard, Copenhagen, pp 20-51 (1999)). Two important synchondroses, the spheno-occipital and spheno-ethmoidal, remain open (uncalified) well into childhood and are considered to be sites where bone growth occurs, as the cranial base develops, carrying the maxilla forward, as it is attached onto the inferior part of the ethmoidalbone. Interestingly, although craniofacial development begins early in embryogenesis, as large portion of total bone growth occurs postnatally. (Enlow & Hans Essentials of facial growth. W.B. Saunders Co, New York. (1996)). Evidently, any aberrant factors that may disturb this delicate process can potentially result into abnormal skeletal development (Rosenberg A In Robbins Pathologic Basis of Disease, 6^{th} edition; Cotran, Kumar & Collins (Ed), W.B. Saunders Co, New York (1999)), including transcription factor anomalies (homeobox genes: i.e. PAX-3), inter-cellular signaling (growth factor receptors: i.e. FGFR2) or bone matrix abnormalities (collagen mutations: i.e. COLL1A1). Depending on the severity of the genetic anomaly, a phenotype may include bone agenesis (i.e. missing clavicle), malformation (craniorachischisis) or abnormal bone growth (growth plates and synchondroses). Clinically, one can identify a number of disorders resulting from different genetic anomalies that share similar phenotypes, including abnormal size and shape of the jaws, frontal bossing and midface hypoplasia, due to aberrant abnormal growth of the bones of the cranial and facial skeleton. Therefore, we hypothesize that genetic anomalies causing dysfunction of cells associated directly (chondrocytes and osteocytes) or indirectly (neurons) with craniofacial development can adversely affect skeletal development.

5. Craniofacial Development & Neuronal Innervation

An increasing body of evidence is consistent with the nervous system playing an important role in craniofacial development, and that aberrant neuronal function and/or innervation may contribute to abnormal craniofacial growth and development. From the early stages of embryogenesis, the neural crest, cellular derivatives of which contribute to the formation of the face, develops in close interaction with the primitive neural tube. Moreover, conditions that affect the development of the brain often involve craniofacial anomalies, such as holoprocencephely, cyclopia and other syndromes or disorders. For example, a retrospective study by Cohen and Kreiborg (Am J Med Genet. 35: 36-45 (1990) reported mental retardation, positive neurological and histopathological findings in patients with Apert's syndrome (craniosynostosis). The authors suggested that central nervous system anomalies may be responsible for the development of craniofacial abnormalities. Kjaer (Crit Rev Oral Biol Med 9: 224-244 (1998)) suggested an association between the central nervous system and the axial skeleton, as well as between the peripheral nervous system and bony jaw formation; her findings were based on temporal and spatial correlations of nerve and jaw development. Hoffman and McCarthy (Plast Recostr Surg 93: 1236-1240 (1994)) performed unilateral facial nerve resections in perinatal rabbits that were allowed to grow to maturity (6 months old). This procedure resulted in complete loss of motor innervation and paralysis of the facial muscles, which led to aberrant nasomaxillary and mandibular growth ipsilaterally to this cranial nerve ablation. Similar results were also reported by Sinsel et al. (Past Reconstr Surg 102: 1894-1912 (1998)). Ben-Shachar et al. examined the role of γ-aminobutyric acid (GABA) in craniofacial development by administering picrotoxin, a GABA receptor antagonist, to neonatal rabbits (Ben-Shachar et al. J Craniofac Genet Develop Biol 8: 351-361. (1988a); Ben-Shachar et al. J Craniofac Genet Develop Biol 8: 363-372 (1988b)). Treated rabbits that were allowed to grow to young adulthood displayed nasomaxillary and mandibular skeletal anomalies. Their studies suggested that GABA receptor function is essential for normal craniofacial development. Conclusive evidence about the role of GABA receptor function in craniofacial development came from the following experiment by Culiat et al. (Nature Genet 11: 344-346 (1995)): Restitution of the beta-3 subunit of the GABA receptor (Gabrb3) in mice with cleft palate secondary to disruption of the cleft palate 1 locus (cp1) on chromosome 7 prevented the development of clefting. Therefore, it appears that the GABA-mediated inhibitory functions, at the level of cortico- and inter-neuronal projections in the central nervous system, are important for normal craniofacial development. Furthermore, GABA has been shown to have neurotransmitter-like properties in the peripheral nervous system autonomic projections. Although the role of GABA mediated-neurotransmission remains unclear, it is suggested that it plays an important role in peripheral innervation. In an elegant experiment, Byrd et al. (Anat Record 258: 369-383 (2000)) demonstrated that biodegradable microspheres containing glycine or glutamate stereotactically implanted proximal to trigeminal motoneurons in brain stem affected craniofacial growth: Glycine (inhibitory) induced significantly smaller cranial dimensions and mandibular condyles ispilaterally to the treatment, whereas glutamate resulted in larger cranial dimensions. In conclusion, the aforementioned studies indicate a correlation between the nervous system and craniofacial development. Disclosed herein are vectors and methods that address the role of the nervous system in craniofacial development and which can alleviate the debilitating symptoms of the neuro effects craniofacial development.

6. A Mouse Model for the Study of Neuro-Skeleton Interaction.

Disclosed herein, normal neuronal function is required for craniofacial development, and that neuronal dysfunction contributes to aberrant craniofacial development. Disclosed is an animal model characterized by severe craniofacial dysostosis and growth retardation along with brain dysfunction associated with excessive neuronal storage of GM₂ gangliosides and mucopolysaccharides due to β-hexosaminidase deficiency (hexA^{-/-}/hexB^{-/-} or hexA^{-/-}/hexB^{+/-} double knockout mice) that can be used in conjunction with disclosed vectors and methods to reverse the effects of the neuronal effect on craniofacial development.

Structurally, β-hexosaminidase is comprised of 2 subunits, α& β, each encoded by a separate gene, HexA and HexB, respectively. The enzyme exists in 2 major isoforms HEXA (α/β heterodimer) and HEXB (β/β homodimer). Targeted deletion of both hexA & hexB murine loci results in the development of a mouse phenotype characterized by craniofacial dysplasia with facial disfiguration, concomitantly with neuronal anomalies (lysosomal storage of insoluble metabolites) that result in behavioral (motor) impairment and limited life span (Sango et al. Nature Genet 14: 348-352 (1996); Suzuki et al. J Neuropath Exp Neurol 56: 693-703 (1997)). Although, hexA^{-/-}/hexB^{-/-} newborns display only mild anomalies at birth, by 4-5 weeks of age they consistently develop aberrant features that include small physical size, facial dysmorphia, short head, broad snout, frontal bossing, abnormally shaped jaws and midface hypoplasia due to maxillary retrusion. Furthermore, they suffer from kyphosis, abnormally shaped rib cage with broad ribs, and shortened long bones. They are also unsuccessful in breeding and have limited life span (4-5 weeks). Histopathologically, the cells of the brain (neurons and glia) and of the trigeminal and spinal ganglia appear swollen with vaculolated/clear perikarya; biochemical analysis reveals complete lack of β-hexosaminidase activity accompanied by storage of gangliosides and mucopolysaccharides.

7. Animal Models of GM₂ Lysosomal Storage Disorders: Hex Knockout Mice

In the mouse, two genes also encode for β-hexosaminidase (Sango K, et al., Nature Genet 11: 170-176. (1995)). Due to species variation in GM₂ metabolism, targeted deletion of the murine hexB^{-/-} locus is required for the development of GM₂ storage in mice (Sango K, et al., Nature Genet 14: 348-352. (1996); Phaneuf D, et al., Hum Mol Genet 5: 1-14. (1996); Suzuki K, et al., J Neuropath Exp Neurol 56: 693-703. (1997)).

Disruption of the murine hexB locus resulted in a mouse phenotype that closely resembled that of the human disease. The mice displayed storage of GM₂ ganglioside in the CNS, and neurons with membranous cytoplasmic bodies similar to those in Tay-Sachs and Sandhoff patients (Sango K, et al., Nature Genet 14: 348-52. (1996); PhaneufD, et al., Hum Mol Genet 5: 1-14. (1996)). Interestingly, mice with disruption of both the hexA and hexB loci (double knockouts) were devoid of β-hexosaminidase activity, and showed severe GM₂ pathology (Sango K, et al., Nature Genet 14: 348-52. (1996); Suzuki K, et al., J Neuropath Exp Neurol 56: 693-703. (1997)). The phenotypic variation between humans and mice appears to result from differences in the ganglioside degradation pathway between the species. It has been proposed that a second ganglioside degradation pathway exists in the mouse (Sango K, et al., Nature Genet 14: 348-52. (1996)), whereby GM₂ can, at least in the absence of HEXA (α/β), be metabolized by a murine sialidase to asialo-GM₂ and subsequently catabolized by HEXB (β/β). However, human sialidases can not metabolize GM₂ ganglioside (Sango K, et al., Nature Genet 14: 348-52. (1996)). Therefore, hexB disruption in the mouse results in GM₂ gangliosidosis, whereas in the human either HexA (TSD) or HexB (SD) mutations can cause GM₂ storage (Chavani & Jendoubi 1998). For these reasons, the hexB^{-/-} knockout mouse is widely accepted as an appropriate animal model in the study of GM₂ gangliosidosis (Sango K, et al., Nature Genet 14: 348-52. (1996); Phaneuf D, et al., Hum Mol Genet 5: 1-14. (1996); Suzuki K, et al., J Neuropath Exp Neurol 56: 693-703. (1997)).

The hexB^{-/-} knockout mice is characterized by similar clinical, histological and biochemical features to Tay-Sachs (TSD) and Sandhoff (SD) disease (Sango K, et al., Nature Genet 14: 348-52. (1996); Phaneuf D, et al., Hum Mol Genet 5: 1-14. (1996)). Clinically, both the hexB^{-/-} mouse and human patients display near normal phenotype at birth, but quickly develop muscle weakness, rigidity, and motor deterioration typically leading to death (approximately 4 months in the mouse and 2-4 years of age in human patients). At the histopathology level, progressive storage of GM₂ gangliosides in the neurons of the brain and spinal cord is cardinal characteristic followed by cell apoptosis in humans as well as hexB^{-/-} mice (Huang D R, et al., J Exp Med 193: 713-26. (2001); Wada R, et al., Proc Natl Acad Sci USA 97: 10954-10959 (2002); Myerowitz et al., 2002). Recent studies on hexB^{-/-} mice also showed the presence of activated microglia and macrophages in the brain of affected animals, along with upregulation of several inflammation-related genes (Norflus F, et al., J Clin Invest 101: 1881-1888. (1998)). Consequently, it was suggested that this brain inflammation contributes to neurodegeneration: the presence of activated microglia and/or macrophages in the brain preceded neuronal cell death, and were observed proximal to neurons undergoing apoptosis (Wada et al., 2000 Proc Natl Acad Sci USA 97: 10954-10959; Myerowitz et al., Human Molecular Genetics. 11(11):1343-50, 2002 2002). Furthermore, transplantation of healthy bone marrow to hexB−/− pups attenuated the microglia/macrophage activation, reduced the extent of neuronal apoptosis and ameliorated the clinical phenotype (Norflus F, et al., J Clin Invest 101: 1881-1888. (1998)). Although the adverse effects of lysosomal storage on monocyte and macrophages have been previously documented, the mechanism through which peripherally administered (intraperitoneal injections) healthy myeloid-derived cells exert their anti-inflammatory and neuro-protective effect in GM₂ gangliosidosis is unclear.

8. Blood Brain Barrier Formation

The blood-brain barrier (BBB) is a structure unique to the central nervous system and is the result of tight junctions between the brain endothelial cells (Goldstein G W, et al., Ann NY Acad Sci 481:202-13. (1986)). Previous work (Risau W, et al., Devel Biol 117: 537-545. (1986)) on the development of mouse BBB using large protein molecules (horse radish peroxidase) suggested BBB formation during the late days of embryonic life (E17 in mouse). Furthermore, BBB in the adult is not absolute, whereby certain areas of the brain do not develop BBB and thus allow for free exchange of molecules through them. These areas include the median eminence (hypothalamus), pituitary, choroids plexus, pineal gland, sub fomical organ, organum vasculo sum lamina terminalis and area posterma (Risau W. Wolburg H, TINS 13:174-178. (1990)). This allows for the intrusion of FIV(Hex) virions into the brain matter through an incomplete BBB as well as through areas lacking BBB during the first few days after birth as discussed in the examples herein. Disclosed herein a diffuse expression of lacZ throughout the brain of P4 mice injected with FIV(lacZ) versus periventricular only localization following “adult administration” was shown.

Specific immunity in vertebrates is dependent on the host's ability to generate a heterogeneous repertoire of antigen-binding structures that are displayed on the surface of lymphocytes. Immunologic competence arises early in mammalian development. Since the expression of β-Hex therapeutic gene in hexB^{-/-} mice may be perceived as presentation of “non-self” antigens, the possibility of an immune response against human HEXA and HEXB following gene therapy would be considered. In these terms, perinatal administration can offer a unique opportunity in gene therapy application. Specifically, numerous studies have documented that the human and mouse neonate is unable to mount satisfactory responses to various antigenic challenges, which in many instances is delayed well beyond infancy (Schroeder W H, et al., Ann NY Acad Scie 764: 242-260. (1995)). Therefore, due to this “immature” immunologic state of mice and humans early in their postnatal life, disclosed herein, perinatal gene therapy will allow for adequate “training” of the immune system to recognize HEXA and HEXB as “self” antigens circumventing any potential immunologic rejection.

9. Immune System Development

Specific immunity in vertebrates is dependent on the host's ability to generate a heterogeneous repertoire of antigen-binding structures that are displayed on the surface of lymphocytes. Immunologic competence arises early in mammalian development. Since the expression of β-Hex therapeutic gene in hexA^{-/-}/hexB^{-/-} mice may be perceived as presentation of “non-self” antigens, one needs to consider the possibility of an immune response against human HEXA and HEXB following gene therapy. In these terms, perinatal administration can offer a unique opportunity in gene therapy application. Specifically, numerous studies have documented that the human and mouse neonate is unable to mount satisfactory responses to various antigenic challenges, which in many instances is delayed well beyond infancy (Schroeder W H, et al., Ann NY Acad Scie 764: 242-260. (1995)). Therefore, due to this “immature” immunologic state of mice and humans early in their postnatal life, perinatal gene therapy is consistent with adequate “training” of the immune system to recognize HEXA and HEXB as “self” antigens circumventing any potential immunologic rejection. It is understood that the transtherapy can take place in an infant as well.

10. Treatments for GM₂ Gangliosidosis

Receptor-mediated enzyme transfer (cross-correction) is an important characteristic of lysosomal enzymes, including β-hexosaminidase, whereby secreted enzyme can be up-taken by neighboring cells via paracrine pathways. The transport and compartmentalization of soluble lysosomal enzymes to lysosomes depends on the recognition of mannose 6-phosphate (Man-6-P) residues in their oligosaccharide moiety by specific receptors. Two distinct proteins have been thus far identified as capable of interacting with lysosomal enzymes, the Man-6-P receptor (MPR; 270 kDa) which also binds the insulin-like growth factor-II (IGF-II), and the cation-dependent MPR (CD-MPR; 46 kDa; Munier-Lehman et al., 1995). Cross-correction based treatments, such as enzyme replacement therapy (ERT) and bone marrow transplantation (BMT) have been previously employed in some cases without, however, any clinical improvement (von Specht B U, et al., Neurol 29: 848-854. (1979)). Although these therapies address the lack of metabolic activity in peripheral tissues, due to the presence of the blood-brain-barrier, they failed to avert disease progression in the central nervous system. Hence patients and laboratory animals continue to clinically deteriorate due to central nervous system involvement. Recently, drug-mediated substrate (GM₂) deprivation has been considered as an alternative treatment strategy (Liu et al. J Clin Invest 103: 497-505 (1999)). Gene therapy and its preliminary success on other genetic disorders, including Sly disease (mucopolysaccharidosis type VII), offers a logical alternative for the management of β-hexosaminidase deficiency. To this end, Akli et al. Gene Therapy 3: 769-774 (1996) reported successful restoration of β-hexosaminidase in fibroblasts derived from Tay-Sachs patients via adenoviral-mediated gene transfer in vitro. In another study, human HexA and HexB expressing vectors were introduced into neural progenitor cells utilizing retroviral vectors, and subsequently transplanted ex vivo into the brains of E14.5 and newborn mice (Lacorazza et al., 1996). The authors were able to detect a significant increase in β-hexosaminidase protein synthesis and enzyme activity in the brains of these mice. These and other studies indicate that gene therapy modalities may evolve as a feasible treatment in the management of TSD and SD disease.

11. Gene Therapy

Considerable progress has also been made in the field of gene therapy. For example, β-glucuronidase deficient mice displaying characteristics of Sly disease (mucopolysaccharidosis type VII) were administered adeno-associated [T. M. Daly, et al., Proc. Natl. Acad. Sci. U.S.A. 96 (1999) 2296-2300, W. A. Frisella, et al., Mol. Ther. 3 (2001) 351-358] and feline immunodeficiency virus [A. I. Brooks, et al., Proc. Nat. Acad. Sci. U.S.A. 99. (2002) 6216-6221] successfully transducing the human β-glucuronidase gene. Previous studies on the development of gene therapy for β-hexosaminidase disorders reported the restoration of hexosaminidase-A activity in vitro [S. Akli, J et al., Gene Ther. 3 (1996) 769-774, H. D. Lacorazza, et al., Nature Med. 2 (1996) 424-429, S. Martino, et al., J. Biol. Chem. 277 (2002) 20177-20184, M. S. Sands, et al., J. Clin. Invest. 93 (1994) 2324-2331] as well as in hexA deficient mice [J. E. Guidotti, et al., Hum. Mol. Genet. 8 (1999) 831-838]. Akli et al. (Akli S, et al., Gene Therapy 3: 769-774 (1996)) reported successful restoration of β-hexosaminidase activity in fibroblasts derived from Tay-Sachs patients via adenoviral-mediated gene transfer in vitro. In another study, human HexA and HexB expressing vectors were introduced into neural progenitor cells utilizing retroviral vectors, and subsequently transplanted ex vivo into the brains of E14.5 and newborn mice (Lacorazza et al., 1996). The authors were able to detect a significant increase in β-hexosaminidase protein synthesis and enzyme activity in the brains of these mice. Nevertheless, the blood brain barrier appeared to limit viral penetration into the rodent brain following peripheral administration.

Gene therapy is based on the transfer of a therapeutic gene to affected patients or animals, whereby correlate cellular dysfunction is normalized leading to the treatment of a disease. A number of factors are important in β-hexosaminidase gene therapy, including construction of a therapeutic β-hexosaminidase gene, global gene delivery, stable gene expression, appropriate route and timing of administration. The construction of a transgene encoding for both subunits of β-hexosaminidase, HexA and HexB, as disclosed herein, is important, since it would allow for all isoforms, HEXA (α/β), HEXB (β/β) and HEXS (α/α), to be restored. Furthermore, since lysosomal disorders are characterized by pancellular enzyme deficiency, expression of the therapeutic gene should be directed to all tissues and organs. Disclosed herein is a bicistronic gene (HexB-IRES-HexA) that encodes for both human HexA and HexB genes leading to the synthesis of functional β-hexosaminidase (As disclosed herein). Pan-cellular expression can be achieved by the use of universal promoters (as disclosed herein).

Gene therapy can be applied, in general, via local or systemic routes of administration. Local administration includes virus injection directly into the region or organ of interest, versus intravenous (I.V.) or intraperitoneal (I.P.) injections (systemic) aiming at viral delivery to multiple sites and organs via the blood circulation. Previous research on the effects of local administration demonstrated gene expression limited to the site/organ of the injection, which did not extend to the rest of the body Daly et al. Hum Gene Ther 10: 85-94 (1999a); Kordower et al. Exp Neurol 160:1-16 (1999). In contrast, viral I.V. and I.P. injections have resulted in viral gene distribution to multiple tissues and organs in rodents and primates (Daly et al. Proc Natl Acad Sci USA 96: 2296-2300 (1999b); Tarantal et al. Mol Ther 3:128-138 (2001); McCormack et al. Mol Ther 3: 516-525 (2001; Lipschutz et al. Mol Ther 3: 284-92. (2001)).

Stable expression of the therapeutic gene ensures prolonged restoration of the genetic anomaly enhancing treatment efficacy and contributing to long-term therapeutic outcomes. The disclosed lentiviral vectors have been shown to effectively incorporate the transgene of interest into the host's genome, allowing for stable gene expression (Poeschla et al. Nature Medicine 4: 354-357 (1998); data disclosed herein). In addition, the use of mammalian and/or recombinant promoters have allowed researchers to maintain therapeutic levels of gene expression for periods of up to one year in rodents utilizing a chicken β-actin/CMV fusion promoter (Daly et al. Gene Ther 8: 1291-8 (2001)). Disclosed herein are data showing stable expression of the reporter gene lacZ for over 6 months in mice following systemic FIV(lacZ) administration to 2 days old (P2) mouse pups.

The timing of gene therapy is important as it is closely related to the temporal development of the disorder. As mentioned above, patients and mice affected by β-hexosaminidase deficiency display only mild phenotype aberrations at birth, but quickly progress to severe forms by adolescence. Therefore, in certain embodiments, disclosed is the administration of β-hexosaminidase gene therapy to mice and other subjects, such as humans, during neonatal stages of development.

a) Routes of Administration

Gene therapy can be applied, in general, via local or systemic routes of administration. Local administration includes virus injection directly into the region or organ of interest, versus intravenous (I.V.) or intraperitoneal (I.P.) injections (systemic) aiming at viral delivery to multiple sites and organs via the blood circulation. Previous research on the effects of local administration demonstrated gene expression limited to the site/organ of the injection, which did not extend to the rest of the body (Daly T M, et al., Hum Gene Ther 10:85-94. (1999a); K Kordower J H, et al., Exp Neurol 160:1-16. (1999)). In contrast, viral I.V. and I.P. injections have resulted in viral gene distribution to multiple tissues and organs in rodents and primates (Daly et al., 1999b; Tarntal et al., 2001; McCormack J E, et al., (2001) Mol Ther 3: 516-525; Lipschutz G S, et al., Mol Ther 3: 284-92. (2001)). In addition, since GM₂ gangliosidosis develops primarily in neurons, disclosed herein lentiviridia are a preferred vehicle for the disclosed uses because it has been shown to be capable of transducing dividing, growth arrested as well as neurons (Poeschla E M, et al., Nature Medicine 4: 354-357. (1998)); As disclosed herein). For this purpose, disclosed is a VSV-G pseudotyped lentiviral vector derived from the feline immunodeficiency virus (FIV) and it has been shown herein that I.P. injection of FIV(lacZ) in mice of neonatal age (P2) resulted in the transfer and expression of the lacZ gene in the brain and liver of mice in vivo. The levels of expression achieved via intraperitoneal injections were superior to those acquired following local administration directly into the liver. Moreover, intraperitoneal injection of FIV(Hex) to mice of neonatal age (P2) also resulted in the transfer and expression of the HexB-IRES-HexA gene in the brain and liver of mice in vivo (in neonatal paper).

Stable expression of the therapeutic gene ensures prolonged restoration of the genetic anomaly enhancing treatment efficacy and contributing to long-term therapeutic outcomes. The disclosed lentiviral vectors have been shown to effectively incorporate the transgene of interest into the host's genome, allowing for stable gene expression (Poeschla E M, et al., Nature Medicine 4: 354-357. (1998)). In addition, the use of mammalian and/or recombinant promoters has allowed researchers to maintain therapeutic levels of gene expression for periods of up to one year in rodents utilizing a chicken β-actin/CMV fusion promoter (Daly et al., 2001). The data disclosed herein show stable expression of the reporter gene lacZ for over 6 weeks in mice following systemic FIV administration to 2 day old (P2) mouse pups.

The timing of gene therapy is important as it is closely related to the temporal development of the disorder. As mentioned above, patients and mice affected by β-hexosaminidase deficiency display only mild phenotype aberrations at birth, but quickly progress to severe forms by adolescence. Therefore, disclosed herein, is the administration of β-hexosaminidase gene therapy to during neonatal stages of development, including to mice and to humans, including those that are known to carry the genetic deficiency.

In evaluating the effects of viral vectors as the basis for systemic gene therapy in the adult rodent, disclosed herein a VSV-G pseudotyped FIV(lacZ) vector [E. M. Poeschla, et al., Nature Med. 4 (1998) 354-357] was employed and was shown to be capable of transducing dividing, growth arrested as well as post-mitotic cells with the reporter gene lacZ. The vectors were also shown to transducer brain cells, particularly in neonatal injections as well as after adult injections. VSV-G pseudotyping of FIV vectors confers a broad range of host specificity, including human and murine cells, as infection is promoted by the interaction of the viral envelope protein and a phospholipid component of the cell membrane leading to membrane-fusion mediated entry [J. C. Burns, et al., Proc. Natl. Acad. Sci. U.S.A. 90 (1993) 8033-8037, F. A. Carneiro, et al., J. Virol. 76 (2002) 3756-3764]. Although some concerns about potential FIV toxicity have been previously raised [D. C. Bragg, et al., J. Neurovirol. 8 (2002) 225-239], in certain embodiments the VSV-G pseudotyped vectors have alleviated these concerns [M. A. Curran, et al., Transplantation 74; (2002) 299-306. Moreover, FIV belongs to the family of lentiviruses, capable of stable transgene integration into the host's genome; however, the cytomegalovirus promoter that drives the expression of lacZ in our vector is susceptible to silencing, which limits the longevity of transgene expression in vivo.

The efficacy of VSV-G pseudotyped FIV vectors to transduce peripheral tissues following systemic intravenous administration has been previously reported [Y. Kang, et al., J. Virol. 76 (2002) 9378-9388], as well as the brain [U. Bloemer, et al., J. Virology 71 (1997) 6641-6649, A. I. Brooks, et al., Proc. Nat. Acad. Sci. U.S.A. 99 (2002) 6216-6221] and cerebellum [J. M. Alisky, et al., Mol. Neurosci. 11 (2000) 2669-2673] following intracranial injections. However, limited information is available on the ability of FIV to cross the blood brain barrier and infect cells located in the CNS. Herein, adult mice were injected intraperitoneally with FIV(lacZ), and the expression of β-galactosidase was studied 5 weeks following treatment in the brain, liver, spleen and kidney by X-gal histochemistry and immunocytochemistry. Interestingly, relatively low doses of FIV(lacZ) administered intraperitoneally lead to bacterial β-galactosidase expression in the brain and cerebellum. The identity of the cells expressing lacZ was confirmed by double immunofluorescence.

Previous studies on the development of β-hexosaminidase adenoviral vectors demonstrated restoration of HexA activity in cells in vitro (Akli et al., 1996; Lacorazza H D, et al., Nature Med 2: 424-429. (1996)) as well as in hexA deficient mice in vivo (Guidotti J E, et al., Hum Mol Genet. 8: 831-838. (1999)). Disclosed herein systemic administration of a lentiviral vector (Kyrkanides S, et al., Mol Brain Res 119: 1-9. (2003a)) would result in the transfer of the bicistronic transgene βHex, encoding for both isoforms of the human enzyme (Kyrkanides S, et al., Mol Therapy 8: 790-795. (2003b)), to the brain of hexB-deficient (Sandhoff disease) mice. Neonatal administration was elected on the basis that stable transduction of host cells early in post-natal development would lead to timely β-hexosaminidase restoration and ultimately to disease prevention. Furthermore, neonates have an incomplete state of the blood-brain-barrier, and the neonate is unable to elicit satisfactory immunologic responses to various antigenic challenges. Since brain inflammation, neuronal cell death and motor dysfunction are characteristics of hexB-deficiency and GM₂ gangliosidosis, these parameters were employed as experimental outcomes in the disclosed experiments.

12. Methods and Vectors for Neonatal, Perinatal, and Adult Administration

The data disclosed herein indicates that neonatal FIV(Hex) treatment to hexB^{-/-} pups resulted in the expression of the β-hexosaminidase transgene in peripheral organs as well as the brain, ultimately leading to amelioration of the disease model of GM₂ gangliosidosis.

Disclosed herein FIV(Hex) intraperitoneal administration to neonatal pups results in transduction of microglia and other perivascular cells, neurons and PBMC. β-hexosaminidase activity can be restored at therapeutic levels based on data disclosed herein from behavioral assays. Very small amounts of expressed product are need to have a therapeutic effect on the disclosed diseases. Conzelmann et al. (1983) used a sensitive assay to demonstrate a correlation between level of residual β-hexosaminidase activity and clinical severity in GM₂ human patients: Tay-Sachs disease, 0.1% of normal; late-infantile, 0.5%; adult GM₂-gangliosidosis, 2-4%; healthy persons with low hexosaminidase, 11-20%. Therefore, one expects that restoration at, for example, 5% or 10% or greater will be beneficial to the affected subjects, such as mice or humans. Moreover, the data indicate that only a portion of the brain cells become infected by FIV, with glial preponderance (See Examples). However, β-hexosaminidase is capable of cross-correcting (See Examples), whereby lysosomal enzymes have the ability to be released extracellularly and then to be absorbed via paracrine pathways by other cells, appropriately compartmentalized via mannose-6-phosphate receptors, and contribute to GM₂ catabolism (Lacorazza et al., 1996). Similarly, in a mouse model of Sly disease, neonatal intraperitoneal administration of a human β-glucuronidase recombinant adeno-associated viral vector successfully resulted in storage resolution and disease attenuation (Daly T M, et al., Hum Gene Ther 10:85-94. (1999a); Daly T M, et al., Proc Natl Acad Sci USA 96: 2296-2300. (1999b)).

In any situation where there are immune responses to the expressed transgene product, one can move earlier in the developmental stage of the subject, such as a P1 mouse, or P0.5 mouse, and it could even be performed in utero. (Numerous studies report in utero administrations; Lipschutz G S, et al., Mol Ther 3: 284-92. (2001); Tarantal A F, et al., Mol Ther 3: 128-138. (2001)). In addition, the use of pharmacologic agents that suppress the immune system, such as cyclophosphamide, has been previously described as a strategy to alleviate potential adverse immune response following gene therapy (McCormack J E, et al., (2001) Mol Ther 3: 516-525).

a) Peripherally Administered FIV Vectors Enter into the CNS and Directly Transduce Brain Cells in Neonates

Vectors have been constructed and are disclosed herein, such as FIV(Hex), a β-hexosaminidase lentiviral vector based on the feline immunodeficiency system (Poeschla E M, et al., Nature Medicine 4: 354-357. (1998)). When this vector is produced titers typically range between 5×10⁷-5×10⁸ infectious particles/mL. Disclosed is the success use of the disclosed vectors on wild type and TSD fibroblasts in vitro (See Examples) as well as in hexB^{-/-} mice in vivo (See examples). The results show that FIV(Hex) is capable of transducing murine cells with the β-hexosaminidase transgene, leading to disease amelioration as assessed by improvement of locomotive performance (See examples) and decrease of brain inflammation (See examples) after neonatal intraperitoneal administration in vivo.

As disclosed herein, vectors injected systemically to P2 pups can reach into the brain through a partially incomplete BBB and directly transduce cells therein; perivascular and periventricular cells, such as microglia, astrocytes and eppendymal cells are expected to be primarily infected. It is also likely that specific subsets of neurons can be infected that are considered proximal to vessels and/or ventricular spaces.

Disclosed herein are vectors and methods for neonatal gene therapy, as well as perinatal, and adult, that have been shown to be successful in vector transfer into the CNS via systemic administration. Disclosed herein is data showing the amelioration of the clinical symptoms and the neuro-inflammation associated with GM₂ gangliosidosis after systemic administration of FIV(Hex) to hexB^{-/-} neonates (see examples). Interestingly, normal bone marrow transplantation (BMT) to hexB^{-/-} mice at neonatal stages of development was also shown to ameliorate neuro-inflammation, in a fashion similar to the FIV(Hex) treatment, and to extend life expectancy without achieving therapeutic levels of β-hexosaminidase activity in the brain of treated animals (Norflus F. et al., Journal of Clinical Investigation. 101(9):1881-8, 1998). The authors concluded that provision of normal bone marrow-derived cells limited the attendant brain inflammation, ultimately leading to disease amelioration. However, disclosed herein, in neonatal administrative situations, the use of the disclosed vectors as a platform of gene therapy cause transduction of the target brain cells, whereas young (4-5 weeks old) adult mice resulted in transduction of peripheral immune cells (monocytes, lymphocytes) that appeared to infiltrate and possibly engraft into the brain (As disclosed herein).

b) Peripherally Administered FIV Vectors can Transduce Blood Cells Systemically in Adults

As disclosed herein bone marrow-derived cells transduced with the β-hexosaminidase transgene can infiltrate into the CNS of hexB^{-/-} mice following intraperitoneal administration when administered systemically to an adult. The bone marrow contains myeloid progenitor cells capable of yielding a multitude of peripheral immune cells. Since FIV is a lentivirus capable of transgene integration, the cells derived from these progenitor cells can also carry the β-hexosaminidase transgene, and therefore will be detectable in the hexB^{-/-} brain. Therefore, detection of β-hexosaminidase-positive neurons in the brains of hexB^{-/-} mice can suggest that either PBMC can give rise to neurons de novo, or that PBMC are capable of transferring their genetic material to neurons following cell-cell fusion. Cell-cell fusion can be detected by fluorescent in situ hybridization (FISH). Disclosed herein, PBMC, can be used as vehicles for the transfer of therapeutic genes into the CNS, particularly when delivered to adult, in particular, subjects having a fully developed immune system and BBB.

Disclosed is the interaction of neuron-microglia in a mouse model of GM₂ gangliosidosis, and its role in disease development. Disclosed herein GM₂ neuronal storage induces microglia activation, which in turn elicits a cascade of proinflammatory cytokines, powerful mediators of CNS inflammation. Consequently, PBMC are recruited into the brain which further exacerbate brain inflammation and neurodegeneration. Since many of the lysosomal storage disorders share to some degree common pathogenetic pathways, vectors and methods disclosed herein can be used for other storage disorders that display CNS pathology using their cognate genes of interest. It is also interesting that “storage”-induced microglia activation and brain inflammation have also been implicated in the development of other neurodegenerative disorders, such as Alzheimer's disease (Lombardi et al. J Neurosci Res 54: 539-53 (1998); Szpak et al., 2001).

Disclosed are nucleic acids comprising sequence encoding HEX-α and sequence encoding HEX-β. Also disclosed are nucleic acids, wherein the nucleic acid further comprises an IRES sequence, wherein the nucleic acids express more than one IRES sequence, wherein the vectors express an IRES sequence after each Hex nucleics acid, wherein the nucleic acid further comprises a promoter sequence, wherein the nucleic acid further comprises a promoter sequence, wherein the HEX-β has at least 80% identity to the sequence set forth in SEQ ID NO:3 and the HEX-α has at least 80% identity to the sequence set forth in SEQ ID NO:1, wherein the HEX-β has at least 85% identity to the sequence set forth in SEQ ID NO:3 and the HEX-α has at least 80% identity to the sequence set forth in SEQ ID NO:1, wherein the HEX-β has at least 90% identity to the sequence set forth in SEQ ID NO:3 and the HEX-α has at least 80% identity to the sequence set forth in SEQ ID NO:1, wherein the HEX-β has at least 95% identity to the sequence set forth in SEQ ID NO:3 and the HEX-α has at least 80% identity to the sequence set forth in SEQ ID NO:1, wherein the HEX-β has the sequence set forth in SEQ ID NO:3 and the HEX-α has the sequence set forth in SEQ ID NO:1, wherein the sequence encoding the HEX-β is orientated 5′ to the sequence encoding HEX-α, wherein the sequence encoding the HEX-β is orientated 5′ to the IRES sequence and the IRES sequence is located 5′ to the sequence encoding HEX-α, wherein the promoter is located 5′ to the sequence encoding the HEX-β and the sequence encoding the HEX-β is orientated 5′ to the IRES sequence and the IRES sequence is located 5′ to the sequence encoding HEX-α.

Also disclosed are vectors comprising the disclosed nucleic acids. Also disclosed are cells comprising the disclosed nucleic acids and vectors.

Also disclosed are non-human mammal comprising the disclosed nucleic acids, vectors, and cells disclosed herein.

Also disclosed are methods of providing HEX-α in a cell comprising transfecting the cell with the nucleic acids, also disclosed are methods of providing HEX-β in a cell comprising transfecting the cell with the nucleic acids, also disclosed are method of providing HEX-α and HEX-β in a cell comprising transfecting the cell with the nucleic acid of claims 1-4.

Also disclosed are method of delivering the disclosed compositions, wherein the transfection occurs in vitro or in vivo.

Disclosed are methods of making a transgenic organism comprising administering the disclosed nucleic acids, vectors and/or cells.

Disclosed are methods of making a transgenic organism comprising transfecting a lentiviral vector to the organism at during a perinatal stage of the organism's development.

Also disclosed are methods of treating a subject having Tay Sachs disease and/or Sandoff disease comprising administering any of the disclosed compounds and compositions.

Also disclosed are vectors, that have for example, the beta globin promoter, the COLL1 promoter, or the NSE promoter, such as —FIV(βact-Hex), a vector that will restore β-hexosaminidase in all types of cells (pancellular promoter), FIV(COLL1-Hex), a vector that will restore β-hexosaminidase in osteoblasts/osteocytes, chondrocytes, fibroblasts and other mesenchymal-derived cells or -FIV(NSE-Hex), a vector that will restore β-hexosaminidase selectively in neurons, respectively.

Also disclosed are compositions where the promoter comprises a cell specific promoter. The cell specific promoter can comprise the Nuclear enolase specific (NSE) promoter (SEQ ID NO:69) or the COLL1A1 promoter (SEQ ID NO:70 and 71).

Disclosed are methods of delivering a nucleic acid to a brain central nervous system cell comprising systemically administering a vector to the subject, wherein the vector transduces a blood cell, and wherein the blood cell fuses with a brain cell.

Also disclosed are methods wherein the blood cell comprises a blood progenitor cell, a marker for a blood progenitor cell, an endothelial cell, a marker for an endothelial cell, endothelial cell comprises a marker, wherein the marker is CD31, a microglia cell, a marker for a microglia cell, a monocyte cell, a marker for a monocyte cell, a macrophage, a marker for a macrophage cell, a marker wherein the marker is CD11b a lymphocyte cell a marker for a lymphocyte cell, or wherein the marker is CD3.

Herein, “blood cell” refers to any cellular structure which is typically present in the blood. Such cells can include, for example, erythrocytes (i.e., red blood cells), thromobocytes (i.e., platelets), and leukocytes (i.e., white blood cells) which includes monocytes, lymphocytes (including B and T lymphocytes), and granulocytes (e.g., basophils, eosinophils, and neutrophils). It is understood that blood cells may not only be resident in the blood also being present in other tissues or organs such as the spleen, lymph nodes, liver, thymus, bone marrow. For example, T lymphocytes may be present in the blood and the spleen.

Herein, “blood progenitor cell” refers to any cellular structure that has the potential to develop into a “blood cell.” Such cells include but are not limited to hematopoietic stem cells or other such pluripotent cells, lymphoid progenitor cells, myeloid progenitor cells, megakaryocyte/erythroid progenitors cells, and granulocyte/macrophage progenitor cells. Thus, a blood progenitor cell is not a terminal stage cell. Blood progenitor cells can express various surface markers depending on the particular cell. In one example, human hematopoietic stem cells and pluripotent progenitors are CD34+ (positive) and CD38-(negative) and notably negative for markers that are specific for particular developmental lineages as well as being negative for CD33, CD45RA, and HLA-DR. However, though largely possessing all the characteristics of HSC, lymphoid progenitor cells are CD34+ (positive) and CD38+ (positive). Developmentally committed cells express lineage specific markers such as, for example, CD3 (T lymphocytes), CD19 and CD20 (B cells), CD14 (Monocytes), and CD66b (granulocytes). It is understood that many lineage specific markers are known in the art and those of skill in the art will recognize the presence or absence of such markers.

Herein, “endothelial cell” refers to cells of the endothelium that serve as a selective barrier to molecules moving between the blood and surrounding tissue. Endothelial cells, in addition, to regulating transmission of molecules into and out of the blood, also serve to help regulate the movement of lymphocytes. Endothelial cells can be identified by the presence of cell surface markers CD144 (VE Cadherin) and CD141 (thrombomodulin). Other examples of cell surface markers may be present in conjunction with CD141 or CD144 such as, for example, CD33 and CD54.

“Monocytes” refers to a lineage stage of leukocytes that gives rise to “macrophage,” myeloid dendritic cells, osteoclasts, and “microglia cells.” Herein, macrophages are tissue resident cells providing nonspecific cytokine production, phagocytosis, and antigen presentation to lymphocytes. Some surface markers typically associated with macrophages include but are not limited to CD11b and CD45. Herein, “microglia cell” refers to neuronal resident monocyte lineage cells capable of phagocytosis and cytokine secretion. “Microglia” serve to remove apoptotic cells in neuronal tissue and can serve to provide immune protection. Typically, microglia cells are CD45+ (positive) CD11b+ (positive) cells.

Herein, “lymphocytes” refers to lymphoid lineage cells that are responsible for the acquired immune responses within a subject. Lymphoid cells include B cells, which provide humoral immune responses (i.e., antibody production) and both CD4 and CD8 T cells, which are responsible for cell-mediated responses (i.e., cytokine secretion and CTL activity). B lymphocytes are associated with co-stimulatory signaling necessary to activate T cells as well as providing humoral immune responses via the antibody secretion of plasma cells. Markers for B cells include but are not limited to surface immunoglobulin, CD19, CD20, CD21 (CR2), CD40, CD81 (TAPA-1), B7-1, and B7-2. T lymphocytes (i.e., CD4 and CD8 T cells) recognize intracellular antigen in the context of peptide-Major Histocampatibility Complex (MHC) complexes. T lymphocytes provide cytolytic activity in the form of perforin and granzyme secretion, and secrete cytokines such as IFN-γ, TNF-α, IL-2, IL-4, and IL-10 in response to antigen. Markers for T cells include but are not limited to CD3, CD4, CD8, CD40L, CTLA-4, and CD28.

Disclosed are methods where the brain cell comprises a purkinje cell or a marker for a purkinje cell, wherein the markers are calbindin for Pukinje cerebellar cells and neurofilaments, NeuN for neurons; GFAP for astrocytes; CD11b & MHC-II for microglia; or PECAM-1 for endothelial cells.

Also disclosed are methods further comprising, adding the vector to a blood cell ex vivo producing a transduced blood cell, and administering the transduced blood cell to the subject.

Also disclosed are methods, wherein the blood cell comprises a blood cell obtained from the subject or is derived from a blood cell obtained from the subject.

Disclosed are methods for delivering a vector to a brain cell comprising, administering the vector to a subject, wherein the vector directly transduces the brain cell.

Disclosed are methods wherein the compositions are administered to a perinatal or neonatal subject, or adult subject, such as a young adult subject, such as a mouse or a human subject. In a mouse perinatal means from the last 2 embryonic days to seven days postnatal. In humans, perinatal means from 22 weeks of gestation to 28 days of life after birth. In mice, for example, a neonate can be from birth to 7 days of life. In a human, a neonatal is from birth to 28 days after birth. In a mouse, a young mouse can be 3-5 weeks of age. In a human a young adult can be from 18-25 years of age. Neonatal is included in Perinatal. It is the latter's postnatal period. Perinatal includes, therefore, prenatal gene therapy. Thus, also included would be 2^{nd} trimester gene therapy for pregnant women carrying an affected child.

It is understood that in certain embodiments, methods are performed prior to a fully functioning immune system as described herein. This can correlate somewhat with, for example, being a neonate. Also disclosed are methods, wherein the compostions are administered prior to a fully functioning or formed BBB, as described herein. This to, can for example, correlate with, for example, a neonate stage of development. For example, in humans, the immune system takes about 6 months to one year to mature, whereas in the mouse it may mature in 7-10 days. The blood brain barrier in the humans is typically formed almost completely by birth, as in the mouse. However, disclosed herein the data show that disclosed compositions, such as the FIVHEX vectors, can enter even at the second day of life of the mouse, indicating that the BBB is not yet completely formed or is inhibitory to the disclose vectors.

Also disclosed are methods, wherein the brain cell is a brain cortex cell, a brain basal ganglia cell, a brain thalamus cell, a brain cerebellum cell, or a brain stem cell.

Also disclosed are methods, wherein the administration of the vector comprises less than or equal to 10³, 10⁴, 10⁵, 10⁶, 10⁷, 10⁸, infectious particles, for example. Also disclosed are methods, wherein the administration of the vector comprises greater than or equal to 10³, 10⁴, 10⁵, 10⁶, 10⁷, 10⁸, infectious particles, for example.

Also disclosed are methods, wherein the administration of the vector comprises a m.o.i of about 1, 2, or 3, for example. This refers to the multiplicity of infection: the number of virions per cell: i.e. 10E5 virions treating 5×10E4 cells is m.o.i.=2.

Also disclosed are methods, wherein the vector reduces the inflammation of the brain. This can be determined, by for example, looking for cells positive for inflammatory markers, such as GFAP and MHC-II, as well as by the transcript levels of inflammatory genes (TNFα, IL-1β, IL-6) as discussed herein.

Also disclosed are methods, wherein the vector reduces the deteriation of motor function due to a lysomal storage disease. This can be determined, by for example, by two methods: (1) motorod performance and (2) inverted mesh test discussed herein.

13. GM2 Storage in Microglia Activation and Brain Inflammation

Neuronal GM₂ gangliosidosis secondary to β-hexosaminidase deficiency leads to microglia activation and brain inflammation, critical factors associated with neurodegeneration and disease development. This can be shown by restoring β-hexosaminidase deficiency selectively in neurons of transgenic mice while the neuron specific enolase promoter drives the expression of the therapeutic gene NSE-βHex (See examples). This strategy can lead to resolution of GM₂ neuronal storage on a hexB^{-/-} background. Consequently, deterrence of GM₂ storage will attenuate microglia activation and prevent brain inflammation, and subsequently ameliorate neuronal degeneration and disease development.

The NSE promoter has been successfully employed in the past and results in neuronal expression at readily detectable levels (Kearne et al., 2001). The NSE promoter can restore β-hexosaminidase expression in neurons at constitutive levels similar to those produced by the human β-hexosaminidase promoter (Norflus et al., 1996). Should the levels of transgene expression prove lower than desired to achieve GM₂ clearance in neurons, the mating strategy can be modified to produce mice homozygous for the transgene (instead of the heterozygous state set forth in the Examples, simply by breeding heterozygous founders and selecting for homozygous progeny). Based on data disclosed herein, the expression of human HexB and HexA by the βHex transgene in mammalian cells, such as murine or human, cells can result in synthesis of functional β-hexosaminidase capable of metabolizing GM₂ ganglioside.

14. Determining the Role Peripheral Blood Mononuclear Cells in GM₂ Gangliosidosis

Disclosed herein GM₂-induced microglia activation results in the recruitment of PBMC into the brain parenchyma, whereby brain inflammation is further enhanced and disease exacerbated. The former can be additionally shown by determining whether PBMC are recruited in the brain following GM₂ neuronal storage. This can further provide information on the role of PBMC in exacerbating disease development by entering into the brain parenchyma, or whether PBMC mode of action is in fact a peripheral effect. Lastly, disclosed herein one can determine whether PBMC transduced with the therapeutic gene βHex can have anti-inflammatory and neuro-protective effects (ex vivo therapy) similar to what has been previously described in hexB−/− mice following normal bone marrow transplantation (Norflus et al. J Clin Invest 101: 1881-1888 (1998). It is also interesting to determine the level of β-hexosaminidase restoration in the brain of treated and control mice in relation to the wild type animals, and its effects on GM₂ neuronal storage and disease development.

Inhibition of PBMC infiltration into the brain of animals with GM₂ storage will ameliorate disease development by attenuating brain inflammation. This is consistent with the critical role of MCP-1 in recruiting PBMC to central and peripheral sites of inflammation, including the brain (Koch et al. J Clin Invest 90: 772-9 (1992); Ransohoff et al. FASEB 7: 592-600 (1993); Izikson et al. Clin Immunol 103:125-131 (2002)). Absence of MCP-1 chemokine results in amelioration of experimental acute encephalitis in the mouse, similarly to the use of anti MCP-1 neutralizing antibodies (Huand et al., 2001). Furthermore, data disclosed herein showed significantly decreased recruitment of myeloid-derived cells following brain “trauma” in a CCR2^{-/-} knockout mouse (CCR2 is the receptor for MCP-1 chemokine). Due to the known redundancy in the chemoattraction system that significant PBMC infiltration may occur in the brain despite the lack of MCP-1. In this case, one can inhibit the inflammatory action of PBMC by the systemic administration of minocycline, a second generation tetracycline derivative with known anti-inflammatory effects and neuroprotective effects in addition to its anti-microbial action (Yrjanheikki et al. Proc Natl Acad Sci USA 95: 15769-74 (1998); Yrjanheikki et al. Proc Natl Acad Sci USA 96: 13496-500 (1999); Popovic et al. Ann Neurol 51: 215-23 (2002)).

Disclosed herein βHex-transduced PBMC will enter into the brain parenchyma, become engrafted and express β-hexosaminidase in therapeutic levels, attenuating brain inflammation and disease development in a manner similar to that observed after normal bone marrow transplantation Norflus et al. J Clin Invest 101: 1881-1888 (1998). Priller et al. (2003) recently demonstrated that gene-modified hematopoietic cells by a retrovirus infiltrated into the brain parenchyma insignificant numbers, where they expressed the reporter gene gfp and ultimately became engrafted. However, it is possible that the number of transduced cells or the level of β-Hex expression in bone marrow cells are not adequate for disease amelioration due to low transduction efficacy. Data disclosed herein FIV vectors indicates high levels of infectivity for microglia/monocytes (data not shown) by lentiviral vectors, it is possible to increase transduction efficacy by employing an HIV-derived lentiviral vector (Invitrogen) that allows for selection of successfully transduced cells by the drug blasticidin. Disclosed herein is the HIV(βact-Hex), and this vector can be used in combination with blasticidin. Moreover, one can increase recruitment of transduced PBMC into the brain by adding a third open reading frame to βHex encoding for the C-C receptor on the cell membrane (HexB-IRES-HexA-IRES-CCR). (See vector descriptions herein).

Promoter selection is also important. Disclosed are CMV—as well as a chicken β-actin/CMV fusion (βact)—driven βHex gene. Although CMV is a very strong promoter. The βact promoter also has shown high levels of HexA and HexB expression disclosed herein (See Examples), and in addition offers the advantage of long-term expression in rodents (4-12 months; Daly et al. Hum Gene Ther 10: 85-94 (1999); Daly et al. Gene Ther 8: 1291-8 (2001)). The CD11b promoter is an alternative choice, which is also characterized by high levels of expression selectively in monocytes/macrophages (Dziennies et al., 1995).

### C. COMPOSITIONS

1. β-Hexosaminidase Transgene (β-Hex)

The β-Hexosaminidase protein is a protein comprised of two subunits, one subunit is encoded by the HexA gene and a second subunit encoded by the gene HexB. The human HexA Exon 1 can be found 316 bp upstream of MstII site; chromosome 15q11-15qter. The human HexA gene can be found at human chromosomal region 15q23----q24. The human HexB gene can be found on chromosome 5, map 5q13.

Disclosed are constructs capable of expressing both the HexA gene product and the HexB gene product, from a single construct. Any construct capable of expressing both the HexA and HexB gene products is referred to as a β-Hex construct herein. The β-Hex construct allows for synthesis of all β-hexosaminidase protein isoforms, HEXA (α/β heterodimer), HEXB (β/β homodimer) and HEXS (α/α homodimer). Disclosed are nucleic acid constructs comprising a cytomegalovirus (CMV) promoter-driven bicistronic gene (β-Hex) that encodes for both human HexA and HexB genes, which can lead to the synthesis of functional β-hexosaminidase isoenzymes.

The β-Hex construct typically comprises four parts: 1) a promoter, 2) the HexA coding sequence, 3) the HexB coding sequence, and 4) an IRES sequence (integrated ribosomal entry site). These four parts can be integrated into any vector delivery system. In preferred embodiments, the orientation of the four parts is 5′-promoter-HexB-IRES-HexA-3′.

The promoter can be any promoter, such as those discussed herein. It is understood as discussed herein that there are functional variants of the HexA and HexB which can be made. Furthermore, it is understood that that there are functional variants of the IRES element, for example as discussed herein. Typically the genes to be expressed are placed on either side of the IRES sequence.

The IRES element is an internal ribosomal entry sequence which can be iosolated from the encephalomyocarditis crius (ECMV). This element allows multiple genes to be expressed and correctly translated when the genes are on the same construct. IRES sequences are discussed in for example, U.S. Pat. No. 4,937,190 which is herein incorporated by reference at least for material related to IRES sequences and their use.

HexA and HexB cDNA can be obtained from the American Tissue Culture Collection. (American Tissue Culture Collection, Manassas, Va. 20110-2209; Hex-α: ATCC# 57206; Hex-β ATCC# 57350) The IRES sequence can be obtained from a number of sources including commercial sources, such as the pIRES expressing vector from Clonetech (Clontech, Palo Alto Calif. 94303-4230).

Also disclosed are tricistronic constructs encoding for both isoforms of human β-hexosaminidase, hHexA & hHexB, as well as the β-galactosidase reporter gene (lacZ), or some other marker or reporter gene.

Global delivery of the disclosed constructs is also disclosed. Disclosed is a pseudotyped feline immunodeficiency virus (FIV) for global β-Hex delivery. Stable expression of the therapeutic gene aids prolonged restoration of the genetic anomaly enhancing treatment efficacy and contributing to long-term therapeutic outcomes. The backbone FIV system has been shown to effectively incorporate, due to its lentiviral properties, the transgene of interest into the host's genome, allowing for stable gene expression (Poeschla E M, et al., Nature Medicine 4: 354-357. (1998)). Disclosed herein is stable expression of the reporter gene lacZ for over 3 months in mice following perinatal systemic FIV(lacZ) administration.

A model system for the study of these vectors is a mouse that is knockout mouse deficient in both HexA and HexB, since the hexA^{-/-}/hexB^{-/-} mouse is characterized by global disruption of the hexA and hexB genes. Gene disruption in this mouse is global, and therefore, can be used as a model for global replacement. The timing of gene therapy is important as it is closely related to the temporal development of the disorder. HexA^{-/-}/hexB^{-/-} mice display mild phenotype aberrations at birth and quickly develop craniofacial dysplasia by 4-5 weeks of age. Similarly, it is not uncommon for patients suffering from this class of genetic disorders to display only mild degree of the disease at infancy, and to progress to severe forms by adolescence.

In certain embodiments it is envisioned that diagnosis of children can occur in utero and a determination of affected children of a congenital disorder will be confirmed perinatally by molecular biology methods. Furthermore, these neonates could be treated with the disclosed constructs and methods at the initial stages of the disorder, so that the young patient would resume normal development.

2. Delivery of the Compositions to Cells

Delivery can be applied, in general, via local or systemic routes of administration. Local administration includes virus injection directly into the region or organ of interest, versus intravenous (IV) or intraperitoneal (IP) injections (systemic) aiming at viral delivery to multiple sites and organs via the blood circulation. Previous research on the effects of local administration demonstrated gene expression limited to the site/organ of the injection, which did not extend to the rest of the body (Daly T M, et al., Hum Gene Ther 10:85-94. (1999a); Kordower J H, et al., Exp Neurol 160:1-16. (1999)). Furthermore, previous studies have demonstrated successful global gene transfer to multiple tissues and organs in rodents and primates following viral IV and IP injections (Daly T M, et al., Proc Natl Acad Sci USA 96: 2296-2300. (1999b); Tarntal et al., 2001; McCormack et al., 2001; Lipschutz G S, et al., Mol Ther 3: 284-92. (2001)). Disclosed herein IP injection of FIV(lacZ) in mice of adult (3 months old) as well as of perinatal age (P4) resulted in global transfer and expression of the reporter gene lacZ in brain, liver, spleen and kidney. Also disclosed, the levels of expression achieved via IP injections were superior to those acquired following local administration directly into the liver.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Rain et al. Cancer Res. 53:83-88, (1993)).

As used herein, plasmid or viral vectors are agents that transport the disclosed nucleic acids, such as the β-Hex construct into the cell without degradation and include a promoter yielding expression of the HexA and HexB encoding sequences in the cells into which it is delivered. In some embodiments the vectors for the β-Hex constructs are derived from either a virus, retrovirus, or lentivirus. Viral vectors can be, for example, Adenovirus, Adeno-associated virus, Herpes virus, Vaccinia virus, Polio virus, AIDS virus, neuronal trophic virus, Sindbis and other RNA viruses, including these viruses with the HIV backbone, and lentiviruses. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene, such as, the disclosed β-Hex constructs or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector, which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B139 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Lentiviral Vectors

The vectors can be lentiviral vectors, including but not limited to, SIV vectors, HIV vectors or a hybrid construct of these vectors, including viruses with the HIV backbone. These vectors also include first, second and third generation lentiviruses. Third generation lentiviruses have lentiviral packaging genes split into at least 3 independent plasmids or constructs. Also vectors can be any viral family that share the properties of these viruses which make them suitable for use as vectors. Lentiviral vectors are a special type of retroviral vector which are typically characterized by having a long incubation period for infection. Furthermore, lentiviral vectors can infect non-dividing cells. Lentiviral vectors are based on the nucleic acid backbone of a virus from the lentiviral family of viruses. Typically, a lentiviral vector contains the 5′ and 3′ LTR regions of a lentivirus, such as SIV and HIV. Lentiviral vectors also typically contain the Rev Responsive Element (RRE) of a lentivirus, such as SIV and HIV.

(a) Feline Immunodeficiency Viral Vectors

One type of vector that the disclosed constructs can be delivered in is the VSV-G pseudotyped Feline Immunodeficiency Virus system developed by Poeschla et al. (Poeschla E M, et al., Nature Medicine 4: 354-357. (1998)). This lentivirus has been shown to efficiently infect dividing, growth arrested as well as post-mitotic cells. Furthermore, due to its lentiviral properties, it allows for incorporation of the transgene into the host's genome, leading to stable gene expression. This is a 3-vector system, whereby each confers distinct instructions: the FIV vector carries the transgene of interest and lentiviral apparatus with mutated packaging and envelope genes. A vesicular stomatitis virus G-glycoprotein vector (VSV-G; Burns J C, et al., Proc Natl Acad Scie USA 90: 8033-8037. (1993)) contributes to the formation of the viral envelope in trans. The third vector confers packaging instructions in trans (Poeschla E M, et al., Nature Medicine 4: 354-357. (1998)). FIV production is accomplished in vitro following co-transfection of the aforementioned vectors into 293-T cells. The FIV-rich supernatant is then collected, filtered and can be used directly or following concentration by centrifugation. Titers routinely range between 10⁴-10⁷ bfu/ml.

The use of defective FIV vectors is not associated with production of disease. The amounts of defective particles typically used in each experiment is less than 10⁶ infection particles per animal, such as mice. The defective virus can be loaded into microsyringes in a laminar flow hood and transferred to the location where animals or subjects can be injected.

(5) Packaging Vectors

As discussed above, retroviral vectors are based on retroviruses which contain a number of different sequence elements that control things as diverse as integration of the virus, replication of the integrated virus, replication of un-integrated virus, cellular invasion, and packaging of the virus into infectious particles. While the vectors in theory could contain all of their necessary elements, as well as an exogenous gene element (if the exogenous gene element is small enough) typically many of the necessary elements are removed. Since all of the packaging and replication components have been removed from the typical retroviral, including lentiviral, vectors which will be used within a subject, the vectors need to be packaged into the initial infectious particle through the use of packaging vectors and packaging cell lines. Typically retroviral vectors have been engineered so that the myriad functions of the retrovirus are separated onto at least two vectors, a packaging vector and a delivery vector. This type of system then requires the presence of all of the vectors providing all of the elements in the same cell before an infectious particle can be produced. The packaging vector typically carries the structural and replication genes derived from the retrovirus, and the delivery vector is the vector that carries the exogenous gene element that is preferably expressed in the target cell. These types of systems can split the packaging functions of the packaging vector into multiple vectors, e.g., third-generation lentivirus systems. Dull, T. et al., “A Third-generation lentivirus vector with a conditional packaging system” J. Virol 72(11):8463-71 (1998) Retroviruses typically contain an envelope protein (env). The Env protein is in essence the protein which surrounds the nucleic acid cargo. Furthermore cellular infection specificity is based on the particular Env protein associated with a typical retrovirus. In typical packaging vector/delivery vector systems, the Env protein is expressed from a separate vector than for example the protease (pro) or integrase (in) proteins.

(6) Packaging Cell Lines

The vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals. One type of packaging cell line is a 293 cell line.

(7) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed constructs or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology. 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described herein, the compositions can be administered in a pharmaceutically acceptable carrier and can be delivered to the subjects cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

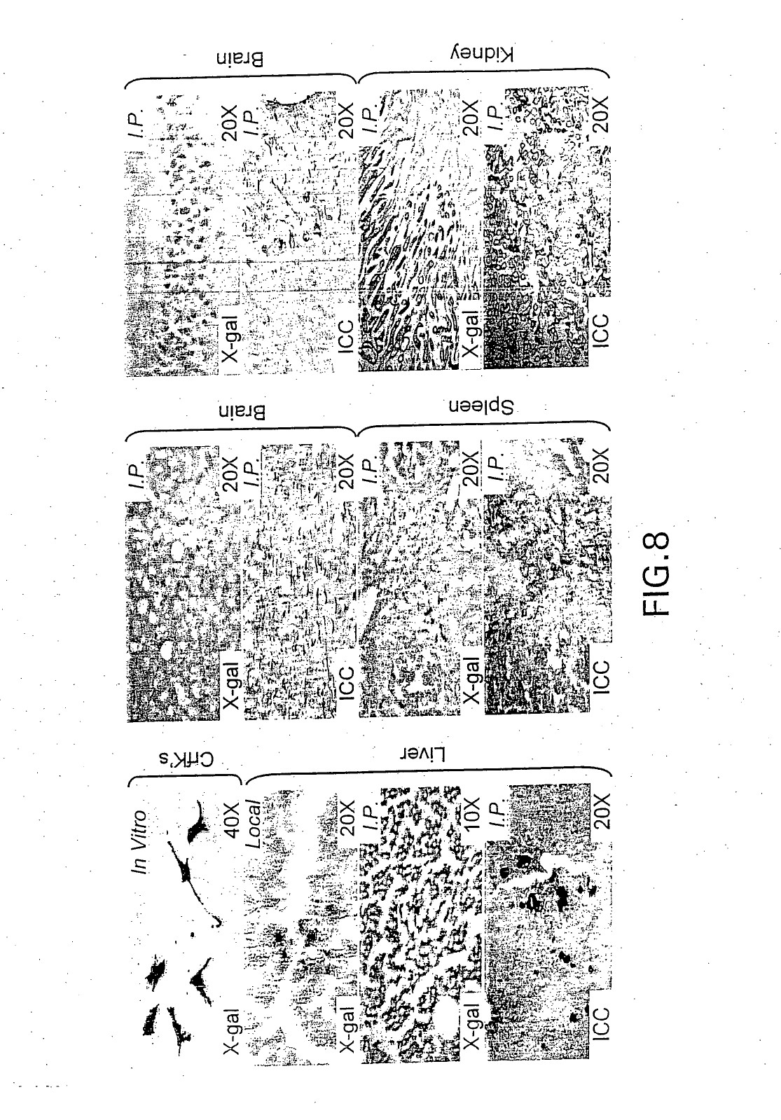

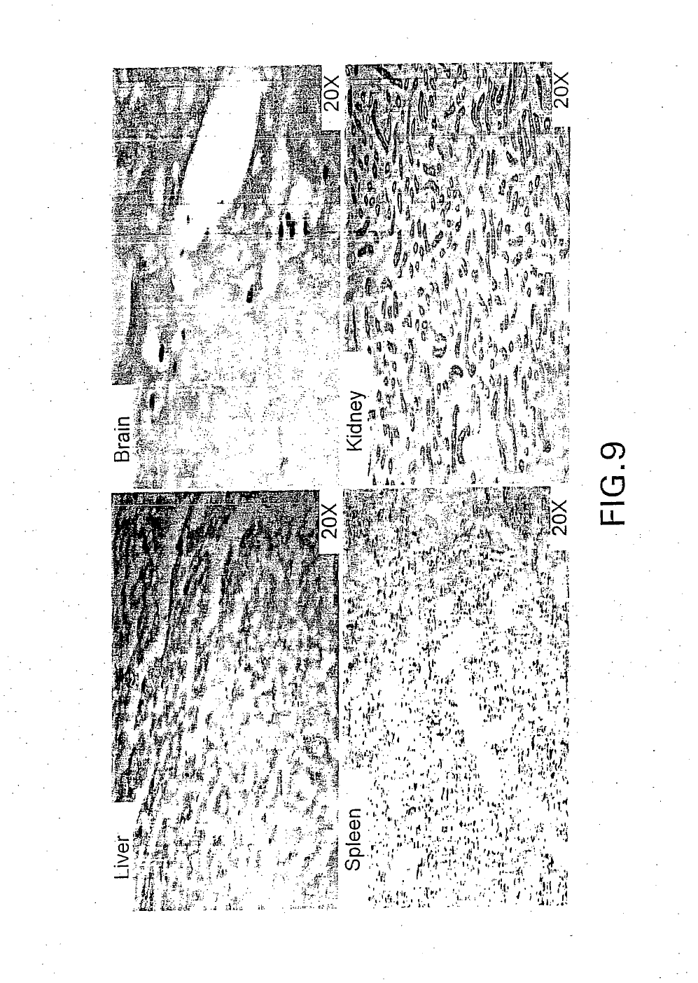

If in vivo delivery methods are performed the methods can be designed to deliver the nucleic acid constructs directly to a particular cell type, via any delivery mechanism, such as intra-peritoneal injection of a vector construct. In this type of delivery situation, the nucleic acid constructs can be delivered to any type of tissue, for example, brain or neural or muscle. The nucleic acid constructs can also be delivered such that they generally deliver the nucleic acid constructs to more than one type of cell. This type of delivery can be accomplished, by for example, injecting the constructs intraperitoneally into the flank of the organism. (See Example 2 and FIGS. 8-10). It in certain delivery methods, the timing of the delivery is monitored. For example, the nucleic acid constructs can be delivered at the perinatal stage of the recipients life or at the adult stage.

The disclosed compositions, can be delivered to any type of cell. For example, they can be delivered to any type of mammalian cell. Exemplary types of cells neuron, glia, fibroblast, chondrocyte, osteocyte, endothelial, and hepatocyte.

3. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Constitutive Promoters

In certain embodiments the promoters are constitutive promoters. This can be any promoter that causes transcription regulation in the absence of the addition of other factors. Examples of this type of promoter are the CMV promoter and the beta actin promoter, as well as others discussed herein. In certain embodiments the promoter can consist of fusions of one or more different types of promoters. For example, the regulatory regions of the CMV promoter and the beta actin promoter are well known and understood, examples, of which are disclosed herein. Parts of these promoters can be fused together to, for example, produce a CMV-beta actin fusion promoter, such as the one shown in SEQ ID NO:23. It is understood that this type of promoter has a CMV component and a beta actin component. These components can function independently as promoters, and thus, are themselves considered beta actin promoters and CMV promoters. A promoter can be any portion of a known promoter that causes promoter activity. It is well understood that many promoters, including the CMV and Beta Actin promoters have functional domains which are understood and that these can be used as a beta actin promoter or CMV promoter. Furthermore, these domains can be determined. For example, SEQ ID NO:s 21-41 display a number of CMV promoters, beta actin promoters, and fusion promoters. These promoters can be compared, and for example, functional regions delineated, as described herein. Furthermore, each of these sequences can function independently or together in any combination to provide a promoter region for the disclosed nucleic acids.

Other human cytomegalovirus promoter regions can be found in accession numbers M64940, Human cytomegalovirus IE-1 promoter region, M64944 Human cytomegalovirus IE-1 promoter region, M64943 Human cytomegalovirus IE-1 promoter region, M64942 Human cytomegalovirus IE-1 promoter region, M64941 Human cytomegalovirus IE-1 promoter region (All of which are herein incorporated by reference at least for their sequence and information)

c) Non-Constitutive Promoters

The promoters can also be non-constitutive promoters, such as cell specific promoters. These are promoters that are turned on at specific time in development or stage or a particular type of cell, such as a cardiac cell, or neural cell, or a bone cell. Some examples of cell specific promoters are, the neural enolase specific promoter, (NSE) the COLL1A1 procollagen promoter, and the CD11b promoter (PBMC-microglia/macrophage/monocyte specific promoter.

It is understood that the recombinant systems can be expressed in a tissue-specific manner. It is understood that tissue specific expression can occur due to the presence of a tissue-specific promoter. Typically, proteins under control of a tissue-specific promoter are transcribed when the promoter becomes active by virtue of being present in the tissue for which it is specific. Therefore, all cells can encode for a particular gene without global expression. As such, labeled proteins can be shown to be present in certain tissues without expression in other nearby tissues that may complicate results or expression of proteins in tissues where expression may be detrimental to the host. Disclosed are methods wherein the cre recombinase is under the control of the EIIA promoter, a promoter specific for breast tissue, such as the WAP promoter, a promoter specific for ovarian tissue, such as the ACTB promoter, or a promoter specific for bone tissue, such as osteocalcin. Any tissues specific promoter can be used. Promoters specific for prostate, testis, and neural are also disclosed. Examples of some tissue-specific promoters include but are not limited to MUC1, EIIA, ACTB, WAP, bHLH-EC2, HOXA-1, Alpha-fetoprotein (AFP), opsin, CR1/2, Fc-γ-Receptor 1 (Fc-γ-R1), MMTVD-LTR, the human insulin promoter, Pdha-2, rat neuron-specific enolase. For example, use of the AFP promoter creates specificity for the liver. Another example, HOXA-1 is a neuronal tissue specific promoter, and as such, proteins expressed under the control of HOXA-1 are only expressed in neuronal tissue.

d) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

e) Post Transcriptional Regulatory Elements

The disclosed vectors can also contain post-transcriptional regulatory elements. Post-transcriptional regulatory elements can enhance mRNA stability or enhance translation of the transcribed mRNA. An exemplary post-transcriptional regulatory sequence is the WPRE sequence isolated from the woodchuck hepatitis virus. (Zufferey R, et al., “Woodchuck hepatitis virus post-transcriptional regulatory element enhances expression of transgenes delivered by retroviral vectors,” J Virol; 73:2886-92 (1999)). Post-transcriptional regulatory elements can be positioned both 3′ and 5′ to the exogenous gene, but it is preferred that they are positioned 3′ to the exogenous gene.

f) Transduction Efficiency Elements

Transduction efficiency elements are sequences that enhance the packaging and transduction of the vector. These elements typically contain polypurine sequences. An example of a transduction efficiency element is the ppt-cts sequence that contains the central polypurine tract (ppt) and central terminal site (cts) from the HIV-1 pSG3 molecular clone (SEQ ID NO:1 bp 4327 to 4483 of HIV-1 pSG3 clone).

g) 3′ Untranslated Regions

Expression vectors used in eukaryotic host cells (yeast, fungi, insect, plant, animal, human or nucleated cells) may also contain sequences necessary for the termination of transcription which may affect mRNA expression. These 3′ untranslated regions are transcribed as polyadenylated segments in the untranslated portion of the mRNA encoding the exogenous gene. The 3′ untranslated regions also include transcription termination sites. The transcription unit also can contain a polyadenylation region. One benefit of this region is that it increases the likelihood that the transcribed unit will be processed and transported like mRNA. The identification and use of polyadenylation signals in expression constructs is well established. Homologous polyadenylation signals can be used in the transgene constructs. In an embodiment of the transcription unit, the polyadenylation region is derived from the SV40 early polyadenylation signal and consists of about 400 bases. Transcribed units can contain other standard sequences alone or in combination with the above sequences improve expression from, or stability of, the construct.

4. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

5. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

6. Nucleic Acids

There are a variety of molecules disclosed herein that are nucleic acid based, including for example the nucleic acids that encode, for example HexA and HexB, or functional nucleic acids. The disclosed nucleic acids can be made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

a) Sequences

There are a variety of sequences related to the HexA, HexB, IRES sequences, and promoter sequences. For example, the HexA and hexB genes have the following Genbank Accession Numbers: M16411 and NM_{—}000520 for HexA and NM_{—}000521 for HexB, these sequences and others are herein incorporated by reference in their entireties as well as for individual subsequences contained therein. It is understood that there are numerous Genbank accession sequences related to HexA and HexB, all of which are incorporated by reference herein.

One particular sequence set forth in SEQ ID NO:4 and having Genbank accession number NM_{—}000521, which is a sequence for human HexB cDNA, is used herein, as an example, to exemplify the disclosed compositions and methods. It is understood that the description related to this sequence is applicable to any sequence related to HexA or HexB unless specifically indicated otherwise. Those of skill in the art understand how to resolve sequence discrepancies and differences and to adjust the compositions and methods relating to a particular sequence to other related sequences. Primers and/or probes can be designed for any of the sequences disclosed herein given the information disclosed herein and that known in the art.

It is also understood for example that there are numerous bicistronic vectors that can be used to create the β-Hex construct nucleic acids See for example, Genbank accession no Y11035 and Y11034.

b) Primers and Probes

Disclosed are compositions including primers and probes, which are capable of interacting with, for example, the β-Hex construct nucleic acids, as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with, for example, the β-Hex construct nucleic acid, or region of the β-Hex construct nucleic acids or they hybridize with the complement of the β-Hex construct nucleic acids or complement of a region of the β-Hex construct nucleic acids.

7. Peptides

a) Protein Variants

As discussed herein there are numerous variants of the HEX-α and HEX-β proteins that are known and herein contemplated. In addition, to the known functional species and allelic variants of HEX-α and HEX-β there are derivatives of the HEX-α and HEX-β proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| alanine | Ala | A |
| allosoleucine | AIle | |
| arginine | Arg | R |
| asparagine | Asn | N |
| aspartic acid | Asp | D |
| cysteine | Cys | C |
| glutamic acid | Glu | E |
| glutamine | Gln | Q |
| glycine | Gly | G |
| histidine | His | H |
| isolelucine | Ile | I |
| leucine | Leu | L |
| lysine | Lys | K |
| phenylalanine | Phe | F |
| proline | Pro | P |
| pyroglutamic acid | pGlu | |
| serine | Ser | S |
| threonine | Thr | T |
| tyrosine | Tyr | Y |
| tryptophan | Trp | W |
| valine | Val | V |

TABLE 2
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:1 sets forth a particular sequence of HEX-α and SEQ ID NO:3 sets forth a particular sequence of a HEX-protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:3 is set forth in SEQ ID NO:4. Another nucleic acid sequence that encodes the same protein sequence set forth in SEQ ID NO:3 is set forth in SEQ ID NO:11. In addition, for example, a disclosed conservative derivative of SEQ ID NO:3 is shown in SEQ ID NO:12, where the valine (V) at position 21 is changed to a isoleucine (I). It is understood that for this mutation all of the nucleic acid sequences that encode this particular derivative of the SEQ ID NO:3 polypeptide are also disclosed. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

8. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

9. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

10. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

11. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

The disclosed viral vectors can be made using standard recombinant molecular biology techniques. Many of these techniques are illustrated in Maniatis (Maniatis et al., “Molecular Cloning—A Laboratory Manual,” (Cold Spring Harbor Laboratory, Latest edition) and Sambrook et al., Molecular Cloning: A Laboratory Manual, 2nd Ed., Cold Spring Harbor Laboratory, Cold Spring Harbor, N.Y., 1989.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W. H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Processes for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a promoter element, a HexB element, a IRES element, and a HexA element.

Disclosed are nucleic acid molecules produced by the process comprising linking in an operative way nucleic acid molecules comprising sequences set forth in SEQ ID NO:10 and SEQ ID NO:4.

Also disclosed are nucleic acid molecules produced by the process comprising linking in an operative way nucleic acid molecules comprising sequences having 80% identity to sequences set forth in SEQ ID NO:10 and SEQ ID NO:4.

Also disclosed are nucleic acid molecules produced by the process comprising linking in an operative way nucleic acid molecules comprising sequences that hybridizes under stringent hybridization conditions to sequences set forth in SEQ ID NO: 10 and SEQ ID NO:4.

Disclosed are nucleic acid molecules produced by the process comprising linking in an operative way a nucleic acid molecule comprising a sequence encoding HEX-β and HEX-α peptides and a sequence controlling an expression of the sequence encoding HEX-β and HEX-α.

Disclosed are nucleic acid molecules produced by the process comprising linking in an operative way a nucleic acid molecule comprising a sequence encoding HEX-β and HEX-α peptides wherein the HEX-β and HEX-α peptides have 80% identity to the peptides set forth in SEQ ID NO:1 and SEQ ID NO:3 and a sequence controlling expression of the sequences encoding the peptides.

Disclosed are nucleic acid molecules produced by the process comprising linking in an operative way a nucleic acid molecule comprising a sequence encoding HEX-β and HEX-α peptides wherein the HEX-β and HEX-α peptides have 80% identity to the peptides set forth in SEQ ID NO:1 and SEQ ID NO:3, wherein any change from the sequences set forth in SEQ ID NO:1 and SEQ ID NO:3 are conservative changes and a sequence controlling expression of the sequences encoding the peptides. Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate. Also disclosed are mammals wherein mammal is a murine, ungulate, or non-human primate.

Also disclose are animals produced by the process of adding to the animal any of the cells disclosed herein.

### E. METHODS OF USING THE COMPOSITIONS

1. Methods of Using the Compositions as Research Tools

The disclosed compositions can be used in a variety of ways as research tools. For example, the disclosed compositions, the β-Hex constructs, and other nucleic acids, such as SEQ ID NOs:10 and 4 can be used to produce organisms, such as transgenic or knockout mice, which can be used as model systems for the study of Tay Sachs and Sandoffs disease.

2. Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination event occurs at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

3. Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

Following administration of a disclosed composition, such as the disclosed constructs, for treating, inhibiting, or preventing Tay Sachs or Sandoffs disease, the efficacy of the therapeutic construct can be assessed in various ways well known to the skilled practitioner. For instance, one of ordinary skill in the art will understand that a composition, such as the disclosed constructs, disclosed herein is efficacious in treating Tay Sachs or Sandoffs disease or inhibiting or reducing the effects of Tay Sachs or Sandoffs disease in a subject by observing that the composition reduces the onset of the conditions associated with these diseases. Furthermore, the amount of protein or transcript produced from the constructs can be analyzed using any diagnostic method. For example, it can be measured using polymerase chain reaction assays to detect the presence of construct nucleic acid or antibody assays to detect the presence of protein produced from the construct in a sample (e.g., but not limited to, blood or other cells, such as neural cells) from a subject or patient.

Disclosed herein, the administration, such as systemic administration of the disclosed vectors, including the vectors expressing the biscistronic HEX construct, can be delivered at anytime of the development of the subject. However, there are differences depending upon the timing of the developemental administration. For example, when the disclosed vectors are administered to a neonate, the vectors directly transfect neuronal cells. However, when the vectors are administered to an adult, the vectors typically transduce circulating immune progenitor cells which then fuse cross the blood brain barrier and fuse with neuronal cells.

These two mechanisms can account for the presence of transduced cells in the brain parenchyma after intraperitoneal FIV injection, shown herein. This can be shown by administering increasing doses of β-hexosaminidase FIV vectors, FIV(Hex), to hexB^{-/-} knockout mice intraperitoneally and examining the transduction of brain cells acutely after treatment (3-5-7 days) in order to avoid any secondary effects derived from infiltrating peripheral immune cells into the brain (response to dose). Since commencement of the murine BBB occurs during post-natal stages of development, the effects of FIV vector administration in neonatal versus adult mice can be compared.

Hematopoietic progenitor cells that are infected by FIV in the periphery and give rise to a multitude of transduced circulating immune cells, can enter and engraft into the CNS via the BBB. This can be shown by examining the fate of bone marrow-derived cells harvested from hexB^{-/-} donors as well as wild type mice after being transduced by FIV(Hex) and transplanted to hexB^{-/-} mice ex vivo. The sphere of distribution and the persistence of β-hexosaminidase expression can be examined in vivo by evaluating the presence and identity of transduced cells in the CNS and periphery (bone marrow and spleen) at 2, 4, 6, 12 and 16 weeks after treatment.

As disclosed herein therapeutic levels of β-hexosaminidase expression were achieved in the brain following systemic administration of lentiviral vectors. Since GM2 gangliosidoses are progressive disorders, whereby affected patients display mild abnormalities at infancy, but progress to severe forms in childhood, the results disclosed herein, are consistent with there being a critical window in post-natal development, during which systemic administration of the disclosed vectors can effectively restore β-hexosaminidase activity in the brain of hexB^{-/-} mice before irreparable damage in the brain occurs (neurodegeneration). This critical window will include neonates and earlier postnatal mice as shown herein. Administration to adults past this window, would not be without effect, but the effect will be much less. The spatial distribution of the β-hexosaminidase transgene in hexB^{-/-} knockout mice following intraperitoneal FIV(Hex) administration at post-natal day P2, 3 weeks and 3 months of age can be characterized. The effects of FIV(Hex) administration on lysosomal storage, neuronal cell death and behavioral performance in hexB−/− knockout mice after FIV(Hex) treatment in relation to the sphere of transgene distribution and the levels β-hexosaminidase expression can also be assessed as described herein. Furthermore, since the introduction of novel proteins to immunocompetent mice is anticipated to elicit an immunologic response, it is possible that FIV(Hex) injection may result in the generation of host antibodies against the transgenic proteins (HexB) that potentially can reduce therapeutic efficacy by neutralizing β-hexosaminidase activity. Therefore, the presence of antibodies against viral and transgenic proteins in the serum of hexB−/− knockout mice following FIV administration can be monitored.

### F. EXAMPLES

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Making #-Hex Constructs

a) Construction of Bicistronic β-Hex Construct

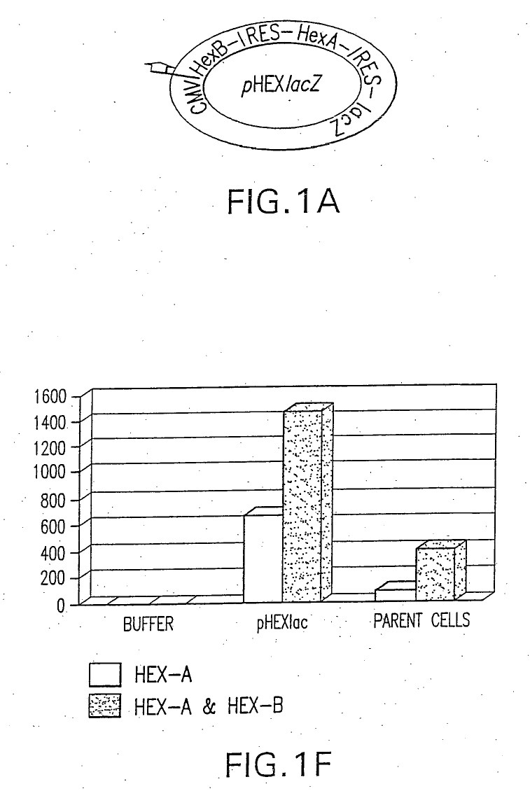

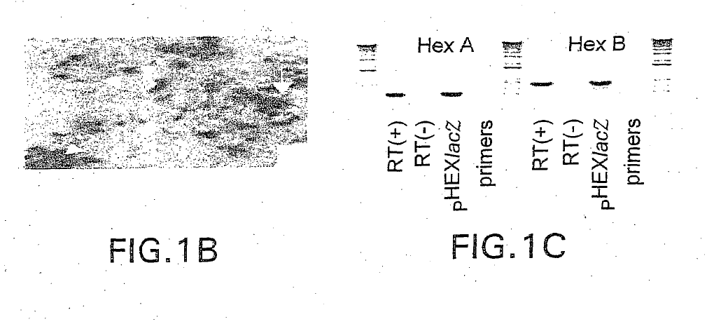

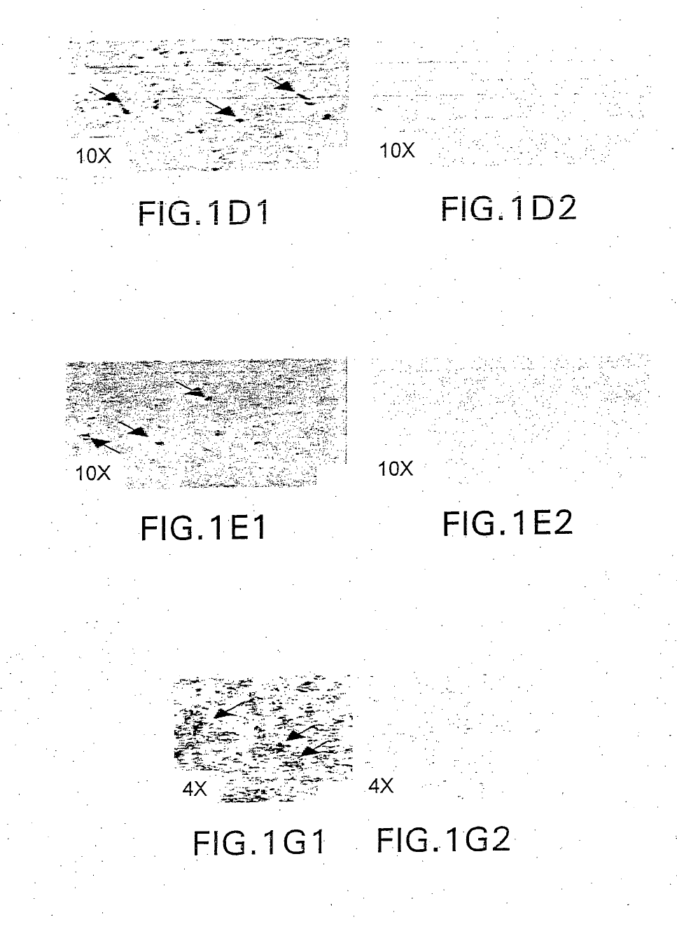

A bicistronic construct encoding for both isoforms of human β-hexosaminidase, hHexA and hHexB was made (FIG. 1). hHexB cDNA was isolated following Xho I digestion of pHexB43 (ATCC, Manassas Va.) and cloned into the Xho I site of pIRES (Clonetech Laboratories, Palo Alto Calif.) downstream of the vector's cytomegalovirus (CMV) promoter sequence. The HexA cDNA was isolated from pBHA-5 (ATCC, Manassas Va.) by Xho I digestion and was subsequently inserted into the Xba I site of pIRES(HexB) downstream of the vectors IRES cassette by blunt ligation. In this construct, the cytomegalovirus promoter (CMV) drives transgene expression, and the translation of the second open reading frame, HexB, is facilitated by an internal ribosomal entry sequence (IRES). In addition to above, a custom made IRES-lacZ cassette was also inserted downstream to HexA. In this construct, the cytomegalovirus promoter (CMV) drives transgene expression, and the expression of the second and third open reading frames (ORF), HexA and lacZ, respectively, are facilitated by an internal ribosomal entry sequence (IRES). Nevertheless, IRES-mediated translation has been shown to be reduced by about 40-50%. Hence, since HexB is necessary in the synthesis of both HEXA (α/β) and HEXB (β/β), the HexB ORF was cloned first in the transgene.

b) Results

(1) The Bicistronic Transgene Corrects Deficiencies In Vitro

The HEXlacZ encodes for both isoforms of human β-hexosaminidase, HexA & HexB. (FIG. 1) The vector pHEXlacZ is shown in FIG. 1(A). BHK^{HexlacZ} are developed by stable HexlacZ transduction. FIG. 1(B) shows that the cells transfected with the pHEXlacZ vector stain positively by X-gal histochemistry. Furthermore, HexA & HexB mRNA was detected by RT-PCR in total RNA extracts (FIG. 1(C)). Likewise, not only was transcript of pHEXlacZ vector identified, human HEXA and human HEXB proteins were detected in the transfected BHK^{HexlacZ} cells by immunocytochemistry. (FIGS. 1(D₁) and 1(E₁). This data indicates that the disclosed constructs can be expressed in target cells and that sufficient levels of protein are produced within these cells.

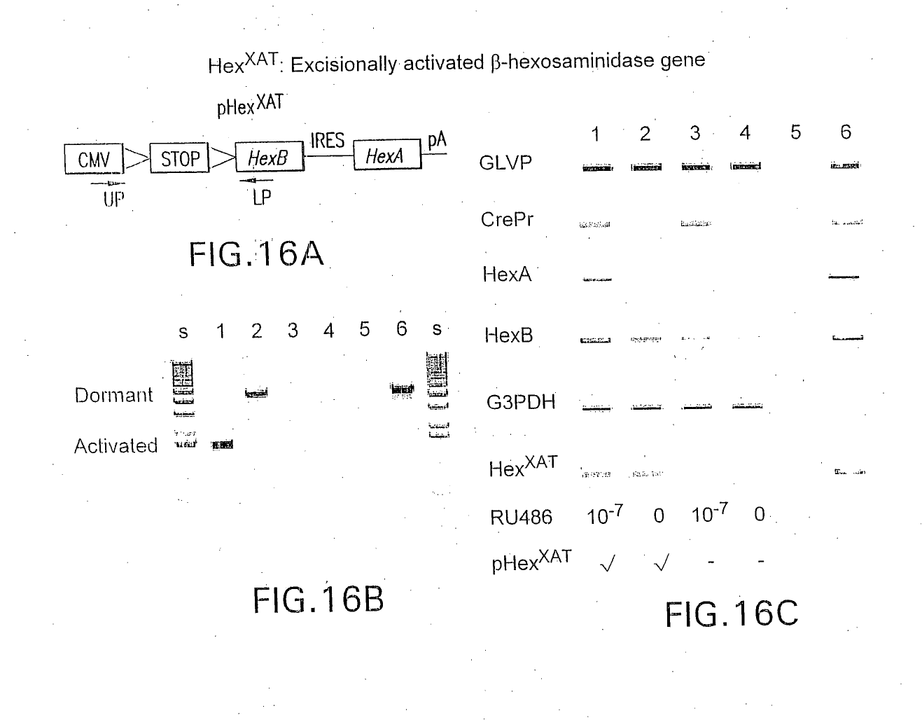

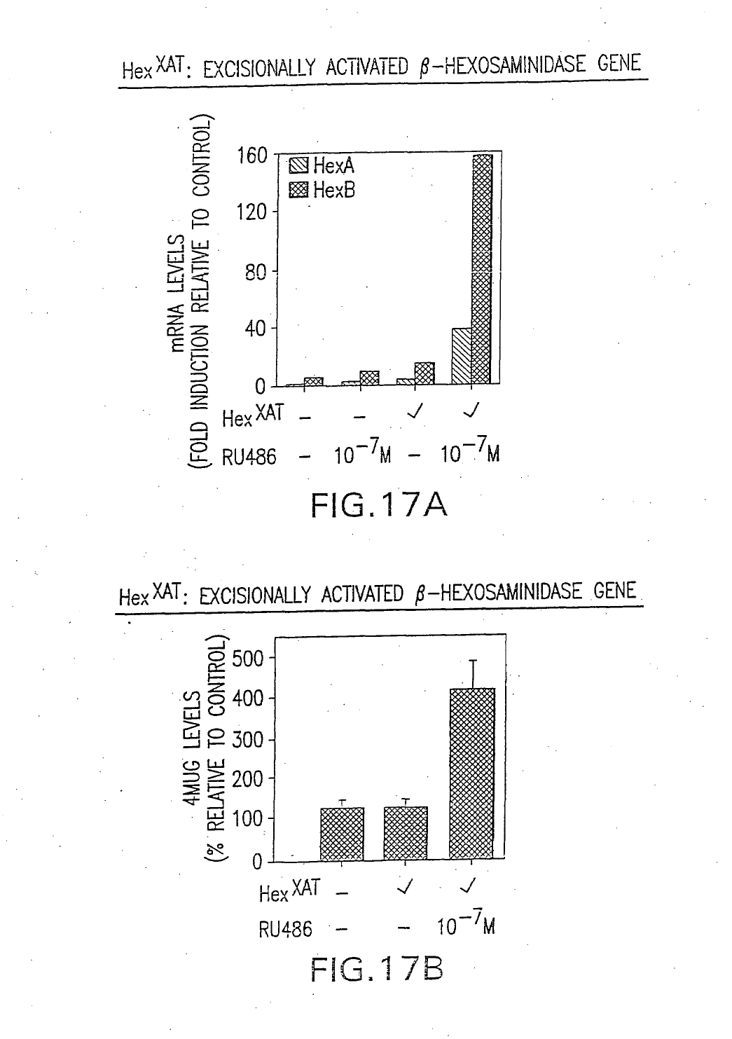

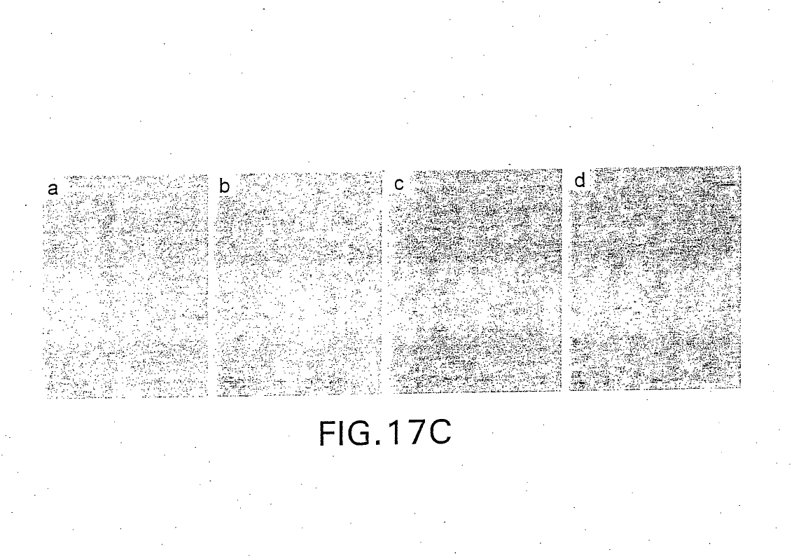

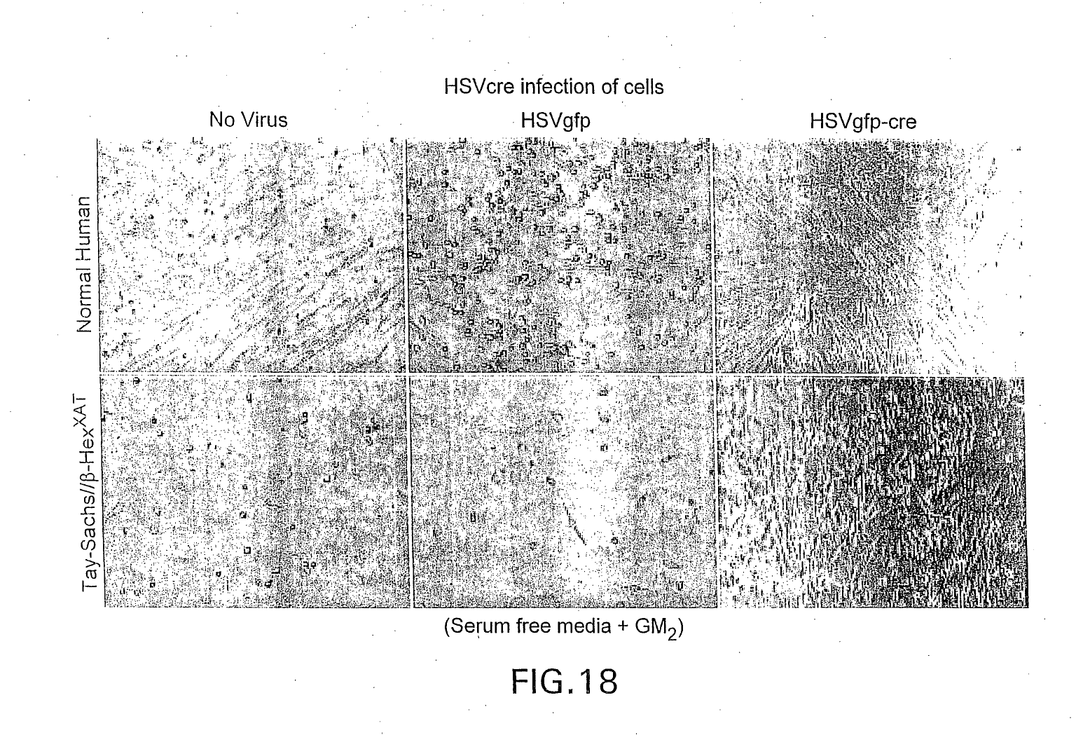

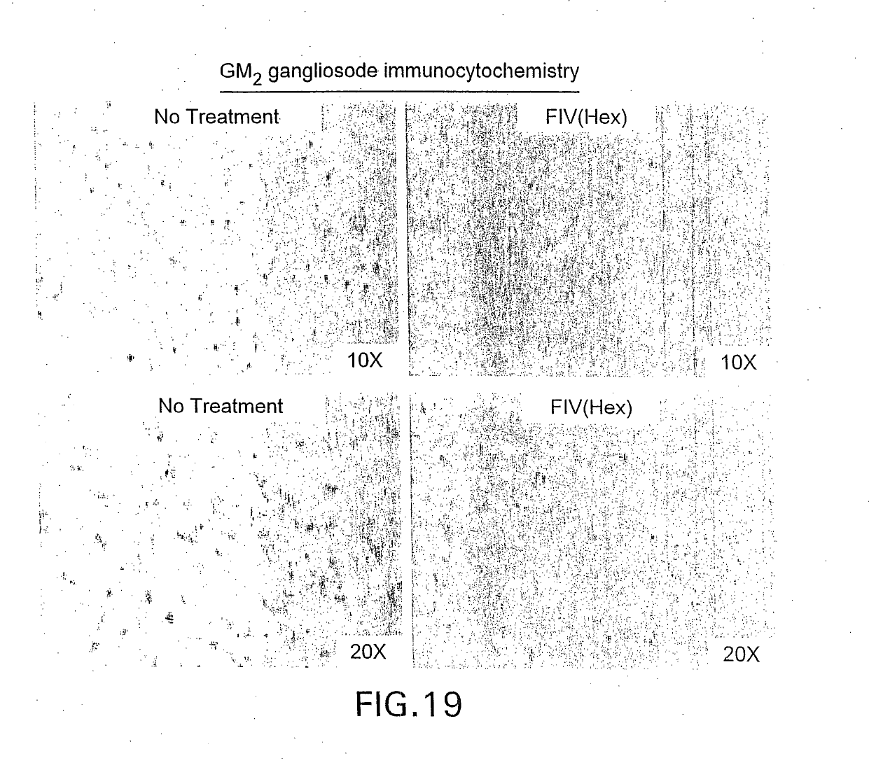

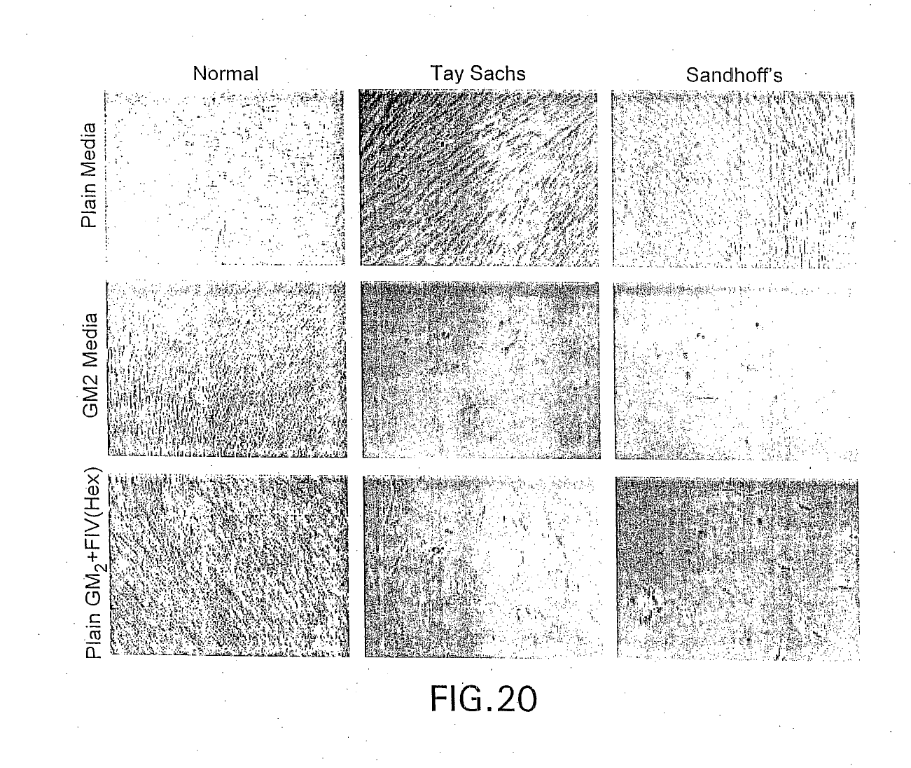

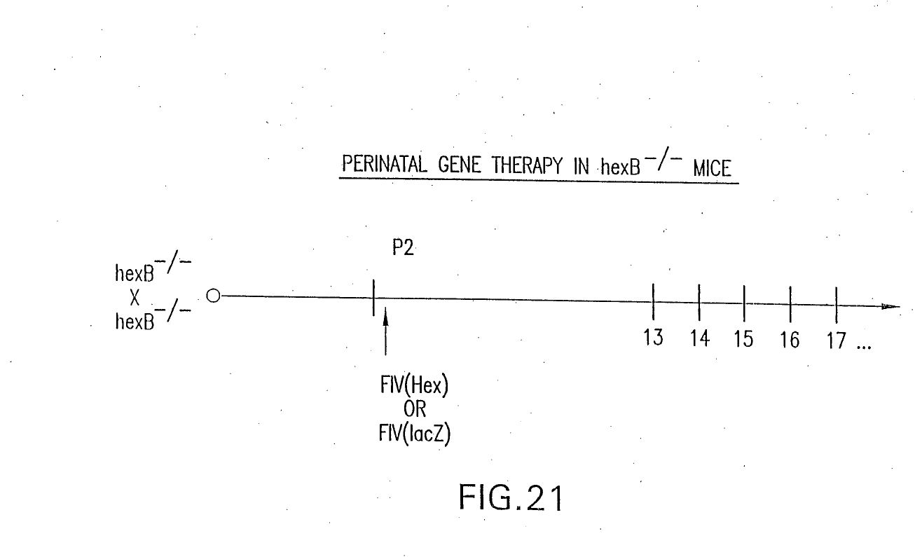

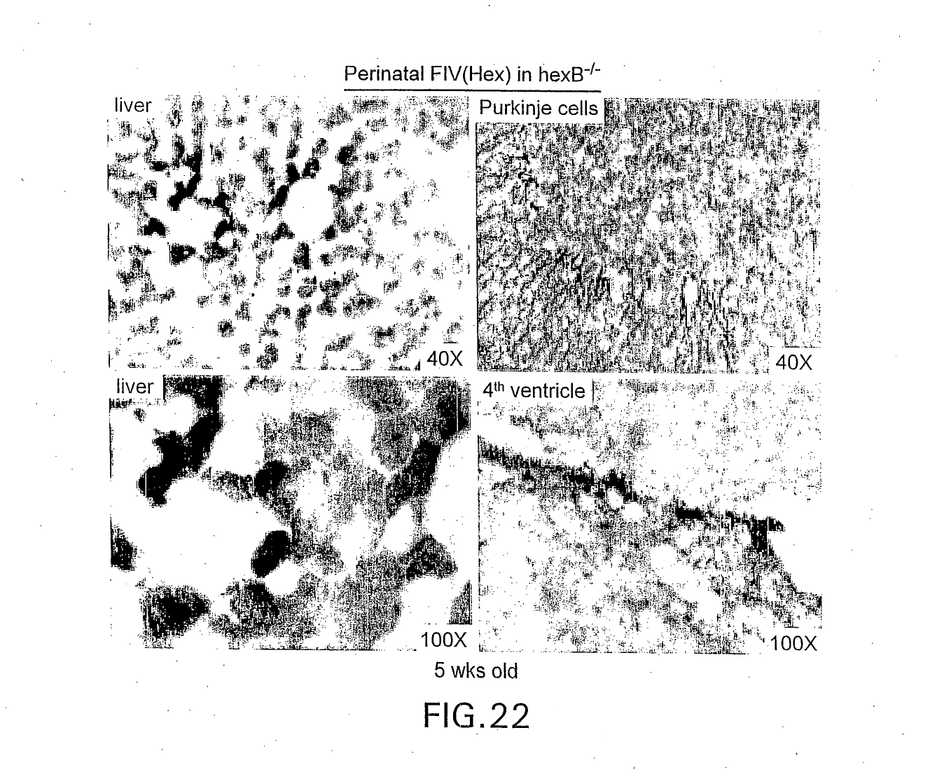

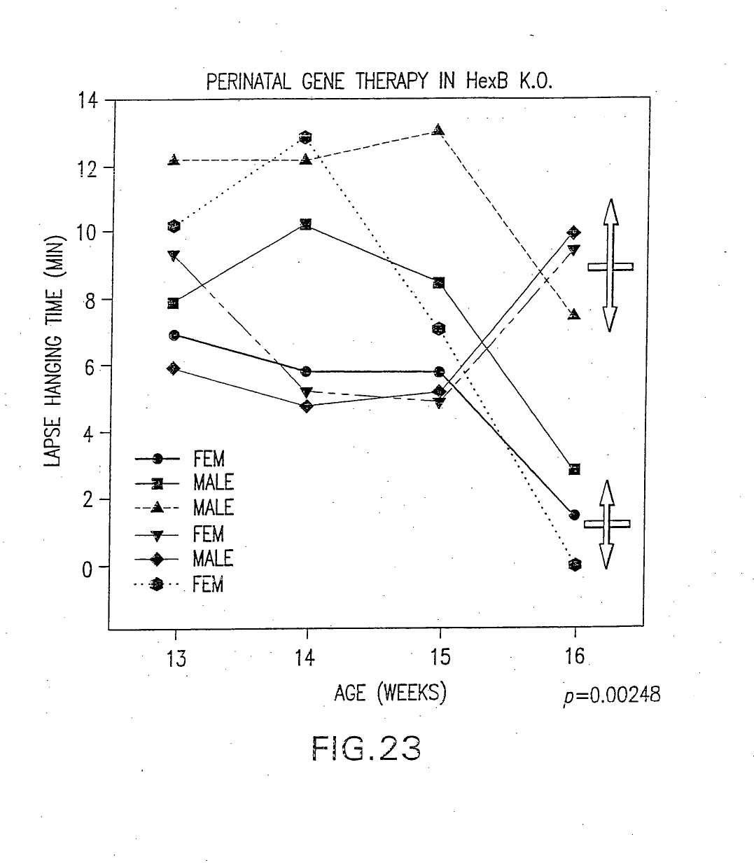

Tay-Sachs (TSD) disease is characterized by deficiency of HEXA (α/β), a lysosomal hydrolase necessary for the catabolism of GM₂ ganglioside in humans, due to mutations at the HexA locus. Although HEXA deficiency is present in all tissues, GM₂ lysosomal storage primarily occurs in neurons because of the high concentration of gangliosides in these cells, ultimately leading to dysfunction and cell death. The aim of this experiment was to analyze the properties of the βHex gene at a functional level in β-hexosaminidase deficient cells. Human primary fibroblasts from a patient with Tay-Sachs disease were obtained (Coriell Institute for Medical Research, Camden N.J.), and cultured in the laboratory. Fibroblasts are the only cell type with β-hexosaminidase deficiency commercially available. Since fibroblasts are normally characterized by minimal concentration of gangliosides, unlike neurons that have hundred fold higher ganglioside concentration, TSD fibroblasts do not display GM₂ storage or any other pathology in vitro. However, when GM₂ is added to their culture medium under serum free conditions, GM₂ is absorbed by the TSD fibroblasts resulting in storage and eventually cell death. For this purpose, we constructed a dormant Hex^{XAT} gene that can be activated by the bacterial cre recombinase following loxP directed excisional recombination (As disclosed herein): a transcription termination cassette (STOP) flanked by loxP sequences was inserted between the CMV promoter and the first open reading frame in βHex. Stable Hex^{XAT} cell lines were developed in human primary fibroblasts (FIGS. 16-18 derived from a Tay-Sachs disease patient:

CMV-IoxP-STOP-loxP-HexB-IRES-HexA-pA (TSD^{HexXAT})

The cells were cultured in serum free media (OptiMEM; Invitrogen, Carlsbad Calif.) and challenged by the addition of GM₂ ganglioside to the culture medium. The Hex^{XAT} gene was activated by transferring the cre recombinase gene to the cells via an HSVcre Amplicon vector. Activation of HexB-IRES-HexA in TSD fibroblasts rescued these cells from GM₂ induced cell death (1,000 cells/field) compared with cells treated with the control vector HSVgfp (2-5 cells/field) after the GM₂ challenge. In one exemplary set of data collection, the HexB-IRES-HexA transgene confers survivability to Tay-Sachs cells in vitro. For example, it was shown that normal human fibroblasts were resistant to the GM₂ challenge whereas TSD^{HexXAT} cells underwent cell death and lifted off the plate. Normal and TSD^{HexXAT} cells were treated with HSVgfp, an Amplicon vector capable of transducing cells with the gfp reporter gene (green fluorescent protein). Also Normal and TSD^{HexXAT} cells were treated with HSVcre. In this experiment, activation of the Hex^{XAT} gene by cre recombinase resulted in attenuation of the GM₂ deleterious effects, and conferred survivability to the TSD cells at levels comparable to that of normal human fibroblasts.

Transduction of murine cells by FIV(Hex) in vitro was also shown. For example, cultured primary murine fibroblasts were treated with FIV(Hex) at m.o.i.˜1. After 48 hours, DNA and RNA extracts were collected, and other samples were processed by X-Hex histochemistry. FIV(Hex) treated cells showed increased levels of X-Hex staining compared to naïve fibroblasts. Viral DNA was detected by polymerase chain reaction (PCR) employing primers specifically designed for human HexB. Gene expression was assessed in total mRNA extracts by Reverse Transcription (RT)-PCR employing the aforementioned primers for the human HexB gene. These results demonstrate the ability of FIV(Hex) to successfully transduce murine cells with βHex in vitro.

FIV(Hex) successfully restores cellular function in vitro. As mentioned previously, Tay-Sachs fibroblasts do not display GM₂ storage or any other pathology when cultured in vitro; however, when GM₂ ganglioside is added to serum-free culture medium, GM₂ is absorbed by the cells resulting in storage and eventually cell death. In one exemplary experiment normal and TSD fibroblasts were cultured in serum-free medium (OptiMem). Addition of GM₂ ganglioside to the culture medium had minimal effect on the normal fibroblasts, whereas it caused TSD fibroblasts to lose their spindle-like appearance, die and list off the plate (2-3 cells per visual field). FIV(Hex) treatment of cells two days after GM₂ ganglioside challenge (800 μg/mL) resulted in complete recovery of the normal cells and significant improvement of the TSD cells (over 100 cells per visual field). These results demonstrate that FIV(Hex) can effectively transduce healthy and stressed cells with βHex, and rescue them from GM₂ induced cell death.

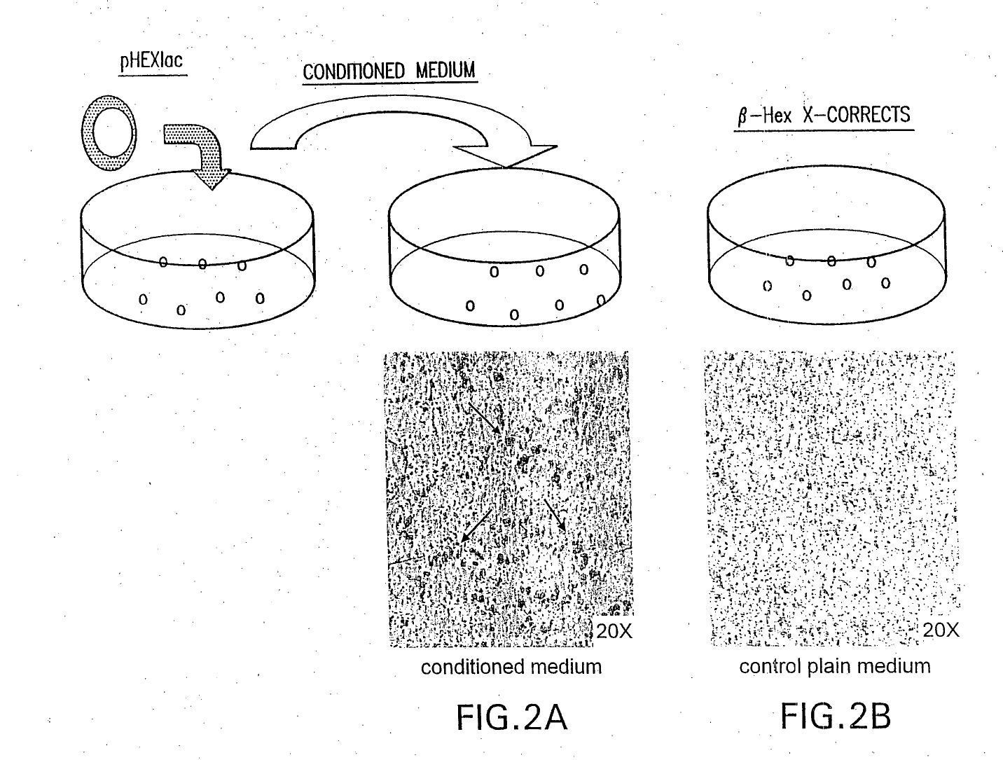

The β-Hex therapeutic gene is capable of correcting deficiencies in cells that are not transfected through cross-correction. (FIG. 2) An important property of the β-Hex transgene is the products hHEXA & hHEXB have the ability to cross-correct, specifically, to be released extracellularly and then to be absorbed via paracrine pathways by other cells whereby they contribute to β-hexosaminidase activity. BHK^{HexlacZ} cells were cultured and the supernatant was collected (conditioned medium), filtered (0.45 mm) and applied on normal mouse kidney fibroblasts in culture. Forty-eight hours later, the cells were washed thoroughly with phosphate buffered saline, and briefly treated with a trypsin solution to remove extracellular proteins from the cell surfaces. Following trypsin inactivation with Tris/EDTA buffer, the cells were fixed with 4% paraformaldehyde solution and processed by Fast Garnet histochemistry for β-hexosaminidase activity. Fast Garnet histochemistry of murine fibroblasts exposed to (FIG. 2A) conditioned medium collected from BHK^{HexlacZ} cells compared to cells exposed to medium from normal parent BHK-21 cells (FIG. 2B). These results demonstrate that hHEXA & hHEXB, products of the β-Hex transgene, are released into the extracellular medium and can be absorbed by other cells via paracrine pathways resulting in induction of the cellular β-hexosaminidase.

### 2. Example 2

### Transfecting Constructs

a) Construction of the Tricistronic #1-Hex Construct

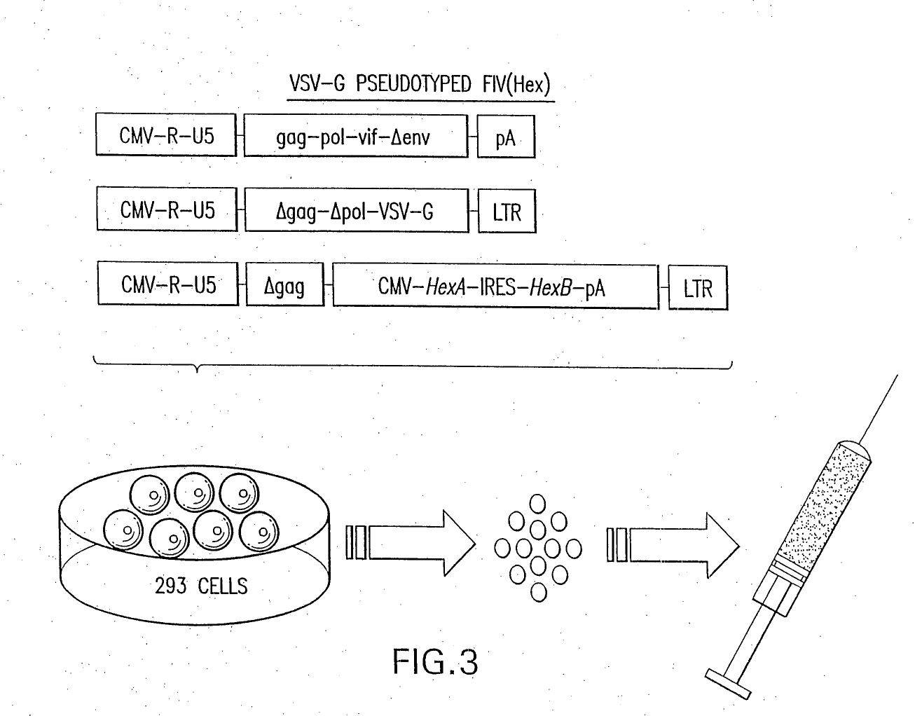

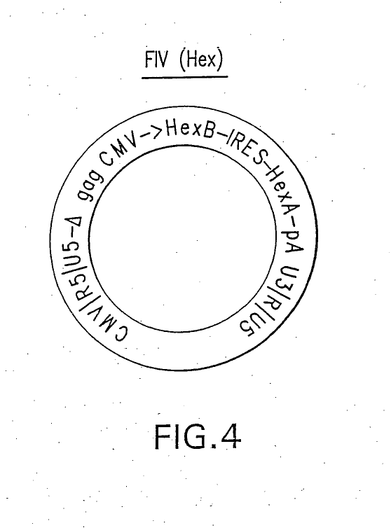

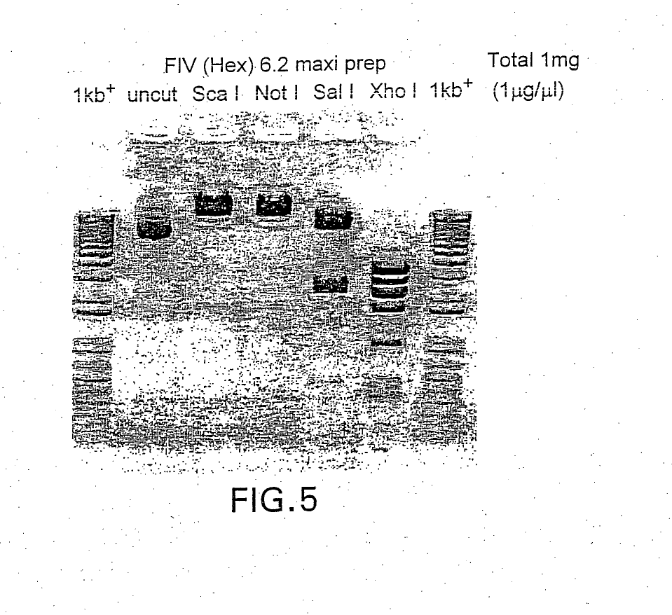

A tricistronic construct encoding for both isoforms of human β-hexosaminidase, hHexA & hHexB, as well as the α-galactosidase reporter gene (lacZ) was also made. hHexB cDNA was isolated following Xho I digestion of pHexB43 (ATCC, Manassas Va.) and cloned into the Xho I site of pIRES (Clonetech Laboratories, Palo Alto Calif.) downstream of the vector's cytomegalovirus (CMV) promoter sequence. The HexA cDNA was isolated from pBHA-5 (ATCC, Manassas Va.) by Xho I digestion and was subsequently inserted into the Xba I site of pIRES(HexB) downstream of the vector's IRES cassette by blunt ligation. A IRES-lacZ cassette was obtained from Dr. Howard J. Federoff, University of Rochester School of Medicine and Dentistry, but can be produced using standard recombinant techniques with known reagents and was inserted downstream to HexA into the Sal I site of pHexB-IRES-HexA by blunt ligation. In this construct, the cytomegalovirus promoter (CMV) drives transgene expression, and the translation of the second and third open reading frames (ORF), HexB and lacZ, respectively, are facilitated by an internal ribosomal entry sequence (IRES). The FIV(Hex) vector was constructed by isolating the HexB-IRES-HexA (β-Hex) fragment of pHexlacZ with NheI-NotI digestion is present and it was cloneed into the FIV backbone (Poeschla E M, et al., Nature Medicine 4: 354-357. (1998)), derived after excising the lacZ cassette from pFIV(lacZ) with Bpu1102I, leading to the successful construction of pFIV(Hex) (See FIGS. 3 and 4). Restriction fragment analysis indicated that pFIV(Hex) was constructed as designed. (FIG. 5).

The viral derived IRES sequence can effectively drive the expression of second genes in bicistronic constructs in vitro and in vivo, (Gurtu et al., 1996; Geschwind et al., 1996; Havenga et al. 1998). Nevertheless, IRES-mediated transcription in bicistronic constructs has been shown to reduce the levels of expression of the second ORF by about 40-50%. Hence, since HexB is necessary in the synthesis of both HEXA (α/β) and HEXB (α/α), it was cloned first in the tricistronic construct. Confirmation of the construct has been achieved by multiple restriction enzyme digestions as well as direct DNA sequencing.

The systems used for delivery include packaing and envelop vectors. (See FIG. 3). A three vector system was used, whereby each plasmid confers distinct instructions: the transfer vector carries the transgene of interest and lentiviral apparatus with mutated packaging and envelope genes. (FIG. 3). A vesicular stomatitis virus G-glycoprotein vector (VSV-G; Burns J C, et al., Proc Natl Acad Scie USA 90: 8033-8037. (1993)) contributes to the formation of the viral envelope in trans. The third vector confers packaging instructions in trans (Poeschla E M, et al., Nature Medicine 4: 354-357. (1998)). FIV production is accomplished as previously described (As disclosed herein). Titers routinely range between 10⁷-10⁸ infectious particles/mL. Two different FIV transfer vectors were constructed for the HexB-IRES-HexA transgene. The first contains a transgene driven by the cytomegalovirus promoter CMV, whereas the second one is driven by the chicken β-actin promoter fused to CMV enhancer elements (βact; Daly T M, et al., Hum Gene Ther 10:85-94. (1999a)) (FIG. 3). The βact promoter was kindly given to us by Dr. Nicholas Muszyka (University of Florida at Gainesville (Daly T M, et al., Gene Ther 8: 1291-8 (2001); Daly T M, et al., Hum Gene Ther 10: 85-94 (1999)) and was chosen based on its ability to drive transgene expression for prolonged periods (6-12 months) as previously reported (Daly T M, et al., Gene Ther 8: 1291-8 (2001)). The packaging and VSV-G vectors are described in (Poeschla E M, et al., Nature Medicine 4: 354-357. (1998)).

b) Results

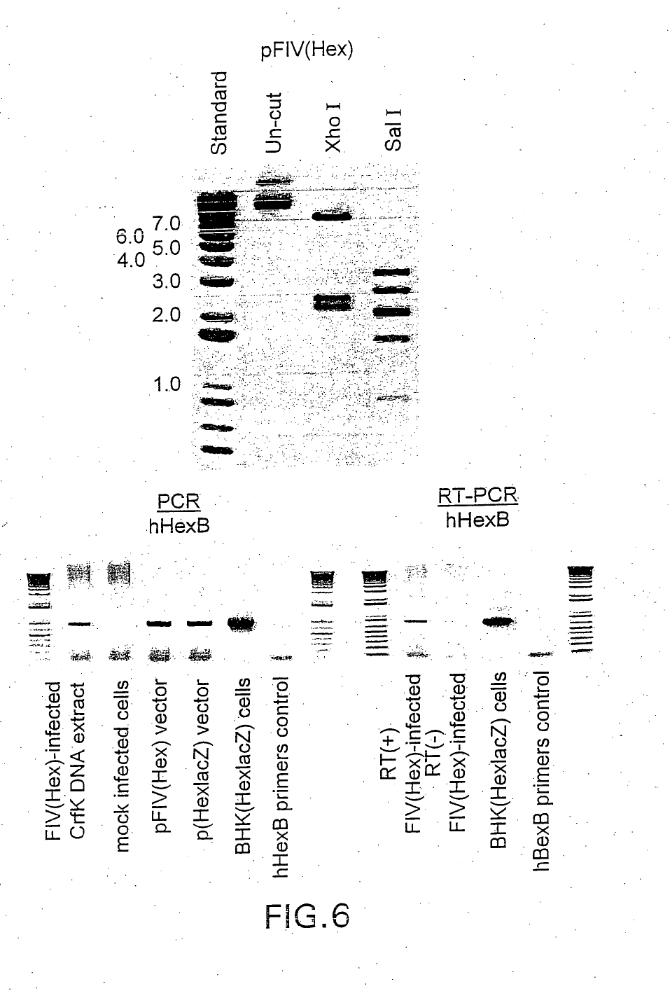

The FIV backbone vector was isolated from the FIV(lacZ) vector following Sst II & Not I digestion. The bicistronic transgene HexB-IRES-HexA was extracted from the pHexlacZ vector following Nhe I & Not I digestion, and was cloned into the FIV backbone by blunt ligation. FIV(Hex) digestion with the restriction enzymes Xho I and Sal I confirmed the cloning. (FIG. 6) FIV(Hex) virus was prepared using established methods and was tested in vitro as follows. Cultured murine fibroblasts (CrfK cell line) were exposed to FIV(Hex) for 12 hours, followed fresh media change. After 48 hours, cellular DNA and RNA extracts were collected. The presence of viral DNA was assessed by PCR with primers sets specifically designed for HexB (FIG. 6A). HexB expression was assessed by RT-PCR (FIG. 6B). These results demonstrate the ability of FIV(Hex) to transduce mouse fibroblasts with β-Hex, resulting in transgene mRNA expression. (FIG. 6).

The tricistronic vector pHEXlacZ was stably expressed in embryonic hamster kidney fibroblasts (BHK-21; ATCC) following standard transfection laboratory techniques using the LIPOFECTAMINE® reagent (Gibco BRL) per manufacturer's instructions. Forty-eight hours post-transfection, the cells were treated with 800 μg/mL G418 (Gibco BRL) for 10 days, and cell lines were selected, expanded and analyzed for expression of our tricistronic gene as follows. Analysis of the transfected cells showed that cell lines (Crfk, spleen, brain, liver, and kidney) stained positively for X-gal, indicating expression of and translation of the expressed product from the tricistronic vector. (FIG. 6)

### 3. Example 3

### In Vivo Use of FIV HEX Vectors

FIV(Hex) was constructed by inserting the bicistronic gene HexB-IRES-HexA in the place of the reporter gene lacZ in the FIV backbone vector using standard molecular biology techniques. FIV(Hex) was prepared in vitro by transient co-transfection of the transfer vector along with the packaging and envelop plasmids into 293H cells. The virus-rich supernatant was centrifuged and the viral pellet was reconstituted in normal saline, and was then titered in CrfK cells by the X-Hex histochemical method (10⁷-10⁸ infectious particles/ml). The viral solution was injected intraperitoneally to 2 days old HexB^{-/-} knockout mouse pups, which were allowed to reach the critical age of 16 weeks, when they displayed full signs of the lysosomal storage disease. For control, litermates were injected with the FIV(lacZ) virus, which is identical to FIV(Hex), but instead of carrying the HexB-IRES-HexA gene it carries the reporter gene lacZ. Locomotive performance was evaluated by placing the mice on a wire mesh attached on a clear plexiglass cylinder, and turning the wire mesh up-side-down. The lapse time until the mice fell off the wire mesh was recorded on weekly basis until the mice were terminated. It is important to state that at the critical time point of 16 weeks, the FIV(Hex) injected mice showed statistically better locomotive performance compared to FIV(lacZ) injected mice (controls). Furthermore, the FIV(Hex) mice had an extended life span for at least 2-3 additional weeks, at which point they were also terminated because they were showing signs of the disease.

### 4. Example 4

### HIV HEX Vectors

The HexB-IRES-HexA therapeutic gene was cloned into the Lenti6/V5D-TOPO vector commercially available by Invitrogen (Carlsbad, Calif.), whereby the cytomegalovirus promoter CMV drives gene expression [in a manner similar to FIV(Hex)]. A virus was constructed whereby the expression of HexB-IRES-HexA is driven by a promoter, such as that show in SEQ ID NO:23, which consists of a beta-actin portion and a CMV portion. This type of promoter has high expression in mammalian cells.

### 5. Example 5

### Systemic FIV Vector Administration

### Transduction of CNS Immune Cells and Purkinje Neurons

The systemic effects of gene therapy have been previously described in a variety of peripheral organs following intravenous administration or intraperitoneal inoculation of viral vectors, as well as in the brain following intracranial administration. However, limited information is available on the ability of viral vectors to cross the blood brain barrier and infect cells located within the CNS. Disclosed herein was the successful use of a VSV-G pseudotyped FIV(lacZ) vector capable of transducing dividing, growth arrested as well as post-mitotic cells with the reporter gene lacZ. Adult mice were injected intraperitoneally with FIV(lacZ), and the expression of β-galactosidase was studied 5 weeks following treatment in the brain, liver, spleen and kidney by X-gal histochemistry and immunocytochemistry. Interestingly, relatively low doses of FIV(lacZ) administered intraperitoneally lead to β-galactosidase detection in the brain and cerebellum. The identity of these cells was confirmed by double immunofluorescence, and included CD31-, CD3- and CD11b-positive cells. Fluorescent microspheres co-injected with FIV(lacZ) virus were identified within mononuclear cells in the brain parenchyma, suggesting infiltration of peripheral immune cells in the CNS. Cerebellar Purkinje neurons were also transduced in all adult-injected mice. Our observations indicate that relatively low doses of FIV(lacZ) administered intraperitoneally resulted in transduction of immune cells in the brain as well as a specific subset of cerebellar neurons.

a) Materials and Methods

(1) Viral Solution Preparation and Animal Injections

The defective, VSV-G pseudotyped FIV(lacZ) and packaging vectors are described in E. M. Poeschla, et al., Nature Med. 4 (1998) 354-357 which is herein incorporated by reference at least for material related to vectors including there sequence and construction. The vectors were transiently transfected into 293H cells (Invitrogen, Carlsbad Calif.) cultured in DMEM (Invitrogen) plus 10% FBS (Gemini, Woodland Calif.) using the Lipofectamine 2000 reagent per manufacturer's instructions (Invitrogen), and followed by a fresh media change supplemented by non-essential amino acids (Invitrogen). Sixty hours post-transfection, the supernatant was collected, filtered through 0.45 mm Surfil®-MF filter (Corning Seperations Division, Acton Mass.), aliquoted and frozen until further use. Titering was performed on CrfK cells (American Tissue Culture Collection; Manassas, Va.) cultured in 24 well tissue culture plates, and assessed at 5×10³ blue forming units (bfu)/mL by X-gal histochemistry per manufacturer's instructions (Invitrogen).

All methods pertinent to animal utilization were submitted for review and approved by the University of Rochester Committee on Animal Resources. Several doses of FIV(lacZ) were injected into a total of 15 male, 6 weeks old C57BL/6 mice. Specifically, 5 groups of 3 mice each were anesthetized with ketamine (60 mg/Kg) and xylazine (5 mg/Kg) I.P., and received one of the following FIV(lacZ) injections I.P.: 100 μL, 500 μL, 1,000 μL and 2,000 μL of FIV(lacZ) stock solution (5×10³ bfu/mL), as well as 2,000 μL normal saline that served as control. Five weeks after treatment, the mice were deeply anesthetized by pentobarbital (100 mg/Kg), and transcardially perfused with 50 mL of 4% paraformaldehyde in phosphate buffered saline solution (PBS), and their brain, liver, spleen and kidneys were harvested, post-fixed in the same solution for 3 hrs and frozen over dry ice until further use. The tissue from the various organs was cut into 20 μm thick sections on a freezing cryotome, and sequentially collected onto 10 glass slides whereby each slide contained representative sections of each organ (each tissue section was 2 mm apart of each other). The slides were stored at −20° C. until further analysis.

The brains of FIV injected-mice were examined for possible infiltration of peripheral circulating immune cells as follows: 1 mL FIV(lacZ) solution containing 10% v/v of 1 pm diameter fluorescent polystyrene microspheres (FluoSpheres®, F-13083; Molecular Probes) was administered intraperitoneally to 3 adult mice. An additional group of 3 mice received intraperitoneal injections of an equal volume of FluoSpheres in normal saline. After 5 weeks, the animals were deeply anesthetized and sacrificed via transcardial perfusion, and their brains were collected, frozen and sectioned on a freezing cryotome (20 μm thick sections). Sections collected onto glass histology slides, rinsed in PBS, cover-slipped utilizing the DPX mounting media, and color fluorescent images were captured at 594 nm wave-length.

(2) X-Gal Histochemistry

Histology sections were processed by X-gal histochemistry (Invitrogen) and evaluated under light microscopy. Specifically, the sections were washed in 0.15M phosphate buffered saline (PBS) containing 0.05% Triton-X (pH 7.2) for 60 min, followed by overnight processing for X-gal staining (Invitrogen). The tissue was then washed in 0.15M PBS for 30 min, briefly rinsed with dH₂O, and counterstained with nuclear Fast Red. The tissue sections were examined under light microscopy using a BX51 Olympus microscope (Tokyo, Japan), and microphotographic images were captured using a digital camera attached to the microscope.

(3) Immunocytochemistry

For immunocytochemical detection of E. coli β-galactosidase, methods previously described were adapted [S. Kyrkanides, et al., J. Neuroimmunol. 95 (1999) 95-106; S. Kyrkanides, et al., Mol. Brain. Res. 104 (2002) 159-169; S. Kyrkanides, et al., J. Neuroimmunol. 119 (2001) 269-277 which are herein incorporated at least for material related to detection]. All procedures were performed at room temperature. In brief, mounted tissue sections were washed in PBS for 60 min, followed by blocking with 4% normal goat serum (NGS) for 30 min, and overnight incubation in primary antibody (1:5,000 dilution of rabbit anti-β-galactosidase IgG polyclonal antibody; Chemicon, Temacula Ca; AB1211) solution containing 4% NGS (Invitrogen), 1% Bovine Serum Albumin (BSA; Sigma, St. Louis Mo.) and 0.5% Triton-X (Sigma) in PBS. The next morning, the tissue was rinsed with PBS containing 0.05% Triton-X for 60 min, followed by a second blocking step in 4% NGS for 20 min, and a 90 min incubation in secondary antibody (goat anti-rabbit IgG biotin-conjugated polyclonal antibody, 1:2,000 dilution; Jackson ImmunoResearch, West Grove Pa.) solution containing 1.5% NGS and 0.5% Triton-X in PBS. The tissue was rinsed with PBS for 30 min followed by a 90 min incubation in an avidin-biotin complex solution (ABC kit; Vector Laboratories, Burlingame Calif.) in PBS containing 0.05% Triton-X, and was then washed in 0.1M sodium acetate buffered solution (pH 7.4) for 30 min. The tissue was then reacted in a DAB (3,3′ diaminobenzidine)—Nickel solution in 0.1M sodium acetate buffered solution (pH 7.4) for 5 min, followed by a 15 min wash in PBS. The glass slides were then dehydrated, cleared through xylene and cover-slipped using DPX permanent mounting medium (Fluka; Neu-Ulm, Switzerland). Controls included staining of brain sections where the primary antibody was omitted. The tissue sections were viewed under a BX51 Olympus light microscope and color microphotographic images were captured as described above.

Using the same protocol, brain sections for the vascular cellular adhesion molecule-1 (VCAM-1; CD106), as well as the inducible isoform of prostaglandin H₂ synthase widely known as cycloxygenase-2 (COX-2), employing a rat anti-murine VCAM-1 monoclonal antibody (BD Pharmingen, San Jose Calif.; cat# 550547; dilution 1:100) and a rabbit anti-murine COX-2 polyclonal antibody (Cayman Chemical Co., Ann Arbor Mich.; cat# 16026; dilution 1:1,000) were also stained. Blood brain barrier leakage was evaluated by staining brain histology section for IgG immunoglobulins using a goat anti-mouse IgG polyclonal antibody (1:1,000 dilution; Jackson ImmunoResearch). The tissue sections were studied under a BX51 Olympus light microscope and black & white microphotographic images were captured as described above.

(4) Double Immunofluorescence

To confirm the identity of transduced cells expressing β-galactosidase, double immunofluorescence methods were adopted previously described [S. Kyrkanides, et al., J. Neuroimmunol. 119 (2001) 269-277 which is herein incorporated by reference at least for material related to detection]. All procedures were performed at room temperature. Specifically, for the detection of β-galactosidase, unless otherwise stated, we employed a rabbit anti-β-galactosidase IgG polyclonal antibody (Chemicon; AB1211 at 1:5,000 dilution), coupled with the Alexa⁴⁸⁸ fluorescent secondary antibody (1:500 dilution; Molecular Probes, Eugene Oreg.). Furthermore, to confirm β-galactosidase expression, an additional rabbit anti-β-galactosidase polyclonal antibody was employed (Biodesign Intl, Saco Me.; 1:5,000 dilution). For the detection of neurons [L. Zhou, et al., Ann. Neurol. 44 (1998) 99-109], a rat anti-neurofilament medium 145 KD (NFM) monoclonal antibody (RM055 at 1:400 dilution) was employed. This antibody was developed and kindly donated to us by Dr. Virginia Lee (University of Pennsylvania, Philadelphia Pa.) (Zhou L., et al., J. Neurosci 18: 7200-15 (1998)), and was coupled with the goat anti-rabbit Alexa⁵⁹⁴ fluorescent secondary antibody (1:500 dilution; Molecular Probes). Furthermore, the mouse monoclonal anti-neuron specific nuclear protein (NeuN) antibody (Chemicon) was utilized for identification of neurons, as well as the mouse anti-calbindin-D-28D monoclonal antibody for the detection of Purkinje cerebellar neurons (C-9848, Sigma), both coupled with goat anti-mouse Alexa⁵⁹⁴ fluorescent secondary antibody (Molecular Probes). T lymphocytes were identified by a rat anti-human CD3 monoclonal antibody (MCA 1477; Serotec Inc, Raleigh N.C.), with cross reactivity for mouse. For brain microglia we utilized a rat anti-mouse CD11b monoclonal antibody (MCA74; Serotec Inc, Raleigh N.C.), which also detects granulocytes, monocytes, NK cells and resident macrophages. Endothelial cells were detected with a rat anti-mouse CD31 monoclonal antibody (cat# 550274; BD Pharmingen, San Diego Calif.). For astrocytes we utilized a rat anti-GFAP (glial fibrilary acidic protein) antibody (Boehringer Mannheim, Indianapolis Ind.). All rat primary antibodies were coupled with a goat anti-rat Alexa⁵⁹⁴ fluorescent secondary antibody (Molecular Probes). Color fluorescent images were captured separately under 488 nm (green) and 594 nm (red) wave lengths. The images were then deconvoluted utilizing the Slidebook software package (Intelligent Imaging, Denver Colo.) and were overlapped by Adobe Photoshop v5.0 software package. The total number of CD11b⁺, CD3⁺ and galbindin⁺ cells were assessed in 10 random microscopic fields (40×) as follows: In each field, the number of βgalactosidase-positive cells were counted, as well as the total number of cells for each type, and averages & standard deviations were calculated.

b) Results

Representative histology sections from the brain, liver, spleen and kidney of mice treated intraperitoneally with FIV(lacZ) depicting expression of β-galactosidase after X-gal histochemistry (they show as a blue stain) and immunocytochemistry (they show as as a black stain). In the brain, small-diameter cells appearing as glia or endothelial cells were observed in thalamic nuclei, as well as large-diameter cells in the cerebellar cortex of the vermis appearing as neurons. Mice that were injected with normal saline did not show any β-galactosidase activity. X-gal positive cells were also identified in liver, spleen and kidney sections. Comparable sections were analyzed by immunocytochemistry employing a commercial antibody raised against bacterial β-galactosidase, depicting positive cells in thalamic nuclei of the brain and cerebellal cortex of the vermis compared to saline-injected mice. A typical Bar size was 100 μm.

In the brain, X-gal positive cells were primarily identified as circular, small-diameter cells located in vascularized areas, histologically appearing as microglia or endothelial cells. In addition, X-gal positive cells of larger diameter that appeared histologically as neurons were located within the cerebellar cortex. In the liver, X-gal positive cells were also localized at the hepatic portal triads and the white pulp of the spleen. Mammalian β-galactosidases were not detected by the histochemical staining method as evidenced by brain sections from saline-injected animals. Bacterial β-galactosidase protein expression was confirmed by immunocytochemistry; small-diameter cells that histologically appeared as glia, stained positively, as well as larger-diameter cells that appeared as neurons. Analysis of brain sections from saline-injected animals by immunocytochemistry revealed background levels of mammalian galactosidases.

The identity of the transduced cells was confirmed by double immunofluorescence following intraperitoneal FIV(lacZ) administration. In the brain, β-galactosidase expression was detected in cerebellar Purkinje neurons as evidenced by labeling with calbindin staining [R. Nakamura, et al., 1999. Acta Neuropathol. 97:196-200] (FIGS. 2A, 2B and 2C). These cells were primarily located in the cerebellar cortex of the vermis, and also stained positively for neurofilament-medium (data not shown). These cerebellar Purkinje neurons did not stain for NeuN (data not shown) as previously reported [R. J. Mullen, et al., Development 116 (1992) 201-211]. β-galactosidase immunofluorescence in Purkinje neurons was robustly observed in animals that received 10⁴ FIV(lacZ) i.p., the intensity of which decreased in animals that received 5×10³ i.p, and was undetectable in animals that received 5×10² i.p and saline injections. Furthermore, β-galactosidase expression was confirmed with a second polyclonal antibody. Overall, the location, histologic appearance, and profile of immunohistochemical markers suggested transduction of Purkinje neurons with the reporter gene lacZ.

The identity of brain cells expressing β-galactosidase following intraperitoneally FIV(lacZ) administration to adult mice was examined by double immunofluorescence. β-galactosidase expression was detected by Texas-red immunofluorescence in Calbindin-positive cerebellar Purkinje neurons, CD31-positive endothelial cells, CD11b-positive microglia/monocytes, and CD3-positive cells in cortical areas of the cerebrum. Microscopic overlays were also obtained and a typical Bar size was 100 μm.

β-galactosidase expression was also observed in cells expressing CD31, an endothelial cell marker. In addition, β-galactosidase was localized in smaller-diameter cells stained for CD11b, a marker for microglia, monocytes and macrophages. These were interspersed in vascularized areas of the brain. In addition CD3-positive cells, a marker often employed for lymphocytes, were found in vascularized areas of the brain in FIV(lacZ) treated mice expressing bacterial β-galactosidase. However, no GFAP-positive cells (astrocytes) were detected staining for α-galactosidase (data not shown). Moreover, large numbers of α-galactosidase expressing CD3-positive cells were found in the spleen (data not shown). α-galactosidase cell numbers were enumerated and presented in Table 3.

Intraperitoneal FIV(lacZ) administration transduced brain immune cells and cerebellar Purkinje neurons in adult mice. The number of lacZ-transduced Purkinje neurons in the cerebellum, and hematopoietic immune cells (CD11b- & CD3-positive) in the cerebrum were counted on histology sections (20 μm thick) and compared to total cell numbers per marker. The data are presented as the average (standard deviation) of 10 random microscopic fields (40×) in FIV and saline injected mice.

TABLE 3

| | Purkinje Neurons | | CD11b⁺ cells | | CD3⁺ cells | |
|---|---|---|---|---|---|---|
| | βgal⁺ | Calbindin⁺ | βgal⁺ | CD11⁺ | βgal⁺ | CD3⁺ |
| FIV | 8.1 (4.5) | 45.5 (3.2) | 3.7 (0.8) | 14.3 (6.8) | 1.4 (0.8) | 5.4 |
| | | | | | | (3.4) |
| saline | 0 | 50.7 (4.3) | 0 | 1.8 (0.7) | 0 | 0.7 |
| | | | | | | (0.6) |

Representative histology sections depicting thalamic nuclei of mice treated intraperitoneally with FIV(lacZ) were obtained. Vascular cell adhesion molecule-1 (VCAM-1; CD106) immunoreactivity was increased in FIV treated mice compared to saline injected animals (20×). Similar induction of COX-2 immunoreactivity was observed in this region of the brain following FIV versus saline injection. Furthermore, mice that were injected with FIV(lacZ) solution enriched with fluorescent microspheres displayed mononuclear cells in brain parenchyma containing these microspheres. These can be performed with a typical Bar being 100 μm.

Increased levels of VCAM-1 as well as COX-2 immunoreactivity were observed in endothelial cells of FIV(lacZ)-injected mice compared to controls, primarily located in vascularized areas of the brain. To determine whether FIV intraperitoneal administration leads to infiltration of circulating immune cells in the brain parenchyma of adult mice, fluorescent polysterine spheres were co-injected with the viral vector. Analysis revealed the presence of cells harboring red-fluorescent microspheres in vascularized paraventricular and cortical areas of the brain parenchyma in FIV(lacZ) injected mice. Administration of fluorescent microspheres in the saline solution alone rarely resulted in microsphere presence in the brain (data not shown). Higher magnification views revealed microsphere-containing mononuclear cells located adjacent to vessels.

Relatively low doses of VSV-G pseudotyped FIV(lacZ) were injected to young adult male mice intraperitoneally, and expression of the reporter gene β-galactosidase was analyzed 5 weeks post-treatment. This time was selected because preliminary data showed thereafter a considerable decrease in β-galactosidase expression from the CMV-driven transgene after 6 weeks in vivo, presumably due to silencing of the viral CMV promoter. Previous analysis on systemic viral vector injections to animals have included prenatal [G. S. Lipschutz, et al., Mol. Ther. 3 (2001) 284-292; .D. Porada, et al., Hum. Gene Ther. 9 (1998) 1571-1585; A. F. Tarantal, et al., Mol. Therapy. 3 (2000) 128-138], neonatal [T. M. Daly, et al., Gene Ther. 8 (2001) 1291-1298, T. M. Daly, et al., Proc. Natl. Acad. Sci. U.S.A. 96 (1999) 2296-2300] and adult [J. Huard, et al., Gene Ther. 2 (1995) 107-115, Y. Kang; et al., J. Virol. 76 (2002) 9378-9388; W. McCormack, et al., Mol. Ther. 3 (2001) 516-525] administration with limited attention on brain or cerebellar tissues.

(1) FIV Intraperitoneal Administration to Mice Results in Widespread Distribution of the Viral Vector and Gene Expression.

FIV(lacZ) was administered (5×10³ infectious particles in 1 mL) intraperitoneally to 5 week-old mice, which were then sacrificed 3 weeks post-treatment. Bacterial β-galactosidase (lacZ) expression was first assessed by X-gal histochemistry in brain histologic sections. X-gal positive small-diameter cells were identified in the brain, as well as larger-diameter neuron-like cells in the cerebellum. Saline treated mice did not display any X-gal staining in the brain. Furthermore, Bacterial β-galactosidase was also localized by immunocytochemistry. Small diameter cells stained positively for lacZ, as well as larger cerebellar cells, similar to the X-gal staining results. Saline-injected mice displayed modest levels of staining. The identity of the cerebellar neuron-like cells was confirmed by double immunocytochemistry, using antibodies against calbindin, a specific marker for Purkinje cerebellar neurons, coupled with an antibody for β-galactosidase.

(2) Peripheral Blood Mononuclear Cells Infiltrate the Brain in FIV(lacZ) Treated Mice

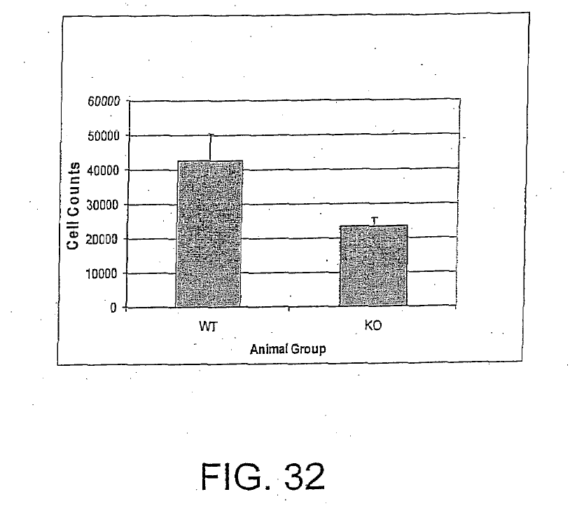

Young adult mice (5 weeks old) were injected with relatively small doses of FIV(lacZ) intraperitoneally (5×10³ infectious particles in 1 mL of normal saline). After one month, β-galactosidase expressing cells that were identified as CD11b⁺-positive and cells (microglia-monocytes-macrophages) were seen in the brain parenchyma. In addition CCR2−/− knockout mice display decreased infiltration of PBMC in the brain after injury. It was demonstrated that the MCP-1/C-C receptor system is important in the recruitment of PBMC in the brain following injury. Wildtype and CCR2 knockout C57B1/6 mice were injected into the striatum with a HSV amplicon vector expressing lacZ. Five days following injection, the animals were perfused and stained immunocyto-chemically for MHC class II in order to identify activated macrophages and microglia. Immuno-positive cells were in the striatum were counted using a unbiased stereological method. Significantly fewer MHC II+cells were observed in the CCR2 knockout animals compared to wildtype (p<0.03) (FIG. 32). This suggests that few circulating macrophages have entered the CNS in the knockout animal.

The results demonstrate X-gal positive cells in the brain and liver of mice following adult FIV(lacZ) administration. In fact, previous reports suggest highly efficient transduction of liver cells following intravenous and intraperitoneal injections in adult rodents [J. Huard, et al., Gene Ther. 2 (1995) 107-115; Y. Kang, et al., J. Virol. 76 (2002) 9378-9388; W. McCormack, et al., Mol. Ther. 3 (2001) 516-525]; however, information regarding CNS expression is lacking. The results show transduction of CD31-positive cells (endothelial cells), presumably infected directly from circulating virions, as well as CD11b-positive and CD3-positive cells in vascularized areas of the brain. Two potential mechanisms may account for the presence of transduced cells in the brain parenchyma: Direct transduction and/or trafficking of transduced peripheral cells in the CNS. In the first scenario FIV(lacZ) may successfully enter into the CNS and directly infect resident cells, such as microglia and/or astrocytes. Microglia activation could then induce an inflammatory cascade resulting in recruitment of additional circulating peripheral immune cells; FIV(lacZ) readily transduces primary microglia cultures in vitro (data not shown). Although it is difficult to precisely calculate the number of FIV vectors that entered into the brain parenchyma following systemic FIV(lacZ) administration in relatively small numbers of virus, it is estimated that only a small percentage of virions entered the CNS since there were not any astrocytes expressing the reporter gene lacZ identified. Previous in vitro studies suggest that VSV-G pseudotyped FIV lentiviral vectors readily transduce primary murine astrocyte cultures (data not shown). These considerations make trafficking of transduced peripheral cells more likely. In support was the observation that a considerable number of transduced CD11b⁺ (microglia-monocytes-macrophages) and CD3⁺ cells (lymphocytes) expressing the reporter gene β-galactosidase were detected, along with increased total numbers of CD11⁺ and CD3⁺ cells in the brain of FIV treated mice compared to saline-injected controls. In our experiment, the presence of cells containing fluorescent microspheres in the brain parenchyma further support the assertion that FIV(lacZ) systemic administration in the adult mouse results in enhanced infiltration of circulating immune cells into the brain. In addition, a considerable number of transduced CD3⁺ and CD 11b⁺ cells were observed in peripheral tissues, including the spleen and liver. When histologic sections were evaluated for increased presence of immunoglobulins (IgG) in the brain parenchyma [W. F. Hickey, GLIA 36 (2001) 118-124], no differences were observed between the FIV treated and control animals (data not shown). In conclusion, these data are more consistent with the mechanism whereby FIV systemic administration resulted in stable transduction of hematopoietic precursors in vivo, which gave rise to a multitude of lacZ-transduced peripheral immune cells that then entered into the brain parenchyma possibly through an intact BBB [J. Priller, et al., Nat. Med. 7 (2001) 1356-1361]. Previous work [G. W. Goldstein, et al., Ann. N.Y. Acad. Sci. 481 (1986) 202-213; W. Risau, H. Wolburg, T.I.N.S. 13 (1990) 174-178; W. Risau, et al., Devel. Biol. 117 (1986) 537-545] on the development of mouse BBB using large protein molecules suggested initial formation of the BBB during the late days of rodent embryonic life with full completion after birth. Furthermore, BBB in the adult rodent is not uniform, since certain areas of the brain do not develop a BBB and thus allow for free exchange of molecules. These areas include the median eminence (hypothalamus), pituitary, choroid plexus, pineal gland, subformical organ, organum vasculosum lamina terminalis and area posterma [W. Risau, et al., Devel. Biol. 117 (1986) 537-545]. In the past peripheral circulating immune cells possibly could have entered the CNS through an intact BBB [W. F. Hickey, et al., J. Neurosci. 28 (1991) 254-260; W. F. Hickey, GLIA 36 (2001) 118-124; W. F. Hickey, et al., J. Neuropath. Epx. Neurol. 51 (1992) 246-256]. Cell surface adhesion molecules, such as VCAM-1; expressed by endothelial cells possibly mediate the recruitment of circulating monocytes and lymphocytes via interaction with the very late activation antigen (VLA-4) on their surface [J. Greenwood, et al., Immunol. 86 (1995) 408-415]. In fact, increased levels of brain VCAM-1 expression in the FIV-treated mice were observed. Moreover, COX-2, a key inflammatory mediator [S. Kyrkanides, et al., Mol. Brain. Res. 104 (2002) 159-169, M. K. O'Banion, Crit. Rev. Neurobiol. 13 (1999) 45-82], also showed increased brain immunoreactivity in treated animals. Therefore, it is possible that FIV systemic administration induced the infiltration of peripheral immune cells into the brain parenchyma. Similarly, in the case of experimental autoimmune encephalitis, intraperitoneal irritant administration routinely results in infiltration of activated monocytes and lymphocytes in the brain of adult mice [M. Bradl, A. Flugel. 265 (2002) 141-162, L. Izikson, et al., Clin. Immunol. 103 (2002) 125-131, V. K. Kuchroo, et al., Ann. Rev. Immunol. 20 (2002) 101-123, R. H. Swanborg, et al., Immunol. Rev. 184 (2001) 129-135]. Unexpectedly, adult mice injected with FIV(lacZ) I.P. were observed to have Purkinje cerebellar neurons expressing β-galactosidase. Although FIV is capable of neuronal retrograde transport from distal sites (data not shown), Purkinje neurons participate in isolated cerebellar circuits, whereby their axons selectively project to the dentate nucleus and other deep cerebellar nuclei. Previous studies reported efficient transduction of Purkinje neurons by VSV-G pseudotyped FIV vectors following direct intracerebellar injections [J. M. Alisky, et al., Mol. Neurosci. 11 (2000) 2669-2673]. This raises the possibility that Purkinje neurons may directly communicate with vascular elements through previously unrecognized processes. In fact, previous studies have identified close anatomic relationship of Purkinje neurons to cerebellar microvasculature [M. Akima, et al., Acta Neuropathol. 75 (1987) 69-76; H. Duvernoy, et al., Brain Res. Bull. 11 (1983) 419-480]. In addition, recent reports provide indirect evidence of possible direct connection of Purkinje neurons with the vasculature. In a report by Tian et al. [Tian, et al., Eur. J. Neurosci. 8 (1996) 2739-2747], dystroglycan is shown to be expressed at the glial-vascular interface in the cerebellum, as well as in Purkinje neurons. In another study, the CXCR3 chemokine receptor was found expressed by astrocytes, endothelial cells, smooth muscle cells, as well as Purkinje neurons [S. H. Goldberg, et al., Neuropath. Appl. Neurobiol. 27 (2001) 127-138], which may suggest that Purkinje cells may contribute to the recruitment of peripheral circulating cells, including T-lymphocytes into the cerebellum, although this hypothesis is yet to be experimentally confirmed. An alternative hypothesis can also be considered, whereby lacZ-transduced Purkinje neurons derive directly from or following an interaction with hematopoietic precursors. In a recent study on post-mortem human tissues derived from patients that had undergone radiation treatment and transplantation at an earlier age, it was suggested that hematopoietic precursors may in fact enter into the human cerebellum and fuse to existing Purkinje cells [M. Weinmann, et al., Proc. Natl. Acad. Sci. U.S.A. 100 (2003) 2088-2093]. However, this study did not exclude the possibility of direct differentiation of bone marrow precursors into neurons. Recent evidence suggests that hematopoietic precursors can directly differentiate into cerebellar Purkinje neurons. Priller et al. [J. Priller, et al., J. Cell Biol. 155 (2001) 733-738] reported that bone marrow derived cells transduced with the reporter gene green fluorescent protein (GFP) by a retroviral vector in vitro differentiated into Purkinje neurons that were engrafted into the cerebellum of adult mice. Disclosed herein it is likely that FIV(lacZ) stably transduced hematopoietic precursors in the periphery, which in turn differentiated into Purkinje cells and became engrafted into the cerebellar cortex; alternatively, FIV-transduced hematopoietic cells transferred the reporter gene lacZ to Purkinje cells following cell-cell fusion.

### 6. Example 6

### Neonatal Administration of a β-Hexosaminidase Lentiviral Vector Ameliorates GM₂ Storage and Brain Inflammation in a Mouse Model of GM₂ Gangliosidosis

Brain inflammation caused by the infiltration of peripheral immune cells in GM₂ gangliosidosis has been recently realized as a key factor in disease development. Disclosed herein are the effects of a FIV β-hexosaminidase vector in the brain of hexB-deficient (Sandhoff disease) mice following intraperitoneal administration of FIV(Hex) to pups of neonatal age. Since brain inflammation, neuronal cell death and motor dysfunction are characteristics of hexB-deficiency and the attendant GM₂ gangliosidosis, these parameters were employed as experimental outcomes. Neonatal gene transfer was first evaluated in mice that received a single β-galactosidase FIV intraperitoneal injection at post-natal day P2, demonstrating transduction of brain, liver and spleen cells. The ability of FIV(Hex) to infect murine cells was initially demonstrated with success in normal mouse fibroblasts and human Tay-Sachs cells in vitro. Furthermore, systemic transfer of FIV(Hex) to P2 hexB^{-/-} knockout pups lead to transduction of peripheral and central nervous system tissues. Specifically, β-hexosaminidase expressing cells were immunolocalized in periventricular areas of the cerebrum as well as in the cerebellar cortex. FIV(Hex) neonatal treatment resulted in reduction in GM₂ storage along with attenuation of the brain inflammation and amelioration of the attendant neurodegeneration and clinical motor deterioration. In conclusion, these results demonstrate the effective transfer of a β-hexosaminidase lentiviral vector to the brain of Sandhoff mice and resolution of the GM₂ gangliosidosis after neonatal intraperitoneal administration.

a) Materials and Methods

(1) Development of Viral Vectors

Construction of the bicistronic transgene βHex encoding for both isoforms of the human β-hexosaminidase, HexB-IRES-HexA, was performed as described herein. The defective FIV vector CTRZLb, named FIV(lacZ) herein, was initially described by Poeschla et al. (Poeschla E M, et al., Nature Medicine 4: 354-357. (1998)) along with the pseudotyping (vesicular stomatitis virus-G glycoprotein; Burns J C, et al., Proc Natl Acad Scie USA 90: 8033-8037. (1993)) and packaging plasmids. A Nhe 1-Not I segment containing the bicistronic β-hexosaminidase gene was cloned in the place of lacZ in the CTRZLb vector (SstII-Not I) by blunt-cohesive ligation to generate the FIV(Hex) transfer vector. FIV(lacZ) and FIV(Hex) virus was prepared and titered as previously described (Kyrkanides S, et al., Mol Therapy 8: 790-795. (2003b)); titers were calculated at 5×10⁶ and 5×10⁷ infectious particles/mL for FIV(lacZ) and FIV(Hex), respectively. FIV(Hex) was initially tested in normal murine fibroblasts (donated by Dr. M. Kerry O'Banion, University of Rochester) and human Tay-Sachs (GM11853) cells purchased from the Coriell Institute for Medical Research (Camden N.J.).

(2) Animal Injections

All protocols employing laboratory animals were reviewed and approved by the University Committee on Animal Resources. Nine C57B6/J (Harlan Laboratories, Indianapolis Ind.) were injected with 100 μL aqueous solution containing a total of 10⁵ infectious particles of FIV(LacZ) intraperitoneally at post-natal day 2 (P2). An equal number of mice were injected with 100 μL of normal sterile saline and served as controls. The mice were returned to their mothers, and were sacrificed thereafter at 3, 6 and 13 weeks post-treatment. In addition, hexB^{+/-} knockout breeder pairs (originally developed and kindly provided by Dr. Richard Proia, Genetics Division, NIDDK/NIH, Bethesda Md.) were mated to produce homozygous hexB^{-/-} mice at a 0.25 expectancy ratio. Genotyping was performed by PCR of DNA extracts from tail biopsies employing the following primer sets: ⁵′ATT TTA AAA TTC AGG CCT CGA^{3′}, (SEQ ID NO:42) ^{5′}CAT AGC GTT GGC TAC CCG TGA^{3′} (SEQ ID NO:43) and ^{5′}CAT TCT GCA GCG GTG CAC GGC^{3′} (SEQ ID NO:44). The latter were allowed to grow to maturity (60 days old) and were than employed as breeders to deliver hexB^{-/-} pups at a 1.00 expectancy ratio that were injected intraperitoneally (N=5) with 10⁵ infectious FIV(Hex) particles in 100 μL normal saline. An equal number of mice received saline treatment and served as controls. This latter group was sacrificed 5 weeks after treatment, and the expression of the β-hexosaminidase transgene was evaluated in the brain, liver and spleen by immunocytochemistry. Ten additional hexB^{-/-} mice were administered FIV(Hex) or FIV(lacZ) intraperitoneally (10⁷ infectious particles) at post-natal day P2, and were sacrificed thereafter at 12 and 16 weeks of age. Heterozygous hexB^{+/-} littermates, which lack any histological or clinical pathology, were also employed as controls.

### (3) PCR & RT-PCR

Samples were rinsed with sterile 0.15M phosphate buffered saline pH 7.2 (PBS), and total RNA/DNA extracts were collected and precipitated utilizing the TRIzol reagent per manufacturer's instructions (Invitrogen). RNA samples were reconstituted in DEPC treated ddH₂O, and 260 nm/280 nm readings were spectrophotometrically obtained. A total of 2 μg RNA were treated with DNase I (Invitrogen) for complete destruction of deoxyribonucleotides per manufacturer's instructions, followed by reverse transcription reaction employing the First cDNA Strand Synthesis kit also per manufacturer's instructions (Invitrogen). RT conditions included reactions with and without the presence of the reverse transcriptase enzyme. Subsequently, polymerase chain reaction was performed employing Platinum Taq DNA polymerase (Invitrogen) with PCR primers specifically designed for the human HexA and HexB, ⁵′GAA TCC CAG TCT CAA TAA TAC C^{3′} & ^{5′}CAT ACA AGC CTC TCC ACC^{3′} (Ta=58° C.) and ^{5′}AGT CCT GCC AGA ATT TGA TAC C^{3′} & ^{5′}ATT CCA CGT TCG ACC ATC C³′ (Ta=58° C.), respectively (Kyrkanides S, et al., Mol Therapy 8: 790-795. (2003b)). PCR products were then analyzed by agarose gel electrophoresis and stained with ethidium bromide, images of which were captured using an EDAS Imaging analysis system (Kodak, Rochester N.Y.). Samples included RT(+) and RT(−) products, pHex vector DNA (100 nM) that served as positive PCR control, as well as primers control (no template). In addition, murine IL-1β (^{5′}GAGAACCAAGCAACGACAAAATACC^{3′} (SEQ ID NO:45) & ^{5′}GCATTAGAAACAGTCCAGCCCATAC^{3′} (SEQ ID NO:46)), TNFα (^{5′}CGAGTGACAAGCCTGTAGCC^{3′} (SEQ ID NO:47) & ^{5′}GGTTGACTTTCTCCTGGTATGAG^{3′} (SEQ ID NO:48)), IL-6 (^{5′}ATGTTCTCTGGGAAATCGTG^{3′} (SEQ ID NO:49) & ^{5′}GAAGGACTCTGGCTTTGTCTT^{3′} (SEQ ID NO: 50)) and ICAM-1 (^{5′}CAGTCGTCCGCTTCCGCTAC^{3′} (SEQ ID NO:51) & ^{5′}AGAAATTGGCTCCGTGGTCCC^{3′} (SEQ ID NO:52)) mRNA levels were evaluated by semi-quantitative RT-PCR genes by methods previously described (Kyrkanides S, et al., J Neuroimmunol 95:95-106. (1999)).

(4) X-gal & X-Hex Histochemistry

Tissue sections processed by X-gal histochemistry were washed in 0.15M phosphate buffered saline (PBS) containing 0.05% Triton-X (pH 7.2) for 60 min, followed by overnight processing for X-gal staining per manufacturer's instructions (Invitrogen). The tissue was then washed in 0.15M PBS for 30 min, briefly rinsed with dH₂O, and counterstained with nuclear Fast Red. For histochemical detection of β-hexosaminidase, the aforementioned protocol was modified by employing 5-bromo-4-chloro-2-indonyl-β-D-glucosaminide (Sigma, St. Louis Mo.; Oya Y, et al., Acta Neuropathol 99: 161-168. (2000)). The tissue sections were studied under light microscopy using a BX51 Olympus microscope (Tokyo, Japan), and microphotographic images were captured in TIFF 16-bit format using a SPOT RT Color CCD digital camera attached onto the microscope and connected to a DELL computer.

(5) β-Galactosidase Chemiluminescence Assay

To quantitatively evaluate β-galactosidase expression in the brain, liver, spleen and kidney following FIV(lacZ) systemic administration, we employed a 1,2-Dioxetane β-galactosidase substrate chemiluminescence assay. In brief, tissue sections for the various organs were collected into 1.5 mL Eppendorf tubes and were treated with Galactolight™ lysis buffer, briefly centrifuged, and the supernatant was collected and stored in −80° C. until further use. Total protein concentration was assessed (expressed in μg/mL) for all samples by employing the BCA protein assay reagent kit (Pierce, Rockford Ill.) and using a Benchmark microplate reader (Bio-Rad, Hercules Calif.) per manufacturer's instructions. β-galactosidase expression levels were then assessed using the Galactolight™ luminescent assay system (Applied Biosystems, Bedford Mass.) per manufacturer's instructions utilizing a Packard Lumnicount BL1000 plate reader (Meriden, Conn.). Results were normalized for total protein and expressed as luminescence units. Chemiluminescence activity in the saline-injected animals was evaluated, representing endogenous mammalian galactosidases, and subsequently subtracted from that of FIV(lacZ)-injected mice.

(6) Histology

For immunocytochemical detection of antigens in the brain, previously described methods were adapted (Kyrkanides S, et al., Mol Brain Res 104: 159-169. (2002)). In brief, for the detection of β-hexosaminidase, a goat polyclonal IgG antibody (Proia et al., 1984) raised against human HEXB protein (1:1,000) kindly provided by Dr. Richard Proia (Genetics Division, NIDDK/NIH; Bethesda Md.) was employed. Activated astrocytes were identified by a mouse anti-glial fibrillary acidic protein (GFAP) monoclonal antibody (1:400 dilution; Chemicon INTL, Temecula Calif.; clone GA-5). Activated denditric cells/microglia/macrophages were stained with a rat anti-major histocompatibility complex class-II (MHC-II; Bachem, Torrance, Calif.; clone ER-TR3). GM₂ ganglioside was immunolocalized employing a mouse anti-N-acetyl GM₂ monoclonal IgM antibody (Seikagaku Corp.; East Falmouth Mass.; clone MK1-16). These antibodies were coupled with appropriate secondary antibodies: rabbit anti-goat IgG biotin-conjugated, goat anti-mouse IgG Fab′ biotin conjugated, goat anti-rat IgG biotin-conjugated antibodies, and goat anti-mouse IgM biotin conjugated, respectively (Jackson ImmunoResearch, West Grove Pa.).Visualization was performed utilizing DAB (3,3′ diaminobenzidine)-Nickel as chromagen. The glass slides were then dehydrated through multiple ethanol solutions, cleared through xyaline and cover-slipped using DPX permanent mounting medium (Fluka; Neu-Ulm, Switzerland). The tissue sections were then studied under a BX51 Olympus light microscope and color microphotographic images were captured as described above. The total number of GFAP⁺ and MHC-II⁺ cells were counted in 10 random microscopic fields (40×) as follows: In each field, the number of positive cells were counted and averages & standard deviations were calculated for each area of the brain.

Cell death was determined by the terminal uridine nick-end labeling (TUNEL) assay per manufacturer's instructions (ApoTag® Plus Peroxidase In Situ Apoptosis Detection Kit; Chemicon INTL, Tamacula Calif.). Visualization of apoptotic cells was performed by DAB (3,3′ diaminobenzidine) which precipitated a red-brown color. Neurodegeneration was also evaluated by Fluoro-Jade® histofluorescent labeling (Histo-Chem Inc., Jefferson Ariz.) developed by Schmued et al. (1997) at the National Center for Toxicological Research, Food & Drug Administration, Jefferson Ariz.). In brief, tissue sections were immersed in 100% ethanol for 3 min/70% ethanol for 1 min/dH₂0 for 1 min/0.06% potassium permanganate (KMnO₄) for 15 min/0.001% Fluoro-Jade® plus 0.1% acetate in dH₂0. Slides were dehydrated in ethanol solutions, cleared in xylene and covered slipped with DPX mounting media (Fluka, Neu-Ulm, Switzerland).

b) Results

The effectiveness of systemic FIV vector administration on neonatal mouse pups to transduce brain cells was evaluated following intraperitoneal injection of FIV(lacZ) at post-natal day P2. The mice were then sacrificed at 3, 6 and 13 weeks post treatment and β-galactosidase expression was evaluated by X-gal histochemistry and the Galactolight™ chemiluminescence assay. In the brain, X-gal positive cells were observed mainly in blood vessels, perivascular as well as perventricular tissues that appear histologically as macrophages/microglia (FIG. 24A). X-gal positive cells were also detected in the liver and spleen. (FIGS. 24B & 24C, respectively). Galactolight™ chemiluminescence revealed increasing levels of β-galactosidase enzyme activity in the brain with time (FIG. 24D), as well as in the liver and spleen (FIGS. 24E & 24F, respectively). These results indicate stable transduction of brain cells after neonatal FIV intraperitoneal administration in vivo. Interestingly, the number of transduced cells, as assessed by X-gal histochemistry, increased from 3-to-6 weeks and may account for the observed elevation in Galactolight activity (FIGS. 24D-24E). Moreover, the number of transduced cells plateaued after 6 weeks, suggesting that the observed increase in Galactolight activity from 6-to-13 weeks was due an elevation in β-galactosidase expression.

Transduction of brain and liver cells in hexB^{-/-} mice following FIV(Hex) neonatal administration was shown to occur. Targeted deletion of the hexB locus in the mouse leads to the development of an animal model of GM₂ gangliosidosis (hexB^{-/-}) that is characterized by Tay-Sachs and Sandhoff disease-like pathology, including βhexosaminidase deficiency, neuronal storage, neuronal cell death, locomotive deterioration and death at 4 months of age. FIV(Hex) was administered systemically to 2 days old hexB^{-/-} pups (neonates) via intraperitoneal injection of 10⁷ infectious particles in a of 100 μL aqueous solution. The pups were sacrificed at 5 weeks of age. HexB expression was assessed by immunocytochemistry with a specific antibody against human HEXB protein. HEXB positive cells were identified at the portal triads of the liver. HEXB expression was also identified in circumvetricular areas of the brain as well as in perivascular cells.

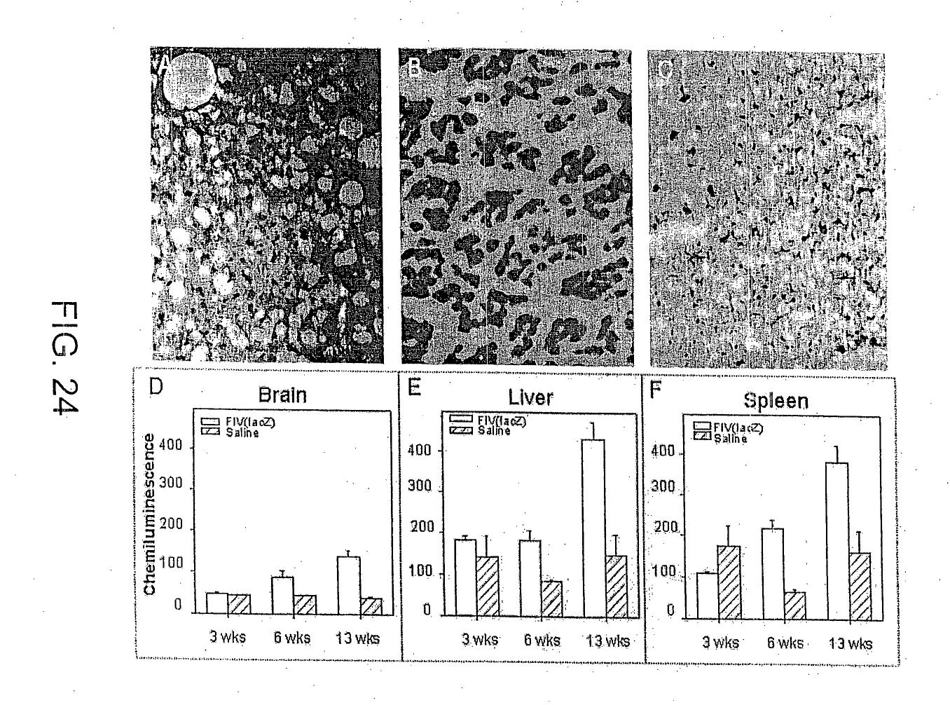

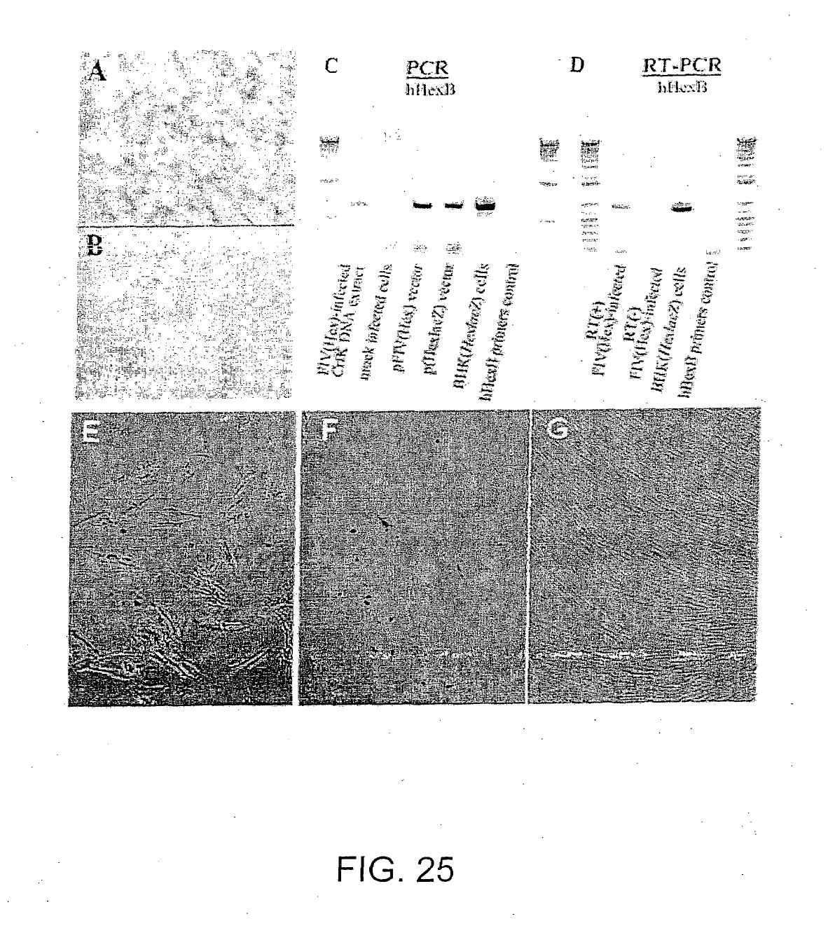

The ability of the β-hexosaminidase lentiviral FIV(Hex) vector to transduce murine cells was initially determined in normal mouse and subsequently in human Tay-Sachs fibroblasts. Murine wild type fibroblasts were infected with FIV(Hex) at 5×10⁷ infectious particles/mL (m.o.i.˜2) in vitro, and β-hexosaminidase expression was found increased compared to FIV(lacZ) infected cells as assessed by X-Hex histochemistry (FIG. 25A-B). Transgene incorporation was confirmed by PCR (FIG. 25C), and gene expression was evaluated at the transcription level by RT-PCR (FIG. 25D). Moreover, Tay-Sachs cultured primary fibroblasts were treated with FIV(Hex) at 5×10⁷ infectious particles/mL (m.o.i.˜2) 24 hours after being challenged with exogenously administered GM₂ ganglioside at cytotoxic concentrations (1 mg/mL) under serum-free conditions. FIV(Hex) administration to GM₂-challenged Tay-Sachs fibroblasts resulted in rescue of their spindle-like morphology and allowed their proliferation in vitro as soon as 2 days following FIV treatment (FIG. 25E-25G).

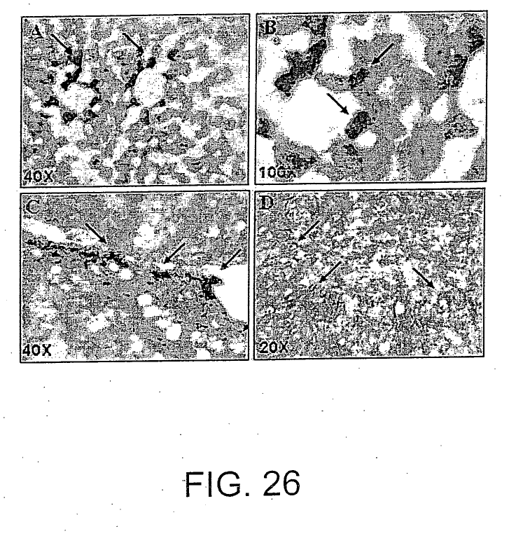

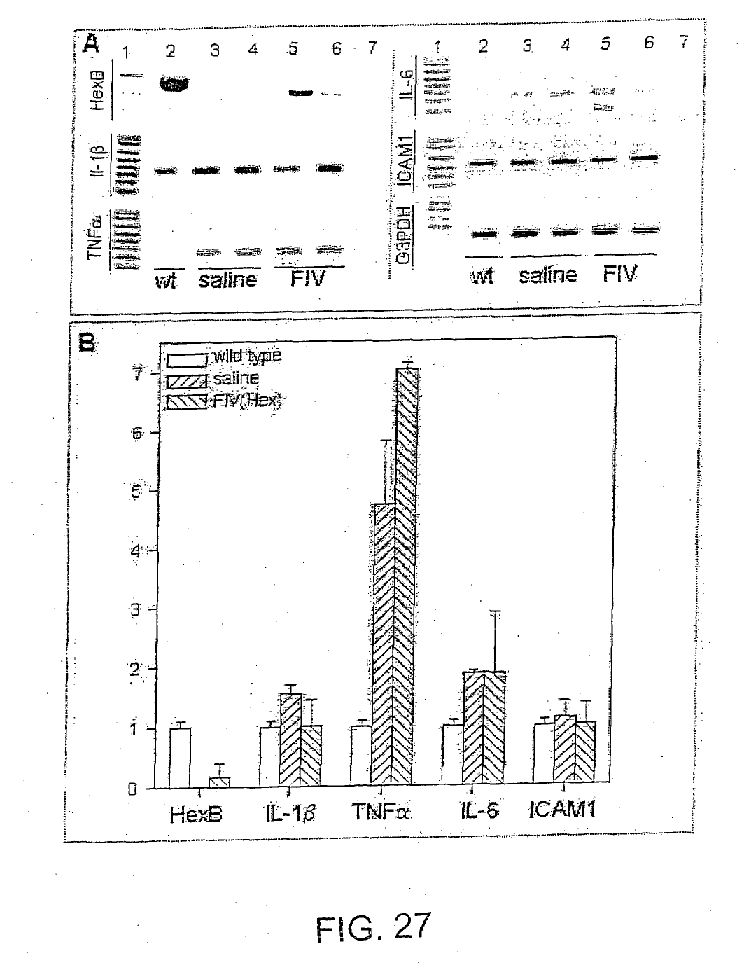

FIV(Hex) was also tested in vivo by injecting hexB^{-/-} pups at post-natal day P2 intraperitoneally (single dose of 10⁵ infectious particles) and evaluating β-hexosaminidase expression by HEXB immunocytochemistry. In the liver (FIG. 26A), transduced cells were primarily localized surrounding the portal triads (FIG. 26B). In the brain, β-hexosaminidase positive cells were located in periventricular areas of the cerebrum (FIG. 26C), which histologically appeared as eppendymal as well as glial cells. Moreover, β-hexosaminidase expression was also localized in cerebellar cortical cells that appeared as Purkinje neurons (FIG. 26D). HexB transgene mRNA expression was also evaluated, along with a number of inflammation-related genes, in the brain of hexB^{-/-} mice treated with FIV(Hex), as well as in hexB^{+/-} heterozygous littermates that served as controls. FIV(Hex) treatment of hexB^{-/-} pups resulted in detectable expression of HexB mRNA in the brain, at levels approximately 21% of that in hexB^{+/-} littermates. Transduction with FIV(Hex) resulted in reduction of IL-1β mRNA levels compared to hexB^{-/-} mice treated with saline. In contrast, TNFα was elevated following FIV(Hex) administration compared to hexB^{-/-} littermates as well as wild type mice. Overall (FIG. 27B), FIV(Hex) treatment resulted partial restoration of HexB normal brain activity, attenuation of EL-1β (p<0.05) and interestingly further exacerbation of TNFα expression (p<0.05).

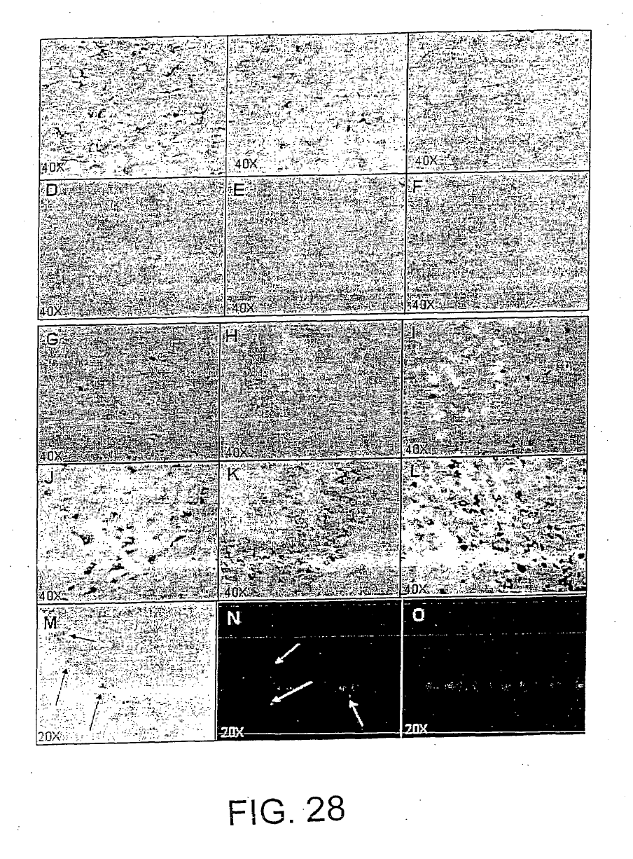

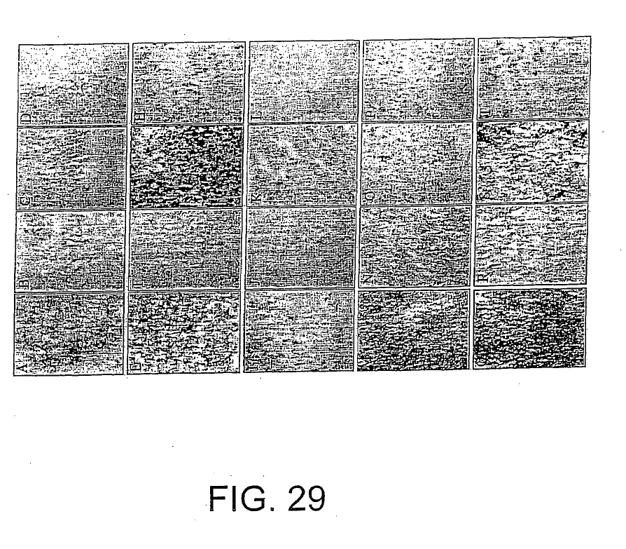

The degree of microglia and astrocyte activation was employed as a measure of brain inflammation, and was assessed by MHC-II and GFAP immunocytochemistry, respectively. Significant numbers of GFAP-positive astrocytes in the brain of 3-month-old hexB^{-/-} mice (FIG. 28A) in the thalamus, basal ganglia and cerebellum were found. FIV(Hex)-treated hexB^{-/-} mice showed reduced numbers of GFAP-positive cells in all the aforementioned areas of the brain (FIG. 28B), which were higher than the immunostaining of hexB^{+/-} heterozygous mice (FIG. 28C). Comparable brain sections revealed lack of MHC-II staining, suggesting the absence of activated microglia/macrophages/monocytes. Moreover, the degree of GM₂ storage in comparable brain sections by immunocytochemistry utilizing a commercially available monoclonal antibody (FIG. 28G-L) were examined, and reduced levels of GM₂ immunostaining in the brain stem (FIG. 28G), hippocampus (FIG. 28H) and thalamus (FIG. 5I) of hexB^{-/-} mice treated with FIV(Hex) compared to saline-treated animals (FIG. 28 J-L) were found. Heterozygous hexB^{+/-} as well as wild type mice did not display any positive GM₂ immunostaining (data not shown). Cell death, as assessed by the TUNEL method, was present in saline-treated hexB^{-/-} mice only; there was lack of TUNEL-positive cells in the FIV(Hex)-treated hexB^{-/-}, as well as heterozygous hexB^{+/-} mice (FIG. 28M). The cell death results were confirmed by the Fluoro-Jade method often employed for the identification of neurons undergoing degeneration (FIG. 28N-28O).

Similarly, the numbers of MHC-II and GFAP-positive cells were found reduced in FIV(Hex)-treated hexB^{-/-} mice 4 months after treatment compared to FIV(lacZ)-treated animals (Table 4), whereas wild type animals lacked any MHC-II or GFAP positive cells in the brain (data not shown).

Table 4 The number of GFAP⁺ and MHC-II⁺ positive cells were counted in 10 random microscopic fields (40×) in the cortex, basal ganglia, thalamus, cerebellum and brain stem of FIV(Hex)- and FIV(lacZ)-treated mice. In each field, the number of positive cells were counted, and average +/− standard deviation were calculated for each area of the brain.

TABLE 4

| | | Cortex | Basal ganglia | Thalamus | Cerebellum | Brain Stem |
|---|---|---|---|---|---|---|
| GFAP | FIV(lacZ) | 35 +/− 7 | 293 +/− 45 | 80 +/− 10 | 52 +/− 12 | 225 +/− 15 |
| | FIV(Hex) | 10 +/− 4 | 40 +/− 8 | 10 +/− 5 | 5 +/− 3 | 85 +/− 12 |
| MHC-II | FIV(lacZ) | 27 +/− 5 | 125 +/− 25 | 148 +/− 32 | 80 +/− 14 | 118 +/− 12 |
| | FIV(Hex) | 7 +/− 4 | 35 +/− 12 | 30 +/− 7 | 45 +/− 9 | 45 +/− 7 |

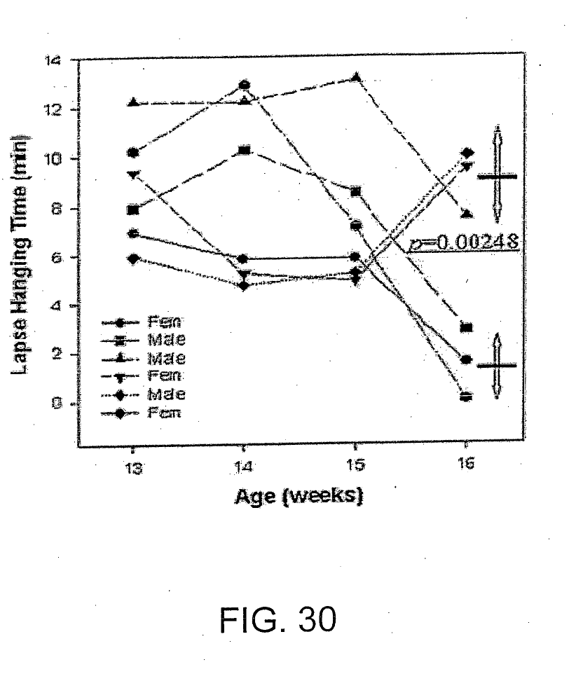

Specifically, GFAP-immunostaining was decreased in the cerebellum, thalamus, cortex, brain stem and basal ganglia of hexB^{-/-} mice treated with FIV(Hex) compared to FIV(lacZ)-treated littermates (FIGS. 28A-B, 28E-F, 28I-J, 28M-N and 28Q-R, respectively). MHC-II immunostaining was also decreased in all the aforementioned areas of the brain following FIV(Hex) treatment (FIGS. 28C-D, 28G-H, 28K-L, 28O-P, 28S-T). In addition, FIV(Hex) neonatal administration resulted in amelioration of the motor defect commonly seen in hexB^{-/-} mice at 4 months of age (FIG. 30A) without any significant effects on the overall development of the mice, as assessed by total weight (data not shown).

(1) FIV(Hex) Therapy Ameliorates the Brain Inflammation in hexB^{-/-} Adult Mice

As discussed herein, FIV(Hex) was administered intraperitoneally to 2 day old hexB^{-/-} pups (N=5) at 10⁷ infectious particles in a volume of 100 μL aqueous solution. In addition, an equal number of hexB^{-/-} littermates received injections of FIV(lacZ) serving as controls. Moreover, wild type pups received saline injections. The pups were then returned to their mother, until sacrificed at 16 weeks of age, a critical stage in disease development. The presence of activated microglia and/or monocytes/macrophages in the brain was assessed by immunocytochemistry (ICC) utilizing antibodies raised against major histocompatibility complex class II antigens (MHC-II), the expression of which is induced in inflammation. Brain sections from hexB^{-/-} mice treated with FIV(Hex), FIV(lacZ) and wild type controls were obtained. Immunocytochemistry for glial fibrillary acidic protein (GFAP), a marker of astrogliosis (activation of astrocytes), revealed an impressive decrease in the number of positive cells in the FIV(Hex) treated group versus the FIV(lacZ) group. There was minimal staining in normal mouse brain. The presence of large proteins in the brain parenchyma (IgG immunoglobulins) was employed as an indirect measure of blood brain barrier integrity. FIV(Hex) treatment prevented BBB leakage.

(2) Neonatal FIV(Hex) Transfer Attenuates Locomotive Deterioration in hexB^{-/-} Mice

FIV(Hex) was administered intraperitoneally to 2 day old hexB^{-/-} pups (neonates) at a dose of 10⁷ infectious particles in 100 μL aqueous solution. In addition, an equal number of hexB⁴ littermates received injections of FIV(lacZ) and served as control. The pups were then returned to their mother. Their body weight was monitored weekly, as well as their locomotive performance by the righting reflex and the inverted mesh test (mice are placed on a wire mesh in up-side-down position and the lapse time until mice fall from the mesh is recorded). Interestingly, at 16 weeks of age, there was significant difference between the two groups in locomotive performance (p=0.00248) as shown in FIG. 30.

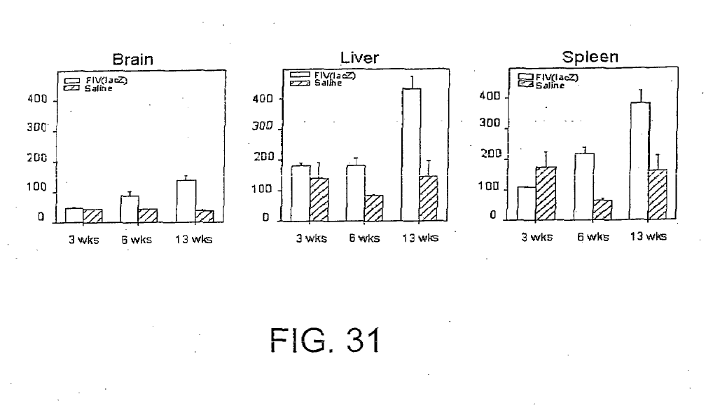

A quantitative analysis of neonatal FIV(lacZ) to P2 mice was performed. To quantitatively evaluate the efficacy of FIV as a platform for gene transfer in vivo, neonatal mice (P2) were injected intraperitoneally with 100 μL of 5×10³ infectious particles/mL, as well as normal saline, and were sacrificed at various ages: 3-6-12 weeks of age. Consequently, β-galactosidase expression (product of lacZ) was evaluated quantitatively in the brain, liver and spleen by an enzyme substrate chemiluminescence assay (Galactolight®; Tropix). Results were normalized for total protein and expressed as luminescence units. The results are shown in FIG. 31. Bone marrow Transplantation was also shown. The purpose was to demonstrate the level of competency with bone marrow transplantation (BMT) in mice. Six weeks following BMT of GFP expressing marrow, mice were injected into the striatum with a HSV viral vector expressing lacZ. Large numbers of green fluorescing cells were observed in the injected striatum and the non-injected striatum shows few or no GFP+ cells.

The purpose of this Example was to investigate the effects of a recombinant β-hexosaminidase FIV vector in the brain of hexB-deficient (Sandhoff disease) mice following intraperitoneal administration to pups of neonatal age. Since brain inflammation, neuronal cell death and motor dysfunction are characteristics of hexB-deficiency and the attendant GM₂ gangliosidosis, these parameters were employed as experimental outcomes in the Example. Systemic neonatal administration of FIV(Hex) resulted in restoration of HexB in the brain of affected mice, leading to reduction of brain inflammation, GM₂ storage and cell death along with amelioration of motor dysfunction.

It was not until recently that brain inflammation was realized as an important feature of GM₂ gangliosidosis (Wada R, et al., Proc Natl Acad Sci USA 97: 10954-10959. (2002); Oya Y, et al., Acta Neuropathol 99: 161-168. (2000); Myerowitz R, et al., Hum Mol Genet. 11: 1343-1350. (2002); Jeyakumar M, et al., Brain 126: 974-987. (2003)). It was also suggested that the aforementioned brain inflammation contributes to neurodegeneration: the presence of activated microglia and/or macrophages in the brain preceded neuronal cell death, and were observed proximal to neurons undergoing apoptosis. In support is the study by Norflus et al. (Norflus F, et al., J Clin Invest 101: 1881-1888. (1998)) that described attenuation of brain inflammation and amelioration of the disease phenotype following normal bone marrow transplantation to hexB^{-/-} pups, suggesting a critical role of peripheral myeloid derived cells in disease pathophysiology. Disclosed herein, systemic FIV vector administration suggested transduction of peripheral immune cells and subsequent infiltration of peripheral blood mononuclear cells (monocytes/macrophages) into the brain parenchyma in adults.

Initially, the efficacy of neonatal FIV administration to infect brain and peripheral tissues was evaluated by examining the expression of the reporter gene β-galactosidase over time in mice treated intraperitoneally with FIV(lacZ) at post-natal day P2. The results demonstrated the presence of β-galactosidase positive cells in the brain, spleen and liver, the expression of which increased with time, suggesting stable transduction of precursor cells by FIV(lacZ). Previous investigations on the effectiveness of perinatal systemic administration as the basis for global gene therapy have demonstrated detectable transgene expression for several months after the initial injection that varied between the tissues examined (Porada et al., 1998; Tarantal A F, et al., Mol Ther 3: 128-138. (2001); McCormack J E, et al., (2001) Mol Ther 3: 516-525. (2001); Lipschutz G S, et al., Mol Ther 3: 284-92. (2001)). Disclosed herein, the majority of the X-gal positive brain cells appeared to be microglia, monocytes, macrophages and/or endothelial cells based on their localization and histologic appearance. Also disclosed herein, FIV(lacZ) intraperitoneal administration results in transduction of CD31-(endothelial cells), CD3-(lymphocytes) and CD11b-(monocytes/macrophages) positive cells by means of double immunofluorescence. Similarly, Daly et al. (Daly T M, et al., Hum Gene Ther 10:85-94. (1999a)) reported widespread distribution of a β-glucuronidase transgene in a mouse model of Sly disease following perinatal systemic administration of an adeno-associated viral vector. Moreover, in a subsequent study, the authors reported that neonatal gene transfer of β-glucuronidase viral vectors lead to long-term attenuation of mucopolysaccharidosis along with amelioration of the aberrant phenotype in the Sly disease mouse (Daly T M, et al., Gene Ther 1291-1298. (2001)).

Administration of FIV(Hex) to hexB^{-/-} pups also resulted in transduction of brain cells that were primarily localized in periventricular areas and to a lesser extent in perivascular areas of the cerebrum. A previous study (Oya Y, et al., Acta Neuropathol 99: 161-168. (2000)) demonstrated that β-hexosaminidase bearing cells were mostly detected in the leptomeninges and choroids plexus, and to a lesser extent in perivascular areas of the brain and spinal cord after normal bone transplantation to hexB^{-/-} pups. This periventricular pattern of β-hexosaminidase expression in hexB^{-/-} brain after FIV(Hex) treatment is somewhat different from that observed in wild type mice following FIV(lacZ) administration, both in terms of location as well as number of cells. It is possible that these differences are a result of abnormal immune function in the hexB^{-/-} mice, whereby cellular immunity, and the monocyte-macrophage system in particular, are impaired by the β-hexosaminidase deficiency. In fact, Kieseier et al. (Kieseier B C, et al., Acta Neuropathol 94: 359-362. (1997)) reported that the monocyte-macrophage system, in addition to T- and B-lymphocytes, is affected in a number of storage human disorders. Moreover, abnormal immune function has been described in human patients suffering from Sly disease (Daly Tm, et al., Pediatr Res 47: 757-762. (2000)) as well as Gaucher disease (Burstein Y, et al., J Clin Lab Immunol 23: 149-151). Therefore, possible immune system anomalies in GM₂ gangliosidosis may result in impaired response to FIV(Hex) administration, leading to decreased numbers of transduced immune cells.

Two In adults, wherein the immune system is complete and the BBB is complete, disclosed herein, delivery of a vector to a brain cell, appears to be due substantially to fusion of a blood cell or development of a progenitor cell that have been transfected with the vector. In situations where the BBB is not completely developed or where the immune system is not completely developed, such as in neonates and in perinates, the situation is that direct transfection of brain cells seems to be the substantial mechanism for delivery of the vector to the brain cell.

The blood-brain barrier (BBB) is a structure unique to the central nervous system and is the result of tight junctions between the brain endothelial cells (Goldstein G W, et al., Ann NY Acad Sci 481:202-13. (1986)). Previous work (Risau et al., 1986) on the development of mouse BBB using large protein molecules (horse radish peroxidase) suggested BBB formation during the late days of embryonic life (E17 in mouse). Furthermore, BBB in the adult is not absolute, whereby certain areas of the brain do not develop BBB and thus allow for free exchange of molecules through them. These areas include the median eminence (hypothalamus), pituitary, choroids plexus, pineal gland, subformical organ, organum vasculosum lamina terminalis and area posterma (Risau W, Wolburg H, TINS 13: 174-178. (1990)). Hence, one could visualize the intrusion of virions into the brain matter through an incomplete BBB as well as through areas lacking BBB during the first few days after birth.

Receptor-mediated enzyme transfer (cross-correction) is an important characteristic of lysosomal enzymes, including β-hexosaminidase, whereby secreted enzyme can be up-taken by neighboring cells via paracrine pathways. The transport and compartmentalization of soluble lysosomal enzymes to lysosomes depends on the recognition of mannose 6-phosphate (Man-6-P) residues in their oligosaccharide moiety by specific receptors. Two distinct proteins have been thus far identified capable of interacting with lysosomal enzymes, the Man-6-P receptor (MPR; 270 kDa) which also binds the insulin-like growth factor-II (IGF-1), and the cation-dependent MPR (CD-MPR; 46 kDa). (Munier-Lehmann H, et al., Biochem Soc Trans 24: 33-36. (1996)). To this end, previous studies demonstrated that neural cell lines stably expressing HexA when transplanted into normal fetal and newborn brains of mice resulted in significant levels of active hexosaminidase protein throughout the engrafted brain (Lacorazza H D, et al., Nature Med 2: 424-429. (1996)). Moreover, unilateral intra-cerebral injection of an FIV vector encoding for the human β-glucuronidase gene in adult Sly disease mice resulted in bi-hemispheric reduction of lysosomal storage and attenuation of the clinical pathology (Brooks A I, et al., Proc Natl Acad Sci USA 99: 6216-6221 (2002)). Collectively, these studies indicate that successful transduction of a subset of brain cells with a therapeutic gene can lead to lysosomal storage resolution throughout the brain parenchyma, even at areas distant to the site of transgene expression.

In conclusion, the data presented herein indicate that neonatal intraperitoneal administration of FIV(Hex) to hexB^{-/-} pups resulted in transduction of brain cells with the therapeutic β-hexosaminidase gene, leading to reduction in neuro-inflammation, attenuation of the GM₂ storage and amelioration of the attendant neurodegeneration and motor behavioral deterioration. The applicability of these findings can also extend beyond the specific lysosomal storage disorder, since brain inflammation has been considered as a contributing factor in other neurodegenerative brain disorders as well (Lombardi V R, et al., J Neurosci Res 54: 539-53. (1998); Ohmi K, et al., Proc Natl Acad Sci USA 18: 1902-1907. (2003)).

### 7. Example 7

### Dose Response Testing, Bone Marrow Testing, and Additional Therapeutic Verification

As previously described (Sango K, et al., Nature Genet. 14: 348-52. (1996)) the hexB^{-/-} mice suffer from β-hexosaminidase deficiency (murine HEXA and HEXB), which results in progressive storage of GM₂ ganglioside in the lysosomes of neurons, ultimately leading to brain inflammation and neurodegeneration by 3 months of age. At 4 months, the mice present with severe motor behavioral deterioration and die soon thereafter. Two breeding pairs (hexB^{+/-}) can be used to maintain a colony on a heterozygous background for any experiments. These mice can be obtained (Sango K, et al., Nature Genet 11: 170-176 (1995)) Mouse genotypes can be determined by employing established PCR methods from tail biopsy DNA extracts. The following primers can be utilized:

TABLE 5

| | | | |
|---|---|---|---|
| Locus | Primers | T_{A} | Product |

| | | | |
|---|---|---|---|
| murine | ^{5′}ATT TTA AAA TTC AGG CCT | 58° C. | 120 bp |
| hexB | CGA^{3′} | | (wild type) |
| | ⁽SEQ ID NO:54) | | v/s |
| | ^{5′}CAT AGC GTT GGC TAC CCG | | 220 bp |
| | TGA^{3′} | | (knockout) |
| | (SEQ ID NO:43) | | |
| | ^{5′}CAT TCT GCA GCG GTG CAC | | |
| | GGC^{3′} | | |
| | (SEQ ID NO:44) | | |

a) FIV Systemic Administration: Dose Response

Stock virus can be prepared (at 10⁸ infectious particles/mL) as disclosed herein). Two-day old pups (P2) and 3 month old mice can be injected intraperitoneally with 100 μL and 1,000 μL of aqueous solution, respectively, containing increasing doses of FIV(Hex). Since neonates usually weigh 2.5 gr, whereas adults about 25 gr, FIV(Hex) administration can be adjusted for total body weight (See Table 6 for details), so that both receive comparable numbers of FIV vectors. A second group of mice can receive saline injections and can serve as controls.

Table 6 shows an exemplary transduction of brain cells with the β-hexosaminidase transgene after FIV systemic administration to mouse neonates experiment for determining a dose response. Four groups of 30 hexB^{-/-} mice each can be treated with increasing doses of FIV(Hex) intraperitoneally (listed as total number of infectious particles) at post-natal day P2. An additional 4 groups of equal number of mice can be treated at 3 months of age. The total number of infectious particles injected can be adjusted for body mass (see above). Additional mice (30 pups and 30 adults) can receive saline injections and can serve as controls. At each time point (3, 5 & 7 days after treatment), 40 pups and 40 adult mice (10 animals for each FIV dose) can be terminated and histologically evaluated by stereology as described below.

TABLE 6

| Age | Type | Treatment | Volume | Dose | Time Points | N |
|---|---|---|---|---|---|---|
| P2 | hexB^{-/-} | FIV(Hex) | 100 μL | 10⁶-10⁵-10⁴-10³ | 3, 5 & 7 days | 10 × 4 × 3 = 120 |
| P2 | hexB^{-/-} | saline | 100 μL | — | 3, 5 & 7 days | 10 × 3 = 30 |
| 3 months | hexB^{-/-} | FIV(Hex) | 1,000 μl | 10⁷-10⁶-10⁵-10⁴ | 3, 5 & 7 days | 10 × 4 × 3 = 120 |
| 3 months | hexB^{-/-} | saline | 1,000 μl | — | 3, 5 & 7 days | 10 × 3 = 30 |

| | |
|---|---|
| Total number of mice . . . | 300 |

The effects of FIV(Hex) treatment to brain cells after systemic administration to neonatal and adult mice can be evaluated acutely (within days after treatment) on histology sections employing histochemical, immunocytochemical and in situ hybridization methods. In brief, experimental (FIV-treated) and control (saline injected) mice can be deeply anesthetized and terminated via transcardial perfusion of 4% paraformaldehyde in phosphate buffered saline. The brain, brain stem and spinal cord can be harvested, frozen, cut in 20 μm thick sections and collected onto glass slides. The histology sections can first be analyzed by X-Hex histochemistry (As disclosed herein). In addition, localization of transgene expression can be evaluated by in situ hybridization (ISH) for HexA & HexB mRNA. For this purpose, small probes can be constructed that specifically hybridize to the human isoforms. (Brouxhon S M, et al., Brain Beh Immun 12: 107-122, 1998; Bellinger et al., 2001). In addition, HEXA and HEXB protein expression can be studied by immunocytochemistry (ICC) employing antibodies specifically raised against the human proteins (Proia R L, et al., J Biol Chem 259: 3350-3354. (1984)). Cellular identity can be confirmed by coupling ISH with immunocytochemistry (ICC) as previously described (Brouxhon S M, et al., Brain Beh Immun 12: 107-122, 1998; Bellinger et al., 2001) employing antibodies raised against markers for brain cells, including neurons (cerebral neurons stain with NeuN nuclear protein, Mullen R. J., et al., 116 (1992) 201-211. (1992); cerebellar Purkinje neurons stain with calbindin, Fournet N, et al., Brain Res. 399 (1986) 310-316), astrocytes (GFAP, Kyrkanides S, et al., Mol Brain Res 104: 159-9. (2002)), microglia/monocytes/macrophages (CD11b+/Mac1, Kyrkanides S, et al., Mol Brain Res 104:159-9. (2002)) and endothelial cells (PECAM-1, As disclosed herein). Similarly, cellular identity of cells expressing β-hexosaminidase can be assessed by double immunofluorescence as previously described (Kyrkanides S, et al., J Neuroimmunol 95:95-106. (1999), As disclosed herein) employing the same antibodies as listed above. The total numbers of transduced cells as well as the number of transduced cells for each cell type can be quantitatively determined for each FIV(Hex) dose administered in neonates and adult mice by stereology on alternate tissue sections as previous described (Olschowka et al., 2003).

Collectively, the aforementioned experiments can provide information on the distribution of the β-hexosaminidase vector after systemic administration of FIV to neonatal pups and young adult mice. The mice can be grouped according to age and type of treatment: 40 pups and 40 adult mice (N=10 for each of the FIV treatments) can be terminated at various time points (3, 5 & 7 days after treatment) for histologic evaluation by stereology (outcome measures are numerical variables). In addition, 10 pups and 10 adults injected with saline can also be terminated and analyzed at each time point. Any differences between the various groups will be assessed by multi-variable parametric statistical methods: two-way ANOVA (alpha=0.05) with Tukey post-hoc analysis, and the level of significance set at p<0.05.

Mannitol, a reagent known for its ability to “open” the blood brain barrier (BBB) can be systemically administered to mice in preparation for FIV injection in the case that BBB is suspected as a limiting factor in brain cell transduction. Therefore, an additional group of mice can be treated with FIV following mannitol administration. (Deng S X, et al., J Neurosci Methods 83: 159-164 (1998)).

Disclosed herein, in adult systemic injection, the involvement of the innate immune system following FIV systemic administration, whereby peripherally-derived immune cells infiltrate into the brain following transduction by the viral vector. A staining technique for VSV-G pseudotyped FIV vectors which can be utilized for determining the cells transduced by direct HIV infection versus those that were produced by mitosis. This method is based on covalently marking the viral envelop with the Cy3⁺ dye prior to administration (Bartlett J S, Samulski R J, Nat Medicine 4: 635-637 (1998)). Details on the use of this method have been previously reported (Bartlett J S, et al., J Virology 74: 2777-2785. (2000)).

b) FIV-Transduced Peripheral Immune Cells Enter into the CNS

Results disclosed herein on the long-term effects of FIV systemic administration indicate that in adult systemic injection transduced peripheral immune cells, including monocytes/macrophages and lymphocytes, can infiltrate and possibly engraft into the brain (As disclosed herein). The fate of FIV(Hex)-transduced bone marrow-derived cells transplanted to irradiated hexB^{-/-} mice ex vivo can be examined. Bone marrow can be harvested from hexB^{-/-} and wild type mice, which can then be transduced by FIV(Hex) in vitro and subsequently administered to hexB^{-/-} mice intraperitoneally. The presence and identity of transduced cells in the CNS and periphery (bone marrow and spleen) can be examined over time, and can provide data on the ability of myeloid-derived cells, including hematopoetic progenitors, to mediate gene transfer to the CNS.

(1) Bone Marrow Harvest

Donor bone marrow (BM) can be collected by flushing the femurs of wild type as well as hexB^{-/-} mice with Hank's balanced salt solution (Invitrogen, Carlsbad Calif.) as previously described (Suzuki K, et al., Lab Investigator 58: 302-309. (1998); Norflus F, et al., J Clin Invest 101: 1881-1888. (1998)). In brief, the cells can be collected by centrifugation, resuspended in a volume of 500 μl in Opti-MEM (Invitrogen) serum free culture media. The cells can then be infected with VSV-G pseudotyped FIV(Hex) or FIV(lacZ) at a multiple of infectivity of m.o.i.˜1. We routinely prepare FIV(Hex) at titers of 5×10⁸ infectious particles/mL, which can allow for proper final m.o.i. in this experiment. After 12 hours of incubation, the cells can be collected by centrifugation, resuspended in Hank's balanced salt solution. Two sets of transduced BM will be prepared for transplantation: wild type and hexB^{-/-}.

(2) Bone Marrow Transplantation

Recipient hexB^{-/-} mice (12 days old) can receive whole body irradiation of 9 Gy from a ¹³⁷CS source 1 day before transplantation (Kyrkanides S, et al., J Neuroimmunol 95:95-106. (1999); 2001). A total volume of 200 μl containing 5×10⁷ transduced cells derived from (A) wild type or (B) hexB^{-/-} donors can be injected intraperitoneally to the recipient mice at 14 days of age, which can then be returned to their mothers. In addition, a third group of mice can receive (C) non-transduced bone marrow from hexB^{-/-} donors and a fourth group (D) non-transduced bone marrow from wild type donors. The mice can be then terminated and analyzed at various time points after treatment (Table 7).

Table 7 shows an exemplary bone marrow transplantation in hexB−/− mice experiment set up. Bone marrow (BM) can be harvested from hexB^{-/-} or wild type mice and can be treated by FIV(Hex), the control vector FIV(lacZ) or normal saline in vitro prior to being administered to 12 days old hexB^{-/-} mice. The recipient mice can be grouped according to BM donor and treatment (50 mice in each group). At each time point, 10 mice from each group can be terminated and analyzed for brain β-hexosaminidase.

TABLE 7

| | | # Of | | Time | |
|---|---|---|---|---|---|
| Bm Donor | Treatment | Cells | Recipients | Points | N |
| hexB^{-/-} | FIV(Hex) | 5 × 10⁷ | hexB^{-/-} | 2, 4, 8, | 10 mice × 5 = 50 |
| hexB^{-/-} | FIV(lacZ) | 5 × 10⁷ | hexB^{-/-} | 12, 16 | 10 mice × 5 = 50 |
| hexB^{-/-} | saline | 5 × 10⁷ | hexB^{-/-} | wks | 10 mice × 5 = 50 |
| wild type | FIV(Hex) | 5 × 10⁷ | hexB^{-/-} | | 10 mice × 5 = 50 |
| wild type | FIV(lacZ) | 5 × 10⁷ | hexB^{-/-} | | 10 mice × 5 = 50 |
| wild type | saline | 5 × 10⁷ | hexB^{-/-} | | 10 mice × 5 = 50 |
| | | | | TOTAL | 300 mice |

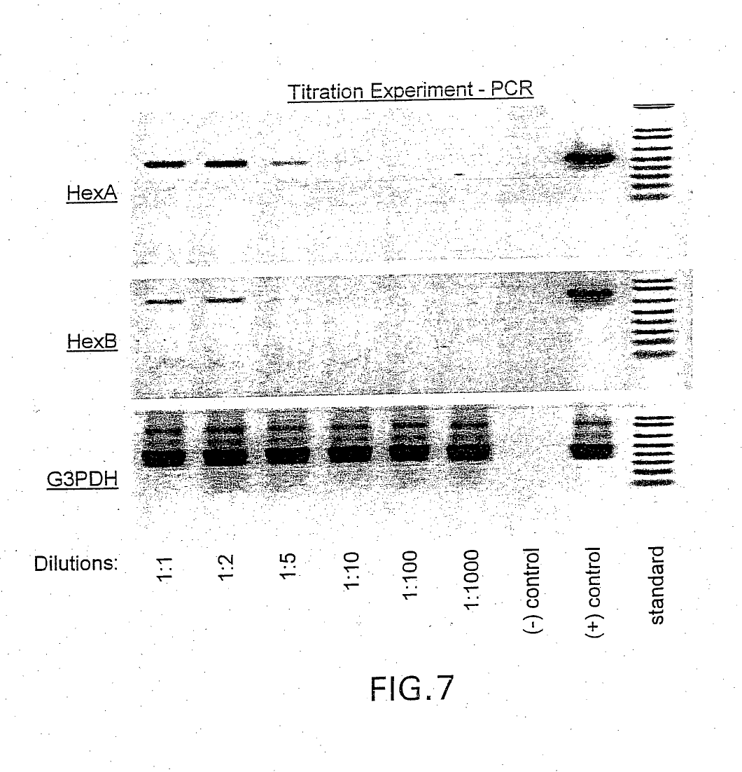

The presence and identity of transduced cells in the brain and peripheral tissues of recipient animals, including bone marrow and spleen, can be examined at various times points following bone marrow transplantation. The distribution of transduced cells in brain and peripheral tissues can be examined by immunocytochemistry (ICC) employing antibodies against the human HEXA and HEXB proteins (Proia R L, et al., J Biol Chem 259: 3350-3354. (1984); and as disclosed herein in other examples). β-hexosaminidase activity can also be confirmed by X-Hex histochemistry (FIG. 4; As disclosed herein). The identity of the transduced cells can be confirmed by double immunofluorescence by adopting methods previously described (Kyrkanides S, et al., J Neuroimmunol 95:95-106. (1999); 2003b). In brief, the goat anti-HEXA IgG and goat anti-HEXB IgG antibodies can be combined with antibodies raised against specific cellular markers: neurons (cerebral neurons stain with NeuN nuclear protein, Mullen et al., 1992; cerebellar Purkinje neurons stain with calbindin, Fournet N, et al., Brain Res. 399 (1986) 310-316), astrocytes (GFAP, Kyrkanides S, et al., Mol Brain Res 104: 159-9. (2002)), microglia/monocytes/macrophages (CD11b⁺/Mac-1, Kyrkanides S, et al., J Neuroimmunol 95:95-106. (1999); also MHC-II, FIG. 7), endothelial cells (PECAM-1, Kyrkanides et al., 2003b), astrocytes (GFAP; Kyrkanides S, et al., Mol Brain Res 104: 159-9. (2002)), lymphocytes (CD3⁺, As disclosed herein). The total number of transduced cells can be quantitatively determined by stereology on alternate tissue sections as previous described (Olschowka et al., 2003), as well as the total number of transduced cells for each cell type examined. In addition, the presence of the β-hexosaminidase transgene in brain cells can be confirmed by in situ hybridization (ISH) using whole length probes for human HexA and HexB mRNAs (Brouxhon S M, et al., Brain Beh Immun 12: 107-122, 1998; Bellinger D L. et al., Journal of Neuroimmunology 119(1):37-50, 2001). By coupling ISH with ICC, we will determine the identity of cells expressing HexA & HexB by employing cell specific markers as described above.

The presence of β-hexosaminidase vectors can be confirmed at the molecular level in the brain spleen and bone marrow DNA extracts of experimental and controls mice by polymerase chain reaction (PCR) utilizing primers designed specifically for our transgene (See for example, the Examples, herein). The number of β-hexosaminidase vectors can be quantitatively assessed by quantitative QPCR using the disclosed transgene specific primers by adopting methods previously described (Olschowka et al. 2003; As disclosed herein).

Collectively, the aforementioned experiments can provide information on the distribution of the β-hexosaminidase vector after systemic transplantation of ex vivo transduced bone-marrow derived cells over time. The mice can be grouped according to BM donor and treatment. For example, sixty (60) mice (N=10 from each subgroup) can be terminated at the various time points (2-4-8-12-16 weeks after treatment) for evaluation by stereology and QPCR (outcome measures are numerical variables). Any differences between the various animal groups can be assessed by multi-variable parametric statistical methods: two-way ANOVA (alpha=0.05) with Tukey post-hoc analysis, and the level of significance set at p<0.05.

c) Additional Data Regarding Therapeutic Levels of β-Hexosaminidase Expression being Achieved in the Brain Following Systemic Administration of Lentiviral Vectors

(1) Determine the Distribution, Level and Persistence of β-Hexosaminidase Expression after FIV(βact-Hex) Systemic Administration

The spatial distribution of the β-hexosaminidase transgene in hexB^{-/-} knockout mice following systemic administration of FIV(βact-Hex) at post-natal day P2, as well as 3 weeks and 3 months of age can be characterized. FIV(βact-Hex) is a transfer vector whereby the β-hexosaminidase transgene is driven by the chicken β-actin promoter (Daly T M, et al., Hum Gene Ther 10:85-94. (1999a); Daly T M, et al., Proc Natl Acad Sci USA 96: 2296-2300. (1999b)) which ensures prolonged expression in vivo (Daly et al. 2000). The mice can then be terminated at the critical age of 16 weeks old, whereby hexB^{-/-} mice display severe GM₂ ganlgiosidosis accompanied by clinical deterioration (Sango K, et al., Nature Genet 14: 348-52. (1996)). The level of HexA and HexB expression, at the mRNA, protein and activity levels, can be quantitatively assessed in the brain and vital organs, and can be correlated to the timing of administration.

(2) FIV(βact-Hex) administration

FIV(βact-Hex) can be administered in experimental (hexB^{-/-}) and control (hexB^{+/-}) mice at post-natal day P2, as well as 3 weeks and 3 months of age. Additional groups of mice can receive injections of the control vector FIV(lacZ) or normal saline. Litters that are derived from crossing hexB^{-/-} X hexB^{+/-} breeding pairs can be utilized, so that each litter consists of 50% experimental mice (hexB^{-/-}) and 50% “internal” controls (hexB^{+/-} heterozygous). Since the injections can be performed prior to genotyping, each litter can receive a single treatment.

The hexB^{+/-} heterozygous mice are characterized by reduced β-hexosaminidase activity but display near normal pathology and behavior (Sango K, et al., Nat. Genet. 1995 October; 11(2):170-6). In fact, Conzelmann et al. (1983) used a sensitive assay to demonstrate a correlation between level of residual β-hexosaminidase activity and clinical severity of GM₂ gangliosidosis: disease ≦0.1% of normal activity; late-infantile≈0.5% of normal activity; adult GM₂-gangliosidosis≈2-4%; clinically healthy persons ≧10% of normal activity. Therefore, the hexB^{+/-} heterozygotes can be employed as control animals

Specifically, a total of 10⁷ infectious particles of FIV(βact-Hex) or FIV(lacZ) can be injected intraperitoneally into each P2 pup (in a total volume of 100 μl). Saline-treated animals can receive 100 μl of sterile saline solution. Furthermore, the older mice can receive FIV(βact-Hex) or FIV(lacZ) injections at titers adjusted for total mass. For example, a mouse neonate weighs on average 2.5 gr, whereas a 3 month old mouse is approximately 25 gr in weight: Therefore, the older mouse can receive a total of 10⁸ infectious particles (in a total volume of 1,000 μl). The FIV(lacZ) group of mice can provide information on the effects of the viral vector itself, whereas the saline injections can control for the procedure (please refer to Table 8).

Table 8 shows an exemplary FIV(Hex) administration for the treatment of β-hexosaminidase deficiency. Fifteen (15) β-hexosaminidase deficient mice (hexB^{-/-}) can be administered FIV(Hex), FIV(lacZ) or normal saline intraperitoneally at post-natal day P2, as well as at 3 weeks and 3 months (12 wks) of age. In addition, 15 heterozygous “normal” littermates (hexB^{+/-}) can receive normal saline injections and can serve as controls. All mice can be terminated and analyzed at the critical age of 16 weeks, when the hexB^{-/-} mice display severe GM₂ gangliosidosis and clinical deterioration.

TABLE 8

| | | | | | | Analysis (per |
|---|---|---|---|---|---|---|
| TYPE | PATHOLOGY | TREATMENT | Time Points | KILLED | N | group) |
| hexB^{-/-} | Affected | FIV(βact-Hex) | P2, 3 & 12 wks | 16 wks | 45 | Molecular (n = 5) |
| hexB^{-/-} | Affected | FIV(lacZ) | P2, 3 & 12 wks | 16 wks | 45 | Biochemical (5) |
| hexB^{-/-} | Affected | saline | P2, 3 & 12 wks | 16 wks | 45 | Histology (n = 5) |
| hexB^{+/-} | “normal” | saline | P2, 3 & 12 wks | 16 wks | 45 | Behavioral (all) |

| | |
|---|---|
| Total mice . . . | 180 |

The sphere of β-hexosaminidase transgene distribution can be quantitatively assessed by determining the number of gene copies in the brain (cerebrum & cerebellum), liver, spleen and bone marrow by employing established QPCR methods (Olschowka et al., 2003). The levels of (HexA and HexB expression of the mRNA in brain (cerebrum & cerebellum), liver, spleen and bone marrow RNA extracts can be determined by QRT-PCR protocols as previously described (As disclosed herein; Olschowka et al., 2003). HEXA and HEXB protein expression levels can be evaluated by western immunoblotting (Tsuji et al., 2002; Proia R L, et al., J Biol Chem 259: 3350-3354. (1984)). In addition, we can quantitatively determine the levels of HexA and HexB enzymatic activity by 4MUG/S fluorometry (please refer Examples herein for examples of how this was done). These data can be analyzed relative to treatment and timing of administration by the two-way analysis of variance (ANOVA) with alpha=0.05. Differences between the various groups can be determined by the Tukey post-hoc analysis (p<0.05).

HexA, HexB and lacZ mRNA localization can be investigated by in situ hybridization (ISH) on brain, liver, spleen and bone marrow tissue sections as previous described (Brouxhon S M, et al., Brain Beh Immun 12: 107-122, 1998; Bellinger D L. et al., Journal of Neuroimmunology 119(1):37-50, 2001) using probes for the human HexA and HexB. By coupling ISH with immunocytochemistry (ICC), one can determine the identity of cells expressing HexA & HexB by employing cell specific markers, including cerebral neurons (NeuN), cereberal neurons (calbindin), microglia (CD11b), astrocytes (GFAP), endothelial cells (PECAM-1), monocytes/macrophages (CD11b), lymphocytes (CD3) as described herein. We can quantitatively assess the transduction of the various cell types by the β-hexosaminidase transgene on alternate tissue sections utilizing stereology methods as previous described (Olschowka et al., 2003; Methods in Detail). The data can be statistically analyzed as described herein, and collectively can provide information about the mRNA levels in the various tissues, as well as indicate any cellular preference for FIV vectors.

In addition, localization of transgene expression at the protein level can be qualitatively assessed by immunocytochemistry (ICC) on histology sections harvested from brain (cerebrum & cerebellum), liver, spleen and bone marrow utilizing antibodies against human HEX-A and HEX-B as described herein. The identity of the cells can be confirmed by double immunofluorescence as described herein (adopted from Kyrkanides S, et al., J Neuroimmunol 95:95-106. (1999; 2003b). The total number of transduced cells, as well as the number of neurons, glia, endothelial cells, macrophages and lymphocytes can be quantitatively determined by stereology on alternate brain, liver, spleen and femour histology sections as previous described (Olschowka et al., 2003). A histochemical method for the visualization of β-hexosaminidase activity on histology tissue, X-Hex histochemistry, can also be used (see Examples herein).

Persistence of β-hexosaminidase expression can be assessed as follows. Based on the data derived from the aforementioned experiments, one can temporally analyze HexA & HexB expression (mRNA, protein and activity levels) in relation to the number of transgene copies present in the various organs over time. Interpretation of the data can provide a measure of persistence (versus silencing). Any differences between the various groups can be assessed by multi-variable parametric statistical methods: two-way ANOVA (alpha=0.05) with Tukey post-hoc analysis, with the level of significance set at p<0.05. Moreover, one can calculate the percent of β-hexosaminidase recovery in hexB^{-/-} treated animals relative to hexB^{+/-} heterozygous littermates, normalized to total protein, in the various tissues following treatment. This analysis will provide information pertinent to the efficacy of FIV gene therapy in restoring β-hexosaminidase activity. Any differences between the various groups can be assessed by multi-variable parametric statistical methods: two-way ANOVA (alpha=0.05) with Tukey post-hoc analysis; level of significance set at p<0.05.

(3) Evaluation of the Effects of FIV(βact-Hex) Vector Administration on Disease Development

The hexB^{-/-} knockout mice are characterized by biochemical, pathologic and clinical features similar to those seen in TSD and SD patients (Sango K, et al., Nat. Genet. 1995 October; 11(2): 170-6). The effects of FIV(βact-Hex) systemic administration on lysosomal storage, neuronal cell death, and behavioral performance in hexB^{-/-} knockout mice can be obtained (See Table 8), and one can determine the sphere of transgene distribution and the levels HexA & HexB expression required for efficacious gene therapy.

Storage of insoluble metabolites can be first evaluated in histology brain sections by periodic acid Schiff histochemical staining (Sango K, et al., Nat. Genet. 1995 October; 11(2):170-6; Suzuki K, et al., J Neuropath Exp Neurol 56: 693-703. (1997)). In addition, GM₂ storage levels can be assessed by immunocytochemistry employing commercially available monoclonal anti-GM₂ antibodies (Sakuraba et al., 1997; Seikagaku, Falmouth Mass.). GM₂ staining differences in the various animal groups can be evaluated by semi-quantitative analysis as previously described (Olschowka et al., 2003). For quantitative analysis of GM₂ in the brain, one can employ immuno-thin layer chromatography (As disclosed herein).

Neuronal degeneration can first be evaluated histochemically utilizing the Fluoro-Jade agent (Fluoro-Jade; Histo-Chem. Inc., Jefferson Ariz.), a fluorescent agent that stains neurons undergoing degeneration (Schmued et al., 1997; Methods in Detail). The number of neurons undergoing apoptosis can be assessed by the fluorescin terminal uridine nick-end labeling method (TUNEL detects cellular apoptosis) on alternate brain sections. In addition, one can confirm apoptosis by double immunofluorescence utilizing antibodies raised against caspase-3 or -8. The total number of neurons can be determined also by double immunofluorescence with antibodies against NeuN nuclear protein (calbindin for cerebellar Purkinje neurons). The total number of nuclei can be determined by Hoechst nuclear staining. The number of cells undergoing cell death can be counted by stereology in alternate brain sections normalized for total number of nuclei (Hoechst staining).

Since the introduction of non-self proteins is anticipated to elicit an immunologic response in immunocompetent mice treated with FIV vectors, which potentially may neutralize β-hexosaminidase enzyme activity, one can characterize the host's immunologic response following perinatal treatment. To this end, one can quantitatively assess the presence (titers) of mouse antibodies against viral and transgenic proteins in the serum of mice. To this end, IgG and IgM titers for HEXA and HEXB, as well as the FIV p24 antigen can be assessed by customized ELISA method. In brief, ELISA plates can be coated with 5 mg of human HEX-A, HEX-B (Sigma; St. Louis Mo.) or p24 recombinant proteins (IDEXX Laboratories Inc.; Westbrook Me.). After incubation with the sera, the plates can be incubated with alkaline phosphatase-conjugated goat anti-mouse IgG and IgM (Southern Biotechnology Associates, Inc; Birmingham Ala.). Antibody titers can be established as the serum dilution that reached absorbance levels (at 405 nm) of saline injected mice assuming linear extrapolation (Kang Y, et al., J Virol 76: 9378-88. (2002)).

Evaluation of behavioral performance is also an important treatment outcome measure. Animal weight of experimental and control mice will be monitored weekly throughout the experiment. Motor competency will be assessed by the ability to maintain balance on a rotating cylinder (rotorod) by measuring the latency of each animal to fall off. In addition, their motor activity will be assessed by placing the mice on a wire mesh fixed on one end of a clear plastic cylinder, and turning the cylinder with the mesh and mice attached up side down. Motor strength will be evaluated in the experiment by measuring the latency of each animal to fall off. Using these two methods, we will assess motor behavior experimental and control mice on a weekly basis. Life span will also be recorded, since the affected mice suffer from significantly shortened life span (4-5 months). For that purpose, we will allow a subgroup of experimental mice to fully complete their life cycle and hence evaluate the efficacy of FIV in attenuating the most perhaps devastating of the features of this class of disorders (in humans and animals). Life span will be calculated as the total number of days an animal survived.

Compilation of the above data, in conjunction with the data on β-hexosaminidase expression, can provide information on the level of β-hexosaminidase levels required for clinical correction of the disease relative to wild type littermates. The aforementioned experiments, collectively, can provide additional data as to the efficacy of FIV gene therapy in restoring f-hexosaminidase activity in a mouse model of GM₂ gangliosidosis, and other subjects, such as humans. Any differences between the various groups can be assessed by multi-variable parametric statistical methods: two-way ANOVA (alpha=0.05) with Tukey post-hoc analysis; level of significance set at p<0.05.

### 8. Example 8

### Determining the Role of Neuronal GM2 Storage in Microglia Activation and Brain Inflammation, Determining the Role Peripheral Blood Mononuclear Cells in GM2 Gangliosidosis, and Providing Additional Evidence of the Efficacy of βHex Vectors in Transducing Neurons, Microglia and PBMCs to Intercept the Development of GM₂ Gangliosidosis

Human and animal studies suggest that β-hexosaminidase disorders involve a brain inflammatory process which develops secondary to neuronal storage of GM₂ ganglioside. Interestingly, brain inflammation has been considered as a contributing factor in other neurodegenerative brain disorders such as Alzheimer's disease (Lombardi et al. J Neurosci Res 54: 539-53 (1998); Szpak et al. Quantitative study. Folia Neuropathol 39: 181-92. (2001)). Disclosed herein are the roles of neuron-microglia interaction in GM₂ gangliosidosis and its effects on disease development in the hexB^{-/-} knockout mouse and how it relates to human subjects.

a) Determining the Role of Neuronal GM₂ Storage in Microglia Activation and Brain Inflammation

Neuronal GM₂ gangliosidosis is considered a cardinal feature in the development of TSD and SD disease. Recent reports suggest that microglia, the resident macrophages of the CNS, may play an important yet undefined role in disease pathology. GM₂ lysosomal storage in neurons results in likely results in activation of microglia, which in turn elicits a deleterious inflammatory process in the brain. Disclosed herein are methods of restoring β-hexosaminidase activity selectively in the neurons of hexB^{-/-} mice and thereby attenuating the neuronal GM₂ lysosomal storage and neuro-inflammation. Disclosed bicistronic genes (β-Hex) encoding for both subunits of the human β-hexosaminidase, HexA & HexB, the expression of which will be driven by the neuron specific enolase (NSE) promoter can be used. Crossing of transgenic mice characterized by NSE-βHex germline transmission with the hexB^{-/-} knockouts can be performed. Transgene expression can be analyzed in brain tissues as well as primary neuronal cultures employing biochemical, histological and molecular methods. The effects of neuronal rescue from GM₂ storage can be evaluated in vivo by molecular, histological and clinical (behavioral) methods, with particular interest in microglia activation and brain inflammation.

(1) Construction of the NSE-βHex Transgene

Disclosed herein and tested is a tricistronic gene (βHex) encoding for both subunits of the human β-hexosaminidase, HexA & HexB, as well as the reporter gene β-galactosidase (lacZ): HexB-IRES-HexA-IRES-lacZ (pHEXlacZ). Expression of the second and third open reading frames is facilitated by an internal ribosomal entry sequence (IRES). This transgene allows for the synthesis of HEX-A (α/β heterodimer) protein. In its current format, pHEXlacZ is driven by the ubiquitous cytomegalovirus promoter (CMV) or beta actin promoter (FIG. 1). Expression of the transgene can be targeted selectively to neurons by cloning a neuron specific promoter, such as the NSE promoter, in place of the existing CMV promoter—melanocortin-4 receptor promoter (Liu H. et al., Journal of Neuroscience. 23(18): 7143-54, 2003); tyrosine hydroxylase promoter (Kessler M A. et al., Brain Research. Molecular Brain Research. 112(1-2):8-23, 2003); myelin basic protein promoter (Kessler M A. et al Biochemical & Biophysical Research Communications. 288(4):809-18, 2001); glial fibrillary acidic protein promoter (Nolte C. et al., GLIA. 33(1):72-86, 2001); neurofilaments gene (heavy, medium, light) promoters (Yaworsky P J. et al., Journal of Biological Chemistry. 272(40):25112-20, 1997) (All of which are herein incorporated by reference at least for the sequence of the promoters and related sequences.) The NSE promoter is disclosed in Peel A L. et al., Gene Therapy. 4(1):16-24, 1997) (SEQ ID NO:69) (pTR-NT3myc; Powell Gene Therapy Center, University of Florida, Gainesville Fl.).

The following construct was successfully produced.

NSE [symbol] HexB-IRES-HexA-IRES-lacZ (NSE-Hex).

The BglI-BamHI segment of pTR-NT3myc containing the NSE promoter into the XhoI-HindIII sites of pBS KS +/− plasmid (Strategene; La Jolla Calif.) was cloned. Next, the NheI-NotI segment of pHEXlacZ was inserted into the EcoRV-NotI sites of pBS downstream to NSE by 5′-blunt and 3′-sticky ligation (pNSE-Hex). The function of NSE-Hex was analyzed in neurons, the neuronal N2α cell line (American Tissue Culture Collection, Manassas Va.; cat.# CCL-131). NSE-Hex was transiently transfected in cultured N2c's employing the Lipofectamine 2000 reagent per manufacturer's instructions (Invitrogen; Carlsbad, Calif.).

The NSE-Hex vector was tested as the NSE-HexB-IRES-HexA-IRES-lacZ transgene in vitro. The function of NSE-Hex was evaluated in the neuronal N2α cell line (American Tissue Culture Collection, Manassas Va.), whereby NSE-Hex was transiently expressed. The CMV-Hex gene was employed as positive. β-galactosidase expression was assessed by X-gal histochemistry: NSE-Hex and CMV-Hex transfected cells showed X-gal positive staining (black), whereas cells transfected with a vector devoid of an open reading frame (NSE-) had no staining. HexA and HexB mRNA expression was detected by RT-PCR, as well as lacZ. The housing-keeping gene G3PDH was used as control. RT(−) denotes the absence of reverse transcriptase enzyme in the reaction and controlled for possible DNA contamination by DNA carry-over during RNA extraction.

One can quickly assess transgene expression in transfected cells by X-gal histochemistry. Expression of HexA and HexB can be tested at the mRNA, protein and activity levels, by quantitative reverse transcriptase polymerase chain reaction (QRT-PCR; Olschowka et al., 2003), immunocytochemistry (See examples herein), X-Hex histochemistry (See Examples herein) and 4MUG/S fluorometry (See examples herein). A NSE-lacZ vector can be employed as control in the transfection experiment. Results can be normalized for transfection efficiency at the DNA level by PCR utilizing primers designed specifically for bacterial plasmid sequences. Based on previous experiments, NSE-Hex transfection in N2α cells will very likely result in induction of HexA and HexB levels of expression.

A variation of the NSE-βHex mouse would be a mouse expressing the βHex from a CD11b promoter (Dziennis et al., 1995) This promoter is microglia/macrophage/monocyte specific. This mouse would be useful when after clearance of neuronal GM₂ storage, microglia become activated and PBMC infiltration persists, indicating that GM₂ storage may be affecting microglia and perhaps PBMC directly. Results contained with this mouse can be compared with the results of selective restitution of β-hexosaminidase in neurons versus microglia/monocyte/macrophages. At this point, however, it is widely believed that neurons are the primary cell type suffering from GM₂ storage due to their relatively high levels of ganglioside concentration. Nevertheless, it is possible that microglia also develop GM₂ storage due to their inability to degrade endocytosed glycolipids. This latter scenario can be addressed by developing a transgenic mouse whereby βHex is ubiquitously expressed by universal promoters, such as β-actin or ROSA26.

(2) Development of the NSE-Hex Transgenic Mouse

The NSE-Hex transgene was prepared for microinjection and injected into fertilized C57B6/J oocytes followed by re-implantation of the surviving eggs into a pseudopregnant mother. The NSE-HexB-IRES-HexA-IRES-lacZ construct was linearized following Bgl II-Not I digestion from the backbone vector and checked for purity and size. Accuracy was confirmed by direct DNA sequencing. Nine pups were delivered representing founder lines that were analyzed for transgene incorporation, germline transmission and function. PCR amplification of DNA obtained from mouse tail biopsies of the nine founder mice using primers for the HexB transgene was performed. Two mice, #2 and #8 were positive for the NSE-Hex transgene. In addition, housekeeping G3PDH gene controls were performed. Analysis revealed two NSE-Hex positive mice. The following protocols can also be performed to produce NSE-Hex mice, as well as Hex mice, utilizing other promoters, by altering the protocols for the specific promoter desired.

Transgenic mouse lines can be made by injecting linearized NSE-Hex into fertilized mouse oocytes (C57BL/6) followed by re-implantation of surviving eggs into pseudopregnant recipient females. The NSE promoter has been previously employed with success in directing the expression of transgenes selectively in the neurons of transgenic mice (Kearne et al., 2001). The transgenic mouse lines can be analyzed for transgene incorporation and function, as well as germline transmission. The transgene can be maintained in a heterozygous state on the C57BL/6 background. Transgene incorporation (1) can be tested in tail DNA extracts by PCR using human-specific HexB, HexA and lacZ primers (genotyping). Primers have been designed that selectively detect the human HexB & HexA (See examples herein), as well as bacterial lacZ, but not the murine isoforms (see Methods in Detail for primer sequences). (2) Transgene incorporation can then be confirmed in brain DNA extracts by Northern blotting using whole length probes designed specifically for the human HexB and HexA.

Transgene function can be evaluated in vivo as follows. (1) First, lacZ expression can be readily assessed in brain histologic sections by X-gal histochemistry (See examples herein). (2) HexA and HexB expression at the mRNA level can be evaluated by QRT-PCR in total mRNA extracts from mouse brain, and can be compared to wild type littermates. Localization of HexA & HexB mRNA can be achieved on brain histology sections by in situ hybridization (ISH); cellular identity can be confirmed by coupling ISH with immunocytochemistry (ICC) employing antibodies raised against NeuN nuclear protein (cerebral neurons; Mullen et al. Development 116: 201-211 (1992); Chemicon INTL, Temacula Ca) as well as calbindin (Purkinje cerebellar neurons; Fournet et al. Brain Res. 399 310-316 (1986); Brouxhon et al. Beh Immun 12: 107-122 (1998); Bellinger et al. Journal of Neuroimmunology 119(1):37-50 (2001)). (3) HEX-A and HEX-B protein expression can be analyzed by ICC in brain sections employing antibodies raised specifically against the human HEX-A and HEX-B proteins (Proia et al. J Biol Chem 259: 3350-3354 (1984)) These antibodies for HEX-A and HEX-B have been successfully used. (See examples herein). The identity of HEX-A and HEX-B expressing cells can be confirmed by double immunofluorescence employing antibodies against NeuN for cerebral and calbindin for cerebellar neurons coupled with the aforementioned anti HEX-A & anti HEX-B antibodies. (See examples herein and Kyrkanides et al. J Neuroimmunol 95:95-106. (1999)). Quantification of HEX-A and HEX-B protein levels in NSE-Hex and wild type littermates can also be assessed by western immunoblotting utilizing the aforementioned antibodies (Utsumi et al. Acta Neurol Scand 105:427-30 (2002); Proia et al. J Biol Chem 259: 3350-3354 (1984)). (4) HexA and HexB enzyme activity can be first evaluated on brain histology sections by X-Hex histochemistry (FIG. 4). In addition, HexA & HexB activities can be quantified by 4MUGS and 4MUG fluorometry (FIG. 1 of Preliminary Data) in NSE-Hex and wild type littermates. (5) The data derived from at least two generations can be compared to ensure germline transmission and persistence of the expected phenotype.

TABLE 9
Transgene expression in NSE-Hex transgenic mouse lines

| Analysis | Animals (N) | Methods |
|---|---|---|
| mRNA | 5 × 7 lines = 35 | QRT-CPR |
| protein | 5 × 7 lines = 35 | western immunoblotting |
| | | β-hexosaminidase activity |
| histology | 5 × 7 lines = 35 | immunocytochemistry (ICC) |
| | | in situ hybridization (ISH) |
| | | X-gal & X-Hex histochemistry |

(3) Determining Whether GM₂ Storage in Neurons Induce Microglia Activation and Subsequent Brain Inflammation

One can rescue neurons from developing GM₂ storage in vivo by restoring neuronal β-hexosaminidase activity in hexB^{-/-} mice as follows. First, the NSE-Hex transgenic (Tg) mouse can be crossed into the hexB^{-/-} background, and the offspring can be back-crossed into hexB^{-/-}: based on this strategy, each litter can consist of hexB^{-/-}/Tg^{+/-} experimental and hexB^{-/-}/Tg^{-/-} control mice. Since the NSE-Hex transgenic mouse can be developed on the C57BL/6 background, whereas the hexB^{-/-} mouse is on a mixed C57BL/6 & 129S background, littermate controls can be employed in the studies to ease any concerns related to potential strain differences. One can confirm the expression of HexA & HexB in neurons of the brain of these mice at the mRNA, protein and enzyme levels, as well as evaluate neuronal GM₂ storage and cell death. In addition, one can assess microglia activation and brain inflammation. Finally, one can evaluate disease development at the behavioral level.

NSE-Hex expression in neurons can be determined at the mRNA, protein and enzyme levels as described above. In brief; the levels of human HexA and HexB mRNA can be quantitatively assessed by QRT-PCR in brain RNA extracts obtained from hexB^{-/-}/Tg^{+/-} experimental as well as hexB^{-/-}/Tg^{-/-} control littermates. In addition, brain samples from appropriate wild type mice (B6129SF2; Jax, stock# 101045) as well as hexB^{-/-} knockouts can be included in the analysis and can serve as additional controls. HexA & HexB mRNA localization can be performed by ISH and neuronal identity can be confirmed by ISH/ICC utilizing antibodies against NeuN nuclear protein (cerebral neurons) as well as calbindin (cerebellar Purkinje Neurons). HEX-A and HEX-B protein expression can be evaluated by ICC in brain sections, and neuronal identity can be confirmed by double immunofluorescence as described above. HEX-A & HEX-B proteins can be quantitatively evaluated by western immunoblotting (Utsumi et al., 2002). Furthermore, X-gal histochemistry can provide additional confirmation of transgene expression. HexA & HexB activity can be assessed on histology brain sections by X-Hex histochemistry, and can be quantitatively analyzed by 4MUGS & 4MUG fluorometry (See examples herein).

GM₂ neuronal storage is a cardinal histopathologic feature and can be readily detected by simple histochemical methods, including periodic acid Schiff (PAS) and Alcian blue staining (Sango K. Nature Genet 14: 348-352 (1996); Suzuki et al. J Neuropath Exp Neurol 56: 693-703 (1997)) on fixed brain, brain stem, cerebellum and spinal cord sections. Using a monoclonal antibody against GM₂ ganglioside (Seikagaku, Falmouth Md.), one can process brain sections by ICC. In brief, alternate brain sections (20 μm thick) can be stained by GM₂ ICC and mounted onto glass slides and cover-slipped. The number of positive cells can be quantitatively assessed by stereology methods as previously described (Olschowka et al., 2003; see Examples herein). The levels of GM₂ ganglioside in the brain can be assessed quantitatively by immune-thin layer chromatography (see Examples herein) Neuronal degeneration is also cardinal in GM₂ gangliosidosis and can be evaluated by processing alternate brain sections with Fluoro-Jade (Histo-Chem. Inc., Jefferson Az.), a fluorescent agent that stains neurons undergoing degeneration (Schmued et al. Brain Res 751: 3746 (1997). In addition, neuronal cell death can be evaluated by the fluorescin terminal uridine nick-end labeling method (TUNEL). The sections can then be counter-stained with propidium iodide. To confirm apoptosis, one can double stain with antibodies against caspase-3 or -8. The total number of nuclei can be determined by Hoechst nuclear counter-staining. The number of Fluoro-Jade, TUNEL as well as caspase-3 & -8 positive neurons can be quantitatively determined employing the aforementioned stereology method.

Brain inflammation can be assessed by studying the levels of inflammation-related genes in the brain of experimental, control and wild type mice. First, the presence of activated microglia can be assessed in brain histology sections by immunocytochemistry employing antibodies against MHC class II antigens, the expression of which are upregulated in inflammation, and can be quantified by stereology protocols in alternate brain sections (See examples herein). The total number of microglia can be assessed by staining for CD11b (Mac-1) antigen. Comparison between the numbers of MHC 1′-positive and CD11b-positive cells can provide information on the total number of microglia in the brain, and the percentage that become activated. In addition, tumor necrosis factor-α (TNFα) mRNA levels can be determined by QRT-PCR and can be employed as an additional measure of microglia activation. Astrogliosis (activation of astrocytes) can be assessed by staining for glial fibrillary acidic protein (GFAP), as well as quantitative measurement of GFAP mRNA levels in brain. Moreover, an array of inflammatory genes (Il-1β, ICAM-1, MCP-1, INF-γ, IP-10) can be utilized to evaluate brain inflammation previously described in detail (Examples herein; Kyrkanides et al., 2001, 2002).

Lack of motor competency is an important behavioral finding secondary to GM₂ gangliosidosis in hexB^{-/-} mice; weakening begins in the third month of age, after which the mice quickly deteriorate. Clinical disease development can be assessed weekly in experimental, control and wild type mice by evaluating motor competency as follows. First, the animals can be placed on a rotorod, where their ability to maintain balance on a rotating cylinder can be measured as the latency of each animal to fall off (Columbus Instruments, Columbus Ohio). Their motor strength can be evaluated by placing the mice on a wire mesh fixed on the one end of a clear plastic cylinder, and turning the cylinder with the mesh and mice attached up side down, and measuring the latency of each animal to fall off. In addition, weight gain can be monitored weekly.

TABLE 10
Does GM₂ storage in neurons induce microglia activation summary

| GROUP | TYPE | Animals (N) | Time Points (4) | Analysis | Methods |
|---|---|---|---|---|---|
| A | hexB^{-/-}/ | 80 | 6, 12, 16 wks, | Molecular (n = 10) | QRT-PCR & |
| | Tg+ | | and/or | | Western |
| B | hexB^{-/-}/ | 80 | end of life span | Biochemical (n = 5) | enzyme |
| | Tg− | | | | activity |
| C | hexB^{-/-} | 80 | | Histology (n = 5) | ICC & ISH |
| D | B6129SF2 | 80 | | Behavioral (all) | rotorod & |
| | | | | | inverted mesh |

(4) Statistical Analysis

The aforementioned experiments can provide information on the role of neuronal GM₂ gangliosidosis in microglia activation and brain inflammation as it relates to disease development in the hexB^{-/-} knockout mouse. Any differences between the various groups can be assessed by multi-variable parametric statistical methods: two-way ANOVA (independent variables: animal type and experimental time point) with Tukey post-hoc analysis. Alpha can be set at 0.05 and the level of significance at p<0.05.

b) Determining the Role Peripheral Blood Mononuclear Cells in GM₂ Gangliosidosis

Microglia activation and increased levels of proinflammatory cytokines have been recently described in the brain of adult hexB^{-/-} mice. (Wada R, et al., Proc Natl Acad Sci USA 97: 10954-10959 (2002)) and other lysosomal storage disorders (Ohmi et al. Proc Natl Acad Sci USA 18: 1902-1907 (2003)). Moreover, peripheral blood mononuclear cells (PBMC) are reportedly affected in lysosomal storage diseases (Kiesser et al. Acta Neuropathol 94: 359-362 (1997)), although their status is not well characterized in GM₂ gangliosidosis. Disclosed herein, PBMC infiltrate into the brain parenchyma following microglia activation, whereby they further exacerbate the attendant CNS inflammatory process.

(1) Do PBMC Exacerbate Brain Inflammation and Disease Development in hexB^{-/-} Adult Mice

Monocyte chemoattractant protein-1 (MCP-1), a chemokine expressed by endothelial cells as well as perivascular microglia, mediates the infiltration of PBMC into the brain parenchyma. Although peripheral monocytes normally enter the brain for brief periods of time (surveillance functions), PBMC infiltration is significantly induced in cases of brain inflammation, further exaggerating the inflammatory process. Disclosed herein the hexB^{-/-} mouse can be crossed into the MCP-1^{-/-} knockout mouse, which display minimal PBMC recruitment after injury, and the effects of restrained PBMC infiltration in the brain at the molecular, histological and clinical levels in double hexB^{-/-}/MCP-1^{-/-} knockout mice can be assayed.

First, one can address the role of PBMC by inhibiting PBMC infiltration in the brain of hexB^{-/-} mice. Monocyte chemoattractant protein-1 (MCP-1), a chemokine expressed by endothelial cells as well as perivascular microglia and astrocytes, mediates the infiltration of PBMC into the brain parenchyma (Izikson et al., 2002). Although PBMCs normally enter the brain for brief periods of time and provide a function described as surveillance (Hickey et al. 1: 97-105 (1991), PBMC infiltration is significantly induced in cases of brain inflammation, further exaggerating the inflammatory process by secreting inflammatory mediators (Hickey et al. 1: 97-105 (1991) as well as contributing to neurotoxicity (Minghetti et al. Prog in Neurobiol 54: 99-125 (1998)). Disclosed herein is the crossing of the hexB^{-/-} into the MCP-1^{-/-} knockout mouse, which shows minimal PBMC recruitment after injury (Lu et al, 1998), and studying the effects of restrained PBMC infiltration at the molecular, histological and clinical levels in the double knockout mice.

(a) Development of hexB^{-/-}/MCP-1^{-/-} Double Knockout Mouse

The MCP-1-knockout mouse was originally developed by Dr. Barrett Rollins (Lu et al. J. Exp Med. 187(4):601-8 (1998)) and are commercially available on a C57BL/6 background by The Jackson Laboratory (stock# 4434; Bar Harbor Me.). As previously mentioned, the hexB^{-/-} mice were originally developed and kindly donated to our laboratory by Richard Proia (NIH/NIDDK) (Sango K, et al., Nature Genet 11: 170-176 (1995)). For the development of the desired double knockout genotype, one can first cross the hexB^{-/-} mouse into the MCP-1^{-/-} knockout, which will result in hexB^{+/-}/MCP-1^{+/-} F1 litters. These pups can be weaned and following confirmation of their genotype by established PCR-based methods, they can be inter-crossed as follows: hexB^{+/-}/MCP-1^{+/-} X hexB^{+/-}/MCP-1^{+/-}, resulting in the desired double knockout genotype (hexB^{-/-}/MCP-1^{-/-}) at 0.25 frequency, along with hexB^{+/+}/MCP-1^{+/+} (25%) and hexB^{+/-}/MCP-1^{+/-} (50%). This mating strategy can allow for employment of littermate controls, therefore easing any concerns regarding potential strain differences. After weaning and genotyping, the mice can be monitored weekly for behavioral signs and symptoms of GM₂ gangliosidosis.

Specifically, affected mice (hexB^{-/-}) are characterized by reduced locomotive performance as early as 3 months of age. To this end, each litter consisting of hexB^{-/-}/MCP-1^{-/-} double knockout, heterozygous (hexB^{+/-}/MCP-1^{+/-}) and wild type (hexB^{+/+}/MCP-1^{+/+}) littermates can be placed on a rotorod apparatus, where their ability to maintain balance on a rotating cylinder can be measured as the latency of each animal to fall. Their motor strength can also be assessed by placing the mice on a wire mesh fixed on the one end of a clear plastic cylinder, and turning the cylinder with the mesh and mice attached up side down, and measuring the latency of each animal to fall off. In addition, age matched hexB^{-/-} and MCP-1^{-/-} knockouts will be included in the study and will serve as controls.

At 6, 12, 16 weeks of age, and/or end of life span, experimental and control mice can be sacrificed and analyzed at the molecular and histological levels. In regards to brain inflammation, the total number as well as the number of activated MHC class II-positive microglia/macrophages/monocytes in the brain parenchyma can be quantitatively assessed by the aforementioned stereology methods utilizing antibodies against CD11b (See Examples herein) and MHC-II (See Examples herein) antigens. Astrogliosis can be evaluated based on the number of GFAP-positive cells in the brain (Examples herein). In addition, the mRNA levels of TNFα and other inflammation-related genes can be quantitatively determined by QRT-PCR (See examples herein; Kyrkanides et al., '01, '02; Olschowka et al., 2003).

TABLE 11
Do PBMCs exacerbate brain inflammation and disease development

| Group | TYPE | Animals (N) | Time Points (4) | Analysis | Methods |
|---|---|---|---|---|---|
| A | hexB^{-/-}/MCP-1^{-/-} | 80 | 6, 12, 16 wks, | Molecular | QRT- |
| | | | and/or | (n = 10) | PCR & |
| | | | end of life span | | Western |
| B | hexB^{+/-}/MCP- | 80 | | Biochemical | enzyme |
| | 1^{+/-} | | | (n = 5) | activity |
| C | hexB^{+/+}/MCP- | 80 | | Histology | ICC & |
| | 1^{+/+} | | | (n = 5) | ISH |
| D | MCP-1^{-/-} | 80 | | Behavioral | rotorod & |
| | | | | (all) | inverted |
| | | | | | mesh |
| E | hexB^{-/-} | 80 | | | |

(b) Statistical Analysis

The effect of reduced PBMC recruitment on disease development can be evaluated by the two-way analysis of variance (ANOVA), whereby locomotive performance can be analyzed in relation to the number of microglia/monocytes/macrophages in the brain as well as the animal's age (independent variables). The level of significance can be set at alpha=0.05. Moreover, one can analyze locomotive performance in relation to the number of activated PBMC in the brain to investigate the potential relationship between gliosis and disease development (regression and/or association analysis).

(2) Can #Hex-Expressing PBMC Attenuate GM₂ Gangliosidosis

Neonatal transplantation of normal bone marrow to hexB^{-/-} mice results in attenuation of the disease phenotype (Norflus et al. J Clin Invest 101: 1881-1888 (1998), similar to what has been reported in some TSD patients, along with amelioration of the inflammatory process in the brain. Disclosed herein PBMC expressing the therapeutic gene β-Hex can enter the brain parenchyma and contribute to the resolution of GM₂ storage via β-hexosaminidase cross-correction. To additionally show this, one can harvest bone marrow from hexB^{-/-} mice which were stably transduced with βHex in vitro and subsequently transplanted to hexB^{-/-} pups. The effects of this ex vivo therapy can be analyzed temporally at the molecular, histological and clinical (behavioral) level.

Donor bone marrow can be collected by flushing the femurs of hexB^{-/-} mice with Hank's balanced salt solution (Invitrogen, Carlsbad Calif.) as previously described (Suzuki et al. Lab Investigator 58: 302-309 (1988); Norflus et al. J Clin Invest 101: 1881-1888 (1998)). In brief, the cells can be collected by centrifugation, resuspended in a volume of 500 μl in Opti-MEM (Invitrogen) serum free culture media. The cells will then be infected with VSV-G pseudotyped FIV(βact-Hex) at a multiple of infectivity of m.o.i.˜2. FIV(βact-Hex) at titers of 10⁷-10⁸ infectious particles/mL have been routinely produced, which can allow for proper final m.o.i. in this experiment. FIV(βact-Hex) is capable of transducing cells with the βHex gene driven by the chicken β-actin/CMV enhancer element fusion promoter (Daly et al., 1999). After 12 hours of incubation, the cells can be collected by centrifugation and resuspended in Hank's balanced salt solution. Recipient mice (12 days old) can receive whole body irradiation of 9 Gy from a ¹³⁷CS source 1 day before transplantation. (Kyrkanides et al. J Neuroimmunol 95:95-106. (1999); (Kyrkanides et al. J Neuroimmunol 119: 269-77 (2001)). A total volume of 200 μl containing 5×10⁷ cells can be injected intraperitoneally to the recipient mice, which can then be returned to their mothers. In addition, a group of mice can receive non-transduced bone marrow from hexB^{-/-} donors and another group bone marrow from B6.129SF2 donors (JAX; stock# 101045), which can serve as additional controls. The mice can be then analyzed weekly for changes in behavioral performance, and can be sacrificed at 6, 12, 16 weeks of age and/or at the end of their life span (see Table 12).

Locomotive performance can be evaluated by the rotorod and inverted mesh methods. In brief, BMT-treated hexB^{-/-}, control, as well as wild type littermates can be placed on a rotorod, where their ability to maintain balance on a rotating cylinder can be measured as the latency of each animal to fall. Their motor activity can also be assessed by placing the mice on a wire mesh fixed on the one end of a clear plastic cylinder, and turning the cylinder with the mesh and mice attached up side down, and measuring the latency of each animal to fall off. The animals can be followed on a weekly basis and the locomotive data can be collected along with weight gain.

One can assess the distribution of βHex transduced bone marrow-derived cells in the brain of treated and control mice by immunocytochemistry (ICC) on brain histology sections utilizing antibodies against human HEX-A and HEX-B (FIG. 1; Methods in Detail). The identity of the cells will be confirmed by double immunofluorescence as previously described (See Examples herein; Kyrkanides et al. J Neuroimmunol 95:95-106. (1999)). The total number of βHex transduced cells expressing HEX-A and HEX-B, as well as the total number of microglia/monocytes/macrophages (CD11b positive cells) can be quantitatively determined by stereology on alternate tissue sections as previous described (Olschowka et al., 2003). Western immunoblotting will be employed for quantitative analysis of HEX-A and HEX-B levels at the various experimental time points.

In addition, one can confirm the distribution of βHex transduced bone marrow-derived cells in the brain of treated and control mice by in situ hybridization (ISH) using whole length probes for human HexA and HexB mRNAs (Brouxhon et al. Beh Immun 12: 107-122 (1998); Bellinger et al. Journal of Neuroimmunology 119(1):37-50 (2001)). By coupling ISH with immunocytochemistry (ICC), one can determine the identity of cells expressing HexA & HexB by employing cell specific markers for monocytes/macrophages (CD11b). One can quantitatively evaluate the number of transduced cells on alternate brain tissue sections utilizing stereology methods as previous described (Olschowka et al., 2003; Examples herein). Moreover, quantitative mRNA analysis of HexA and HexB in the brain will be determined by QRT-PCR as previously described (Olschowka et al., 2003).

Lastly, one can quantitatively determine the levels of HexA and HexB enzymatic activity at the various experimental time points and different tissues by 4MUGS and 4MUG fluorometry, respectively (Examples herein). In addition, two fairly simple histochemical methods for visualization of total β-hexosaminidase activity on histology tissue sections, including Fast Garnet staining (Examples herein) and X-Hex histochemistry have been successfully used (Examples herein).

TABLE 12
Can βHex-expressing PBMC attenuate GM₂ gangliosidosis in hexB^{-/-} mice

| | | | | Time | Analysis | |
|---|---|---|---|---|---|---|
| Group | Donor | Treatment | Mice (N) | Points (4) | (N = 20) | Methods |
| A | hexB^{-/-} | FIV (Hex) | 80 | 6, 12, 16 wks, | Molecular | QRT-PCR |
| | | | | and/or | (n = 10) | & Western |
| B | hexB^{-/-} | none | 80 | end of life | Biochemical | enzyme |
| | | | | span | (n = 5) | activity |
| C | B6129SF2 | none | 80 | | Histology | ICC & ISH |
| | | | | | (n = 5) | |
| | | | | | Behavioral | rotorod- |
| | | | | | (all 20) | inverted |
| | | | | | | mesh |

(a) Statistical Analysis

Collectively, the aforementioned experiments can provide data on the level of hexosaminidase restoration achieved over time by normal bone marrow transplantation and ex vivo therapy employing βHex-transduced bone marrow-derived cells. Any differences between the various groups can be assessed by multi-variable parametric statistical methods: two-way ANOVA (alpha=0.05) with Tukey post-hoc analysis, with the level of significance set at p<0.05. Moreover, one can analyze treatment outcomes (Table 12) in relation to the percent of β-hexosaminidase recovery in the brains of ex vivo treated animals relative to normal bone marrow treated mice and wild type littermates at the different time points.

c) Providing Additional Evidence of the Efficacy of βHex Vectors in Transducing Neurons, Microglia and PBMCs to Intercept the Development of GM₂ Gangliosidosis

GM₂ storage diseases are progressive disorders, whereby affected patients display near normal phenotype at infancy, but progress to severe forms in childhood. Depending on the clinical severity, patients may reach a vegetative state in early childhood, followed by death as early as 3-4 years of age. Disclosed herein, systemic administration of lentiviral vectors at neonatal stages of development can effectively restore β-hexosaminidase activity in the brain and peripheral tissues of hexB^{-/-} mice, leading to disease attenuation. To achieve this a replication defective β-Hex lentiviral vector derived from the feline immunodeficiency virus, FIV(Hex), which facilitates transduction of dividing, growth arrested as well as terminally differentiated cells, including neurons was made and adminstered. Disclosed herein 2 transfer vectors have been constructed: one employing the CMV promoter and the other utilizing the chicken β-actin promoter with CMV enhance elements, named FIV(βact-Hex). The FIV(βact-Hex) vector can be packaged and administered to hexB^{-/-} mice as described herein, in order to take advantage of the relatively strong and long-lasting levels of gene expression derived from the β-actin/CMV fusion promoter Daly et al. Hum Gene Ther 10: 85-94 (1999); (Daly et al. Gene Ther 8: 1291-8 (2001)).

(1) Determine the Distribution, Level and Persistence of βHex Expression

The spatial distribution of A Hex in hexB^{-/-} knockout mice following systemic administration of FIV(Hex), such as FIV(βact-Hex) at neonatal stages of development, with special emphasis in neurons, glia and PBMCs, can be characterized. The level of HexA and HexB expression, at the mRNA, protein and activity levels, can be analyzed temporally in the brain and vital organs, and can be correlated to the dose and route of administration.

FIV(βact-Hex) can be administered in experimental and control animals at neonatal stages of development. Since the hexB^{-/-} colony is maintained in heterozygous state, each litter will consist of 25% hexB^{-/-}, 50% hexB^{+/-} and 25% hexB^{+/+} wild types. Since the injections can be performed prior to genotyping, each litter can receive a single treatment. The hexB^{-/-} mice can serve as experimental whereas the hexB^{+/+} as controls. Specifically, for the systemic administration, a total of 5×10⁶ infectious FIV(βact-Hex) particles in 100 μl of sterile saline solution can be injected intraperitoneally (I.P.) to each pup at 2 days of age. In addition, other litters can receive I.P. injections of FIV(lacZ) at equivalent doses (5×10⁶ infectious particles in 100 μl of saline solution), or 100 μl vehicle (saline). Overall, the FIV(lacZ) group of animals can provide information on the effects of the viral vector itself, whereas the saline injections can control for the procedure. In total, one can have the following 4 groups of animals summarized in Table 13.

Neonatal FIV(Hex) administration for the treatment of β-hexosaminidase deficiency. Neonatal mice (P2) suffering from β-hexosaminidase deficiency (hexB^{-/-}) or wild type littermates (hexB^{+/+}) can be administered FIV(βact-Hex), FIV(lacZ) or normal saline systemically.

TABLE 13

| | | | | Animals | |
|---|---|---|---|---|---|
| Group | Type | Treatment | Time Points | (N) | Analysis |
| A | hexB^{-/-} | FIV(βact- | 6, 12, 16 wks, | 80 | Molecular |
| | | Hex) | end of life | | (n = 10) |
| | | | span | | |
| B | hexB^{-/-} | FIV(lacZ) | 6, 12, 16 wks, | 80 | Biochemical |
| | | | end of life | | (n = 5) |
| | | | span | | |
| C | hexB^{-/-} | saline | 6, 12, 16 wks, | 80 | Histology |
| | | | end of life | | (n = 5) |
| | | | span | | |
| D | W.T. | saline | 6, 12, 16 wks, | 80 | Behavioral |
| | | | end of life | | (all) |
| | | | span | | |

The sphere of βHex distribution at the various time points (6, 12, 16 weeks and/or end of life span) can be quantitatively assessed by determining the number of gene copies in the brain (cerebrum & cerebellum), liver, spleen and bone marrow by employing established QPCR methods in our laboratories (Olschowka et al., 2003). The data can be analyzed relative to time and treatment by the 2-way ANOVA with alpha=0.05. Differences between the various groups can be determined by the Tukey post-hoc analysis. Similarly, one can analyze the distribution of the reporter gene lacZ in the FIV(lacZ) groups.

One can assess the level of βHex expression at the mRNA level by quantitatively determining HexA and HexB mRNA levels in brain (cerebrum & cerebellum), liver, spleen and bone marrow RNA extracts by QRT-PCR protocols as previously described (Olschowka et al., 2003). HexA and HexB levels can be assessed at various time points (6, 12, 16 weeks and/or end of life span) following FIV(βact-Hex) perinatal administration. The mRNA data can be analyzed relative to time and treatment by the 2-way ANOVA with alpha=0.05. Differences between the various groups can be determined by the Tukey post-hoc analysis; level of significance set at p<0.05.

HexA, HexB and lacZ mRNA localization can be investigated by in situ hybridization (ISH) on brain, liver, spleen and bone marrow tissue sections as previous described (Brouxhon et al. Beh Immun 12: 107-122 (1998); Bellinger et al. Journal of Neuroimmunology 119(1):37-50 (2001)) using whole length probes for the human HexA and HexB. Coupling ISH with immunocytochemistry (ICC), one can determine the identity of cells expressing HexA & HexB by employing cell specific markers, including cerebral neurons (NeuN), cereberal neurons (calbindin), microglia (CD11b), astrocytes (GFAP), endothelial cells (PECAM-1), monocytes/macrophages (CD11b), lymphocytes (CD3). One can quantitatively assess the transduction of the various cell types by βHex on alternate tissue sections utilizing stereology methods as previous described (Olschowka et al., 2003; Examples herein). The data can be analyzed as described herein and collectively can provide information about the levels of βHex mRNA expression in the various tissues, as well as indicate any cellular preference for FIV vectors.

In addition, the expression of βHex at the protein level can be qualitatively assessed by immunocytochemistry (ICC) on histology sections harvested from brain (cerebrum & cerebellum), liver, spleen and bone marrow utilizing antibodies against human HEX-A and HEX-B (see Examples herein). The identity of the cells can be confirmed by double immunofluorescence as shown herein (See Examples herein and Kyrkanides et al. J Neuroimmunol 95:95-106. (1999)). The total number of transduced cells, as well as the number of neurons, glia, endothelial cells, macrophages and lymphocytes can be quantitatively determined by stereology on alternate brain, liver, spleen and femour histology sections as previous described (Olschowka et al., 2003). Western immunoblotting can be employed for semi-quantitative analysis of HEX-A and HEX-B levels (Tsuji et al., 2002; Proia et al. J Biol Chem 259: 3350-3354 (1984) at the various experimental time points (6, 12, 16 weeks and/or end of life span) and tissues (brain, liver, spleen, bone marrow/femur).

One can quantitatively determine the levels of HexA and HexB enzymatic activity in relation to time in the various experimental time points and different tissues by 4MUGS and 4MUG fluorometry, respectively ((As Disclosed herein). In addition, 2 simple histochemical methods for visualization of total β-hexosaminidase activity on histology tissue sections, including Fast Garnet staining (Examples herein) and X-Hex histochemistry (Examples herein) have been routinely used as disclosed herein.

Persistence of βHex expression can be evaluated as follows. Based on the data derived from the aforementioned experiments, one can temporally analyze HexA & HexB expression (mRNA, protein and activity levels) in relation to the number of βHex gene copies present in the various organs over time. Interpretation of the data can provide a measure of expression persistence (versus silencing). Any differences between the various groups can be assessed by multi-variable parametric statistical methods: two-way ANOVA (alpha=0.05) with Tukey post-hoc analysis, with the level of significance set at p<0.05. Moreover, one can calculate the percent of β-hexosaminidase recovery in FIV(βact-Hex) treated animals relative to wild type littermates, normalized to total protein, in the various tissues and at the different time points following treatment. This analysis can provide information pertinent to the efficacy of FIV(βact-Hex) perinatal therapy in restoring β-hexosaminidase activity. Any differences between the various groups can be assessed by multi-variable parametric statistical methods: two-way ANOVA (alpha=0.05) with Tukey post-hoc analysis; level of significance set at p<0.05.

(2) Evaluate the Effects of β-Hex Vector Administration on Disease Development

The hexB^{-/-} knockout mouse is characterized by biochemical, pathologic and clinical features similar to those seen in TSD and SD patients. One can analyze the effects of FIV(Hex), such as FIV(βact-Hex) administration on lysosomal storage, neuronal cell death, and behavioral performance in hexB^{-/-} knockout mice (Table 13) in relation to the sphere of β-Hex distribution and the levels HexA & HexB expression. Furthermore, since the introduction of novel proteins may elicit an immunologic response, one can assess the presence of antibodies against viral and transgenic proteins in blood serum following FIV administration.

Storage of insoluble metabolites can be first evaluated in histology brain sections by periodic acid Schiff histochemical staining (Sango K. Nature Genet 14: 348-352 (1996); Suzuki et al. J Neuropath Exp Neurol 56: 693-703 (1997)). In addition, GM₂ storage levels can be assessed by immunocytochemistry employing commercially available monoclonal anti-GM₂ antibodies (Sakurada et al. Clin Chim Acta 265: 263-266 (1997); Seikagaku, Fahnouth Mass.). GM₂ staining differences in the various animal groups can be evaluated by semi-quantitative analysis as previously described (Olschowka et al., 2003). For quantitative analysis of GM2 in the brain, one can employ immuno-thin layer chromatography (see Examples herein).

Neuronal degeneration can first be evaluated histochemically utilizing the Fluoro-Jade agent (Fluoro-Jade (Histo-Chem Inc., Jefferson Ariz.), a fluorescent agent that stains neurons undergoing degeneration (Schmued et al. Brain Res 751: 3746 (1997) Methods in Detail). The number of neurons undergoing apoptosis can be assessed by the fluorescin terminal uridine nick-end labeling method (TUNEL detects cellular apoptosis) on alternate brain sections. In addition, one can confirm apoptosis by double immunofluorescence utilizing antibodies raised against caspase-3 or -8. The total number of neurons can be determined also by double immunofluorescence with antibodies against NeuN nuclear protein (calbindin for cerebellar Purkinje neurons). The total number of nuclei can be determined by Hoechst nuclear staining. The number of cells undergoing cell death can be counted by stereology in alternate brain sections normalized for total number of nuclei (Hoechst staining).

Since the introduction of novel proteins may elicit an immunologic response in mice treated with FIV vectors, one can characterize the host's immunologic response following perinatal treatment. To this end, one can quantitatively assess the presence (titers) of antibodies against viral and transgenic proteins in blood serum at the different experimental time points (6, 12, 16 weeks and/or end of life span). To this end, IgG and IgM titers for HEXA and HEXB, as well as the FIV p24 antigen can be assessed by customized ELISA method. In brief, ELISA plates can be coated with 5 mg of human HEX-A, HEX-B (Sigma; St. Louis Mo.) or p24 recombinant proteins (IDEXX Laboratories Inc.; Westbrook Me.). After incubation with the sera, the plates can be alkaline phosphatase-conjugated goat anti-mouse IgG and IgM (Southern Biotechnology Associates, Inc; Birmingham Ala.). Antibody titers can be established as the serum dilution that reached absorbance levels (at 405 nm) of saline injected mice assuming linear extrapolation (Kang et al. J Virol 76: 9378-88 (2002)).

Evaluation of behavioral performance is also an important treatment outcome measure. Animal weight of experimental and control mice can be monitored weekly throughout the experiment. Motor competency can be assessed by the ability to maintain balance on a rotating cylinder (rotorod) by measuring the latency of each animal to fall off. In addition, their motor activity can be assessed by placing the mice on a wire mesh fixed on one end of a clear plastic cylinder, and turning the cylinder with the mesh and mice attached up side down. Motor competency can be evaluated in the experiment by measuring the latency of each animal to fall off. Using these two methods, one can assess motor behavior experimental and control mice on a weekly basis. Life span can also be recorded, since the affected mice suffer from significantly shortened life span (4 months). For that purpose, one can allow the experimental mice to fully complete their life cycle and hence evaluate the efficacy of FIV(βact-Hex) in attenuating the most perhaps devastating of the features of this class of disorders (in humans and animals). Life span can be calculated as the total number of days an animal survived.

Compilation of the above data, in conjunction with the data on βHex expression levels, will provide information on the level of β-hexosaminidase levels required for clinical correction of the disease relative to wild type littermates. The aforementioned experiments, collectively, can determine the full therapeutic efficacy of FIV(βact-Hex) perinatal gene therapy in restoring β-hexosaminidase activity in a mouse model of GM₂ gangliosidosis. Any differences between the various groups can be assessed by multi-variable parametric statistical methods: two-way ANOVA (alpha=0.05) with Tukey post-hoc analysis; level of significance set at p<0.05.

Disclosed herein the neonatal FIV(βact-Hex) administration to P2 mouse pups, as well as other age subjects, can result in wide distribution of βHex, including in the brain, resolution of neuronal GM2 storage and brain inflammation, leading to disease amelioration.

FIV(βact-Hex) intraperitoneal administration to neonatal pups can result in transduction of microglia and other perivascular cells, neurons and PBMC. Moreover, β-hexosaminidase activity can be restored at therapeutic levels, leading to attenuation of the disease (See examples herein).

9. Craniofacial Gene Therapy: Neuronal Function in Craniofacial Development, the Role of Bone and Cartilage Cells in Aberrant Craniofacial Development Secondary to Lysosomal Storage, and Therapeutic β-Hexosaminidase Levels by Systemic Administration of Lentiviral Vectors in the Brain and Craniofacial Complex

HexA^{-/-}/HexB^{-/-} mice were made and the cranial facial deformities were analyzed. Caniofacial dysplasia was significant. Affected and control littermates were analyzed at 3 wks of age. The total cranial length was marked by a line tangent to the cranial vault and extended to perpendicular lines drawn tangent to the most anterior and posterior points of the cranium. HexA & HexB genotype was determined by PCR on DNA extracts derived from tail biopsies (control* and affected⁺). The affected mouse was confirmed as hexA^{-/-} and hexB^{-/-}. In this example, the control mouse was hexA^{+/-}/hexB^{+/+}, and it is displayed because hexA mutations do not affect the mouse phenotype (Sango et al. Nature Genet 11: 170-176 (1995)). These and the rest of the mice can be continuously monitored and studied until they expire. 9 founder NSE-Hex mice in the lab were obtained.

An increasing body of evidence suggests that the nervous system plays an important role in craniofacial development. During the early stages of embryogenesis, the neural crest develops in close interaction with the primitive neural tube. Moreover, conditions that affect the development of the brain often involve the craniofacial complex, as seen in cases of anencephaly, holoprocencephaly, cyclopia or other less severe disorders that involve both the nervous system and the craniofacial skeleton. In particular, patients suffering from inherited lysosomal storage diseases, such as mucopolysaccharidoses, often exhibit growth impairment, skeletal abnormalities and craniofacial malformations, along with symptoms from the central nervous system due to neuronal dysfunction and cell death, such as blindness, mental retardation and paralysis. The neurons of the brain, trigeminal and spinal root ganglia display swollen vacuolated perikarya stored with excessive amounts of complex macromolecules, leading to aberrant neuronal function and cell death (apoptosis). Disclosed herein normal neuronal function is required for craniofacial development, and that neuronal dysfunction secondary to lysosomal storage contributes to aberrant craniofacial development. Disclosed vectors and methods can be used to treat disorders related to abnormal craniofacial development. Disclosed are animal models characterized by severe craniofacial dysostosis and growth retardation along with neuronal dysfunction due to β-hexosaminidase deficiency (hexA^{-/-}/hexB^{-/-} double knockout mice).

a) Neuronal Function in Craniofacial Development

One can restore β-hexosaminidase activity selectively in the neurons of hexA^{-/-}/hexB^{-/-} double knockout mice early during embryogenesis and by evaluating its effects on craniofacial development. For this purpose, one can employ a tricistronic transgene that encodes both isoforms of β-hexosaminidase, HexA & HexB, as well as the reporter gene β-galactosidase (lacZ). Transgene expression can be targeted selectively to neurons by the neuron specific enolase promoter: NSE-HexB-IRES-HexA-IRES-lacZ (NSE-Hex). Mice characterized by NSE-Hex germline transmission can be crossed into hexA^{-/-} and hexB^{-/-} knockout mice to develop a transgenic mouse harboring β-hexosaminidase competent neurons on hexA^{-/-}/hexB^{-/-} background.

(1) Construction of the NSE-Hex Transgene

See Example 8

(2) Development of the NSE-Hex Transgenic Mouse

See Example 8

(3) Neuronal Rescue and Craniofacial Development: The hexA^{-/-}hexB^{-/-}/NSE-Hex^{+/-} Mouse

To address the role of the nervous system in craniofacial development, one can restore β-hexosaminidase activity selectively in the neurons of hexA^{-/-}/hexB^{-/-} mice. Affected animals display neurons of the brain, trigeminal and spinal ganglia with swollen, vacuolated/clear perikarya; biochemical analysis reveals a complete lack of β-hexosaminidase activity accompanied by storage of gangliosides and mucopolysac-charides interfering with normal cellular functions and ultimately leading to neuronal cell death (Sango et al., Nature Genet. 14: 348-352 (1996); Suzuki et al. J Neuropath Exp Neurol 56: 693-703 (1997); Huang et al. Hum Mol Genet 6:1879-85 (1997)). One can rescue the neurons from dysfunction and cell death by restoring neuronal β-hexosaminidase activity during embryogenesis in affected animals. To this end, one can undertake a husbandry strategy involving the NSE-Hex, HexA^{-/-} and HexB^{-/-} mice aiming at developing the desired genotype: hexA^{-/-}/hexB^{-/-}/NSE-Hex^{+/-}.

Initially, one can cross the NSE-Hex mouse into the hexA^{-/-} mouse and then backcross it again in order to generate the hexA^{-/-}/NSE-Hex^{+/-} genotype as disclosed herein. Consequently, one can cross the hexA^{-/-}/NSE-Hex^{+/-} into the hexB^{-/-} and can select pups with the following genotype: hexA^{-/-}/hexB^{+/-}/Tg^{+/-}. By intercrossing the latter (hexA^{+/-}/hexB^{+/-}/Tg^{+/-} X hexA^{+/-}/hexB^{+/-}Tg^{+/-}) one can develop a desired breeding type, hexA^{-/-}/hexB^{+/-}/Tg^{+/-}. Mating of hexA^{-/-}/hexB^{+/-}/Tg^{+/-} X hexA^{-/-}/hexB^{+/-} can result in litters including hexA^{-/-}/hexB^{-/-}/Tg^{+/-} (experimental) as well as hexA^{-/-}/hexB^{-/-}/Tg^{-/-} littermates (controls).

(4) Methods of Analysis

After weening the mice can be ear tagged and tail biopsies can be obtained for genotyping as described in the Methods in Detail section. The appropriate experimental and control mice can be housed according to gender and those with unsuitable genotype can be terminated. In addition, age & gender matched B6129SF2 (Jackson Laboratories) can be included as wild type controls. It is important to note at this point that the affected hexA^{-/-}/hexB^{-/-} mice are born with near normal phenotype, but develop craniofacial anomalies by 3 weeks of age. The following groups of mice can be analyzed as summarized in Table 14.

Summary of NSE-Hex in hexA−/−/hexB−/− mice. A total of 80 hexA^{-/-}/hexB^{-/-}/Tg+ (experimental) and 80 hexA^{-/-}/hexB^{-/-}/Tg− (controls) littermates can be employed in this experiment, along with 80 wild type mice (B6129SF2). At each time point, 20 mice of each group can be evaluated by radiography (cephalometric analysis) and behavioral motor analyses. Thereafter, the mice can be terminated and analyzed by molecular (10 mice of each group), biochemical (5 mice of each group), histological (5 mice of each group).

TABLE 14

| Type | Time Points (4) | Animals (N) | Analysis (20) | Methods |
|---|---|---|---|---|
| hexA^{-/-}/hexB^{-/-}/Tg+ | 1, 2, 3, 4 wks | 4 × 20 = 80 | Molecular (n = 10): | QRT- |
| | | | | PCR & |
| | | | | Western |
| hexA^{-/-}/hexB^{-/-}/Tg− | 1, 2, 3, 4 wks | 4 × 20 = 80 | Biochemical | enzyme |
| | | | (n = 5): | activity |
| B6129SF2 | 1, 2, 3, 4 wks | 4 × 20 = 80 | Histology (n = 5): | ICC & |
| | | | | ISH |
| | | | Behavioral (all): | rotorod & |
| | | | | inverted |
| | | | Radiography (all): | mesh |
| | | | | cephalometrics |
| | | Total = 240 mice | | |

(5) Life Span and Weight Gain

Life span can be calculated as the total number of days the animals survived. Animal weights can be monitored and recorded weekly throughout the experiment.

(6) Craniofacial Growth & Development

Cephalometric radiography can provide quantitative information related to the development of the craniofacial skeleton. The analysis can be performed by one examiner who can be blinded as to the mouse type or identity. In brief, the animals can be briefly anesthetized, immobilized on a cephalometric film cassette with their cranial mid-sagittal plane positioned parallel to the cassette and cephalometric radiographs can be obtained utilizing a long-cone X-ray machine at preset distances. (Kyrkanides et al. Molecular & Cellular Biology 13(9):5168-74 (1993), Kyrkanides et al. Cleft Palat Craniofac J32: 428-32 (1995), Kyrkanides et al. Cleft Palat Craniofac J 33: 306-310 (1996), Kyrkanides et al. Cleft Palat Craniofac J 37: 556-561 (2000); also as disclosed herein).

(7) Behavioral Performance

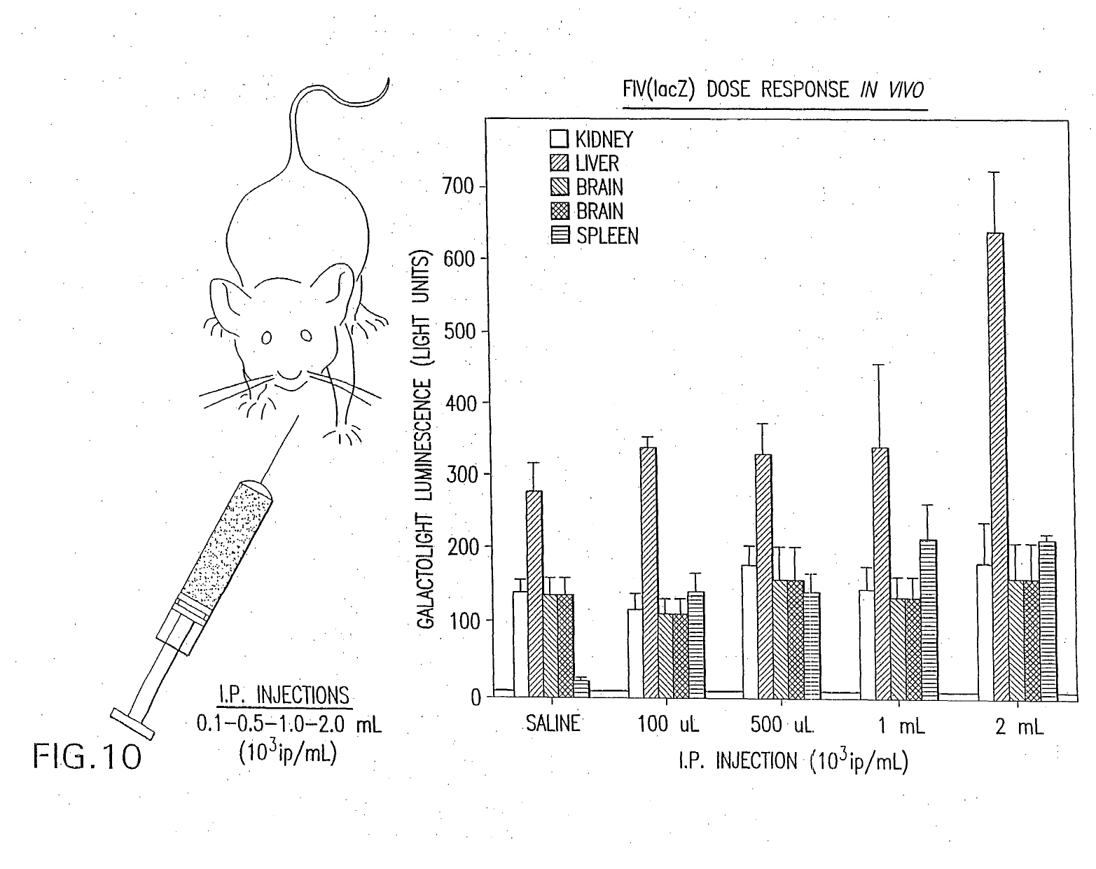

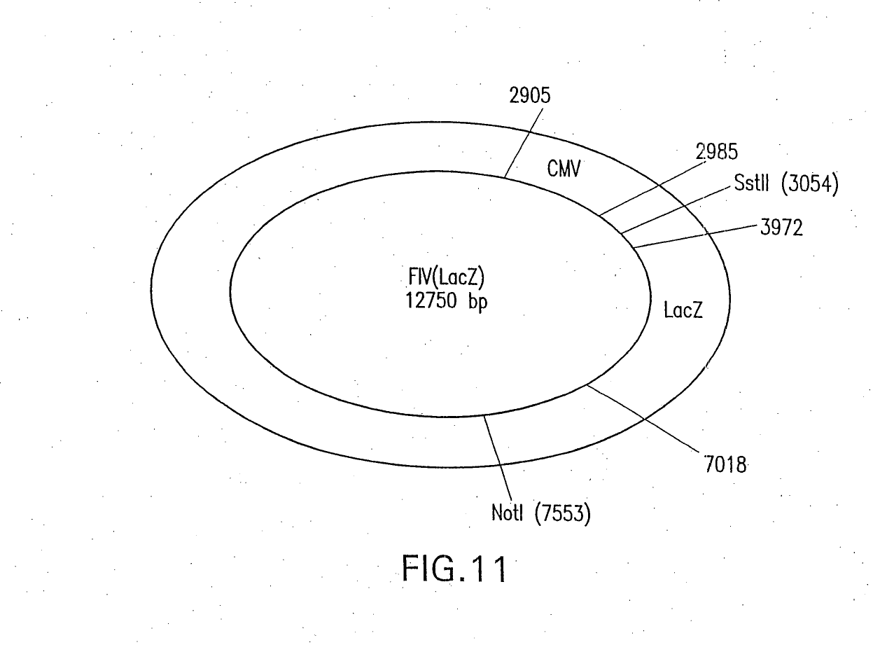

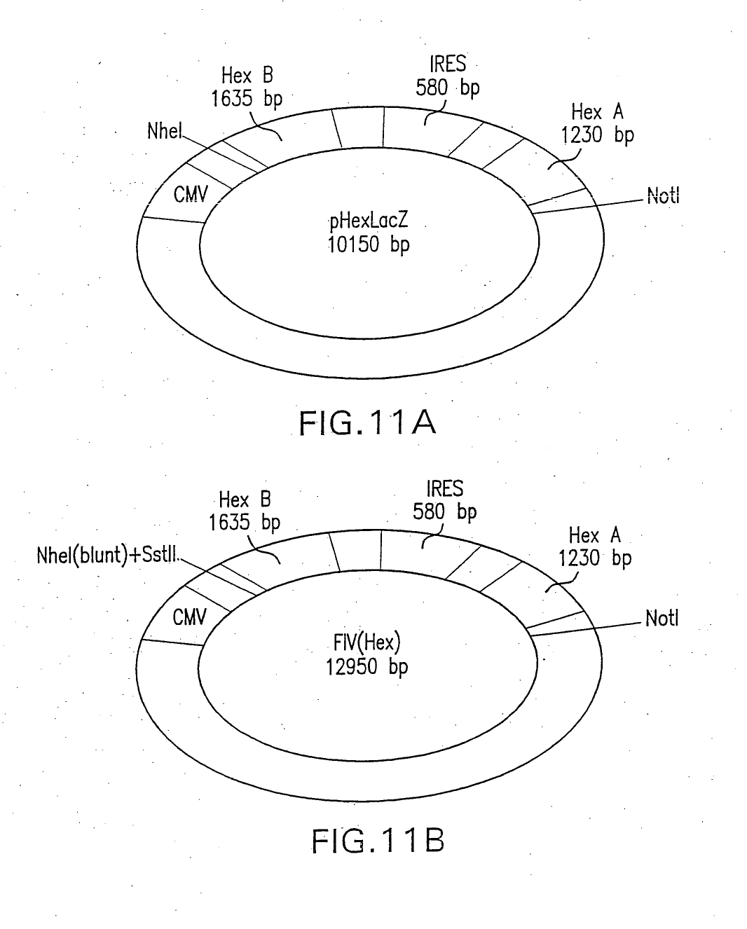

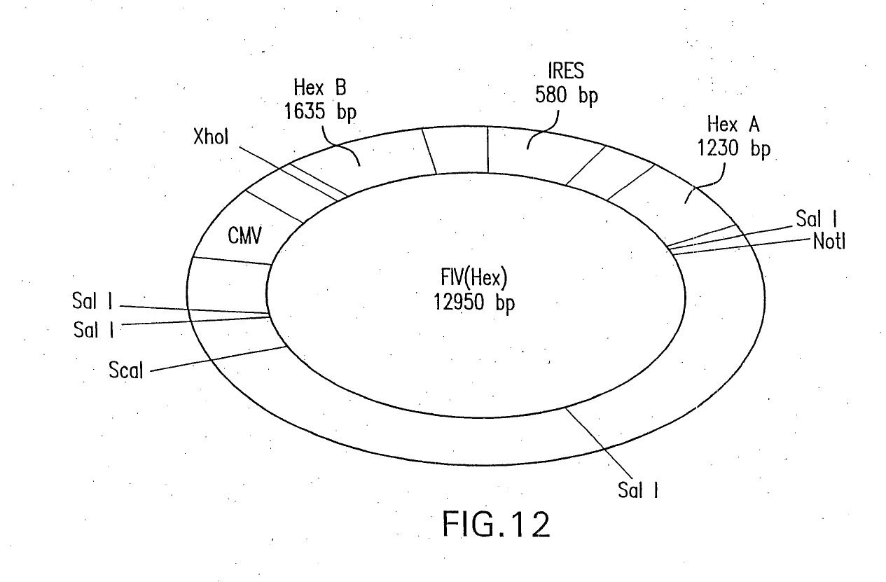

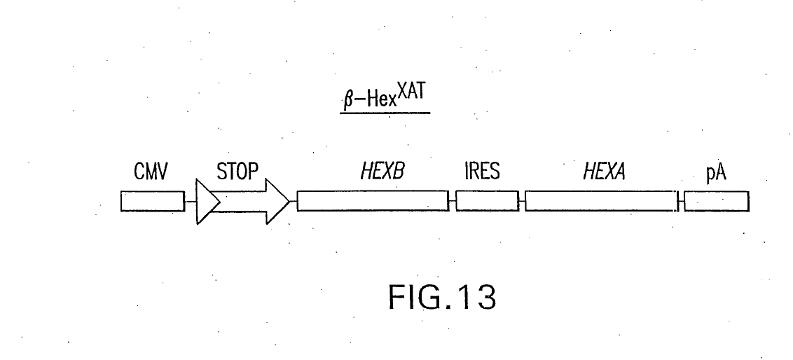

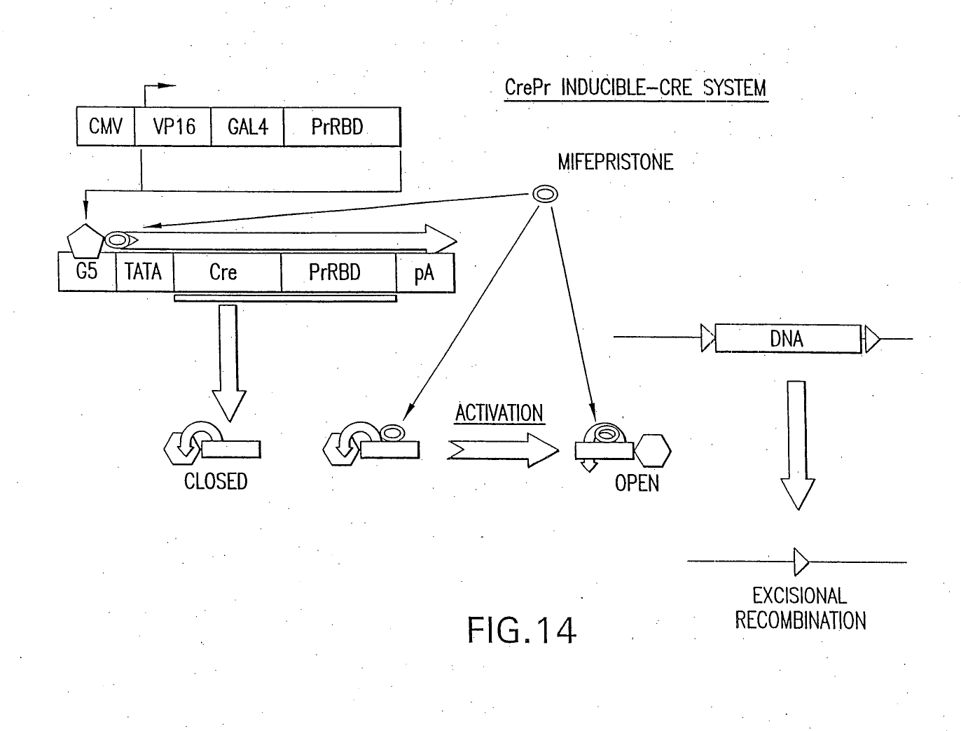

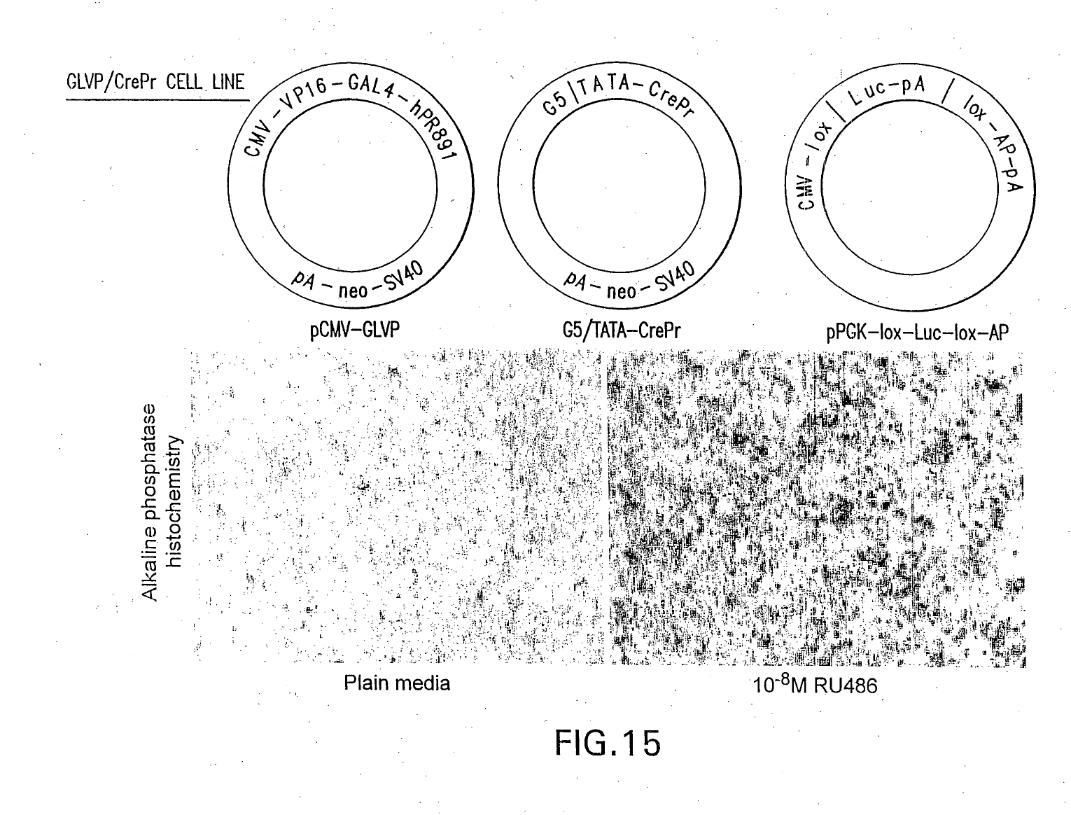

Motor competency can be assessed by the ability of an animal to maintain balance on a rotating cylinder (rotorod), and measuring the latency of each animal to fall off (Sango et al. Sango et al. Nature Genet 14: 348-352 (1996)). In addition, their motor strength can be assessed by placing the mice on a wire mesh fixed on one end of a clear plastic cylinder, and turning the cylinder with the mesh and mice attached up side down. (Please refer to FIG. 10 for example). Motor competency can be evaluated by measuring the latency of each animal to fall off. Using these two methods, one can assess motor behavior in experimental and control mice on a weekly basis.

(8) HexA & HexB mRNA Expression.

One can determine the expression of the β-hexosaminidase therapeutic gene at the transcriptional level by QRT-PCR in brain RNA extracts at the proposed time points (Table 14). (Kyrkanides et al. J Neuroimmunol 119: 269-77 (2001), Kyrkanides et al. Mol Brain Res 104: 159-169 (2002a), Kyrkanides et al. Molecular Therapy, In Press. (attached to this application as part of Appendix) (2003a)).

(9) HEXA & HEXB Protein and Enzyme Activity Levels.

One can quantitatively evaluate the levels of β-hexosaminidase in brain lysates at the protein level by western immunoblotting (Utsumi et al. Acta Neurol Scand 105:427-30 (2002) as well as enzyme activity by 4MUGS and 4MUG fluorometry, respectively (As disclosed herein).

(10) Histology

The following histological methods require fixed, sectioned tissue mounted on glass slides. For this purpose, the animals of this group can be deeply anesthetized, and transcardially perfused with 4% paraformaldehyde in phosphate buffered saline (PBS, pH 8.0). The tissues of interest can be collected and post-fixed in the same solution for 3 hours, followed by immersion in 30% sucrose in PBS overnight. Calcified tissues (nasomaxillary complex/cranial base) can be decalcified by EDTA solution prior to sucrose treatment (As disclosed herein). The tissues can then be frozen over dry ice and cut in 20 μm sections using a freezing cryotome. Data analyses can be performed by one examiner who will be blinded as to the mouse type or identity. The following methods can be employed:

(11) Storage of Insoluble Metabolites

Excessive accumulation of insoluble gangliosides and mucopolysaccharides is a cardinal histopathologic feature in HexA^{-/-}/HexB^{-/-} double knockout mice, developed as a result of the β-hexosaminidase deficiency that can be readily detected by periodic acid Schiff (PAS) and Alcian blue staining (Sango et al. Nature Genet 14: 348-352 (1996); Suzuki & Purpura et al. J Neuropath Exp Neurol 56: 693-703 (1997); As discussed herein). Furthermore, GM₂ ganglioside storage is pathognomonic for β-hexosaminidase deficiency. Therefore, one can semi-quantitatively evaluate GM₂ storage by immunocytochemistry employing rat monoclonal antibodies raised against this antigen (commercially available for Seikagaku, Falmouth Md.). (Kyrkanides et al. J Neuroimmunol 119: 269-77 (2001), Kyrkanides et al. Mol Brain Res 104: 159-169 (2002a), as disclosed herein). In addition, the levels of GM₂ ganglioside in the brain can be assessed quantitatively by immune-thin layer chromatography (As disclosed herein).

(12) 9-Hexosaminidase mRNA Expression

HexA & HexB mRNA localization can be performed by ISH and neuronal identity will be confirmed by ISH/ICC utilizing antibodies against NeuN nuclear protein (cerebral neurons) as well as calbindin (Purkinje cerebellar neurons). (Brouxhon et al. Brain Behav Immun 12:107-122 (1998); Bellinger et al. J Neuroimmunol 119: 37-50 (2001), and as discussed herein).

(13) HEXA & HEXB Protein Expression.

The expression of the β-hexosaminidase gene at the protein level can be semi-quantitatively assessed by immunocytochemistry (ICC) of brain sections employing antibodies raised specifically against human HEXA and HEXB (the products of the therapeutic gene). See Examples herein where DAB was used as a chromagen (and see Kyrkanides et al. J Neuroimmunol 95:95-106 (1999), Kyrkanides et al. J Neuroimmunol 119: 269-77 (2001), Kyrkanides et al. Mol Brain Res 104: 159-169 (2002a), Kyrkanides et al. J Orofac Pain 16:229-235 (2002b)). The sections can be counter-stained with hematoxylin for identification of histological landmarks. The identity of the cells expressing HEXA and HEXB can also be confirmed by double immunofluorescence. (Kyrkanides et al. J Neuroimmunol 95:95-106 (1999)). Neurons can be confirmed by antibodies raised against NeuN nuclear protein (cerebral neurons; Mullen et al. Development 116: 201-211 (1992); Chemicon INTL, Temacula Ca) as well as calbindin (Purkinje cerebellar neurons; Fournet et al. Brain Res 399: 310-316 (1986)).

(14) β-Hexosaminidase Activity.

Histochemical methods for detection and visualization in situ of β-hexosaminidase activity, X-gal staining can be used. This can allow for semi-quantitatively evaluation of hexosaminidase activity (Kyrkanides et al. J Orofac Pain 16:229-235 (2002b)).

(15) Cell Death.

It has been reported that β-hexosaminidase deficiency causes neuronal cell death in Hex knockout mice (Huang et al. Hum Mol Genet 6:1879-85 (1997); Wada et al. Proc Natl Acad Sci U.S.A. 97(20):10954-9 (2000)). It is possible that neuronal cell death is associated with developmental anomalies of the craniofacial skeleton noted on the HexA^{-/-}/HexB^{-/-} mice. Initially, one can process brain histologic sections with Fluoro-Jade (Histo-Chem. Inc., Jefferson Ariz.), a fluorescent agent that stains neurons undergoing degeneration (Schmued et al. Brain Res 751: 37-46 (1997)). In addition, one can detect neuronal apoptosis by the fluorescin terminal uridine nick-end labeling method (TUNEL). To confirm apoptosis, one can double stain with antibodies against caspace-3 or -8. The total number of nuclei can be determined by the Hoechst nuclear staining. The number of cells undergoing cell death (TUNEL, caspace-3 & -8 positive cells) can be counted in 10 random microscopic fields (10×) normalized for total number of nuclei (Hoechst staining). One can employ multiple immunofluorescence methods to confirm cell types and quantify the histology results (Kyrkanides et al. J Neuroimmunol 95:95-106. (1999)).

Statistical analysis of the data can be performed. To investigate differences in life span between animal groups, one can use a log-rank test. The time of death can be treated as censored for mice that were sacrificed, because one cannot know how long those mice would have survived. Therefore, some life span data can be censored at 1, 2, 3 & 4 weeks. For continuous endpoints such as quantitative measures of craniofacial growth and behavioral performance, one can test for differences between animal groups at each time point using one-way ANOVAs. One can also examine whether the means of these endpoints change over time for each group, and whether any differences between groups changes over time. This can be done using linear regression models. One can also calculate the percent of β-hexosaminidase recovery in animals relative to wild type controls normalized to total protein at the different time points following treatment. This analysis can provide information pertinent to the efficacy of the NSE-Hex transgene in restoring β-hexosaminidase activity. Any differences between the various groups can be assessed using two-way ANOVA (alpha=0.05) with Tukey post-hoc analysis; in all analyses the level of significance can be set at 0.05. As the above methods are appropriate for normally distributed data, we can explore whether that distributional assumption is reasonable. For some endpoints, such as those that are highly skewed, one can consider ‘normalizing’ transformations such as log or square-root.

NSE-Hex expression can result in β-hexosaminidase restoration selectively in neurons at therapeutic levels. The NSE-promoter has been previously utilized in targeting transgene expression to neurons at significant levels (Forss-Petter et al. Neuron 5: 187-197 (1990). Data disclosed herein indicated NSE-Hex levels of expression comparable to that of CMV-Hex in N2α cells in vitro. (see example 8) Moreover, since neonatal transfer of the CMV-Hex gene to hexB^{-/-} mice resulted in sustained transgene expression for at least 5 weeks including neurons, NSE-Hex can successfully express HexA and HexB in the brain of hexA^{-/-}/hexB^{-/-} mice.

Although craniofacial development begins during the initial stages of embryogenesis, growth of the craniofacial skeleton actively continues after birth, whilst a significant portion of the facial and skeletal development occurs. In fact, anthropometric studies (reviewed by Gorlin, R J, Cohen M M and Levin L S (Eds). Oxford University Press, New York pp 99-117 (1990) revealed that more than 39% of craniofacial growth occurs postnatally in the saggital plane, 37% in the transverse and 31% in the vertical plane (from birth to adulthood). Cephalometrically, the anterior cranial base (pre-sphenoid-ethmoid-frontal bones) obtains 55% and the maxilla 80% of their total growth potential also postnatally. Therefore, the NSE-Hex mediated neuronal rescue will have ample opportunity to reverse the abnormal craniofacial growth pattern and hence ameliorate the abnormal facial phenotype since the NSE promoter is active as early as E9.5 in embryogenesis (Cinato et al. Genesis: the Journal of Genetics & Development. 31(3):118-25 (2001)).

As far as the effects of neuronal rescue to craniofacial development in the hexA^{-/-}/hexB^{-/-} mice by the NSE-Hex gene a moderate but significant attenuation of the attendant craniofacial anomaly can occur. β-hexosaminidase restoration in the neurons of hexA^{-/-}/hexB^{-/-} mice can result in a significant degree of craniofacial normalization. Notwithstanding the importance of the nervous system in development, it is also expected that peripheral cells directly associated with skeletal growth, such as chondrocytes and osteocytes also contribute to craniofacial development, as discussed below.

b) The Role of Bone and Cartilage Cells in Aberrant Craniofacial Development Secondary to Lysosomal Storage

hexA^{-/-}/hexB^{-/-} double knockout mice are characterized by cellular anomalies of bone and cartilage in addition to that of neurons. One can restore β-hexosaminidase activity selectively in the osteocytes and chondrocytes of hexA^{-/-}/hexB^{-/-} double knockout mice during embryogenesis and evaluate its effects on craniofacial development. The expression of the HexB-IRES-HexA-IRES-lacZ transgene can be driven by the pro-collagen 1 A1 gene promoter that targets gene expression to chondrocytes and osteocytes (COLL1-Hex) (Krebsbach P H, et al., Mol Cell Biol 13: 5168-74 (1993)) (CoLL1 promoter, SEQ ID NO 70) Mice capable of germline transmission of the COLL1-Hex gene can be crossed into hexA^{-/-} and hexB^{-/-} knockout mice to develop a transgenic mouse harboring β-hexosaminidase competent osteocytes and chondrocytes on hexA^{-/-}/hexB^{-/-} background.

(1) Construction of the COLL1-Hex Transgene

A tricistronic gene HexB-IRES-HexA-IRES-lacZ encoding for both subunits of the human β-hexosaminidase as well as the reporter gene lacZ is disclosed herein. Disclosed herein, a version of this construct with the 3.6 Kb pro-collagen 1 A1 (COLL1) gene promoter (Krebsbach et al. Molecular & Cellular Biology 13(9):5168-74 (1993)) driving the Hex construct. In brief, using molecular biology protocols one can excise the CMV promoter and in its place one can clone the 3.6 Kb COLL1 promoter (SEQ ID NO:71)

The following can be constructed:

COLL [symbol] HexB-IRES-HexA-IRES-lacZ (COLL1-Hex).

In brief, the 3.6 Kb COLL1 promoter will be excised from pCOLL1A1PR by BamHI digestion and cloned into the Xho I-Hind III sites of pBS (Stratagene) utilizing custom made linkers: The Xho I-BamH I [5′-] linker carries a Not I site which can be utilized in the final construct for the release of the transgene (see below). Next, the NheI-NotI segment of pHEXlacZ can be inserted into the EcoRV-NotI sites of pBS downstream to COLL1 by 5′-blunt and 3′-sticky ligation (pCOLL1-Hex). The function of COLL1-Hex can be analyzed in the murine fibroblast NIH 3T3 cell line Westerman Kans., Leboulch P. Reversible immortalization of mammalian cells mediated by retroviral transfer and site-specific recombination. Proc. Natl. Acad. Sci. USA 93: 8971-8976, 1996. (America Tissue Culture Collection, Manassas Va.; cat#CRL-1658), whereby the COLL1-Hex can be transiently transfected in cultured cells employing the Lipofectamine 2000 (Invitrogen). Transgene expression can be initially assessed by X-gal histochemistry. Expression of HexA and HexB, as well as lacZ will be evaluated at the mRNA, protein and activity levels, by QRT-PCR (Kyrkanides et al. J Neuroimmunol 119: 269-77 (2001), Kyrkanides et al. Mol Brain Res 104: 159-169 (2002a), immunocytochemistry (Kyrkanides et al. J Neuroimmunol 119: 269-77 (2001), Kyrkanides et al. Mol Brain Res 104: 159-169 (2002a), Kyrkanides et al. J Orofac Pain 16:229-235 (2002b), X-Hex histochemistry and 4MUG/S fluorometry (Kyrkanides et al. Mol Ther. 2003 November; 8(5):790-5). The CMV-Hex vector can be employed as control in the transfection experiment. Results can be normalized for transfection efficiency at the DNA level by PCR utilizing primers designed specifically for bacterial plasmid sequences. COLL1-Hex transfection in NIH 3T3 cells can result in induction of HexA and HexB levels of expression.

(2) Development of the NSE-Hex Transgenic Mouse

Transgenic mouse lines (4-7) can be developed by injecting linearized COLL1-Hex (Not I-Not I segment of pCOLL1-Hex) into fertilized mouse oocytes (C57BL6/J) followed by re-implantation of surviving eggs into pseudopregnant recipient females. The COLL1 promoter has been previously employed with success in directing transgene expression selectively in osteocytes & chondrocytes of transgenic mice (Krebsbach et al. Molecular & Cellular Biology 13(9):5168-74 (1993)). The transgenic mouse lines can be analyzed for gene incorporation and function, as well as germline transmission. The transgene can be maintained in a heterozygous state on the C57BL6/J background. Transgene incorporation (1) can be tested in tail DNA extracts by PCR using human-specific HexB, HexA and lacZ primers (genotyping). Primers that selectively detect the human HexB & HexA, as well as bacterial lacZ, but not the murine isoforms are disclosed herein. (2) Transgene incorporation can then be confirmed in naso-maxillary DNA extracts by Northern blotting using whole length probes designed specifically for the human HexB and HexA.

Transgene function can be evaluated in vivo as follows. (1) First, lacZ expression can be readily assessed in decalcified naso-maxillary histology sections by X-gal histochemistry (see Methods in Detail). (2) HexA and HexB expression at the mRNA level can be evaluated by QRT-PCR in total naso-maxillary mRNA extracts, and can be compared to wild type littermates. Localization of HexA & HexB mRNA can be achieved on naso-maxillary histology sections by in situ hybridization (ISH); cellular identity can be confirmed by coupling ISH with immunocytochemistry (ICC) employing antibodies raised against the following antigens. Osteocytes/osteblasts can be confirmed by the expression alkaline phosphatase, osteocalcin, type I collagen (Liu et al. Exp Cell Res 232: 97-105 (1997); Adamo et al. J Oral Implantol 27: 25-31 (2001)). Chondrocytes can be confirmed by detection of collagen II (Scott-Burden et al. Ann Thorac Surg 73: 1528-33 (2002)). Endothelial cells can be stained with antibodies raised against PECAM-1 (CD 31). Murine macrophages can be confirmed by detection of CD11b (Kyrkanides et al. J Neuroimmunol 95:95-106. (1999) (Brouxhon et al. Brain Behav Immun 12:107-122 (1998); Bellinger et al. J Neuroimmunol 119: 37-50 (2001). (3) HEX-A and HEX-B protein expression can be analyzed by ICC in naso-maxillary histology sections employing antibodies raised specifically against the human HEX-A and HEX-B proteins (Proia et al. J Biol Chem 259: 3350-3354 (1984). The identity of HEX-A and HEX-B expressing cells can be confirmed by double immunofluorescence as described above. Quantification of HEX-A and HEX-B protein levels in NSE-Hex and wild type littermates can also be assessed by western immunoblotting utilizing the aforementioned HEX antibodies (Utsumi et al. Acta Neurol Scand 105:427-30 (2002)). (4) HexA and HexB enzyme activity can be first evaluated on brain histology sections by X-Hex histochemistry (As disclosed herein). In addition, HexA & HexB activities will be quantified by 4MUGS and 4MUG fluorometry in naso-maxillary extracts (As disclosed herein). The data derived from at least two generations (F1 & F2) will be compared to ensure germline transmission and persistence of the phenotype.

Transgene expression in NSE-Hex transgenic mouse lines. One can analyze 3-5 transgenic mouse lines for transgene incorporation and function at the RNA, protein and histology levels in at least two generation, F1 and F2. For this purpose, the founders can be mated with C57BL6/J wild types and their offspring can be analyzed as described.

TABLE 15

| Analysis | Animals (N) | Methods |
|---|---|---|
| mRNA | 5 × 7 lines = 35 | QRT-CPR |
| Protein | 5 × 7 lines = 35 | western immunoblotting & β- |
| | | hexosaminidase activity |
| Histology | 5 × 7 lines = 35 | immunocytochemistry (ICC), in situ |
| | | hybridization (ISH), |
| | Total = 135 mice | X-gal & X-Hex histochemistry |

After weening, the mice can be ear tagged and tail biopsies can be obtained for genotyping as described in the Methods in Detail section. The appropriate experimental and control mice will be housed according to gender and those with unsuitable genotype can be terminated. The following groups of mice can be analyzed as summarized in Table 16. A total of 3 groups (N=80 per group) of mice can be used.

hexA^{-/-}/hexB^{-/-}/COLL1-Hex^{+/-}, hexA^{-/-}/hexB^{-/-}/COLL1-Hex^{-/-} and wild type controls (B6129SF2) can be made. These mice can be analyzed over time employing methods described herein. In brief, the following evaluations can be performed by a blinded examiner: (1) Life span and weight gain; (2) Craniofacial cephalometric radiography; (3) Motor behavioral analysis by the rotorod and inverted mesh methods. In addition, (4) HexA & HexB mRNA expression can be evaluated as described above in nasomaxillary mRNA extracts by QRTPCR, (5) HEXA & HEXB enzyme activity and protein levels in nasomaxillary lysates by 4MUG/S fluorometry and western immunoblotting. (6) Histological analysis of stored insoluble metabolites in nasomaxillary tissue sections can also be performed, along with in situ hybridization for HexA & HexB mRNA and immunocytochemistry for HEXA and HEXB proteins. Cell apoptosis can also be evaluated as described above. These aforementioned results can be quantified as described herein.

Summary of experimental design for Specific Aim 2: COLL1-Hex in hexA−/−/hexB−/− mice. A total of 80 hexA^{-/-}/hexB^{-/-}/Tg+ (experimental) and 80 hexA^{-/-}/hexB^{-/-}/Tg− (controls) littermates can be employed in this experiment, along with 80 wild type mice (B6129SF2). At each time point, 20 mice of each group can be evaluated by radiography (cephalometric analysis) and behavioral motor analyses. Thereafter, the mice can be terminated and analyzed by molecular (10 mice of each group), biochemical (5 mice of each group), histological (5 mice of each group).

TABLE 16

| TYPE | Time Points (4) | Animals (N) | Analysis (20) | Methods |
|---|---|---|---|---|
| hexA^{-/-}/ | 1, 2, 3, & 4 wks | 4 × 20 = 80 | Molecular (n = 10): | QRT-PCR |
| hexB^{-/-}/ | | | | & Western |
| Tg+ | | | | |
| hexA^{-/-}/ | 1, 2, 3, & 4 wks | 4 × 20 = 80 | Biochemical (n = 5): | enzyme |
| hexB^{-/-}/ | | | | activity |
| Tg− | | | | |
| B6129SF2 | 1, 2, 3, & 4 wks | 4 × 20 = 80 | Histology (n = 5): | ICC & ISH |
| | | | Behavioral (all): | rotorod & |
| | | | Radiography (all): | inverted |
| | | | | mesh |
| | | | | cephalometrics |
| | | Total = 240 mice | | |

To investigate differences in life span between animal groups, one can use a log-rank test. The time of death can be treated as censored for mice that were sacrificed, because ond will not know how long those mice would have survived. Therefore, some life span data can be censored at 1, 2, 3 and 4 weeks. For continuous endpoints such as quantitative measures of craniofacial growth and behavioral performance, one can test for differences between animal groups at each time point using one-way ANOVAs. One can also examine whether the means of these endpoints change over time for each group, and whether any differences between groups changes over time. This can be done using linear regression models. One can also calculate the percent of β-hexosaminidase recovery in animals relative to wild type controls normalized to total protein at the different time points following treatment. This analysis can provide information pertinent to the efficacy of the NSE-Hex transgene in restoring β-hexosaminidase activity. Any differences between the various groups can be assessed using two-way ANOVA (alpha=0.05) with Tukey post-hoc analysis; in all analyses the level of significance will be set at 0.05. As the above methods are appropriate for normally distributed data, one can explore whether that distributional assumption is reasonable. For some endpoints, such as those that are highly skewed, one can consider ‘normalizing’ transformations such as log or square-root.

COLL1-Hex expression can result in β-hexosaminidase restoration in osteocytes and chondrocytes. Moreover, since neonatal transfer of the CMV-Hex gene to hexB^{-/-} mice resulted in sustained transgene expression for at least 5 weeks including neurons, COLL1-Hex can successfully express HexA and HexB in the craniofacial skeleton of hexA^{-/-}/hexB^{-/-} mice.

c) Therapeutic β-Hexosaminidase Levels by Systemic Administration of Lentiviral Vectors in the Brain and Craniofacial Complex

Timely restitution of β-hexosaminidase activity in hexA^{-/-}/hexB^{-/-} mice can result in normalization of skeletal development. Neonatal transfer of β-hexosaminidase vectors can restore cellular function in neurons as well as in osteocytes and chondrocytes in hexA^{-/-}/hexB^{-/-} mice, ultimately leading to amelioration of the correlate craniofacial anomalies. Three feline immunodeficiency viral transfer vectors can be developed, each harboring our β-hexosaminidase transgene driven by the NSE, COLL1 or the pancellular chicken β-actin (βact) promoter. Affected pups can receive neonatal intraperitoneal injections and their growth & development can be evaluated over time.

Disclosed is the transfer of β-hexosaminidase therapeutic transgenes to experimental animals, and other subjects, neonatally, prior to the development of significant craniofacial dysplasia.

(1) Construction of Viral Vectors

See examples herein for all vectors and construction.

(2) Use of Vectors

First, FIV(βact-Hex), FIV(COLL1-Hex) and FIV(NSE-Hex) can be tested in vitro as follows. FIV(NSE-Hex) will be evaluated on the N2α cell line (ATCC), FIV(COLL1-Hex) on the D1 multipotent mouse bone marrow stromal precursor cell line (D1 ORL UVA; ATCC, cat# CRL-12424) and FIV(βact-Hex) on the mouse fibroblast NIH 3T3 cell line.

The D1 cells can potentially differentiate into different types, including osteocytes and chondrocytes in the presence of appropriate growth factors per manufacturer's instructions (ATCC). In brief, (1) one can determine the presence of the β-hexosaminidase transgene in DNA extracts of infected cells using established HexA and HexB PCR protocols (As disclosed herein). The HexA and HexB primers are designed to amplify the human gene only, and therefore can detect transgene incorporation in murine cells. (2) One can evaluate HexA and HexB expression at the transcription level in total RNA extracts using established HexA and HexB RT-PCR protocols as disclosed herein (3) one can evaluate the β-hexosaminidase activity by 4MUG/S fluorometry as disclosed herein.

(3) Determine the Distribution, Level and Persistence of β-Hexosaminidase Expression

One can characterize the spatial distribution of the β-hexosaminidase transgene in hexA^{-/-}/hexB^{-/-} double knockout mice following systemic administration of FIV vectors at neonatal stages of development, with special emphasis on the brain and craniofacial skeleton. The level of HexA and HexB expression, at the mRNA, protein and activity levels, can be temporally analyzed in the brain and naso-maxillary complex. Three FIV β-hexosaminidase vectors can be employed in this experiment, FIV(βact-Hex), FIV(COLL1-Hex) and FIV(NSE-Hex), as well as FIV(lacZ) that will serve as control. Please refer to Table 17, below.

Each FIV vector can be administered in the experimental and control mice at neonatal stages of development. As described in Specific Aim 1, hexA^{-/-}/hexB^{-/-} mice can be generated by breeding hexA^{-/-}/hex^{+/-} mice; therefore, each litter of pups can include hexA^{-/-}/hexB^{-/-} (experimental; expectancy 0.25) as well as hexA^{+/-}/hexB^{+/-} mice that do not display any craniofacial pathology (controls; expectancy 0.50). Since the injections can be performed prior to genotyping, each litter can receive a single treatment. The pups can receive a total of 5×10⁶ infectious FIV particles in 100 μl of sterile saline solution intraperitoneally (I.P.) at 2 days of age (P2). In addition, other litters can receive intraperitoneal injections of 100 μl vehicle (saline). The FIV(lacZ) group of animals can provide information on the effects of the viral vector itself, whereas the saline injections can control for the procedure. Moreover, one can employ B6129SF2 mice (Jax stock# 101045) that can be matched for age and gender and can serve as appropriate wild type controls. Please refer to Table 16.

Table 17 Neonatal transfer of FIV vectors to hexA−/−/hexB−/− pups. Five (5) groups of hexA^{-/-}/hexB^{-/-} mice can be employed in this experiment; each group (N=120) can receive one of the following treatments at post-natal day P2: FIV(βact-Hex), FIV(COLL1-Hex), FIV(NSE-Hex), FIV(lacZ) or saline. In addition, a group of hexA^{+/-}/hexB^{+/-} mice (N=120) will receive saline injections (controls), whereas a group of B6129SF2 wild type mice (N=120) can receive no treatment. At each time point, 30 mice of each group can be evaluated by radiography (cephalometric analysis) and behavioral motor analyses. Thereafter, the mice will be terminated and analyzed by molecular (20 mice of each group), biochemical (10 mice of each group), histological (10 mice of each group).

TABLE 17

| Genotype | Pathology | Treatment | Time Points | N | Analyses |
|---|---|---|---|---|---|
| hexA^{-/-}/ | affected | FIV(βact-Hex) | 1, 2, 3 & 4 wks | 30 × 4 = 120 | Molecular |
| hexB^{-/-} | | | | | |
| —//— | affected | FIV(COLL1-Hex) | 1, 2, 3 & 4 wks | 30 × 4 = 120 | Biochemical |
| —//— | affected | FIV(NSE-Hex) | 1, 2, 3 & 4 wks | 30 × 4 = 120 | Histology |
| —//— | affected | FIV(lacZ) | 1, 2, 3 & 4 wks | 30 × 4 = 120 | Behavioral |
| —//— | affected | saline | 1, 2, 3 & 4 wks | 30 × 4 = 120 | Radiography |
| hexA^{+/-}/ | control | saline | 1, 2, 3 & 4 wks | 30 × 4 = 120 | |
| hexB^{+/-} | | | | | |
| B6129SF2 | wild type | — | — | 30 × 4 = 120 | |
| | | | | Total = 840 | |

The sphere of β-hexosaminidase gene distribution at the various time points can be quantitatively assessed by determining the number of gene copies in the brain (cerebrum & cerebellum and naso-maxillary complex) by established QPCR methods. The data can be analyzed relative to time and treatment by the 2-way ANOVA with alpha=0.05. Differences between the various groups can be determined by the Tukey post-hoc analysis. Similarly, one can analyze the distribution of the reporter gene lacZ in the FIV(lacZ) groups.

One can assess the level of β-hexosaminidase gene expression at the mRNA level by quantitatively determining HexA and HexB mRNA levels in brain (cerebrum & cerebellum and naso-maxillary complex) in total RNA extracts by QRT-PCR protocols as previously described. HexA and HexB levels can be assessed over time following neonatal treatment. In addition, HexA, HexB and lacZ mRNA localization can be investigated by in situ hybridization (ISH) on brain and nasomaxillary skeleton as previous described (Brouxhon et al. Brain Behav Immun 12:107-122 (1998); Bellinger et al. J Neuroimmunol 119: 37-50 (2001) using whole length probes for the human HexA and HexB. By coupling ISH with immunocytochemistry (ICC), one can determine the identity of cells expressing HexA & HexB by employing cell specific markers, including cerebral (NeuN) and cerebellar neurons (calbindin). Osteocytes/osteblasts can be confirmed by the expression alkaline phosphatase, osteocalcin, type I collagen (Liu et al. Exp Cell Res 232: 97-105 (1997); Adamo et al. J Oral Implantol 27: 25-31 (2001)). Chondrocytes can be confirmed by detection of collagen II (Scott-Burden et al. Ann Thorac Surg 73: 1528-33 (2002). Endothelial cells can be stained with antibodies raised against PECAM-1 (CD 31), and murine macrophages by CD 11b (mac-1) as disclosed herein. One can semi-quantitatively assess the transduction of the various cell types with the β-hexosaminidase gene on alternate tissue sections utilizing methods previously described (Kyrkanides et al. J Orofac Pain 16:229-235 (2002b)).

In addition, the expression of β-hexosaminidase at the protein level can be qualitatively assessed by immunocytochemistry (ICC) on histology sections harvested from brain (cerebrum & cerebellum) and nasomaxillary complex utilizing antibodies against human HEX-A and HEX-B. The identity of the cells can be confirmed by double immunofluorescence as described in the paragraph above (Kyrkanides et al. J Neuroimmunol 95:95-106. (1999). The total number of transduced cells, as well as the number of cells transduced for each cell type can be semi-quantitatively assessed on brain and nasomaxillary sections by methods previously described (Kyrkanides et al. J Orofac Pain 16:229-235 (2002b). Western immunoblotting will be employed for semi-quantitative analysis of HEX-A and HEX-B levels in the brain and nasomaxillary complex (Utsumi et al. Acta Neurol Scand 105:427-30 (2002); Proia et al. J Biol Chem 259: 3350-3354 (1984)).

One can quantitatively determine the levels of HexA and HexB enzymatic activity in relation to time in the various experimental time points and different tissues by 4MUGS and 4MUG fluorometry, respectively (As disclosed herein)). In addition, a simple histochemical method for visualization of total β-hexosaminidase activity on histology tissue sections, X-Hex, as disclosed herein can be performed.

Persistence of β-hexosaminidase expression can be evaluated as follows. Based on the data derived from the aforementioned experiments, one can temporally analyze HexA & HexB expression (mRNA, protein and activity levels) in relation to the number of transgene gene copies present in the various organs over time. Interpretation of the data can provide a measure of expression persistence (versus silencing). Moreover, one can calculate the percent of β-hexosaminidase recovery in treated animals relative to wild type littermates, normalized to total protein, in the various tissues and at the different time points. This latter analysis can provide information pertinent to the efficacy of FIV therapy in restoring β-hexosaminidase activity.

d) Evaluate the Effects of the Various FIV Vectors on Craniofacial Development

Storage of insoluble metabolites can be first evaluated in histology brain and nasomaxillary tissue sections by periodic acid Schiff histochemical staining (Sango et al. Nature Genet 14: 348-352 (1996); Suzuki et al. J Neuropath Exp Neurol 56: 693-703 (1997)). In addition, GM₂ storage levels can be assessed by immunocytochemistry employing commercially available monoclonal anti-GM₂ antibody (Sakuraba et al. Clin Chim Acta 265: 263-266 (1997); Seikagaku, Falmouth Mass.). GM₂ staining differences in the various animal groups can be evaluated by semi-quantitative analysis (Kyrkanides et al. J Neuroimmunol 119: 269-77 (2001), Kyrkanides et al. J Orofac Pain 16:229-235 (2002b. For quantitative analysis of GM₂ in the brain and nasomaxillary complex, one can employ immuno-thin layer chromatography on lysate lipid extracts (As disclosed herein).

Cell death can be assessed in the brain and craniofacial skeleton. In the brain, neuronal degeneration can be first evaluated histochemically utilizing the Fluoro-Jade agent on histology brain tissue sections (Fluoro-Jade (Histo-Chem. Inc., Jefferson Ariz.), a fluorescent agent that stains neurons undergoing degeneration (Schmued et al. Brain Res 751: 37-46 (1997); Methods in Detail). The number of neurons undergoing apoptosis can be assessed by the fluorescein terminal uridine nick-end labeling method (TUNEL detects cellular apoptosis) on alternate brain sections coupled with immunofluorescence utilizing antibodies raised against NeuN (cerebral neurons) and calbindin (cerebellar cortical neurons). Details on the use of these two antibodies have been described above. In addition, one can confirm apoptosis by multiple immunofluorescence utilizing antibodies raised against caspase-3 or -8 coupled with neuronal markers and/or TUNEL. The total number of nuclei can be determined by Hoechst nuclear staining. The number of cells undergoing cell death can be counted by stereology in alternate brain sections normalized for total number of nuclei (Hoechst staining) adopting methods previously described (Kyrkanides et al. J Orofac Pain 16:229-235 (2002b)). Cell identity can be confirmed by double immunofluorescence as described above.

Since the introduction of novel proteins may elicit an immunologic response in mice treated with FIV vectors, one can characterize the host's immunologic response following perinatal treatment. To this end, one can quantitatively assess the presence (titers) of antibodies against viral and transgenic proteins in blood serum at the different experimental time points. To this end, IgG and IgM titers for HEXA and HEXB, as well as the FIV p24 antigen can be assessed by customized ELISA method (Kang et al. (2002)). In brief, ELISA plates can be coated with 5 mg of human HEX-A, HEX-B (Sigma; St. Louis Mo.) or p24 recombinant proteins (IDEXX Laboratories Inc.; Westbrook Me.). After incubation with the sera, the plates can be alkaline phosphatase-conjugated goat anti-mouse IgG and IgM (Southern Biotechnology Associates, Inc; Birmingham Ala.). Antibody titers can be established as the serum dilution that reached absorbance levels of saline injected mice assuming linear extrapolation (Kang et al. 2002).

Evaluation of behavioral performance is also an important treatment outcome measure. Animal weight of experimental and control mice can be monitored weekly throughout the experiment. Motor competency can be assessed by the ability to maintain balance on a rotating cylinder (rotorod) by measuring the latency of each animal to fall off. In addition, their motor activity can be assessed by placing the mice on a wire mesh fixed on one end of a clear plastic cylinder, and turning the cylinder with the mesh and mice attached up side down. Motor competency can be evaluated in the experiment by measuring the latency of each animal to fall off. Using these two methods, one can assess motor behavior experimental and control mice on a weekly basis. Life span w can ill also be recorded, since the affected mice suffer from significantly shortened life span (4 months). Life span can be calculated as the total number of days an animal survived.

For each outcome, one can first make comparisons between treatments in the affected group. This can be done separately at each time point using ANOVA. One can next test for changes in treatment effects over time using linear regression models. Next, one can compare the affected animals to the control and wild type groups. Again, this can be done at each time point using ANOVA, and across time points using linear regression methods. Significance levels can be set at 0.05. As with the analyses for Aims 1 and 2, we can consider appropriate transformation of outcomes that are non-normal. Compilation of the above data, in conjunction with the data on transgene expression, can provide information on the level of β-hexosaminidase levels required for clinical correction of the anomaly relative to wild type littermates. The aforementioned experiments, collectively, can determine the efficacy of FIV neonatal gene therapy in restoring β-hexosaminidase activity.

Neonatal FIV injection can result in wide vector distribution. β-hexosaminidase activity can be restored at therapeutic levels, leading to attenuation of the disease. Conzelmann et al. (Dev Neurosci 6: 58-71 (198.3)) used a sensitive assay to demonstrate a correlation between level of residual β-hexosaminidase activity and clinical severity in humans and reported that 10% or more residual, activity correlates with healthy phenotype. Therefore, we expect that restoration at 5% or greater in β-hexosaminidase activity can be beneficial to the affected animals.

Perinatal gene therapy can resolve the correlate β-hexosaminidase deficiency in hexA^{-/-}/hexB^{-/-} mice and restore cellular function, thereby allowing normal bone growth and skeletal development to resume postnatally.

10. Example General Methods

There are a number of different methods that are disclosed herein. Provided in this Example, are general methods that can be used in a variety of different protocols or data collection. Many of the methods have been performed herein as described herein.

a) RNA Isolation and cDNA Synthesis (Reverse Transcription; RT)

Tissue can be dissected out, frozen in isopentane chilled with dry ice, and stored in sterile tubes at −80° C. until ready for RNA isolation. RNA can be isolated using Trizol reagent (Invitrogen, Carlsbad, Calif.), precipitated and the concentration determined by spectrophotometry. Two μg of RNA can be DNase-treated (Invitrogen) according to the manufacturer's instructions. First-strand DNA is synthesized by using 2 μg of DNase-treated RNA, random hexamers, and Superscript II (Invitrogen) according to the manufacturer's instructions.

b) Quantification of cDNA (mRNA) and DNA Using Real-Time Real Time Polymerase Chain Reaction (QPCR)

Quantification of mRNA levels can be completed using an iCycler (Bio-Rad, Hercules, Calif.) and real time PCR with SYBR Green as the fluorescent marker (Molecular Probes, Eugene, Oreg.). Prior to PCR of the cDNA samples, PCR conditions are optimized for each mRNA to be analyzed. Standard curve reactions can be performed by varying annealing temperatures, Mg²⁺ concentrations, primer concentrations, and SYBR green concentration. Melt curve analysis can also be completed for each PCR amplification to confirm production of a single product with the expected melting temperature. Serial dilution of the starting cDNA template demonstrates linear amplification over at least 5 orders of magnitude.

PCR reactions can be done in a volume of 25 μl and contain 4.0 mM Mg²⁺, 0.2 μM concentrations of each primer (except ICAM-1 and G3PDH at 0.4 μM), 1 μl of SYBR Green (1:100,000 final dilution), 100 μM nucleotide mix (Stratagene, LaJolla, Calif.), 0.5 U of Platinum Taq in PCR buffer (Invitrogen), and 1 μl of cDNA sample. To ensure consistency, a master mix can be first prepared containing all reagents except the cDNA sample. Primers used were designed (and others can be) using the Oligo 6.82 program (Molecular Biology Insights, Inc., Cascade, Colo.). The primer pairs used can be as follows:

HexA

(SEQ ID NO:72)

^{5′}GAA TCC CAG TCT CAA TAA TAC C^{3′}
and

(SEQ ID NO:73)

^{5′}CAT ACA AGC CTC TCC ACC^{3′};
HexB

(SEQ ID NO:53)

^{5′}AGT CCT GCC AGA ATT TGA TAC C^{3′}
and

(SEQ ID NO:54)

^{5′}ATT CCA CGT TCG ACC ATC C^{3′};
lacZ

(SEQ ID NO:55)

^{5′}TTT TTC CAG TTC CGT TTA TCC^{3′}
and

(SEQ ID NO:56)

^{5′}TTT ATC GCC AAT CCA CAT CT^{3′};
G3PDH

(SEQ ID NO:57)

^{5′}ACC ACA GTC CAT GCC ATC AC^{3′}
and

(SEQ ID NO:58)

^{5′}TCC ACC ACC CTG TTG CTG TA^{3′};
18S

(SEQ ID NO:59)

^{5′}TGG TGG AGC GAT TTG TCT GGT T^{3′}
and

(SEQ ID NO:60)

^{5′}TAG TAG CGA CGG GCG GTG TG^{3′};
IL-1β

(SEQ ID NO:45)

^{5′}GAG AAC CAA GCA ACG ACA AAA TAC C-3′
and

(SEQ ID NO:46)

^{5′}GCA TTA GAA ACA GTC CAG CCC ATA C^{3′};
TNF-α

(SEQ ID NO:47)

^{5′}CGA GTG ACA AGC CTG TAG CC^{3′}
and

(SEQ ID NO:48)

^{5′}GGT TGA CTT TCT CCT GGT ATG AG^{3′};
ICAM-1

(SEQ ID NO:61)

^{5′}CAC CCC AAG GAC CCC AAG GAG AT^{3′}
and

(SEQ ID NO:62)

^{5′}CGA CGC CGC TCA GAA GAA CCA C^{3′};
MCP-1

(SEQ ID NO:63)

^{5′}CAG CAG GTG TCC CAA AGA A^{3′}
and

(SEQ ID NO:64)

^{5′}CTT GAG GTG GTT GTG GAA AAG^{3′};
IP-10

(SEQ ID NO:65)

^{5′}CCC AAG TGC TGC CGT CAT TT^{3′}
and

(SEQ ID NO:66)

^{5′}GAT AGG CTC GCA GGG ATG ATT TC^{3′};
IFN-γ

(SEQ ID NO:67)

^{5′}TCT CTT TCT ACC TCA GAC TCT TTG AA^{3′}
and

(SEQ ID NO:68)

^{5′}GAC TCC TTT TCC GCT TCC TG^{3′};

In general, the PCR reaction conditions can be the following: denaturation at 95° C. for 3 min, followed by 60 cycles of amplification by denaturing at 95° C. for 30 s, annealing at 64° C. for 30 s and extension at 72° C. for 60 s. Annealing temperatures can be optimized and found to be 64° C. for G3PDH and 18S. The optimal annealing temperature for IL-113 is 62° C., 56° C. for MCP-1 and 72° for IP-10. For each real time PCR, a standard curve can be performed to insure direct linear correlation between product yield (expressed as the number of cycles to reach threshold) and the amount of starting template. The correlation can always be greater than r=0.925. PCR reaction efficiency (□) is determined for each reaction. To correct for variations in starting RNA values, the level of G3PDH or ribosomal 18S RNA can be determined for all samples and used to normalize all subsequent RNA determinations. Normalized threshold cycle (Tc) values can then be transformed, using the function, expression=(1+□)^{Tc}, in order to determine the relative differences in transcript expression.

c) Quantification of cDNA (mRNA) and DNA Using Real-Time Real Time Polymerase Chain Reaction (QPCR)

In general, the PCR reaction conditions are the following: denaturation at 95° C. for 3 min, followed by 60 cycles of amplification by denaturing at 95° C. for 30 s, annealing at 64° C. for 30 s and extension at 72° C. for 60 s. Annealing temperatures are optimized and found to be 64° C. for TNF-α, INP-γ, ICAM-1, G3PDH and 18S. The optimal annealing temperature for IL-1β is 62° C., 56° C. for MCP-1 and 72° for IP-10. For each real time PCR, a standard curve is performed to insure direct linear correlation between product yield (expressed as the number of cycles to reach threshold) and the amount of starting template. The correlation is always greater than r=0.925. PCR reaction efficiency (ε) is determined for each reaction. To correct for variations in starting RNA values, the level of G3PDH or ribosomal 18S RNA is determined for all samples and used to normalize all subsequent RNA determinations. Normalized threshold cycle (Tc) values are then transformed, using the function, expression=(1+ε)^{Tc}, in order to determine the relative differences in transcript expression.

d) ICC and Histochemical Quantitative Analysis

Immunocytochemically stained sections can be viewed in a Zeiss Axioplan light microscope equipped with a Prior XYZ motorized stage, Sony video camera, SONY high resolution color monitor and Apple Macintosh G3 computer. Morphometric data can be collected using the Stereologer software program and the optical fractionator method. For immunostained sections, every sixth section can be counted and the number of labeled cells expressed per unit volume (# cells/100 μm³). Confirmation of cells can be accomplished by double immunofluorescent staining, but morphometric data can be collected from glia, neurons, endothelial cells, and pericytes, as appropriate for the antibody used (e.g., GFAP for astrocytes or MHC class II for microglia/macrophages). Variances, homogeneity of variance and tests for normality (Shapiro-Wilk W test) can be assessed by the JMP statistics program (SAS Institute). A probability of P<0.05 can be considered significant.

### e) X-Hex Histochemistry

Total β-hexosaminidase activity can be detected on fixed cells or tissue sections by a solution containing 1 mg/mL 5-bromo-4-chloro-3-indolyl N-acetyl-β-D-glycosaminide in citrate buffer (pH 3.8) including 1.5 mM ZnCl₂, 20 mM K₄Fe(CN)₆, 20 mM K₃Fe(CN)₆, 2 mM MgCl₂ complete. Cells expressing β-hexosaminidase stain blue and are readily identified under the microscope.

f) 4MUG/S Fluorometry

Lysates in 20 mM Tris-HCl/137 mM NaCl/1 mM MgCl₂/1 mM CaCl₂/1 mM vanadate/1 mM aprotinin/0.5 mM PMSF buffer with 1% N-40 (from cultured cells or homogenized tissues) can be briefly sonicated for 20 sec using a Branson Sonifier 450 with attached microtip, and dispensed in 250 μl aliquots which can then be combined with equal volume of 2× citrate reaction buffer (pH 4.4) containing 2 mM of 4MUG (MU-GlcNAc) for HEXA+HEXB detection, or 4MUGS (MU-GlcNAc-6-SO₄) for HEXA detection), and incubated for 2 hrs at 37° C. HEXA+HEXB or HEXA-only activity can be determined semi-quantitatively (experimental versus control) by assessing the fluorescent product of the substrate-enzyme reaction using a Packard Instruments Fluorometer.

g) Western Immunoblotting

Cultured cells or homogenized animal tissues can be lysed in 0.125 M Tris-4% SDS buffer pH 6.8. Cell proteins can be separated on a denaturing 10% SDS-PAGE gel and then transferred to polyvinylidene difluoride membranes in 10 mM CAPS-10% Methanol (pH 11) buffer. Detection of HEXA and HEXB proteins on blots can be performed using polyclonal antibodies (1:1,000 dilution) that have been raised in goat specifically against the human HEXA and human HEXB (primary antibodies) (Proia R L, et al., J Biol Chem 259: 3350-3354. (1984)). Then, the blots can be incubated in secondary antibody solution (HRP-conjugated donkey anti-goat IgG) followed by ECL Plus (Amersham Pharmacia, UK) mediated detection on radiographic film per manufacturer's instructions. Radiographic images can be captured and analyzed by a Kodak digital image analysis system attached to a PC computer.

h) GM₂ Immuno-Thin Layer Chromatography

Mouse brain tissues will be treated by chloroform/methanol solvents for lipid protein extraction. After saponification, the glycosphingolipids can be fractionated into neutral and acidic by DEAE-Sephadex A-2 chromatography. Galgliosides will be separated by HPTLC on Si-60 plates in solvents chloroform/acetone 1:1 v/v (pre-run) followed by chloroform/methanol/0.2% CaCl₂ (55:45:10). GM2 ganglioside will be visualized by immune-thin layer chromatography using monoclonal antibodies against GM₂ (Seikagaku) with purified bovine brain GM₂ as standard curve (Sigma). Quantification can be made within the linear range of calibration curves.

i) FIV Production and Concentration

Cultured 293-T cells can be transfected with a FIV DNA cocktail (20 μg of pFIV, 15 μg of pVSV-G and 5 μg of pPAC) using the Lipofectamine 2000 reagent per manufacturer's instructions (Invitrogen). Sixty hours later, the supernatant can be collected and filtered (0.45 μm). This FIV-rich solution can be used directly or further concentrated to increase titers. The concentration process can be based on an overnight centrifugation of FIV solution at 7,000×g at 4° C. using a Sorvall RC 5B plus centrifuge with a SS-34 rotor. The supernatant can then be decanted and the viral pellet can be reconstituted in sterile saline with 40 mg/ml lactose. Titering can be performed on feline kidney CrfK cells (ATCC) by counting blue forming units after X-gal histochemistry, and routinely range 10⁷-10⁸ infectious particles/mL.

j) Genotyping Mouse Genotypes can be Determined by Employing Established PCR Methods from Biopsy DNA Extracts. The Following Primers Will be Utilized.

TABLE 18

| | | | |
|---|---|---|---|
| Locus | Primers | T_{A} | Product |

| | | | |
|---|---|---|---|
| murine | ^{5′}ATT TTA AAA TTC AGG CCT | 58° C. | 120 bp |
| hexB | CGA^{3′} | | (wild type) |
| | (SEQ ID NO:42) | | v/s |
| | ^{5′}CAT AGC GTT GGC TAC CCG | | 220 bp |
| | TGA^{3′} | | (knockout) |
| | (SEQ ID NO:43) | | |
| | ^{5′}CAT TCT GCA GCG GTG CAC | | |
| | GGC^{3′} | | |
| | (SEQ ID NO:44) | | |

k) ICC and Histochemical Quantitative Analysis

Immunocytochemically stained sections can be viewed in a Zeiss Axioplan light microscope equipped with a Prior XYZ motorized stage, Sony video camera, SONY high resolution color monitor and Apple Macintosh G3 computer. Morphometric data can be collected using the Stereologer software program and the optical fractionator method. For immunostained sections, every sixth section will be counted and the number of labeled cells expressed per unit volume (# cells/100 μm³). Confirmation of cells can be accomplished by double immunofluorescent staining, but morphometric data can be collected from glia, neurons, endothelial cells, and pericytes, as appropriate for the antibody used (e.g., GFAP for astrocytes or MHC class II for microglia/macrophages). Variances, homogeneity of variance and tests for normality (Shapiro-Wilk W test) can be assessed by the JMP statistics program (SAS Institute). A probability of P<0.05 can be considered significant.

l) Fast Garnet Histochemistry

Total β-hexosaminidase activity can be detected on fixed cells or tissue sections by Napthol-AS-BI-N-acetyl-B-glucosaminide (1 mg/ml) in 0.1M citrate buffer with 10% ethylene glycol monomethyl ether plus 0.5% CaCl₂ and 1% polyvinyl alcohol, for 2 hrs at 37° C. The supernatant is then discarded and the cells or tissue thoroughly washed with 0.1M acetate buffer. The enzymatic reaction is then visualized by Fast Garnet incubation (1 mg/ml in 0.1M acetate buffer pH=6.2) at room temperature for 30 min (red colored stain), washed with fresh PBS and temporarily stored at 4° C. Tissue slides can be coverslipped using a PBS/glycerol based mounting media (Supermount; BioGenex).

m) Immunocytochemistry and Immunofluorescence (Single or Double)

The detection of HEXA and HEXB proteins can be performed in fixed cells or tissue sections mounted on slides using the aforementioned polyclonal antibodies by adapting the protocols described in Kyrkanides et al. J Neuroimmunol 95:95-106. (1999) & Kyrkanides et al. J Neuroimmunol 119: 269-77 (2001), Kyrkanides et al. Mol Brain Res 104: 159-169 (2002a), Kyrkanides et al. J Orofac Pain 16:229-235 (2002b).

n) Development and Testing of Transgenic Animals.

NSE-Hex transgenic animals, and any others disclosed herein, can be developed, and 4-7 founder lines per animal can be propagated on C57BL/6 background.

o) Intraperitoneal Injections of Replication Defective FIV(βact-Hex) Vectors

The objective of this experiment is to induce the expression of β-Hex in perinatal mice (P2) by injection of the lentiviral vector FIV(βact-Hex). For this purpose, the pups can be anesthetized with halothane in 70% N₂O and 30% O₂. To verify the induction of surgical anesthesia, a toe is pinched in order to test for reflex withdrawal. If no response can be elicited, the mouse is considered adequately anesthetized. Halothane anesthesia eliminates the possibility of pain and distress due to handling and intraperitoneal injection. All surgical procedures shall only be conducted on completely anesthetized mice.

Intraperitoneal injections. For this purpose, mouse pups can be injected 100 □l of 5×10⁷ ip/ml of FIV(βact-Hex) using a 1 mm syringe with a 25 gage needle. This is a very simple procedure. The pups can recover from anesthesia in quiet and lamp-warmed area. Upon assumption of fully mobility the subjects can be returned to their mothers. The total estimated procedure time is less than 5 minutes.

p) Mouse Identification and Tail Biopsy.

The pups can be identified by ear punching. Mice can be held at base of tail with distal portion of tail situated on surface of nestlet. Using a straight edge blade, one can remove ˜7 mm of distal tail, place mouse in cage and store tail specimen in vial labeled by mouse ID# and sex. Tails can be utilized for total DNA extraction in the laboratory as usual.

q) Euthanasia

The objective of this procedure is to obtain regions from mice that have been prepared as described in the previous sections. Induction of deep anesthesia is performed. The mice are euthanised with sodium pentobarbital (200 mg/kg). Fixation by intracardial transfusion Upon exposure of the heart, the right atrium will be clipped and the left ventricle can be catheterized with a 17 gage needle through which 50 ml of 4% paraformaldehyde solution in phosphate buffered saline can be transfused into the animal. The liver, spleen, kidney and brain can be dissected and post-fixed until sectioned for histology. The middle part of the cranium, including the cranial base (sphenoid, ethmoid, maxilla) can also be dissected, demineralized by immersion into an EDTA solution and section for histology.

### G. REFERENCES

Adamo C T, Mailhot J M, Smith A K, Borke J L (2001) Connexin 43 expression in oral derived human osteoblasts after transforming growth factor-beta and PGE2 exposure. J Oral Implantol 27: 25-31.
Akima, M., H. Nonaka, M. Kagesawa, K. Tanaka, A study on the microvasculature of the cerebellar cortex. The fundamental architecture and its senile change in the cerebellar hemisphere, Acta Neuropathol. 75 (1987) 69-76.
Akli S, Guidotti J-E, Vigne E, Perricaudet M, Sandhoff K, Kahn A, Poenaru L (1996) Restoration of hexosaminidase A activity in human Tay-Sachs fibriblasts via adenoviral vector mediated gene transfer. Gene Therapy 3: 769-774
Alisky, J. M., S. M. Hughes, S. L. Sauter, D. Joly, T. W. Dubensky Jr., P. D. Staber, J. A. Chiorini, B. L. Davidson, Transduction of murine cerebellar neurons with recombinant FIV and AAV5 vectors, Mol. Neurosci. 11 (2000) 2669-2673.
Barranger, J., E. O'Rourke, Lessons learned from the development of enzyme therapy for Gaucher disease, J. Inherit. Metabol. Disord. 24 (2001) 89-96.
Bartlett J S, Samulski R J (1998) Fluorescent viral vectors: A new technique for the pharmacological analysis of gene therapy. Nat Medicine 4: 635-637.

Bartlett J S, Wilcher R, Samulski R J (2000) Infectious entry pathway of adeno-associated virus and adeno-associated virus vectors. J Virology 74: 2777-2785.
Bellinger D L. Felten D L. Lorton D. Brouxhon S. Effects of interleukin-2 on the expression of corticotropin-releasing hormone in nerves and lymphoid cells in secondary lymphoid organs from the Fischer 344 rat. Journal of Neuroimmunology 119(1):37-50, 2001.
Ben-Shachar D, Laufer D, Livne E, Silbermann M (1988a) Picrotoxin, a γ-aminobutyric acid-receptor antagonist, retards craniofacial development in the weaning rat: I. Effect on mandibular bone growth. J Craniofac Genet Develop Biol 8: 351-361.
Ben-Shachar D, Laufer D, Livne E, Silbermann M (1988b). Picrotoxin, a gamma-aminobutyric acid-receptor antagonist, retards craniofacial development in the weaning rat: II. Effect on mandibular condylar cartilage. J Craniof Genet Develop Biol 8: 363-372.
Birkenmeier, E. H., J. E. Baker, C. A. Vogler, J. W. Kyle, W. S. Sly, B. Gwynn, B. Levy, C. Pegors, Increased life span and correction of metabolic defects in murine mucopolysaccharidosis type VII after syngeneic bone marrow transplatation. Blood 78 (1991) 3081-3092.
Bloemer, U., L. Naldini, T. Kafri, D. Trono, I. M. Verma, F. H. Gage, Highly efficient and sustained gene transfer in adult neurons with a lentivirus vector, J. Virology 71 (1997) 6641-6649.
Bowers W J, Howard D F, Federoff H J (2000). Discordance between expression and genome transfer titering of HSV amplicon vectors: recommendation for standardized enumeration. Mol Ther 1:294-9.
Bradl, M., A. Flugel, The role of T cells in brain pathology, Curr. Topics Microbiol. Immunol. 265 (2002) 141-162.
Bragg, D. C., L. C. Hudson, Y. H. Liang, M. B. Tompkins, A. Fernandes, R. B. Meeker, Choroid plexus macrophages proliferate and release toxic factors in response to feline immunodeficiency virus, J. Neurovirol. 8 (2002) 225-239.
Brooks A I, Muhkerjee B, Panahian N, Cory-Slechta D, Federoff H J (1997). Nerve growth factor somatic mosaicism produced by herpes virus-directed expression of cre recombinase. Nat Biothech 15: 57-62.
Brooks A I, Stein C S, Hughes S M, Deth J, McCray P M Jr., Sauter S I, Johnston J C, Cory-Slechta D A, Federoff H J, Davidson B L (2002). Functional correction of established central nervous system deficits in an aniumal model of lysosomal storage disease with feline immunodeficiency virus-based vectors. Proc Natl Acad Sci USA 99: 6216-6221.
Brooks, S. E., L. M. Hoffman, M. Adachi, D. Amsterdam, L. Schneck, Enzyme replacement treatment for Tay-Sachs disease brain cells in culture utilizing concanavalin A-mediated hexosaminidase A uptake: biochemical and morphological evidence of GM2 mobilization, Acta Neuropath. 50 (1980) 9-17.
Brouxhon S M, Prasad A V, Joseph S A, Felten D L, Bellinger D L (1998) Localization of corticotropin-releasing factor in primary and secondary lymphoid organs of the rat. Brain Behav Immun 12: 107-122.
Burns J C, Friedmann T, Driever W, Burrascano M, Yee J-K (1993) Vescicular stomatitis virus G-glycoprotein pseudotyped retroviral vectors Concentration to very high titer and efficient gene transfer into mammalian and nonmammalian cells. Proc Natl Acad Sci USA 90: 8033-8037.
Burstein Y, Zakuth V, Rechavi G, Spirer Z (1987). Abnormalities of cellular immunity and natural killer cells in Gaucher's disease. J Clin Lab Immunol 23: 149-151.
Byrd K E, Yang L, Yancey K W, Teomim D, Domb A J (2000) Increased in vivo levels of neurotransmitters to trigeminal motoneurons: effects on craniofacial bone and TMJ. Anat Record 258: 369-383.
Cannon P M, Anderson W F (2000) Retroviral vectors for gene therapy. In Gene Therapy: therapeutic mechanisms and strategies. N S Templeton and D D Lasic, Editors. Marcel Dekker Inc, New York, pp 1-16.
Carneiro, F. A., L. Bianconi, G. Weissmueller, F. Stauffer, A. T. Da Poian, Membrane recognition by vesicular stomatitis virus involves enthalpy-driven protein-lipid interactions, J. Virol. 76 (2002) 3756-3764.
Chavany C, Jendoubi M (1998) Biology and potential strategies for the treatment of GM₂ gangliosidoses. Mol Med Today 4: 158-65.
Cinato E, Mirotsou M, Sablitzky F (2001). Cre-mediated transgene activation in the developing and adult mouse brain. Genesis: the Journal of Genetics & Development. 31(3):118-25, 2001.
Cohen M M Jr. Kreiborg S. The central nervous system in the Apert syndrome. Am J Med Genet. 35: 36-45.
Cohen-Tannoudji et al. Disruption of murine Hexa gene leads to enzymatic deficiency and to neuronal lysosomal storage, similar to that observed in Tay-Sachs disease. Mamm Genome. 6(12):844-9 (1995)
Conzelmann E, Sandhoff K (1983) Partial enzyme deficiencies: residual activities and the development of neurological disorders. Dev Neurosci 6: 58-71.
Culiat C T, Stubbs L J, Woychik R P, Russell L B, Johnson D K, Rinchik E M (1995). Deficiency of the beta 3 subunit of the type A gamma-aminobutyric acid receptor causes cleft palate in mice. Nature Genet 11: 344-346.
Curran, M. A., M. S. Ochoa, R. D. Molano, A. Pileggi, L. Inverardi, N. S. Kenyon, G. P. Nolan, C. Ricordi, E. S. Fenjves, Efficient transduction of pancreatic islets by feline immunodeficiency virus vectors, Transplantation 74 (2002) 299-306
Daly T M, Lorenz R G, Sands M S (2000). Abnormal immune function in vivo in a murine model of lysosomal storage disease. Pediatr Res 47: 757-762.
Daly T M, Ohlemiller K K, Roberts M S, Vogler C A, Sands M S (2001) Prevention of systemic clinical disease in MPS VII mice following AAV-mediated neonatal gene transfer. Gene Ther 1291-1298.
Daly T M, Okuyama T, Vogler C, Haskins M E, Muzyczka N, Sands M S (1999a) Intracranial injection of recombinant adeno-associated virus improves cognitive function in a murine model of mucopolysaccharidosis type VII. Hum Gene Ther 10:85-94.
Daly T M, Okuyama T, Vogler C, Haskins M E, Muzyczka N, Sands M S (1999) Neonatal intramuscular injection with recombinant adeno-associated virus results in prolonged beta-glucuronidase expression in situ and correction of liver pathology in mucopolysaccharidosis type VII mice. Hum Gene Ther 10: 85-94.
Daly T M, Vogler C, Levy B, Haskins M E, Sands M S (1999) Neonatal gene transfer leads to widespread correction of pathology in a murine model of lysosomal disease. Proc Natl Acad Sci USA 96: 2296-2300.
Daly, T. M., K. K. Ohlemiller, M. S. Roberts, C. A. Vogler, M. S. Sands, Prevention of systemic clinical disease in MPS VII mice following AAV-mediated neonatal gene transfer, Gene Ther. 8 (2001) 1291-1298.
Deng S X, Panahian N, James H, Gelbard H A, Federoff H J, Dewhurst S, Epstein L G (1998) Luciferase: a sensitive and quantitative probe for blood-brain barrier disruption. J Neurosci Methods 83: 159-164.
Duvemoy, H., S. Delon, J. L. Vannson, The vascularization of the human cerebellar cortex, Brain Res. Bull. 11 (1983) 419-480.
Dziennis et al. The CD11b promoter directs high-level expression of reporter genes in macrophages in transgenic mice. Blood 85(2):319-29 (1995)
Eng, C. M., M. Banikazemi, R. E. Gordon, M. Goldman, R. Phelps, L. Kim, A. Gass, J. Winston, S. Dikman, J. T. Fallon, S. Brodie, C. B. Stacy, D. Mehta, R. Parsons, K. Norton, M. O'Callaghan, R. J. Desnick, A phase ½ clinical trial of enzyme replacement in fabry disease: pharmacokinetic, substrate clearance and safety studies, Am. J. Hum. Genet. 68 (2001) 711-722.
Enlow D H, Hans M G (1996) Essentials of facial growth. W.B. Saunders Co, New York.
Forss-Petter S, Danielson P E, Catsicas S, Battenberg E, Price J, Nerenberg M, Sutcliffe J G (1990). Transgenic mice expressing β-galactosidase in mature neurons under neuron-specific enolase promoter control. Neuron 5: 187-197.
Fournet N, Garcia-Segura L M, Norman A W, Orci L (1986) Selective localization of calcium-binding protein in human brainstem, cerebellum and spinal cord. Brain Res 399: 310-316.
Frisella, W. A., L. H. O'Connor, C. A. Vogler, M. Roberts, S. Walkley, B. Levy, T. M. Daly, M. S. Sands, Intracranial injection of recombinant adeno-associated virus improves cognitive function in a murine model of mucopolysaccharidosis type VII, Mol. Ther. 3 (2001) 351-358.
Goldberg, S. H., P. van der Meer, J. Hesselgesser, S. Jaffer, D. L Kolson, A. V. Albright, F. Gonzalez-Scarano, E. Lavi, CXCR3 expression in human central nervous system diseases, Neuropath. Appl. Neurobiol. 27 (2001) 127-138.
Goldstein G W, Betz A L, Bowman P D, Dorovini-Z is K (1986). In vitro studies of the blood-brain barrier using isolated brain capillaries and cultured endothelial cells. Ann NY Acad Sci 481: 202-13.
Gorlin R J (1990). Metabolic Disorders. In Syndromes of the Head and Neck. Gorlin R J, Cohen M M and Levin L S (Eds). Oxford University Press, New York 1990, pp 99-117.
Gravel et al. Biochemistry and genetics of Tay-Sachs disease. Can J Neurol Sci. 18(3 Suppl):419-23 (1991)
Greenwood, J., Y. Wang, V. L. Calder, Lymphocyte adhesion and transendothelial migration in the central nervous system: the role of LFA-1, ICAM-1, VLA-4 and VCAM-1, Immunol. 86 (1995) 408-415.
Guidotti J E, M. A., Haase G, Caillaud C, McDonell N, Kahn A, Poenaru L (1999) Adenoviral gene therapy of the Tay-Sachs disease in hexosaminidase A deficient knockout mice. Hum Mol Genet. 8: 831-838.
Gurtu V, Yan G, Zhang G (1996). IRES bicistronic vectors for efficient creation of stable mammalian cell lines. Bioch Biophys Res Comm 229:295-298.

Halternan M W. Miller C C. Federoff H J. Hypoxia-inducible factor-1 alpha mediates hypoxia-induced delayed neuronal death that involves p53. [Journal Article] Journal of Neuroscience. 19(16):6818-24, 1999.
Havenga M J E, Vogels R, Braakman E, Kroos N, Valerio D, Hagenbeek A, van Es H H G (1998). Second gene expression in bicistronic constructs using short synthetic intercistrons and viral IRES sequences. Gene 222: 319-327.
Hickey W F (1991) Migration of hematogenous cells through the blood-brain barrier and the initiation of CNS inflammation. 1: 97-105.
Hickey, W. F., B. L. Hsu, H. Kimura, T-lymphocyte entry into the central nervous system, J. Neurosci. 28 (1991) 254-260.
Hickey, W. F., Basic principles of immunological surveillance of the normal central nervous system, GLIA 36 (2001) 118-124.
Hickey, W. F., K. Vass, H. Lassmann, Bone marrow derived elements in the central nervous system: an immunocytochemical and ultrastructural survey of rat chimeras, J. Neuropath. Epx. Neurol. 51 (1992) 246-256.
Hoffman W Y and McCarthy J G (1994). The effects of facial nerve ablation on craniofacial skeletal development in neonatal rabbits. Plast Recostr Surg 93: 1236-1240.
Huang D R, Wang J, Kivisakk P, Rollins B J, Ransohoff R M (2001) Absence of monocyte chemoattractant protein 1 in mice leads to decreased local macrophage recruitment and antigen-specific T helper cell type 1 immune response in experimental autoimmune encephalomyelitis. J Exp Med 193: 713-26.
Huang D R, Wang J, Kivisakk P, Rollins B J, Ransohoff R M (2001) Absence of monocyte chemoattractant protein 1 in mice leads to decreased local macrophage recruitment and antigen-specific T helper cell type 1 immune response in experimental autoimmune encephalomyelitis. J Exp Med 193: 713-26.
Huang J Q, Trasler J M, Igdoura S, Michaud J, Hanal N. Gravel R A (1997) Apoptotic cell death in mouse models of GM2 gangliosidosis and observations on human Tay Sachs and Sandhoffs diseases. Hum Mol Genet 6: 1879-1885.
Huard, J., H. Lochmuller, G. Ascadi, A. Jani, B. Massie, G. Karpati, The route of administration is a major determinant of the transduction efficiency of rat tissues by adenoviral recombinants, Gene Ther. 2 (1995) 107-115.
Izikson L, Klein R S, Luster A D, Weiner H L (2002) Targeting monocyte recruitment in CNS autoimmune disease. Clin Immunol 103:125-131.
Jeyakumar M, Thomas E, Elliot-Smith E, Smith D A, van der Spoel A C, d'Azzo A, Hugh Perry V, Butters T D, Dwek R A, Platt F M (2003). Central nervous system inflammation is a hallmark of pathogenesis in mouse models of GM1 and GM2 gangliosidosis. Brain 126: 974-987.
Kang, Y., C. S. Stein, J. A. Heth, P. L. Sinn, A. K. Penisten, P. D. Staber, K. L. Ratliff, H. Shen, C. K. Barker, I. Martins, C. M. Sharkey, D. A. Sanders, P. B. McCray Jr., B. L. Davidson, In vivo gene transfer using a nonprimate lentiviral vector pseudotyped with ross river virus glycoproteins, J. Virol. 76 (2002) 9378-9388.
Kessler M A. et al., The specificity of the myelin basic protein gene promoter studied in transgenic mice. [Journal Article] Biochemical & Biophysical Research Communications. 288(4):809-18, 2001
Kessler M A. Yang M. Gollomp K L. Jin H. Iacovitti L. The human tyrosine hydroxylase gene promoter. [Journal Article] Brain Research. Molecular Brain Research. 112(1-2):8-23, 2003
Kiesser B C, Wisniewski K E, Goebel H H (1997) The monocyte-macrophage system in affected in lysosomal storage disease: an immunoelectron microscopic study. Acta Neuropathol 94:359-362.
Kjaer I (1998) Neuro-osteology. Crit. Rev Oral Biol Med 9: 224-244.
Kjaer I, Keeling J W, Hansen B F (1999) The prenatal human cranium-Normal and pathologic development. Munksgaard, Copenhagen, pp 20-51.
Koch A E, Kunkel S L, Harlow L A, Johnson B, Evanoff H L, Haines G K, Burdick M D, Pope R M, Strieter R M (1992) Enhanced production of monocyte chemoattractant protein-1 in rheumatoid arthritis. J Clin Invest 90: 772-9.
Kordower J H, Bloch J, Ma S Y, Chu Y, Palfi S, Roitberg B Z, Emborg M, Hantraye P, Deglon N, Aebischer P (1999) Lentiviral gene transfer to the nonhuman primate brain. Exp Neurol 160:1-16.
Krebsbach P H. Harrison J R. Lichtler A C. Woody C O. Rowe D W. Kream B E. Transgenic expression of COLL1A1-chloramphenicol acetyltransferase fusion genes in bone: differential utilization of promoter elements in vivo and in cultured cells. [Journal Article] Molecular & Cellular Biology. 13(9):5168-74, 1993.
Kuchroo, V. K., A. C. Anderson, H. Waldner, M. Munder, E. Bettelli, L. B. Nicholson, T cell response in experimental autoimmune encephalomyelitis (EAE): role of self and cross-reactive antigens in shaping, tuning and regulating the autopathogenic T cell repertoire, Ann. Rev. Immunol. 20 (2002) 101-123.
Kyrkanides S, Bellohusen R, Subtelny J D (1995) Skeletal asymmetries of the nasomaxillary complex in non-cleft and post-surgical unilateral cleft lip and palate individuals. Cleft Pal Craniofac J 32: 428-32.
Kyrkanides S, Bellohusen R, Subtelny J D (1996) Asymmetries of the upper lip and nose in non-cleft and post-surgical unilateral cleft lip and palate individuals. Cleft Palate-Craniofacial Journal, 33:306-310.
Kyrkanides S, Kjaer I, Fisher-Hansen B (1993) The basilar part of the occipital bone in normal and pathological fetuses. J Cranio Genet Develop Biol 13: 184-192.
Kyrkanides S, Klambani M, Subtelny J D (2000). Cranial base and facial skeleton asymmetries in unilateral cleft lip & palate individuals. Cleft Palate-Craniofacial Journal 37: 556-561.
Kyrkanides S, Miller J H, Bowers W J, Federoff H J (2003) Transcriptional and post-translational regulation of Cre recombinase by RU486 as the basis for an enhanced inducible expression system. Mol Therapy 8: 790-795.
Kyrkanides S, Miller J H, Federoff H J (2003) Systemic FIV vector administration: Transduction of CNS immune cells and Purkinje neurons. Molecular Brain Research, In Press.
Kyrkanides S, Miller J H, Federoff H J (2003a) Systemic FIV vector administration: Transduction of CNS immune cells and Purkinje neurons. Mol Brain Res 119: 1-9.
Kyrkanides S, Moore A H, Olschowka J A, Williams J P, Hansen J T, O'Banion M K (2002) COX-2 modulates inflammation related genes in CNS radiation injury. Mol Brain Res 104: 159-169.
Kyrkanides S, Moore A H, Olschowka J A, Williams J P, Hansen J T, O'Banion M K (2002a) COX-2 modulates inflammation related genes in CNS radiation injury. Mol Brain Res 104: 159-169.
Kyrkanides S, Olschowka J A, Whitley P, O'Banion M K (2001) Enhanced glial activation and expression of specific CNS inflammation-related molecules in aged versus young rats following cortical stab injury. Journal of Neuroimmunology 119: 269-277.
Kyrkanides S, Olschowka J A, Williams J P, Hansen J T, O'Banion M K (1999). TNFα & IL-1β mediate ICAM-1 induction via microglia-astrocyte interaction in CNS radiation injury. J Neuroimmunol 95: 95-106
Kyrkanides S, Tallents R H, Macher D J, Olschowka J A, Stevens S A (2002b) Temporomandibular Joint nociception: effects of capsaicin on substance P-like immunoreactivity in the rabbit brain stem. J Orofac Pain 16:229-235.
Kyrkanides, S., A. H. Moore, J. A. Olschowka, J. P. Williams, J. T. Hansen, M. K. O'Banion, COX-2 modulates inflammation related genes in CNS radiation injury, Mol. Brain. Res. 104 (2002) 159-169.
Lacorazza H D, Flax J D, Snyder Ey, Jendoubi M (1996) Expression of human beta-hexosaminidase alpha-subunit gene (the gene defect of Tay-Sachs disease) in mouse brains upon engraftment of transduced progenitor cells. Nature Med 2: 424-429.
Lipschutz G S, Gruber C A, Cao Y, Hardy J, Contag C H, Gaensler K M L (2001). In utero delivery of adeno-associated viral vectors: Intraperitoneal gene transfer produces long term expression. Mol Ther 3: 284-92.
Liu F, Malaval L, Aubin J E (1997) The mature osteoblasts phenotype is characterized by extensive plasticity. Exp Cell Res 232: 97-105.
Liu Y, Wada R, Kawai H, Sango K, Deng C, Tai T, McDonald M P, Araujo K, Crawley J N, Bierfreund U, Sandhoff K, Suzuki K, Proia R L (1999) A genetic model of substrate deprivation therapy for a glycosphingolipid storage disorder. J Clin Invest 103: 497-505.
Liu H. Kishi T. Roseberry A G. Cai X. Lee C E. Montez J M. Friedman J M. Elmquist J K. Transgenic mice expressing green fluorescent protein under the control of the melanocortin-4 receptor promoter. [Journal Article] Journal of Neuroscience. 23(18): 7143-54, 2003
Lombardi V R, Garcia M, Cacabelos R (1998) Microglial activation induced by factor(s) contained in sera from Alzheimer-related ApoE genotypes. J Neurosci Res 54: 539-53.
Lu et al. Abnormalities in monocyte recruitment and cytokine expression in monocyte chemoattractant protein 1-deficient mice. J Exp Med. 187(4):601-8 (1998)
Ludwig T, Munier-Lehmann H, Bauer U, Hollinshead M, Ovitt C, Lobel P, Hoflack B (1994) Differential sorting of lysosomal enzymes in mannose 6-phosphate receptor-deficient fibroblasts.] EMBO 13: 3430-3437.
Martino, S., C. Emiliani, B. Tancini, G. M. Severini, V. Chigorno, C. Bordignon, S. Sonnino, A. Orlacchio, Absence of metabolic cross-correction in Tay-Sachs cells, J. Biol. Chem. 277 (2002) 20177-20184.
McCormack J E, Edwards W, Sensintaffer J, Lillegren L, Kozloski M, Brumm D, Karavodin L, Jolly D J, Greengard J (2001). Factors affecting long-term expression of a secreted transgene product after intravenous administration of a retroviral vector. Mol Ther 3: 516-525.
Minghetti L, Levi G (1998) Microglia as effector cells in brain damage and repair: focus on prostanoids and nitric oxide. Prog in Neurobiol 54: 99-125.
Miyazawa et al. Mn-SOD and Bel-2 expression after repeated hyperbaric oxygenation. 76:285-90 (2000)
Mullen R J, Buck C R, Smith A M (1992) NeuN, a neuronal specific nuclear protein in vertebrates. Development 116: 201-211.
Munier-Lehmann H, Mauxion F, Bauer U, Lobel P, Hoflack B (1996) Re-expression of the mannose 6-phosphate receptors in-receptor-deficient fibroblasts. Complementary function of the two mannose 6-phosphate receptors in lysosomal enzyme targeting. J Biol Chem 271: 15166-15174.
Munier-Lehmann H, Mauxion F, Hoflack B (1996). Function of the two mannose 6-phosphate receptors in lysosomal enzyme transport. Biochem Soc Trans 24: 33-36.
Myerowitz R, Lawson D, Mizukami H, Mi Y, Tifft C J, Proia R L (2002) Molecular pathophysiology in Tay-Sachs and Sandhoff diseases as revealed by gene expression profiling. Hum Mol Genet 11: 1343-1350.
Nakamura, R., K. Kurita, T. Kawanami, T. Kato, An immunohistochemical study of Purkinje cells in a case of hereditary cerebellar cortical atrophy, Acta Neuropathol. 97 (1999) 196-200.
Nolte C. Matyash M. Pivneva T. Schipke C G. Ohlemeyer C. Hanisch U K. Kirchhoff F. Kettenmann H. GFAP promoter-controlled EGFP-expressing transgenic mice: a tool to visualize astrocytes and astrogliosis in living brain tissue. GLIA. 33(1):72-86, 2001
Norflus et al. Promoters for the human beta-hexosaminidase genes, HEXA an HEXB. DNA Cell Biol. 15(2):89-97 (1996)
Norflus F, Tiffts C J, McDonald M P, Goldstein G, Crawley J N, Hoffmann A, Sandhoff K, Suzuki K, Proia R L (1998) Bone marrow transplantation prolongs life span and ameliorates neurologic manifestations in Sandhoff disease mice. J Clin Invest 101:1881-1888.
O'Banion, M. K., Cyclooxygenase-2: molecular biology, pharmacology, and neurobiology, Crit. Rev. Neurobiol. 13 (1999) 45-82.
Ohmi K, Greenberg D S, Rajavel K S, Li H H, Neufeld E F (2003) Activated microglia in cortex of mouse models of mucopolysaccharidoses I and IIIB. Proc Natl Acad Sci USA 18: 1902-1907.
Ohmi K, Greenberg D S, Rajavel K S, Li H H, Neufeld E F (2003) Activated microglia in cortex of mouse models of mucopolysaccharidoses I and IIIB. Proc Natl Acad Sci USA 18: 1902-1907.
Olschowka et al. Helper-free HSV-1 amplicons elicit a markedly less robust innat immune response in the CNS. 7(2):218-27 (2003
Oya Y, Proia R L, Norflus F, Tifft C J, Langaman C, Suzuki K (2000) Distribution of enzyme-bearing cells in GM2 gangliosidosis mice: regionally specific pattern of cellular infiltration following bone marrow transplantation. Acta Neuropathol 99: 161-168.
Peel A L. Zolotukhin S. Schrimsher G W. Muzyczka N. Reier P J. Efficient transduction of green fluorescent protein in spinal cord neurons using adeno-associated virus vectors containing cell type-specific promoters. Gene Therapy. 4(1):16-24, 1997
Phaneuf D, Wakamatsu N, Huang J-Q, Borowski A, Peterson A C, Fortunato S R, Ritter G, Igdoura S A, Morales C R, Benoit G, Akerman B R, Leclerc D, Hanai N, Marth J D, Trasler J M, Gravel R A (1996) Dramatically different phenotypes in mouse models of human Tay-Sachs and Sandhoff diseases. Hum Mol Genet 5: 1-14.
Poeschla E M, Wong-Stall F, Looney D L (1998) Efficient transduction of nondividing human cells by feline immunodeficiency virus lentiviral vectors. Nature Medicine 4: 354-357.
Popovic N, Schubart A, Goetz B D, Zhang S C, Linington C, Duncan I D (2002) Inhibition of autoimmune encephalomyelitis by a tetracycline. Ann Neurol 51: 215-23.
Porada, C. D., N. Tran, M. Eglitis, R. C. Moen, L. Troutman, A. W. Flake, Y. Zhao, W. F. Anderson, E. D. Zanjani, In utero gene therapy: transfer and long term expression of the bacterial neo(r) gene in sheep after direct injection of retroviral vectors into preimmune fetuses, Hum. Gene Ther. 9 (1998) 1571-1585.
Priller J, Fluegel A, Wehner T, Boentert M, Haas C A, Prinz M, Femandez-Klett F, Prass K, Bechmann I, de Boer B A, Frotscher M, Kreutzberg G W, Persons D A, Dimagl U (2001a) Targeting gene-modified hematopoietic cells to the central nervous system: Use of green fluorescent protein uncovers microglial engraftment. Nat. Med. 7: 1356-1361.
Priller J, Persons D A, Klett F F, Kempermann G, Kreutzberg G W, Dimagl U (2001b) Neogenesis of cerebellar Purkinje neurons from gene-marked bone marrow cells in vivo. J Cell Biol 155: 733-738.

Priller, J., Grenzganger: adult bone marrow cells populate the brain. Histochem Cell Biol. 120(2):85-91 (2003)

Proia R L, d'Azzo A, Neufeld E F (1984) Association of a- and b-subunits during the biosynthesis of b-hexosaminidase in cultured human fibroblasts. J Biol Chem 259: 3350-3354.
Purpura D P, Suzuki K., Distortion of neuronal geometry and formation of aberrant synapses in neuronal storage disease, Brain Res. 1976 Oct. 29; 116(1):1-21.
Ransohoff R M, Hamilton T A, Tani M, Stoler M H, Shick H E, Major J A, Estes M L, Thomas D M, Tuohy V K (1993) Astrocyte expression of mRNA encoding cytokines IP-10 and JE/MCP-1 in experimental autoimmune encephalomyelitis. FASEB 7: 592-600.
Risau W, Hallmann R, Albrecht U (1986) Differentiatiom-dependent expression of proteins in brain endothelium during development of the blood-brain barrier. Devel Biol 117: 537-45.
Risau W, Wolburg H (1990) Development of blood-brain barrier. TINS 13:174-178.
Rosenberg A (1999) Bones, joints and soft tissue tumors. In Robbins Pathologic Basis of Disease, 6^{th} edition; Cotran, Kumar & Collins (Ed), W.B. Saunders Co, New York.
Sakurada H, Itoh K, Kuroki Y, Kase R, Shimmoto M, Utsumi K, Ozawa H, Tai T, Hara A, Uyama E (1997) Immunocytochemical detection of accumulated substrates in cultured fibroblasts from patients with the infantile and adult forms of Sandhoff disease. Clin Chim Acta 265: 263-266.
Sandell L J and Boyd C D (1990) In Extracellular matrix genes. pp 1-56, Academic Press New York.
Sands, M. S., C. Vogler, J. W. Kyle, J. H. Grubb, B. Levy, N. Galvin, W. S. Sly, E. H. Birkenmeier, Enzyme replacement therapy for murine polysaccharidosis type VII, J. Clin. Invest. 93 (1994) 2324-2331.
Sango K, McDonald M P, Crawley J N, Mack M L, Tifft C J, Skop E, Starr C M, Hoffmann A, Sandhoff K, Suzuki K, Proia R L (1996) Mice lacking both subunits of lysosomal β-hexosaminidase display gangliosidosis and mucopolysaccharidosis. Nature Genet 14: 348-352.
Sango K, Yamanaka S, Hoffman A, Okuda Y, Grinberg A, Westphal H, McDonald M P, Crawley J N, Sandhoff K, Suzuki K, Proia R L (1995) Mouse models of Tay-Sachs and Sandhoff diseases differ in neurologic phenotype and ganglioside metabolism. Nature Genet 11: 170-176.
Schmud L C, Albertson C, Slikker W Jr (1997) Fluoro-Jade: a novel fluorochrome for the sensitive and reliable histochemical localization of neuronal degeneration. Brain Res 751: 37-46.
Schroeder W H, Mortari F, Shiokawa S, Kirkham P M, Elgavish R A, Bertrand F E III (1995) Developmental regulation of the human antibody repertoire. Ann NY Acad Scie 764: 242-260.
Scott-Burden T, Bosley J P, Rosenstrauch D, Henderson K D, Clubb F J, Eichstaedt H C, Eya K, Gregoric I, Myers T J, Radovancevic B, Frazier O H (2002) Use of autologous auricular chondrocytes for lining artificial surfaces: A feasibility study. Ann Thorac Surg 73: 1528-33.
Sinsel N K, Opdebeeck H, Guelinckx P J (1998). The effect of unilateral partial facial paralysis and muscle ablation on craniofacial growth and development: An experimental study in the rabbit. Past Reconstr Surg 102: 1894-1912.
Suzuki K, Hoogerbrugge P M, Poorthuis B J H M, Van Bekkum D W, Suzuki K (1988) The twitcher mouse: central nervous system pathology after bone marrow transplantation. Lab Investigator 58: 302-309.
Suzuki K, Sango K, Proia R L, Langman C (1997) Mice deficient in all forms of lysosomal β-hexosaminidase show mucopolysaccharosis-like pathology. J Neuropath Exp Neurol 56: 693-703.
Swanborg, R. H., Experimental autoimmune encephalitis in the rat: lessons in T-cell immunology and immunoreactivity, Immunol. Rev. 184 (2001) 129-135.
Szpak G M, Lechowicz W, Lewandowska E, Bertrand E, Wierzba-Bobrowicz T, Gwiazda E, Schmidt-Sidor B, Dymecki J (2001) Neurones and microglia in central nervous system immune response to degenerative processes. Part 1: Alzheimer's disease and Lewy body variant of Alzheimer's disease. Quantitative study. Folia Neuropathol 39: 181-92.
Tarantal A F, O'Rourke J P, Case S S, Newbound G C, Li J, Lee C I, Baskin C R, Kohn D B, Bunnell B A (2001) Rhesus monkey model for fetal gene transfer: studies with retroviral-based vector systems. Mol Ther 3: 128-138.
Teixeira, C. A., M. Sena-Esteves, L. Lopes, M. C. Sa Miranda, M. G. Ribeiro, Retrovirus-mediated transfer and expression of β-hexosaminidase α-chain cDNA in human fibroblasts from GM₂-gangliosidosis B1 variant, Hum. Gene Ther. 12 (2001) 1771-1783.
Tian, M., C. Jacobson, S. H. Gee, K. P. Campbell, S. Carbonetto, M. Jucker, Dystroglycan in the cerebellum is a laminin alpha 2-chain binding protein at the glial-vascular interface and is expressed in Purkinje cells, Eur. J. Neurosci. 8 (1996) 2739-2747.
Utsumi K, Tsuji A, Kase R, Tanaka A, Tanaka T, Uyama E, Ozawa T, Sakuraba H, Komaba Y, Kawabe M, Eino Y, Katayama Y (2002) Western blotting analysis of the beta-hexosaminidase alpha- and beta-subunits in cultured fibroblasts from cases of various forms of GM2 gangliosidosis. Acta Neurol Scandin 105: 427-30.
Vanden-Berghe, D. A., Comparison of various density-gradient media for the isolation and characterisation of animal viruses. In D. Rickwood (Ed.), Iodinated density gradient media-a practical approach, Oxford University Press, Oxford, UK, 1983, pp. 175-193.
von Specht B U, Geiger B, Amon R, Passwell J, Keren G, Goldman B, Padeh B (1979) Enzyme replacement therapy for Tay-Sachs disease. Neurol 29: 848-854.
Wada R, Tiffi C J, Proia R L (2002) Microglial activation precedes acute neurodegeneration in Sandhoff disease and is suppressed by bone marrow transplantation. Proc Natl Acad Sci USA 97: 10954-10959.
Wakley, S. U., K. Dobrenis, Bone marrow transplantation for lysosomal diseases, Lancet 345 (1995) 1382-1383.
Walkley S U (1998) Cellular pathology of lysosomal storage disorders. Brain Path 8:175-193.
Walkley S U, Baker H J, Rattazzi M C, Haskins M E, Wu J Y (1991) Neuroaxonal dystrophy in neuronal storage disorders: evidence for major GABAergic neuron involvement. J Neurol Sci 104(1):1-8.
Walkley S U., Pathobiology of neuronal storage disease, Int Rev Neurobiol. 1988; 29:191-244.
Walkley, S. U., M. A. Thrall, K. Dobrenis, M. Huang, P. A. March, D. A. Siegel, S. Wurzelmann, Bone marrow transplantation corrects the enzyme defect in neurons of the central nervous system in a lysosomal storage disease, Proc. Natl. Acad. Sci. U.S.A. 91 (1994) 2970-2974.
Weinmann, J. M., C. A. Charlton, T. R. Brazeltin, R. C. Hackman, H. M. Blau, Contribution of transplanted bone marrow cells to Purkinje neurons in human adult brain, Proc. Natl. Acad. Sci. U.S.A. 100 (2003) 2088-2093.
Westerman K A, Leboulch P. Reversible immortalization of mammalian cells mediated by retroviral transfer and site-specific recombination. Proc. Natl. Acad. Sci. USA 93: 8971-8976, 1996.
Wolff J A, Harding C O (2000) Principles of gene therapy for inborn errors of metabolism. In Gene Therapy: therapeutic mechanisms and strategies. NS Templeton and D D Lasic, Editors. Marcel Dekker Inc, New York, pp 507-533.
Yamanaka et al. Neuropathology of mice with targeted disruption of Hexa gene, kmodel of Tay-Sachs disease. Acta Neuropathol (Berl.) 89(4):296-304 (1995)
Yamanaka et al. Targeted disruption of the Hexa gene results in mice with biochemical and pathologic features of Tay-Sachs disease. 91(21):9975-9 (1994)
Yaworsky P J. Gardner D P. Kappen C. Transgenic analyses reveal developmentally regulated neuron- and muscle-specific elements in the murine neurofilament light chain gene promoter. Journal of Biological Chemistry. 272(40):25112-20, 1997
Yrjanheikki J, Keinanen R, Pellikka M, Hokfelt T, Koistinaho J (1998) Tetracyclines inhibit microglial activation and are neuroprotective in global brain ischemia. Proc Natl Acad Sci USA 95: 15769-74.
Yrjanheikki J, Tikka T, Keinanen R, Goldsteins G, Chan P H, Koistinaho J (1999) A tetracycline derivative, minocycline, reduces inflammation and protects against focal cerebral ischemia with a wide therapeutic window. Proc Natl Acad Sci USA 96: 13496-500
Zhang, B., H. Q. Xia, G. Cleghorn, G. Gobe, M. West, M. Q. Wei, A highly efficient and consistent method for harvesting large volumes of high-titre lentiviral vectors, Gene Therapy 8 (2001) 1745-1751.
Zhou, L., B. L. Miller, C. H. McDaniel, L. Kelly, O. J. Kim, C. A. Miller, Frontotemporal dementia: neuropil spheroids and presynaptic terminal degeneration, Ann. Neurol. 44 (1998) 99-109.
Zhou L., Zhang C. L., Messing A., Chiu S. Y. (1998) Temperature-sensitive neuromuscular transmission in Kv1.1 null mice: Role of potassium channels under the myelin sheath in young nerves. J. Neurosci 18: 7200-15)

1. A method of delivering a nucleic acid to a brain central nervous system cell comprising systemically administering a vector to a blood cell, wherein the vector transduces the blood cell, and wherein the blood cell fuses with the brain central nervous system cell.

2. The method of claim 1, wherein the blood cell comprises a blood progenitor cell, an endothelial cell, a microglia cell, a monocyte cell, a macrophage, a lymphocyte cell, or a combination thereof.

3-19. (canceled)

20. The method of claim 1, wherein the vector is administered to the blood cell ex vivo producing a transduced blood cell, and wherein the transduced blood cell is administered to the subject.

21. The method of claim 20, wherein the blood cell comprises a blood cell obtained from the subject or is derived from a blood cell obtained from the subject.

22. The method of claim 20, wherein the blood cell comprises a progenitor cell.

23. The method of claim 20, wherein the blood cell comprises a marker for a blood progenitor cell.

24. The method of claim 1, wherein the vector comprises a sequence encoding a HEX-α and a sequence encoding a HEX-β, wherein the nucleic acid comprises an integrated ribosomal entry site (IRES), and wherein the nucleic acid comprises a cell specific promoter

25. The composition of claim 24, wherein the cell specific promoter comprises the Nuclear enolase specific (NSE) promoter.

26. The composition of claim 24, wherein the cell specific promoter comprises the sequence set forth in SEQ ID NO:69.

27. The composition of claim 24, wherein the cell specific promoter comprises the COLL1A1 promoter.

28. The composition of claim 24, wherein the cell specific promoter comprises the sequence set forth in SEQ ID NO:70 or SEQ ID NO:71.

29. The method of claim 1, wherein the subject is a perinatal or neonatal subject.

30. (canceled)

31. The method of claim 1, wherein the brain cell is a Purkinje cell, brain cortex cell, a brain basal ganglia cell, a brain thalamus cell, a brain cerebellum cell, or a brain stem cell.

32-33. (canceled)

34. The method of claim 1, wherein the administration of the vector comprises less than or equal to 10⁷ infectious particles.

35. The method of claim 1, wherein the administration of the vector comprises greater than or equal to 10³ infectious particles.

36-37. (canceled)

38. The method of claim 1, wherein the administration of the vector comprises a m.o.i of about 2.

39. The method of claim 1, wherein the vector reduces the inflammation of the brain.

40. The method of claim 1, wherein the vector reduces the deterioration of motor function due to a lysomal storage disease.

41. The method of claim 40, wherein the lysomal storage disease involves GM2 gangliosides.

42. The method of claim 41, wherein the disease is Tay-Sachs disease or Sandoff's disease.

43. (canceled)


SEQUENCE LISTING (73 sequences)

SEQ ID NO: 1 - protein, 409 aa, Artificial Sequence
  Description of Artificial Sequence/Note = Synthetic Construct
MMTSVYSSLRLSGELSEVWRLLASLFGNLLRAQFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 2 - DNA, 2256 bp, Artificial Sequence
  Description of Artificial Sequence/Note = Synthetic Construct
cctccgagaggggagaccagcgggccatgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacagttctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacatagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgaattgctgaggcgaggtgtccaggcccaacccctcaatgtaggcttctgtgagcaggagtttgaacagacctgagccccaggcaccgaggagggtgctggctgtaggtgaatggtagtggagccaggcttccactgcatcctggccaggggacggagccccttgccttcgtgccccttgcctgcgtgcccctgtgcttggagagaaaggggccggtgctggcgctcgcattcaataaagagtaatgtggcatttttctataataaacatggattacctgtgtttaaaaaaaaaagtgtgaatggcgttagggtaagggcacagccaggctggagtcagtgtctgcccctgaggtcttttaagttgagggctgggaatgaaacctatagcctttgtgctgttctgccttgcctgtgagctatgtcactcccctcccactcctgaccatattccagacacctgccctaatcctcagcctgctcacttcacttctgcattatatctccaaggcgttggtatatggaaaaagatgtaggggcttggaggtgttctggacagtggggagggctccagacccaacctggtcacagaagagcctctcccccatgcatactcatccacctccctcccctagagctattctcctttgggtttcttgctgcttcaattttatacaaccattatttaaatattattaaacacatattgttctct

SEQ ID NO: 3 - protein, 544 aa, Artificial Sequence
  Description of Artificial Sequence/Note = Synthetic Construct
MLLALLLATLLAAMLALLTQVALVVQVAEAARAPSVSAKPGPALWPLPLLVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 4 - DNA, 1635 bp, Artificial Sequence
  Description of Artificial Sequence/Note = Synthetic Construct
atgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctcttggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaa

SEQ ID NO: 5 - DNA, 581 bp, Artificial Sequence
  Description of Artificial Sequence/Note = Synthetic Construct
aattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 6 - protein, 528 aa, Artificial Sequence
  Description of Artificial Sequence/Note = Synthetic Construct
MAGCRLWVSLLLAAALACLATALWPWPQYIQTYHRRYTLYPNNFQFRYHVSSAAQGGCVVLDEAFRRYRNLLFGSGSWPRPSFSNKQQTLGKNILVVSVVTAECNEFPNLESVENYTLTINDDQCLLASETVWGALRGLETFSQLVWKSAEGTFFINKTKIKDFPRFPHRGVLLDTSRHYLPLSSILDTLDVMAYNKFNVFHWHLVDDSSFPYESFTFPELTRKGSFNPVTHIYTAQDVKEVIEYARLRGIRVLAEFDTPGHTLSWGPGAPGLLTPCYSGSHLSGTFGPVNPSLNSTYDFMSTLFLEISSVFPDFYLHLGGDEVDFTCWKSNPNIQAFMKKKGFTDFKQLESFYIQTLLDIVSDYDKGYVVWQEVFDNKVKVRPDTIIQVWREEMPVEYMLEMQDITRAGFRALLSAPWYLNRVKYGPDWKDMYKVEPLAFHGTPEQKALVIGGEACMWGEYVDSTNLVPRLWPRAGAVAERLWSSNLTTNIDFAFKRLSHFRCELVRRGIQAQPISVGYCEQEFEQT

SEQ ID NO: 7 - DNA, 1960 bp, Artificial Sequence
  Description of Artificial Sequence/Note = Synthetic Construct
ctgcagaatcctttgcttacggatctctgagatcgagccgccttgcttccctcccgttcacgtgaccctccgattgtcacgcgggcgtccgctcagctgaccggggctcacgtgggctcagcctgctggccggggagctggccggtgggcatggccggctgcaggctctgggtttcgctgctgctggcggcggcgttggcttgcttggccacggcactgtggccgtggccccagtacatccaaacctaccaccggcgctacaccctgtaccccaacaacttccagttccggtaccatgtcagttcggccgcgcagggcggctgcgtcgtcctcgacgaggcctttcgacgctaccgtaacctgctcttcggttccggctcttggccccgacccagcttctcaaataaacagcaaacgttggggaagaacattctggtggtctccgtcgtcacagctgaatgtaatgaatttcctaatttggagtcggtagaaaattacaccctaaccattaatgatgaccagtgtttactcgcctctgagactgtctggggcgctctccgaggtctggagactttcagtcagcttgtttggaaatcagctgagggcacgttctttatcaacaagacaaagattaaagactttcctcgattccctcaccggggcgtactgctggatacatctcgccattacctgccattgtctagcatcctggatacactggatgtcatggcatacaataaattcaacgtgttccactggcacttggtggacgactcttccttcccatatgagagcttcactttcccagagctcaccagaaaggggtccttcaaccctgtcactcacatctacacagcacaggatgtgaaggaggtcattgaatacgcaaggcttcggggtatccgtgtgctggcagaatttgacactcctggccacactttgtcctgggggccaggtgcccctgggttattaacaccttgctactctgggtctcatctctctggcacatttggaccggtgaaccccagtctcaacagcacctatgacttcatgagcacactcttcctggagatcagctcagtcttcccggacttttatctccacctgggaggggatgaagtcgacttcacctgctggaagtccaaccccaacatccaggccttcatgaagaaaaagggctttactgacttcaagcagctggagtccttctacatccagacgctgctggacatcgtctctgattatgacaagggctatgtggtgtggcaggaggtatttgataataaagtgaaggttcggccagatacaatcatacaggtgtggcgggaagaaatgccagtagagtacatgttggagatgcaagatatcaccagggctggcttccgggccctgctgtctgctccctggtacctgaaccgtgtaaagtatggccctgactggaaggacatgtacaaagtggagcccctggcatttcatggtacgcctgaacagaaggctctggtcattggaggggaggcctgtatgtggggagagtatgtggacagcaccaacctggtccccagactctggcccagagcgggtgccgtcgctgagagactgtggagcagtaacctgacaactaatatagactttgcctttaaacgtttgtcgcatttccgttgtgagctggtgaggagaggaatccaggcccagcccatcagtgtaggctactgtgagcaggagtttgagcagacttgagccaccagcgctgaacacccaggaggttgctgtcctttgagtcagctgcgctgagcacccaggagggtgctggccttaagagagcaggtcccggggcagggctaatctttcactgcctcccggccaggggagagcaccccttgcccgtgtgcccctgtgactacagagaaggaggctggtgctggcactggtgttcaataaagatctatgtggcattttctct

SEQ ID NO: 8 - DNA, 12745 bp, Artificial Sequence
  Description of Artificial Sequence/Note = Synthetic Construct
atgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacgggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaatcaaatcaaactaataaagtatgtattgtaaggtaaaaggaaaagacaaagaagaagaagaaagaagaaagccttcagtacatttatattggctcatgtccaatatgaccgccatgttgacattgattattgactagttattaatagtaatcaattacggggtcattagttcatagcccatatatggagttccgcgttacataacttacggtaattggcccgcctgctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccggccccctattgacgtcaatgacggtaaatggcccgcctggcattatgcccagtacatgaccttacgggactttggtacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggttctgttaacggtggagggcagtgtagtctgagcagtactcgttgctgccgcgcgcgccaccagacataatagctgacagactaacagactgttcctttccatgggtcttttctgcagtcaccgtcgtcgaagcttatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataactcgatcgaccagagctgagatcctacaggagtccagggctggagagaaaacctctgaagaggatgatgacagagttagaagatcgcttcaggaagctatttggcacgacttctacaacgggagacagcacagtagattctgaagatgaacctcctaaaaaagaaaaaagggtggactgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattttaacaaaatattaacgtttacaatttaaatatttgcttatacaatcttcctgtttttggggcttttctgattatcaaccggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtg

SEQ ID NO: 9 - protein, 529 aa, Artificial Sequence
  Description of Artificial Sequence/Note = Synthetic Construct
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYVVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 10 - DNA, 2255 bp, Artificial Sequence
  Description of Artificial Sequence/Note = Synthetic Construct
cctccgagaggggagaccagcgggccatgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacgtagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgagttgctgaggcgaggtgtccaggcccaacccctcaatgtaggcttctgtgagcaggagtttgaacagacctgagccccaggcaccgaggagggtgctggctgtaggtgaatggtagtggagccaggcttccactgcatcctggccaggggacggagccccttgccttcgtgccccttgcctgcgtgcccctgtgcttggagagaaaggggccggtgctggcgctcgcattcaataaagagtaatgtggcatttttctataataaacatggattacctgtgtttaaaaaaaaaagtgtgaatggcgttagggtaagggcacagccaggctggagtcagtgtctgcccctgaggtcttttaagttgagggctgggaatgaaacctatagcctttgtgctgttctgccttgcctgtgagctatgtcactcccctcccactcctgaccatattccagacacctgccctaatcctcagcctgctcacttcacttctgcattatatctccaaggcgttggtatatggaaaaagatgtaggggcttggaggtgttctggacagtggggagggctccagacccaacctggtcacaaaagagcctctcccccatgcatactcatccacctccctcccctagagctattctcctttgggtttcttgctgctgcaattttatacaaccattatttaaatattattaaacacatattgttctct

SEQ ID NO: 11 - DNA, 1635 bp, Artificial Sequence
  Description of Artificial Sequence/Note = Synthetic Construct
atgctactggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctcttggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaa

SEQ ID NO: 12 - protein, 544 aa, Artificial Sequence
  Description of Artificial Sequence/Note = Synthetic Construct
MLLALLLATLLAAMLALLTQIALVVQVAEAARAPSVSAKPGPALWPLPLLVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 13 - protein, 529 aa, Artificial Sequence; identical to SEQ ID NO: 9
  Description of Artificial Sequence/Note = Synthetic Construct

SEQ ID NO: 14 - DNA, 739 bp, Artificial Sequence
  Description of Artificial Sequence/Note = Synthetic Construct
ttttaatcctccgtttttctgcttctgaagttacttcagcctggcaagtcctttacctccccgtaggcctggcgagctgcatcacaacattcaagattcaccctagagccatctgggaaactttcttctccaggtcgccctgcgtcctcgcctccccaccccgttcttctcgagtcgggtgagctgtctagttccatcacggccggcacggccgcaggggtggccggttatttactgctctactgggcccgtgagcagtctggcgagccgagcagttgccgacgcccggcacaatccgctgcacgtagcaggagcctcaggtccaggccggaagtgaaagggcagggtgtgggtcctcctggggtcgcaggcgcagagccgcctctggtcacgtgattcgccgataagtcacgggggcgccgctcacctgaccagggtctcacgtggccagccccctccgagaggggagaccagcgggccatgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacaggtgagt

SEQ ID NO: 15 - protein, 556 aa, Artificial Sequence
  Description of Artificial Sequence/Note = Synthetic Construct
MELCGLGLPRPPMLLALLLATLLAAMLALLTQVALVVQVAEAARAPSVSAKPGPALWPLPLSVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 16 - DNA, 1857 bp, Artificial Sequence
  Description of Artificial Sequence/Note = Synthetic Construct
ctgatccgggccgggcgggaagtcgggtcccgaggctccggctcggcagaccgggcggaaagcagccgagcggccatggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctctcggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaaaaaatggaggggaaaaaggccacagcaatctgtactacaatcaactttattttgaaatcatgtaaaataagatattagacttttttgaataaaatatttttattgattgaa

SEQ ID NO: 17 - protein, 536 aa, Artificial Sequence
  Description of Artificial Sequence/Note = Synthetic Construct
MPQSPRSAPGLLLLQALVSLVSLALVAPARLQPALWPFPRSVQMFPRLLYISAEDFSIDHSPNSTAGPSCSLLQEAFRRYYNYVFGFYKRHHGPARFRAEPQLQKLLVSITLESECESFPSLSSDETYSLLVQEPVAVLKANSVWGALRGLETFSQLVYQDSFGTFTINESSIADSPRFPHRGILIDTSRHFLPVKTILKTLDAMAFNKFNVLHWHIVDDQSFPYQSTTFPELSNKGSYSLSHVYTPNDVRMVLEYARLRGIRVIPEFDTPGHTQSWGKGQKNLLTPCYNQKTKTQVFGPVDPTVNTTYAFFNTFFKEISSVFPDQFIHLGGDEVEFQCWASNPNIQGFMKRKGFGSDFRRLESFYIKKILEIISSLKKNSIVWQEVFDDKVELQPGTVVEVWKSEHYSYELKQVTGSGFPAILSAPWYLDLISYGQDWKNYYKVEPLNFEGSEKQKQLVIGGEACLWGEFVDATNLTPRLWPRASAVGERLWSPKTVTDLENAYKRLAVHRCRMVSRGIAAQPLYTGYCNYENKI

SEQ ID NO: 18 - DNA, 1750 bp, Artificial Sequence
  Description of Artificial Sequence/Note = Synthetic Construct
ggagcagtcatgccgcagtccccgcgtagcgcccccgggctgctgctgctgcaggcgctggtgtcgctagtgtcgctggccctagtggccccggcccgactgcaacctgcgctatggcccttcccgcgctcggtgcagatgttcccgcggctgttgtacatctccgcggaggacttcagcatcgaccacagtcccaattccacagcgggcccttcctgctcgctgctacaggaggcgtttcggcgatattacaactatgtttttggtttctacaagagacatcatggccctgctagatttcgagctgagccacagttgcagaagctcctggtctccattaccctcgagtcagagtgcgagtccttccctagtctgtcttcagatgaaacctattctctgcttgtacaagaaccagtagccgtcctcaaggccaacagcgtttggggagcgttacgaggtttagagacgtttagccagttagtttaccaagactctttcgggactttcaccatcaatgaatccagtatagctgattctccaagattccctcatagaggaattttaattgatacatctagacacttcctgcctgtgaagacaattttaaaaactctggatgccatggcttttaataagtttaatgttcttcactggcacatagtggacgaccagtctttcccttatcagagtaccacttttcctgagctaagcaataagggaagctactctttgtctcatgtctatacaccaaacgatgtccggatggtgctggagtacgcccggctccgagggattcgagtcataccagaatttgatacccctggccatacacagtcttggggcaaaggacagaaaaaccttctaactccatgttacaatcaaaaaactaaaactcaagtgtttgggcctgtagacccaactgtaaacacaacgtatgcattctttaacacatttttcaaagaaatcagcagtgtgtttccagatcagttcatccacttgggaggagatgaagtagaatttcaatgttgggcatcaaatccaaacatccaaggtttcatgaagagaaagggctttggcagcgattttagaagactagaatccttttatattaaaaagattttggaaattatttcatccttaaagaagaactccattgtttggcaagaagtttttgatgataaggtggagcttcagccgggcacagtagtcgaagtgtggaagagtgagcattattcatatgagctaaagcaagtcacaggctctggcttccctgccatcctttctgctccttggtacttagacctgatcagctatgggcaagactggaaaaactactacaaagttgagccccttaattttgaaggctctgagaagcagaaacaacttgttattggtggagaagcttgcctgtggggagaatttgtggatgcaactaaccttactccaagattatggcctcgagcaagcgctgttggtgagagactctggagccctaaaactgtcactgacctagaaaatgcctacaaacgactggccgtgcaccgctgcagaatggtcagccgtggaatagctgcacaacctctctatactggatactgtaactatgagaataaaatatagaagtgacagacgtctacagcattccagctatgatcatgttgattctgaaatcatgtaaattaagatttgttaggctgttttttttttaaataaaccatctttttattgattgaatctttctaaaaaaaaa

SEQ ID NO: 19 - DNA, 12263 bp, Artificial Sequence
  Description of Artificial Sequence/Note = Synthetic Construct
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgatatcgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctgcagcccgggggatccactagtccagtgtggtggaattgatcccttcacctaatacgactcactataggctagcctcgagctgatccgggccgggcgggaagtcgggtcccgaggctccggctcggcagaccgggcggaaagcagccgagcggccatggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctctcggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaaaaaatggaggggaaaaaggccacagcaatctgtactacaatcaactttattttgaaatcatgtaaaataagatattagacttttttgaataaactcgagaattcacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctatgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacgtagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgagttgctgaggcgaggtgtccaggcccaacccctcaatgtaggcttctgtgagcaggagtttgaacagacctgaagagtcgacccgggcggccgcttccctttagtgagggttaatgaagggctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 20 - DNA, 11110 bp, Artificial Sequence
  Description of Artificial Sequence/Note = Synthetic Construct
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattgatcccttcacctaatacgactcactataggctagcctcgagctgatccgggccgggcgggaagtcgggtcccgaggctccggctcggcagaccgggcggaaagcagccgagcggccatggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctctcggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaaaaaatggaggggaaaaaggccacagcaatctgtactacaatcaactttattttgaaatcatgtaaaataagatattagacttttttgaataaactcgagaattcacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctatgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacgtagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgagttgctgaggcgaggtgtccaggcccaacccctcaatgtaggcttctgtgagcaggagtttgaacagacctgaagagtcgacccgggcggccgcttccctttagtgagggttaatgaagggctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 21 - DNA, 1278 bp, Artificial Sequence
  Description of Artificial Sequence/Note = Synthetic Construct
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagccatg

SEQ ID NO: 22 - DNA, 1278 bp, Artificial Sequence
  Description of Artificial Sequence/Note = Synthetic Construct
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgcgcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgttgtaccggcggggtttatatcttcccttctctgttcctccgcagccagccatg

SEQ ID NO: 23 - DNA, 1729 bp, Artificial Sequence
  Description of Artificial Sequence/Note = Synthetic Construct
gaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 24 - DNA, 366 bp, Artificial Sequence
  Description of Artificial Sequence/Note = Synthetic Construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggt

SEQ ID NO: 25 - DNA, 1295 bp, Artificial Sequence
  Description of Artificial Sequence/Note = Synthetic Construct
ccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 26 - DNA, 1278 bp, Artificial Sequence; identical to SEQ ID NO: 22
  Description of Artificial Sequence/Note = Synthetic Construct

SEQ ID NO: 27 - DNA, 229 bp, Artificial Sequence
  Description of Artificial Sequence/Note = Synthetic Construct
gtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 28 - DNA, 281 bp, Artificial Sequence
  Description of Artificial Sequence/Note = Synthetic Construct
tggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcag

SEQ ID NO: 29 - DNA, 282 bp, Artificial Sequence
  Description of Artificial Sequence/Note = Synthetic Construct
attatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 30 - DNA, 512 bp, Artificial Sequence
  Description of Artificial Sequence/Note = Synthetic Construct
ttgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 31 - DNA, 308 bp, Artificial Sequence
  Description of Artificial Sequence/Note = Synthetic Construct
tcggcgaagcctcgcgcggccggccaggacgaggagcgccactaggttgaacatccgcacgagccgccgggccaggtctcggacgggctctcgagactcgatctcgtgcatgtcggcggtccgcggtgaggttatagaccatctgctaggcgggtccggggagacaggcacattactggcctcggcgcccagcctaggcgtgtctagagctcgaccgcgcgtccggagcgccattcgaccggcgggtagcgagaagaacgccggagaccgcaggttataacaacgtcatgcataaattaagaatgggc

SEQ ID NO: 32 - DNA, 1848 bp, Artificial Sequence
  Description of Artificial Sequence/Note = Synthetic Construct
ctgcagtgaataataaaatgtgtgtttgtccgaaatacgcgtttgagatttctgtcccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagttgatttttgggcatacgcgatatctggcgatacgcttatatcgtttacgggggatggcgatagacgcctttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgcacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccagcgaatctcgggtacgtgttccggacatggggctcttctccggtagcggcggagcttctacatccagccctgctcccatcctcccactcatggtcctcggcagctccttgctcctaacagtggaggccagacttaggcacagcacgatgcccaccaccaccagtgtgcccacaaggccgtggcggtagggtatgtgtctgaaaatgagctc

SEQ ID NO: 33 - DNA, 1176 bp, Artificial Sequence
  Description of Artificial Sequence/Note = Synthetic Construct
cccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcatgcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcgtagcccccgccaggcccggtgggggctggggcgccattgccggtgcgcgctggtcctttgggcgctaactgcgtgcgcgctgggaattggcgctaattgcgcgtgcgcgctgggactcaaggcgctaattgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacttcggctcacagcgcgcccggctattctcgcagctcaccatggatg

SEQ ID NO: 34 - DNA, 49 bp, Artificial Sequence
  Description of Artificial Sequence/Note = Synthetic Construct
cttctggcgtgtgaccggcggggtttatatcttcccttcccaagcttgg

SEQ ID NO: 35 - DNA, 66 bp, Artificial Sequence
  Description of Artificial Sequence/Note = Synthetic Construct
cttctggcgtgtgaccggcggggtttatatcttcccttctctgttcctccgcagccccaagcttgg

SEQ ID NO: 36 - DNA, 68 bp, Artificial Sequence
  Description of Artificial Sequence/Note = Synthetic Construct
cttctggcgtgtgaccggcggggtttatatcttcccttctctgttcctccgcagccagccaagcttgg

SEQ ID NO: 37 - DNA, 69 bp, Artificial Sequence
  Description of Artificial Sequence/Note = Synthetic Construct
cttctggcgtgtgaccggcggggtttatatcttcccttctctgttcctccgcagccagccatggatgat

SEQ ID NO: 38 - DNA, 1278 bp, Artificial Sequence; identical to SEQ ID NO: 22
  Description of Artificial Sequence/Note = Synthetic Construct

SEQ ID NO: 39 - DNA, 1176 bp, Artificial Sequence; identical to SEQ ID NO: 33
  Description of Artificial Sequence/Note = Synthetic Construct

SEQ ID NO: 40 - DNA, 1345 bp, Artificial Sequence
  Description of Artificial Sequence/Note = Synthetic Construct
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcaagctt

SEQ ID NO: 41 - DNA, 684 bp, Artificial Sequence
  Description of Artificial Sequence/Note = Synthetic Construct
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcgg

SEQ ID NO: 42 - DNA, 21 bp, Artificial Sequence
  Description of Artificial Sequence; note = synthetic construct
attttaaaattcaggcctcga

SEQ ID NO: 43 - DNA, 21 bp, Artificial Sequence
  Description of Artificial Sequence; note = synthetic construct
catagcgttggctacccgtga

SEQ ID NO: 44 - DNA, 21 bp, Artificial Sequence
  Description of Artificial Sequence; note = synthetic construct
cattctgcagcggtgcacggc

SEQ ID NO: 45 - DNA, 25 bp, Artificial Sequence
  Description of Artificial Sequence; note = synthetic construct
gagaaccaagcaacgacaaaatacc

SEQ ID NO: 46 - DNA, 25 bp, Artificial Sequence
  Description of Artificial Sequence; note = synthetic construct
gcattagaaacagtccagcccatac

SEQ ID NO: 47 - DNA, 20 bp, Artificial Sequence
  Description of Artificial Sequence; note = synthetic construct
cgagtgacaagcctgtagcc

SEQ ID NO: 48 - DNA, 23 bp, Artificial Sequence
  Description of Artificial Sequence; note = synthetic construct
ggttgactttctcctggtatgag

SEQ ID NO: 49 - DNA, 20 bp, Artificial Sequence
  Description of Artificial Sequence; note = synthetic construct
atgttctctgggaaatcgtg

SEQ ID NO: 50 - DNA, 21 bp, Artificial Sequence
  Description of Artificial Sequence; note = synthetic construct
gaaggactctggctttgtctt

SEQ ID NO: 51 - DNA, 20 bp, Artificial Sequence
  Description of Artificial Sequence; note = synthetic construct
cagtcgtccgcttccgctac

SEQ ID NO: 52 - DNA, 21 bp, Artificial Sequence
  Description of Artificial Sequence; note = synthetic construct
agaaattggctccgtggtccc

SEQ ID NO: 53 - DNA, 22 bp, Artificial Sequence
  Description of Artificial Sequence; note = synthetic construct
agtcctgccagaatttgatacc

SEQ ID NO: 54 - DNA, 19 bp, Artificial Sequence
  Description of Artificial Sequence; note = synthetic construct
attccacgttcgaccatcc

SEQ ID NO: 55 - DNA, 21 bp, Artificial Sequence
  Description of Artificial Sequence; note = synthetic construct
tttttccagttccgtttatcc

SEQ ID NO: 56 - DNA, 20 bp, Artificial Sequence
  Description of Artificial Sequence; note = synthetic construct
tttatcgccaatccacatct

SEQ ID NO: 57 - DNA, 20 bp, Artificial Sequence
  Description of Artificial Sequence; note = synthetic construct
accacagtccatgccatcac

SEQ ID NO: 58 - DNA, 20 bp, Artificial Sequence
  Description of Artificial Sequence; note = synthetic construct
tccaccaccctgttgctgta

SEQ ID NO: 59 - DNA, 22 bp, Artificial Sequence
  Description of Artificial Sequence; note = synthetic construct
tggtggagcgatttgtctggtt

SEQ ID NO: 60 - DNA, 20 bp, Artificial Sequence
  Description of Artificial Sequence; note = synthetic construct
tagtagcgacgggcggtgtg

SEQ ID NO: 61 - DNA, 23 bp, Artificial Sequence
  Description of Artificial Sequence; note = synthetic construct
caccccaaggaccccaaggagat

SEQ ID NO: 62 - DNA, 22 bp, Artificial Sequence
  Description of Artificial Sequence; note = synthetic construct
cgacgccgctcagaagaaccac

SEQ ID NO: 63 - DNA, 19 bp, Artificial Sequence
  Description of Artificial Sequence; note = synthetic construct
cagcaggtgtcccaaagaa

SEQ ID NO: 64 - DNA, 21 bp, Artificial Sequence
  Description of Artificial Sequence; note = synthetic construct
cttgaggtggttgtggaaaag

SEQ ID NO: 65 - DNA, 20 bp, Artificial Sequence
  Description of Artificial Sequence; note = synthetic construct
cccaagtgctgccgtcattt

SEQ ID NO: 66 - DNA, 23 bp, Artificial Sequence
  Description of Artificial Sequence; note = synthetic construct
gataggctcgcagggatgatttc

SEQ ID NO: 67 - DNA, 26 bp, Artificial Sequence
  Description of Artificial Sequence; note = synthetic construct
tctctttctacctcagactctttgaa

SEQ ID NO: 68 - DNA, 20 bp, Artificial Sequence
  Description of Artificial Sequence; note = synthetic construct
gactccttttccgcttcctg

SEQ ID NO: 69 - DNA, 1986 bp, Artificial Sequence
  Description of Artificial Sequence; note = synthetic construct
tcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaggtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagattgaggcagagagactgagaggcctggggatgtgggcattccggtagggcacacagttcacttgtcttctctttttccaggaggccaragatgctgacctcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaagct

SEQ ID NO: 70 - DNA, 3633 bp, Artificial Sequence
  Description of Artificial Sequence; note = synthetic construct
tctagaatatagaagccaaggatttcaagggtttccttttctctcttcttctttttttttctttttctttttcctgagatggagtttccttttgtagccctgactgtcctggaattcactctatagaccaggctagcctcacacttagtgatctgcctgcctctgcctcttgggtgcctcaggattcaaggcatgaaccaccactacccgaccagggatttcttacacacttctgactggactaaccaggaaagcagagagggagacaggaagaaaatgctcagaaggaaggagtaggattggaggtgagctgggggaacccagactgagccgtgcagaagacaaggaagaagaaagccacccacacacctaggatccacccacagattttgctctgggtacccctgtctggagactgtagggctttgtgatggagggtggggtagtcttcatgccccgtgccctttactccagacctaaatgcccacccccacatacagctgctcgctctctctctcccctgcccttctcccaagagaccagttctccatccctggtctgcagccaaggctgggggcagaagaactttctggaggatttgagtgagaaaagcaagagagcctcaagtagggactggaacctctgggaagggagtgcagaggagacccgggtatgtgccctacctggtacatttatacctgggcagcctctgctcctgttccagacttcagagcccagacgggtcctctccctccctcatgaggggaaacatttggggaaatttggagagagacagaactcagagctcagcactttcctctttctgtttttcttcttgaggaattttttcccccaactgctgatgactttaccattcttgggggtgggggggtggagattctggcttttgctccccctacactccaagtgccggacaaagccctacattccacaagaagccagggcttcagagtttcctaaagatgaggtggcgtggcgagtctcctccctctcccagctccaactccccctcccccagtctccagccctagcctggccagggaggccccgccaggctgggaggagaccccaagcacattcttcctctcgctgtcatgctgcagaaattaaagacacatctctgagctgggtacccgccaatcgtttcaagttgagaagtggcagaggaggtcccgagcttcagctcatgccacgtgtaaaggaagcttggaacccactgcccacaactcctggggcaaaaacctggagtcagacatggggtgaaggctgtcacacggcacagacacgtcaagcaccccccccaattctagtagtctcctagcctccaccagaaccccagacccttgatgtggcagtcaccagtccacacctgttaggctcttgtctcttcttccagatgagcctggggggcgtgggggtgctagatcaggagcagggaaaagtagctttggataagtgcttttcccaatacaaaacccaacaaagagtgggcagatcacactgtgtagtgcttcgtggaaccctaccctagacaactgccttgaacacctattccctctgatgtacaccatccccgtccactgttagggagtgggcatcctttggaactgaccactgtggaaggcaggactttactgagttccggaactaccatctcagcttctcagccccagccttaccctacaggcactggcataggcgggggcagatcctgggccacaagtcactgccacatggttgggataattgatgaagtcctgtccttccattgctgtctccagttctgcttctctggaaactctatatttttccctttaattatagcctctgcagtctccctctgccaccccacccgcaccgcttagcctaactgcccacggccagcgacgtggctccctccccttctgctcccttggtcttttttatttttttttctttgccttcgttgcacaaaactagctcagggagggcgtgaagggggggggagcaatggaatcttggatggtttggaggaggcgggactccttgcttccacgtttacagctctgaagacggctgtgggggaagtgatacaggacgtctatgggccctgagaggagacccctatgcttccctgccacccacacagtttaacaaaatgaagttcctaagtagagtgggggtcaggcagagcacctttgcagggttgatgggagcccagggaaagaaaggacactgtcttttagggacacatttaaatataagccacttttcttgggggacgacaaatgaccctttcctgattgcagaggtggggaacaatggctgagattttcagcaaagaagcgaggacatgaggagtagccttcaaataaagtcactcagctaccaaaaacaagtttctgccacacaccgagttacctaggtgtccccagaccagatccaagtacagtaaggaaagcaggttctctacagagagaacacggctctatggccaatgccttctacctgctctttctggattgatactgctacctaagagggcctctaaccaattcctggctgtagccacagctgacacaagacctttttctaagacatccctggtcacaggcctcctgtagcaaattccagccctgggatggaggtggtcaggaaagagtttatacaagaagacccaggccacagctttaaggactcagaaacccccctgcccacacggctgcccatcataacgcagaaggtttcttctggaaggacaaggatgtcaaacttctccccaagcctaatcctcagagatgtctccctctgttacacctggggctggagaaaggtgggtctttcatggagccacattcatggcagaacagatagccaccccactcctttcaaacaaccacatatctgactcttagtatctgtgaagagatgtctaatttgttcccaaatattcctaccctgcatacctgggcccacaccatgaggtattctcctccctctaacagtcacatctgcttagctgcctggttcttcggatttggagagatgcttgcctaacttattcttccttaggtcttcccaaggatgccagaaagactatgagacatggccaagaggaccttttcccaattgtgcctgacactgaaccctttgtaatgttccccaactcagattcccaattctacatccttctgatttgaggtcccagaaggaaagtgcaaggggcatcccctacccacaatcagtatatcgaggcccagccacactcagtgatagcacctctggcccatgtagatctgggggacaagggtggcagaattgcaaaggggggagggggctgggtggactcctttcccttcctttccctcctcccccctcttcgttccaaattgggggccgggccaggcagttctgattggctgggggccgggctgctggctccccctctccaagaggcagggttcctcccagccctcctccatcaggatggtataaaaggggcccaggccagtcgtcggagcagacgggagtttcacctccggacggagcaggaggcacacggagtgaggccacgcatgagccgaagctaaccccccaccccagccgcaaagagtctacatgtctagggtctag

SEQ ID NO: 71 - DNA, 3633 bp, Artificial Sequence; identical to SEQ ID NO: 70
  Description of Artificial Sequence; note = synthetic construct

SEQ ID NO: 72 - DNA, 22 bp, Artificial Sequence
  Description of Artificial Sequence; note = synthetic construct
gaatcccagtctcaataatacc

SEQ ID NO: 73 - DNA, 18 bp, Artificial Sequence
  Description of Artificial Sequence; note = synthetic construct
catacaagcctctccacc